(19) United States
(12) Patent Application Publication
(10) Pub. No.: US 2004/0220177 A1
(43) Pub. Date: Nov. 4, 2004

(54) Compound for the treatment of abnormal cell growth

(75) Inventors: John Charles Kath, Waterford, CT (US); Michael Joseph Luzzio, Groton, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 10/734,039

(22) Filed: Dec. 11, 2003

Related U.S. Application Data
(60) Provisional application No. 60/435,670, filed on Dec. 20, 2002.

(51) Int. Cl. A61K 31/55; A61K 31/513; A61K 31/506; C 07D 4 3/02

(52) U.S. Cl. 514/214.03; 514/275; 540/579; 544/331; 544/317; 514/269

(57) ABSTRACT

The invention relates to compounds of the formula 1

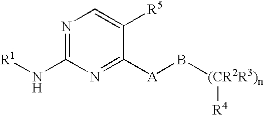

and to pharmaceutically acceptable salts, prodrugs and solvates thereof, wherein R¹, R², R³, R⁴, R⁵, n, A and B are as defined herein. The invention also relates to methods of treating abnormal cell growth in mammals by administering the compounds of formula 1 and to pharmaceutical compositions for treating such disorders, which contain the compounds of formula 1. The invention also relates to methods of preparing the compounds of formula 1.

### BACKGROUND OF THE INVENTION

This invention relates to novel pyrimidine derivatives that are useful in the treatment of abnormal cell growth, such as cancer, in mammals. This invention also relates to a method of using such compounds in the treatment of abnormal cell growth in mammals, especially humans, and to pharmaceutical compositions containing such compounds.

It is known that a cell may become cancerous by virtue of the transformation of a portion of its DNA into an oncogene (i.e., a gene which, on activation, leads to the formation of malignant tumor cells). Many oncogenes encode proteins that are aberrant tyrosine kinases capable of causing cell transformation. Alternatively, the overexpression of a normal proto-oncogenic tyrosine kinase may also result in proliferative disorders, sometimes resulting in a malignant phenotype.

Receptor tyrosine kinases are enzymes which span the cell membrane and possess an extracellular binding domain for growth factors such as epidermal growth factor, a transmembrane domain, and an intracellular portion which functions as a kinase to phosphorylate specific tyrosine residues in proteins and hence to influence cell proliferation. Other receptor tyrosine kinases include c-erbB-2, c-met, tie-2, PDGFr, FGFr, and VEGFR. It is known that such kinases are frequently aberrantly expressed in common human cancers such as breast cancer, gastrointestinal cancer such as colon, rectal or stomach cancer, leukemia, and ovarian, bronchial or pancreatic cancer. It has also been shown that epidermal growth factor receptor (EGFR), which possesses tyrosine kinase activity, is mutated and/or overexpressed in many human cancers such as brain, lung, squamous cell, bladder, gastric, breast, head and neck, oesophageal, gynecological and thyroid tumors.

Accordingly, it has been recognized that inhibitors of receptor tyrosine kinases are useful as selective inhibitors of the growth of mammalian cancer cells. For example, erbstatin, a tyrosine kinase inhibitor, selectively attenuates the growth in athymic nude mice of a transplanted human mammary carcinoma which expresses epidermal growth factor receptor tyrosine kinase (EGFR) but is without effect on the growth of another carcinoma which does not express the EGF receptor. Thus, selective inhibitors of certain receptor tyrosine kinases, are useful in the treatment of abnormal cell growth, in particular cancer, in mammals. In addition to receptor tyrosine kinses, selective inhibitors of certain non-receptor tyrosine kinases, such as FAK (focal adhesion kinase), Ick, src, abl or serine/threonine kinases (e.g.: cyclin dependent kinases, are useful in the treatment of abnormal cell growth, in particular cancer, in mammals. FAK is also known as the Protein-Tyrosine Kinase 2, PTK2.

Convincing evidence suggests that FAK, a cytoplasmic, non-receptor tyrosine kinase, plays an essential role in cell-matrix signal transduction pathways (Clark and Brugge 1995, Science 268: 233-239) and its aberrant activation is associated with an increase in the metastatic potential of tumors (Owens et al. 1995, Cancer Research 55: 2752-2755). FAK was originally identified as a 125 kDa protein highly tyrosine-phosphorylated in cells transformed by v-Src. FAK was subsequently found to be a tyrosine kinase that localizes to focal adhesions, which are contact points between cultured cells and their underlying substratum and sites of intense tyrosine phosphorylation. FAK is phosphorylated and, thus, activated in response to extracellular matrix (ECM)-binding to integrins. Recently, studies have demonstrated that an increase in FAK mRNA levels accompanied invasive transformation of tumors and attenuation of the expression of FAK (through the use of antisense oligonucleotides) induces apoptosis in tumor cells (Xu et al. 1996, Cell Growth and Diff. 7: 413-418). In addition to being expressed in most tissue types, FAK is found at elevated levels in most human cancers, particularly in highly invasive metastases.

Various compounds, such as styrene derivatives, have also been shown to possess tyrosine kinase inhibitory properties. Five European patent publications, namely EP 0 566 226 A1 (published Oct. 20, 1993), EP 0 602 851 A1 (published Jun. 22, 1994), EP 0 635 507 A1 (published Jan. 25, 1995), EP 0 635 498 A1 (published Jan. 25, 1995), and EP 0 520 722 A1 (published Dec. 30, 1992), refer to certain bicyclic derivatives, in particular quinazoline derivatives, as possessing anti-cancer properties that result from their tyrosine kinase inhibitory properties.

Also, World Patent Application WO 92/20642 (published Nov. 26, 1992), refers to certain bis-mono and bicyclic aryl and heteroaryl compounds as tyrosine kinase inhibitors that are useful in inhibiting abnormal cell proliferation. World Patent Applications WO96/16960 (published Jun. 6, 1996), WO 96/09294 (published Mar. 6, 1996), WO 97/30034 (published Aug. 21, 1997), WO 98/02434 (published Jan. 22, 1998), WO 98/02437 (published Jan. 22, 1998), and WO 98/02438 (published Jan. 22, 1998), also refer to substituted bicyclic heteroaromatic derivatives as tyrosine kinase inhibitors that are useful for the same purpose.

Accordingly, a need exists for additional selective inhibitors of certain receptor and non-receptor tyrosine kinases, useful in the treatment of abnormal cell growth, such as cancer, in mammals. The present invention provides for novel pyrimidine derivatives which are selective inhibitors of the non-receptor tyrosine kinase, FAK, and are useful in the treatment of abnormal cell growth.

### SUMMARY OF THE INVENTION

The present invention relates to a compound of the formula 1

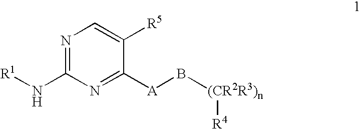

or a pharmaceutically acceptable salt, solvate, hydrate, or prodrug thereof, wherein R¹ has the following formula 2

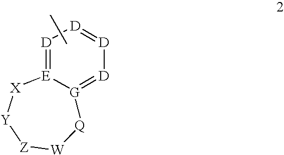

wherein each D is independently selected from the group consisting of CR⁸ and N, with the proviso that R¹ is linked to NH group through a ring carbon atom;

wherein E and G are independently selected from the group consisting of N and C;

wherein X, W and Q are independently selected from the group consisting of N, O, S, SO₂, CO, NR³, CR² and CR²R³;

wherein Y and Z are independently present or absent, if present Y and Z are selected from the group consisting of N, O, S, SO₂, CO, NR³, CR² and CR²R³;

wherein A is present or absent, if present A is selected from the group consisting of O, S and NH and wherein B is present or absent, if present B is selected from the group consisting of CO, SO₂, and NR⁶, with the proviso that when A is O or S that B is absent;

wherein n is an integer from 1 to 3;

wherein each R² is independently selected from the group consisting of H, C₁-C₆ alkyl, C₃-C₇ cycloalkyl, C₄-C₇ heterocycloalkyl, OC₁-C₆ alkyl, OC₃-C₇ cycloalkyl, OC₄-C₇ heterocyloalkyl, NH₂, NHR⁶, NR⁶R⁷, SR⁶, SOR⁶, SO₂R⁶, CO₂R⁶, CONH₂, CONHR⁶, CONR⁶R⁷, SO₂NH₂, SO₂NHR⁶, SO₂NR⁶R⁷, NHCOR⁶, NR⁶CONR⁶, NHCONHR⁶, NR⁶CONHR⁶, NHCONR⁶R⁷, NR⁶CONR⁶R⁷, NHSO₂R⁶, NR⁶SO₂R⁶, with the proviso that O, N or S atom of the foregoing substituents may not be bound to a carbon atom bound to another heteroatom, said alkyl, cycloalkyl, heterocycloalkyl moieties of the foregoing groups are optionally substituted by 1 to 3 substituents independently selected from the group consisting of H, halo, C₁-C₆ alkyl, CN, NH₂, NHR¹⁰, N(R¹⁰)₂, OR¹⁰, C₁-C₆ alkyl, C₃-C₇ cycloalkyl, C₄-C₇ heterocycloalkyl, CO₂R¹¹, CONH₂, CONHR¹¹, and CONR¹¹R¹²;

wherein each R³ is independently selected from the group consisting of H, C₁-C₆ alkyl, C₃-C₇ cycloalkyl, C₄-C₇ heterocycloalkyl, CO₂R⁶, CONH₂, CONHR⁶, CONR⁶R⁷ or R² and R³ taken together with the carbon atom they are linked to can form a 3-7 membered cycloalkyl ring or 4-7 membered heterocycloalkyl ring, wherein each methylene group present in said 3-7 membered cycloalkyl ring and said 4-7 membered heterocycloalkyl ring may be optionally replaced by a C═O group, said alkyl, cycloalkyl, heterocycloalkyl moieties of the foregoing groups are optionally substituted by 1 to 3 substituents independently selected from the group consisting of H, halo, C₁-C₆ alkyl, CN, NH₂, NHR¹⁰, N(R¹⁰)₂, OR¹⁰, C₁-C₆ alkyl, C₃-C₇ cycloalkyl, C₄-C₇ heterocycloalkyl, CO₂R¹¹, CONH₂, CONHR¹¹, and CONR¹¹R¹²;

wherein R⁴ is selected from the group consisting of H, C₁-C₆ alkyl, C₃-C₇ cycloalkyl, C₄-C₇ heterocycloalkyl, C₆-C₁₀ aryl, and 5-10 membered heteroaryl, the alkyl, cycloalkyl, heterocycloalkyl, aryl and heteroaryl moieties of the foregoing groups are optionally substituted by 1 to 3 subsitutents independently selected from the group consisting of H, halo, OH, NO₂, C₁-C₆ alkyl, C(R⁶)═CR⁶R⁷, C≡CR⁶, C₃-C₇ cycloalkyl, C₄-C₇ heterocycloalkyl, OC₁-C₆ alkyl, OC₃-C₇ cycloalkyl, OC₄-C₇ heterocyloalkyl, C═N—OH, C═N—O(C₁-C₆ alkyl), NH₂, NHR⁵, NR⁶R⁶, SR⁶, SOR⁶, SO₂R⁵, CO₂R⁶, CONH₂, CONHR⁶, CONR⁶R⁷, SO₂NH₂, SO₂NHR⁶, SO₂NR⁶R⁷, NHCOR⁶, NR⁶CONR⁶, NHCONHR⁶, NR⁶CONHR⁶, NHCONR⁶R⁷, NR⁶CONR⁶R⁷, NHSO₂R⁶, NR⁶SO₂R⁶, with the proviso that O, N or S atom of the foregoing substituents may not be bound to a carbon atom bound to another heteroatom;

wherein R⁵ is selected from the group consisting of H, Br, Cl, CN, CF₃, CH₂F, CHF₂, SO₂CH₃, CONH₂, cyclopropyl, cyclobutyl, C₆H₅, CONHR⁵, CONR⁶R⁷, CO₂R⁶, C(R⁹)═C(R⁹)₂, and C≡CR⁹;

wherein each R⁶ is independently selected from the group consisting of H, C₁-C₆ alkyl, C₃-C₇ cycloalkyl, C₄-C₇ heterocycloalkyl, C₆-C₁₀ aryl, and 5-10 membered heteroaryl, said alkyl, cycloalkyl, heterocycloalkyl, aryl, and heteroaryl moieties of the foregoing groups are optionally substituted by 1 to 3 substituents independently selected from the group consisting of H, halo, C₁-C₆ alkyl, CN, NH₂, NHR¹⁰, N(R¹⁰)₂, OR¹⁰, C₁-C₆ alkyl, C₃-C₇ cycloalkyl, C₄-C₇ heterocycloalkyl, CO₂R¹¹, CONH₂, CONHR¹¹, and CONR¹¹R¹²;

wherein each R⁷ is independently selected from the group consisting of H, C₁-C₆ alkyl, C₃-C₇ cycloalkyl, C₄-C₇ heterocycloalkyl, C₆-C₁₀ aryl, and 5-10 membered heteroaryl, said alkyl, cycloalkyl, heterocycloalkyl, aryl, and heteroaryl moieties of the foregoing groups are optionally substituted by 1 to 3 substituents independently selected from the group consisting of H, halo, C₁-C₆ alkyl, CN, NH₂, NHR¹⁰, N(R¹⁰)₂, OR¹⁰, C₁-C₆ alkyl, C₃-C₇ cycloalkyl, C₄-C₇ heterocycloalkyl, CO₂R¹¹, CONH₂, CONHR¹¹, and CONR¹¹R¹²;

wherein each R⁸ is independently selected from the group consisting of H, halo, cyano, C₁-C₆ alkyl, C₃-C₇ cycloalkyl, C₄-C₇ heterocycloalkyl, OC₁-C₆ alkyl, OC₃-C₇ cycloalkyl, OC₄-C₇ heterocyloalkyl, NH₂, NHR⁶, NR⁶R⁷, SR⁶, SOR⁶, SO₂R⁶, CO₂R⁶, CONH₂, CONHR⁶, CONR⁶R⁷, SO₂NH₂, SO₂NHR⁶, SO₂NR⁶R⁷, NHCOR⁶, NR⁶CONR⁶, NHCONHR⁶, NR⁶CONHR⁶, NHCONR⁶R⁷, NR⁶CONR⁶R⁷, NHSO₂R⁶, NR⁶SO₂R⁶, said alkyl, cycloalkyl, and heterocycloalkyl moieties of the foregoing groups are optionally substituted by 1 to 3 substituents independently selected from the group consisting of H, halo, C₁-C₆ alkyl, CN, NH₂, NHR³, N(R³)₂, OR³, C¹-C₆ alkyl, C₃-C₇ cycloalkyl, C₄-C₇ heterocycloalkyl, CO₂R⁶, CONH₂, CONHR⁶, and CONR⁶R⁷;

wherein each R⁹ is independently selected from the group consisting of H, CF₃, and C₁-C₆ alkyl, said C₁-C₆ alkyl is optionally substituted by 1 to 6 halo atoms;

wherein each R¹⁰ is independently selected from the group consisting of H, C₁-C₆ alkyl, C₃-C₇ cycloalkyl, C₄-C₇ heterocycloalkyl, CO₂R¹¹, CONH₂, CONHR¹¹, CONR¹¹R¹², SOR¹¹, SO₂R¹¹, SO₂NH₂, SO₂NHR¹¹, SO₂NR¹¹R¹²; said alkyl, cycloalkyl, heterocycloalkyl moieties of the foregoing groups are optionally substituted by 1 to 3 substituents independently selected from the group consisting of H, halo, C₁-C₆ alkyl, CN, NH₂, NHR¹³, N(R¹³)₂, OR¹³, C₁-C₆ alkyl, C₃-C₇ cycloalkyl, C₄-C₇ heterocycloalkyl, CO₂R¹⁴, CONH₂, CONHR¹⁴, and CONR¹⁴R¹⁵

wherein each R¹¹ is independently selected from the group consisting of H, C₁-C₆ alkyl, C₃-C₇ cycloalkyl, C₄-C₇ heterocycloalkyl, C₆-C₁₀ aryl, C₅-C₁₀ membered heteroaryl; said alkyl, cycloalkyl, heterocycloalkyl, aryl, and heteroaryl moieties of the foregoing groups are optionally substituted by 1 to 3 substituents independently selected from the group consisting of H, halo, C₁-C₆ alkyl, CN, NH₂, NHR¹³, N(R¹³)₂, OR¹³, C₁-C₆ alkyl, C₃-C₇ cycloalkyl, C₄-C₇ heterocycloalkyl, CO₂R¹⁴, CONH₂, CONHR¹⁴, and CONR¹⁴R¹⁵;

wherein each R¹² is independently selected from the group consisting of H, C₁-C₆ alkyl, C₃-C₇ cycloalkyl, C₄-C₇ heterocycloalkyl, C₆-C₁₀ aryl, C₅-C₁₀ membered heteroaryl; said alkyl, cycloalkyl, heterocycloalkyl, aryl, and heteroaryl moieties of the foregoing groups are optionally substituted by 1 to 3 substituents independently selected from the group consisting of H, halo, C₁-C₆ alkyl, CN, NH₂, NHR¹³, N(R¹³)₂, OR¹³, C₁-C₆ alkyl, C₃-C₇ cycloalkyl, C₄-C₇ heterocycloalkyl, CO₂R¹⁴, CONH₂, CONHR¹⁴, and CONR¹⁴R¹⁵;

wherein each R¹³ is independently selected from the group consisting of H, C₁-C₆ alkyl, C₃-C₇ cycloalkyl, C₄-C₇ heterocycloalkyl, CO₂R¹⁴, CONH₂, CONHR¹⁴, CONR¹⁴R¹⁵, SOR¹⁴, SO₂R¹⁴, SO₂NH₂, SO₂NHR¹⁴, SO₂NR¹⁴ R⁵;

wherein each R¹⁴ is independently selected from the group consisting of H, C₁-C₆ alkyl, C₃-C₇ cycloalkyl, C₄-C₇ heterocycloalkyl, C₆-C₁₀ aryl, C₅-C₁₀ membered heteroaryl; said alkyl, cycloalkyl, heterocycloalkyl, aryl, and heteroaryl moieties of the foregoing groups are optionally substituted by 1 to 3 substituents independently selected from the group consisting of H, halo, C₁-C₆ alkyl, CN, NH₂, NH C₁-C₆alkyl, N(C₁-C₆alkyl)₂, O—C₁-C₆ alkyl; and

wherein each R¹⁵ is independently selected from the group consisting of H, C₁-C₆ alkyl, C₃-C₇ cycloalkyl, C₄-C₇ heterocycloalkyl, C₆-C₁₀ aryl, C₅-C₁₀ membered hetoroaryl; said alkyl, cycloalkyl, heterocycloalkyl, aryl, and heteroaryl moieties of the foregoing groups are optionally substituted by 1 to 3 substituents independently selected from the group consisting of H, halo, C₁-C₆ alkyl, CN, NH₂, NH C₁-C₆alkyl, N(C₁-C₆alkyl)₂, O—C₁-C₆ alkyl.

In one preferred embodiment of the compounds of formula 1, include those wherein E and G are independently selected from the group consisting of N and C; wherein X, W and Q are independently selected from the group consisting of N, O, CO, NR³, CR² and CR²R³; and wherein Y and Z are independently present or absent, if present Y and Z are selected from the group consisting of N, O, CO, NR³, CR² and CR²R³.

In another preferred embodiment of the compounds of formula 1, include those wherein E and G are independently selected from the group consisting of N and C; wherein X, W and Q are independently selected from the group consisting of N, CO, NR³, CR² and CR²R³; and

wherein Y and Z are independently present or absent, if present Y and Z are selected from the group consisting of N, CO, NR³, CR² and CR²R³.

In a more preferred embodiment of the compounds of formula 1, include those wherein E and G are C; wherein X, W and Q are independently selected from the group consisting of N, CO, NR³, CR² and CR²R³; and wherein Y and Z are independently present or absent, if present Y and Z are selected from the group consisting of N, CO, NR³, CR² and CR²R³.

In a most preferred embodiment of the compounds of formula 1, include those wherein E and G are C; wherein X, W and Q are independently selected from the group consisting of N, NR³, CR² and CR²R³; and wherein Y and Z are independently present or absent, if present Y and Z are selected from the group consisting of N, NR³, CR² and CR²R³.

In one specific embodiment of the compounds of formula 1, include those wherein R² is selected from the group consisting of:

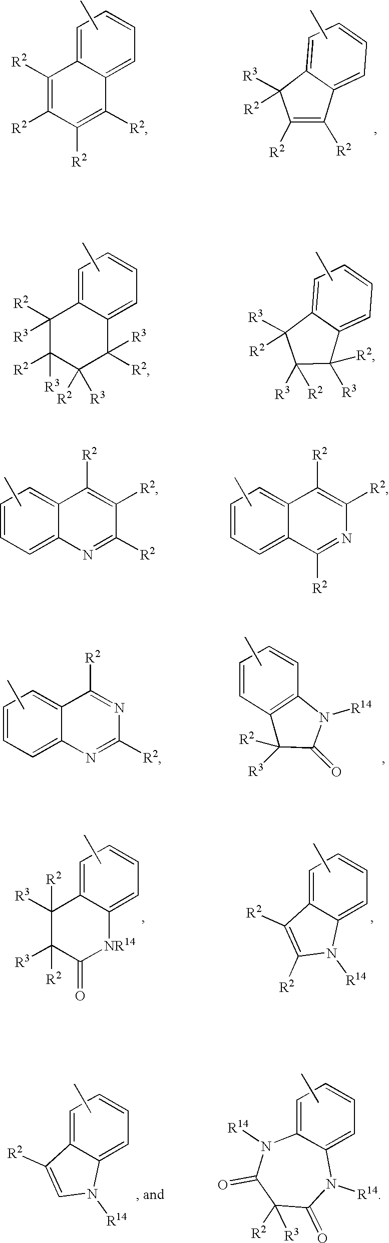

In another specific embodiment of the compounds of formula 1, include those wherein R² is selected from the group consisting of:

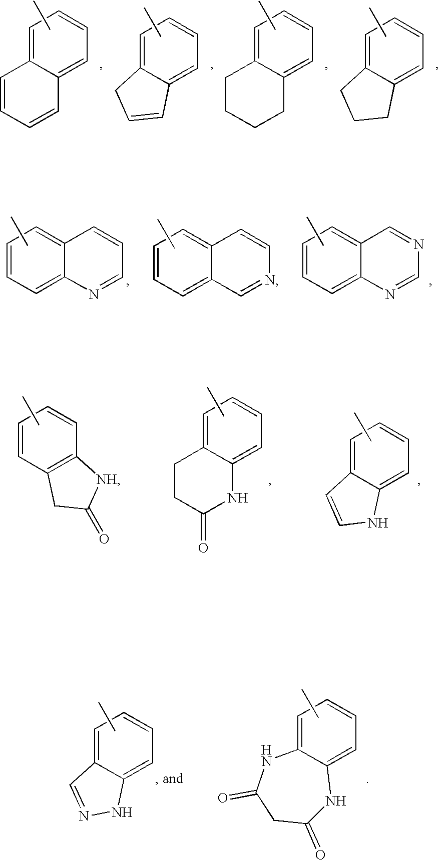

In another specific embodiment of the compounds of formula 1, include those wherein R² is selected from the group consisting of:

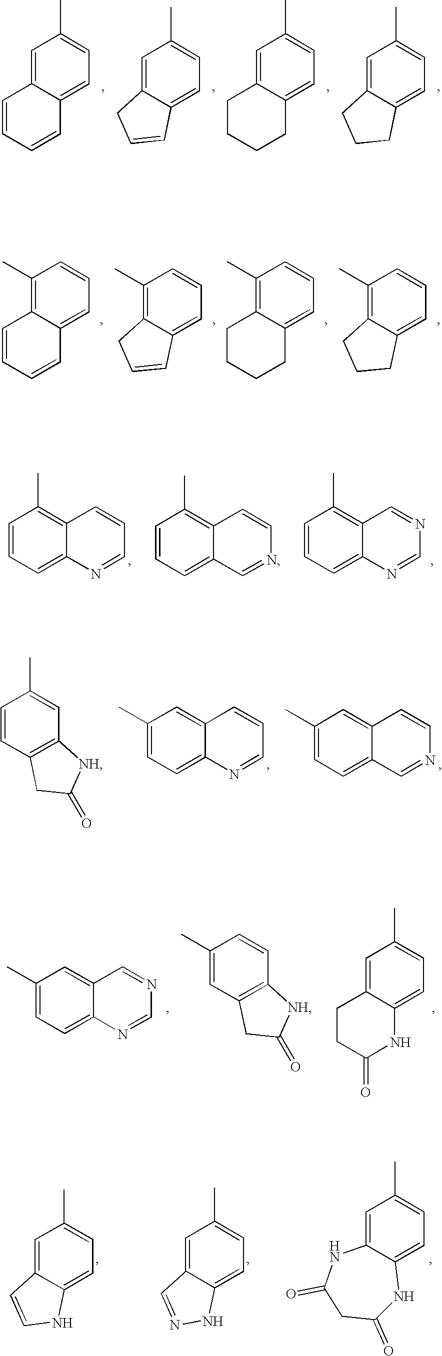

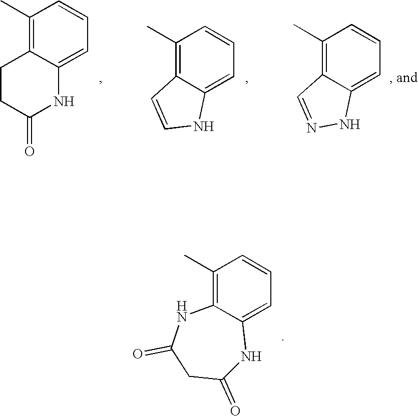

Specific embodiments of the compounds of formula 1 include those wherein R² is selected from the group consisting of:

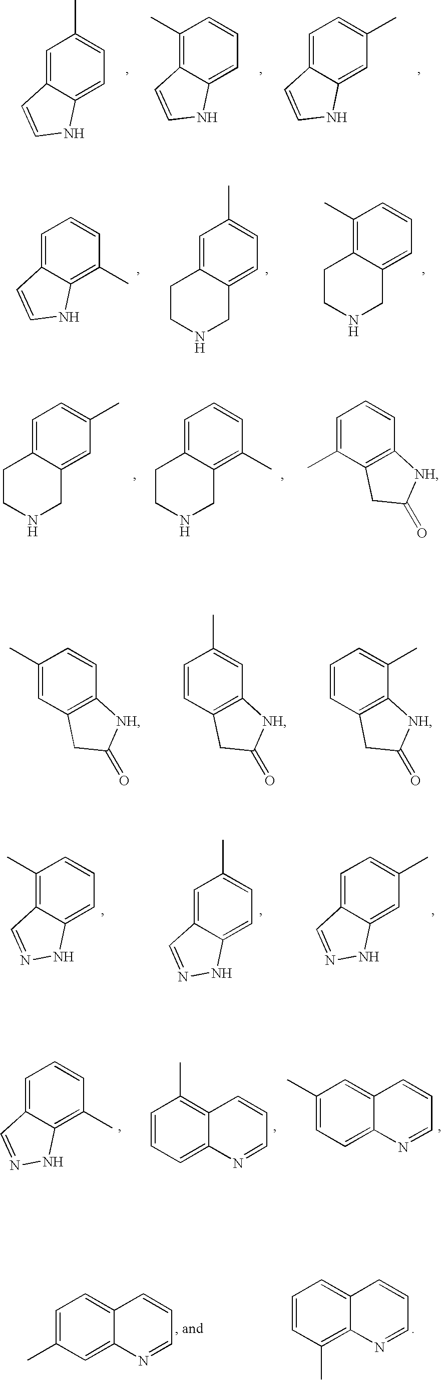

Specific embodiments of the compounds of formula 1 include those wherein A is present or absent, if present A is selected from the group consisting of O and NH and wherein B is present or absent, if present B is selected from the group consisting of CO, SO₂, and NR⁶, with the proviso that when A is O that B is absent.

Specific embodiments of the compounds of formula 1 include those wherein A is present or absent, if present A is NH and wherein B is present or absent, if present B is selected from the group consisting of CO, SO₂, and NR⁶.

Specific embodiments of the compounds of formula 1 include those wherein A is present or absent, if present A is NH and wherein B is present or absent, if present B is selected from the group consisting of CO and NR⁶.

In one preferred embodiment of the compounds of formula 1 include those wherein A is present or absent, if present A is NH and wherein B is present or absent, if present B is CO.

In a more preferred embodiment of the compounds of formula 1 include those wherein A is present or absent, if present A is NH and wherein B is absent.

In a most preferred embodiment of the compounds of formula 1 include those wherein A is NH and wherein B is absent.

Specific embodiments of the compounds of formula 1 include those each R² is independently selected from the group consisting of H, C₁-C₆ alkyl, C₃-C₇ cycloalkyl, C₄-C₇ heterocycloalkyl, OC₁-C₆ alkyl, OC₃-C₇ cycloalkyl, OC₄-C₇ heterocyloalkyl, NH₂, NHR⁶, NR⁶R⁷, SR⁶, SOR⁶, SO₂R⁶, CO₂R⁶, CONH₂, CONHR⁶, CONR⁶R⁶, NHCOR⁶, NR⁶CONR⁶, NHCONHR⁶, NR⁶CONHR⁶, NHCONR⁶R⁷, NR⁶CONR⁶R⁷, NHSO₂R⁶, NR⁶SO₂R⁶, with the proviso that O, N or S atom of the foregoing substituents may not be bound to a carbon atom bound to another heteroatom, said alkyl, cycloalkyl, heterocycloalkyl moieties of the foregoing groups are optionally substituted by 1 to 3 substituents independently selected from the group consisting of H, halo, C₁-C₆ alkyl, CN, NH₂, NHR¹⁰, N(R¹⁰)₂, OR¹⁰, C₁-C₆ alkyl, C₃-C₇ cycloalkyl, C₄-C₇ heterocycloalkyl, CO₂R¹¹, CONH₂, CONHR¹¹, and CONR¹¹R¹²; and wherein each R³ is independently selected from the group consisting of H, C₁-C₆ alkyl, C₃-C₇ cycloalkyl, C₄-C₇ heterocycloalkyl, CO₂R⁶, CONH₂, CONHR⁶, CONR⁶R⁷ or R² and R³ taken together with the carbon atom they are linked to can form a 3-7 membered cycloalkyl ring or 4-7 membered heterocycloalkyl ring, wherein each methylene group present in said 3-7 membered cycloalkyl ring and said 4-7 membered heterocycloalkyl ring may be optionally replaced by a C═O group, said alkyl, cycloalkyl, heterocycloalkyl moieties of the foregoing groups are optionally substituted by 1 to 3 substituents independently selected from the group consisting of H, halo, C₁-C₆ alkyl, CN, NH₂, NHR¹⁰, N(R¹⁰)₂, OR¹⁰, C₁-C₆ alkyl, C₃-C₇ cycloalkyl, C₄-C₇ heterocycloalkyl, CO₂R¹¹, CONH₂, CONHR¹¹, and CONR¹¹R¹².

Specific embodiments of the compounds of formula 1 include those each R² is independently selected from the group consisting of H, C₁-C₆ alkyl, C₃-C₇ cycloalkyl, C₄-C₇ heterocycloalkyl, OC₁-C₆ alkyl, OC₃-C₇ cycloalkyl, OC₄-C₇ heterocyloalkyl, NH₂, NHR⁶, NR⁶ R⁷, with the proviso that O, N or S atom of the foregoing substituents may not be bound to a carbon atom bound to another heteroatom, said alkyl, cycloalkyl, heterocycloalkyl moieties of the foregoing groups are optionally substituted by 1 to 3 substituents independently selected from the group consisting of H, halo, C₁-C₆ alkyl, CN, NH₂, NHR¹⁰, N(R¹⁰)₂, OR¹⁰, C₁-C₆ alkyl, C₃-C₇ cycloalkyl, C₄-C₇ heterocycloalkyl, CO₂R¹¹, CONH₂, CONHR¹¹, and CONR¹¹R¹²; and wherein each R³ is independently selected from the group consisting of H, C₁-C₆ alkyl, C₃-C₇ cycloalkyl, C₄-C₇ heterocycloalkyl, CO₂R⁶, CONH₂, CONHR⁶, CONR⁶R⁷ or R² and R³ taken together with the carbon atom they are linked to can form a 3-7 membered cycloalkyl ring or 4-7 membered heterocycloalkyl ring, wherein each methylene group present in said 3-7 membered cycloalkyl ring and said 4-7 membered heterocycloalkyl ring may be optionally replaced by a C═O group, said alkyl, cycloalkyl, heterocycloalkyl moieties of the foregoing groups are optionally substituted by 1 to 3 substituents independently selected from the group consisting of H, halo, C₁-C₆ alkyl, CN, NH₂, NHR¹⁰, N(R¹⁰)₂, OR¹⁰, C₁-C₆ alkyl, C₃-C₇ cycloalkyl, C₄-C₇ heterocycloalkyl, CO₂R¹¹, CONH₂, CONHR¹¹, and CONR¹¹R¹².

Specific embodiments of the compounds of formula 1 include those R⁴ is selected from the group consisting of H, C₁-C₆ alkyl, C₆-C₁₀ aryl, and 5-10 membered heteroaryl, the alkyl, aryl and heteroaryl moieties of the foregoing groups are optionally substituted by 1 to 3 subsitutents independently selected from the group consisting of H, halo, OH, NO₂, C₁-C₆ alkyl, C(R⁶)═CR⁶R⁷, C≡CR⁶, C₃-C₇ cycloalkyl, C₄-C₇ heterocycloalkyl, OC₁-C₆ alkyl, OC₃-C₇ cycloalkyl, OC₄-C₇ heterocyloalkyl, C═N—OH, C═N—O(C₁-C₆ alkyl), NH₂, NHR⁶, NR⁶R⁷, SR⁶, SOR⁶, SO₂R⁶, CO₂R⁶, CONH₂, CONHR⁶, CONR⁶R⁷, SO₂NH₂, SO₂NHR⁶, SO₂NR⁶R⁷, NHCOR⁶, NR⁶CONR⁶, NHCONHR⁶, NR⁶CONHR⁶, NHCONR⁶R⁷, NR⁶CONR⁶R⁷, NHSO₂R⁶, NR⁶SO₂R⁶, with the proviso that O, N or S atom of the foregoing substituents may not be bound to a carbon atom bound to another heteroatom.

Specific embodiments of the compounds of formula 1 include those R⁴ is selected from the group consisting of H, C₁-C₆ alkyl, and C₆-C₁₀ aryl, wherein the alkyl, and aryl moieties of the foregoing groups are optionally substituted by 1 to 3 subsitutents independently selected from the group consisting of H, halo, OH, NO₂, C₁-C₆ alkyl, C(R⁶)═CR⁶R⁷, C≡CR⁶, C₃-C₇ cycloalkyl, C₄-C₇ heterocycloalkyl, OC₁-C₆ alkyl, OC₃-C₇ cycloalkyl, OC₄-C₇ heterocyloalkyl, C═N—OH, C═N—O(C₁-C₆ alkyl), NH₂, NHR⁶, NR⁶R⁷, SR⁶, SOR⁶, SO₂R⁶, CO₂R⁶, CONH₂, CONHR⁶, CONR⁶R⁷, SO₂NH₂, SO₂NHR⁶, SO₂NR⁶R⁷, NHCOR⁶, NR⁶CONR⁶, NHCONHR⁶, NR⁶CONHR⁶, NHCONR⁶R⁷, NR⁶CONR⁶R⁷, NHSO₂R⁶, NR⁶SO₂R⁶, with the proviso that O, N or S atom of the foregoing substituents may not be bound to a carbon atom bound to another heteroatom.

Specific embodiments of the compounds of formula 1 include those R⁵ is selected from the group consisting of H, Br, Cl, CN, CF₃, CH₂F, CHF₂, SO₂CH₃, CONH₂, C₆H₅, CONHR⁶, CONR⁶R⁷, CO₂R⁶, C(R⁹)═C(R⁹)₂, and C≡CR⁹.

Specific embodiments of the compounds of formula 1 include those R⁵ is selected from the group consisting of H, Br, Cl, CN, CF₃, CH₂F, CHF₂, SO₂CH₃, CONH₂, and C₆H₅.

Specific embodiments of the compounds of formula 1 include those R⁵ is selected from the group consisting of H, Br, Cl, CN, CF₃, CH₂F, CHF₂, SO₂CH₃, and CONH₂.

Other specific embodiments of the compounds of formula 1 include those selected from the group consisting of:

5-Bromo-N²-[3-(1,2,3,6-tetrahydro-pyridin-4-yl)-1H-indol-5-yl]-N⁴-p-tolyl-pyrimidine-2,4-diamine;

5-Bromo-N⁴-pyridin-2-yl-N2-[3-(1,2,3,6-tetrahydro-pyridin-4-yl)-1H-indol-5-yl]-pyrimidine-2,4-diamine;

5-Bromo-N⁴-pyridin-2-ylmethyl-N²-[3-(1,2,3,6-tetrahydro-pyridin-4-yl)-1H-indol-5-yl]-pyrimidine-2,4-diamine;

N⁴-Benzyl-5-bromo-N²-[3-(1,2,3,6-tetrahydro-pyridin-4-yl)-1H-indol-5-yl]-pyrimidine-2,4-diamine;

5-Bromo-N4-(1R-phenyl-ethyl)-N2-[3-(1,2,3,6-tetrahydro-pyridin-4-yl)-1H-indol-5-yl]-pyrimidine-2,4-diamine;

5-Bromo-N⁴-(1rac-phenyl-ethyl)-N²-[3-(1,2,3,6-tetrahydro-pyridin-4-yl)-1H-indol-5-yl]-pyrimidine-2,4-diamine;

5-Bromo-N4-(1S-phenyl-ethyl)-N2-[3-(1,2,3,6-tetrahydro-pyridin-4-yl)-1H-indol-5-yl]-pyrimidine-2,4-diamine;

4-({5-Bromo-2-[3-(1,2,3,6-tetrahydro-pyridin-4-yl)-1H-indol-5-ylamino]-pyrimidin-4-ylamino}-methyl)-benzenesulfonamide

5-Bromo-N²-[3-(1,2,3,6-tetrahydro-pyridin-4-yl)-1H-indol-5-yl]-N⁴-(4-trifluoromethyl-benzyl)-pyrimidine-2,4-diamine;

5-Bromo-N⁴-(4-methoxy-benzyl)-N²-[3-(1,2,3,6-tetrahydro-pyridin-4-yl)-1H-indol-5-yl]-pyrimidine-2,4-diamine;

5-Bromo-N⁴-(4-fluoro-benzyl)-N²-[3-(1,2,3,6-tetrahydro-pyridin-4-yl)-1H-indol-5-yl]-pyrimidine-2,4-diamine;

5-Bromo-N⁴-(3-fluoro-benzyl)-N²-[3-(1,2,3,6-tetrahydro-pyridin-4-yl)-1H-indol-5-yl]-pyrimidine-2,4-diamine;

5-Bromo-N⁴-naphthalen-1-ylmethyl-N²-[3-(1,2,3,6-tetrahydro-pyridin-4-yl)-1H-indol-5-yl]-pyrimidine-2,4-diamine;

5-Bromo-N⁴-(4-fluoro-3-trifluoromethyl-benzyl)-N²-[3-(1,2,3,6-tetrahydro-pyridin-4-yl)-1H-indol-5-yl]-pyrimidine-2,4-diamine;

5-Bromo-N⁴-(3-fluoro-5-trifluoromethyl-benzyl)-N²-[3-(1,2,3,6-tetrahydro-pyridin-4-yl)-1H-indol-5-yl]-pyrimidine-2,4-diamine;

5-Bromo-N⁴-(4-phenoxy-benzyl)-N²-[3-(1,2,3,6-tetrahydro-pyridin-4-yl)-1H-indol-5-yl]-pyrimidine-2,4-diamine;

5-Bromo-N⁴-(3,4-difluoro-benzyl)-N²-[3-(1,2,3,6-tetrahydro-pyridin-4-yl)-1H-indol-5-yl]-pyrimidine-2,4-diamine;

5-Bromo-N²-[3-(1,2,3,6-tetrahydro-pyridin-4-yl)-1H-indol-5-yl]-N⁴-(3-trifluoromethoxybenzyl)-pyrimidine-2,4-diamine;

5-Bromo-N⁴-(4-chloro-benzyl)-N²-[3-(1,2,3,6-tetrahydro-pyridin-4-yl)-1H-indol-5-yl]-pyrimidine-2,4-diamine;

5-Bromo-N²-[3-(1,2,3,6-tetrahydro-pyridin-4-yl)-1H-indol-5-yl]-N⁴-thiophen-2-ylmethyl-pyrimidine-2,4-diamine;

5-Bromo-N4-furan-2-ylmethyl-N2-[3-(1,2,3,6-tetrahydro-pyridin-4-yl)-1H-indol-5-yl]-pyrimidine-2,4-diamine;

5-Bromo-N⁴-(2-methyl-benzyl)-N²-[3-(1,2,3,6-tetrahydro-pyridin-4-yl)-1H-indol-5-yl]-pyrimidine-2,4-diamine;

5-Bromo-N⁴-(3-methyl-benzyl)-N²-[3-(1,2,3,6-tetrahydro-pyridin-4-yl)-1H-indol-5-yl]-pyrimidine-2,4-diamine;

5-Bromo-N⁴-(4-methyl-benzyl)-N²-[3-(1,2,3,6-tetrahydro-pyridin-4-yl)-1H-indol-5-yl]-pyrimidine-2,4-diamine;

5-Bromo-N⁴-(2-fluoro-benzyl)-N²-[3-(1,2,3,6-tetrahydro-pyridin-4-yl)-1H-indol-5-yl]-pyrimidine-2,4-diamine;

N⁴-Biphenyl-2-ylmethyl-5-bromo-N²-[3-(1,2,3,6-tetrahydro-pyridin-4-yl)-1H-indol-5-yl]-pyrimidine-2,4-diamine;

N⁴-Biphenyl-3-ylmethyl-5-bromo-N²-[3-(1,2,3,6-tetrahydro-pyridin-4-yl)-1H-indol-5-yl]-pyrimidine-2,4-diamine;

5-Bromo-N⁴-(2-methoxy-benzyl)-N²-[3-(1,2,3,6-tetrahydro-pyridin-4-yl)-1H-indol-5-yl]-pyrimidine-2,4-diamine;

5-Bromo-N⁴-(3-methoxy-benzyl)-N²-[3-(1,2,3,6-tetrahydro-pyridin-4-yl)-1H-indol-5-yl]-pyrimidine-2,4-diamine;

3-({5-Bromo-2-[3-(1,2,3,6-tetrahydro-pyridin-4-yl)-1H-indol-5-ylamino]-pyrimidin-4-ylamino}-methyl)-N-methyl-benzamide

5-Bromo-N⁴-(2-chloro-benzyl)-N²-[3-(1,2,3,6-tetrahydro-pyridin-4-yl)-1H-indol-5-yl]-pyrimidine-2,4-diamine;

5-Bromo-N⁴-phenethyl-N²-[3-(1,2,3,6-tetrahydro-pyridin-4-yl)-1H-indol-5-yl]-pyrimidine-2,4-diamine;

5-Bromo-N⁴-(2-pyridin-2-yl-ethyl)-N²-[3-(1,2,3,6-tetrahydro-pyridin-4-yl)-1H-indol-5-yl]-pyrimidine-2,4-diamine;

5-Bromo-N⁴-(2-pyridin-4-yl-ethyl)-N²-[3-(1,2,3,6-tetrahydro-pyridin-4-yl)-1H-indol-5-yl]-pyrimidine-2,4-diamine;

5-Bromo-N⁴-(2-pyridin-3-yl-ethyl)-N²-[3-(1,2,3,6-tetrahydro-pyridin-4-yl)-1H-indol-5-yl]-pyrimidine-2,4-diamine;

5-Bromo-N⁴-[2-(3-fluoro-phenyl)-ethyl]-N²-[3-(1,2,3,6-tetrahydro-pyridin-4-yl)-1H-indol-5-yl]-pyrimidine-2,4-diamine;

5-Bromo-N⁴-(2-phenyl-cyclopropyl)-N²-[3-(1,2,3,6-tetrahydro-pyridin-4-yl)-1H-indol-5-yl]-pyrimidine-2,4-diamine;

5-Bromo-N⁴-(2-phenyl-cyclopropyl)-N²-[3-(1,2,3,6-tetrahydro-pyridin-4-yl)-1H-indol-5-yl]-pyrimidine-2,4-diamine; (homo-chiral)

5-Bromo-N⁴-(2-phenyl-cyclopropyl)-N²-[3-(1,2,3,6-tetrahydro-pyridin-4-yl)-1H-indol-5-yl]-pyrimidine-2,4-diamine; (homo-chiral)

5-Bromo-N⁴-[2-(4-chloro-phenyl)-ethyl]-N²-[3-(1,2,3,6-tetrahydro-pyridin-4-yl)-1H-indol-5-yl]-pyrimidine-2,4-diamine;

5-Bromo-N²-[3-(1,2,3,6-tetrahydro-pyridin-4-yl)-1H-indol-5-yl]-N⁴-(2-thiophen-2-yl-ethyl)-pyrimidine-2,4-diamine;

5-Bromo-N⁴-[2-(2-fluoro-phenyl)-ethyl]-N²-[3-(1,2,3,6-tetrahydro-pyridin-4-yl)-1H-indol-5-yl]-pyrimidine-2,4-diamine;

5-Bromo-N⁴-[2-(2-chloro-phenyl)-ethyl]-N²-[3-(1,2,3,6-tetrahydro-pyridin-4-yl)-1H-indol-5-yl]-pyrimidine-2,4-diamine;

5-Bromo-N⁴-[2-(2-methoxy-phenyl)-ethyl]-N²-[3-(1,2,3,6-tetrahydro-pyridin-4-yl)-1H-indol-5-yl]-pyrimidine-2,4-diamine;

N⁴-(2-Benzo[1,3]dioxol-5-yl-ethyl)-5-bromo-N²-[3-(1,2,3,6-tetrahydro-pyridin-4-yl)-1H-indol-5-yl]-pyrimidine-2,4-diamine;

5-Bromo-N⁴-(3-phenyl-propyl)-N²-[3-(1,2,3,6-tetrahydro-pyridin-4-yl)-1H-indol-5-yl]-pyrimidine-2,4-diamine;

5-(5-Bromo-4-phenethylamino-pyrimidin-2-ylamino)-1,3-dihydro-indol-2-one;

5-[5-Bromo-4-(2-chloro-benzylamino)-pyrimidin-2-ylamino]-1,3-dihydro-indol-2-one;

5-(4-Benzylamino-5-bromo-pyrimidin-2-ylamino)-1,3-dihydro-indol-2-one;

5-[5-Bromo-4-(1-phenyl-ethylamino)-pyrimidin-2-ylamino]-1,3-dihydro-indol-2-one;

5-[5-Bromo-4-(3-phenyl-propylamino)-pyrimidin-2-ylamino]-1,3-dihydro-indol-2-one;

5-Bromo-N⁴-(2-methanesulfonyl-ethyl)-N²-[3-(1,2,3,6-tetrahydro-pyridin-4-yl)-1H-indol-5-yl]-pyrimidine-2,4-diamine;

N⁴-Benzyl-N²-[3-(1,2,3,6-tetrahydro-pyridin-4-yl)-1H-indol-5-yl]-pyrimidine-2,4-diamine;

N⁴-Benzyl-N⁴-methyl-N²-[3-(1,2,3,6-tetrahydro-pyridin-4-yl)-1H-indol-5-yl]-pyrimidine-2,4-diamine;

N⁴-Methyl-N⁴-(2-pyridin-2-yl-ethyl)-N²-[3-(1,2,3,6-tetrahydro-pyridin-4-yl)-1H-indol-5-yl]-pyrimidine-2,4-diamine;

[4-(2-Phenyl-morpholin-4-yl)-pyrimidin-2-yl]-[3-(1,2,3,6-tetrahydro-pyridin-4-yl)-1H-indol-5-yl]-amine

5-Methyl-N⁴-(2-pyridin-2-yl-ethyl)-N²-[3-(1,2,3,6-tetrahydro-pyridin-4-yl)-1H-indol-5-yl]-pyrimidine-2,4-diamine;

5-Bromo-N²-(3-piperidin-4-yl-1H-indol-5-yl)-N⁴-(2-pyridin-2-yl-ethyl)-pyrimidine-2,4-diamine;

5-Bromo-N²-[1-methanesulfonyl-3-(1,2,3,6-tetrahydro-pyridin-4-yl)-1H-indol-5-yl]-N⁴-(2-pyridin-2-yl-ethyl)-pyrimidine-2,4-diamine;

5-Bromo-N²-[1-methanesulfonyl-3-(1,2,3,6-tetrahydro-pyridin-4-yl)-1H-indol-5-yl]-N⁴-pyridin-2-yl-pyrimidine-2,4-diamine;

5-Bromo-N²-(2-pyridin-2-yl-ethyl)-N⁴-[3-(1,2,3,6-tetrahydro-pyridin-4-yl)-1H-indol-5-yl]-pyrimidine-2,4-diamine;

3-{4-(2-Pyridin-2-yl-ethylamino)-2-[3-(1,2,3,6-tetrahydro-pyridin-4-yl)-1H-indol-5-ylamino]-pyrimidin-5-yl}-acrylic acid; ethyl ester;

5-{5-Bromo-4-[2-(3-chloro-phenyl)-ethylamino]-pyrimidin-2-ylamino}-1,3-dihydro-indol-2-one;

5-Bromo-N⁴-[2-(3-chloro-phenyl)-ethyl]-N²-[3-(1,2,3,6-tetrahydro-pyridin-4-yl)-1H-indol-5-yl]-pyrimidine-2,4-diamine;

5-Bromo-N⁴-[2-(3-chloro-phenyl)-ethyl]-N²-[3-(1,2,3,6-tetrahydro-pyridin-4-yl)-1H-indol-5-yl]-pyrimidine-2,4-diamine;

5-{5-Bromo-4-[2-(4-methoxy-phenyl)-ethylamino]-pyrimidin-2-ylamino}-1,3-dihydro-indol-2-one;

5-Bromo-N⁴-[2-(4-methoxy-phenyl)-ethyl]-N²-[3-(1,2,3,6-tetrahydro-pyridin-4-yl)-1H-indol-5-yl]-pyrimidine-2,4-diamine;

5-{5-Bromo-4-[2-(3-methoxy-phenyl)-ethylamino]-pyrimidin-2-ylamino}-1,3-dihydro-indol-2-one;

5-Bromo-N⁴-[2-(3-methoxy-phenyl)-ethyl]-N 2-[3-(1,2,3,6-tetrahydro-pyridin-4-yl)-1H-indol-5-yl]-pyrimidine-2,4-diamine;

5-[5-Bromo-4-(2-o-tolyl-ethylamino)-pyrimidin-2-ylamino]-1,3-dihydro-indol-2-one;

5-Bromo-N²-[3-(1,2,3,6-tetrahydro-pyridin-4-yl)-1H-indol-5-yl]-N⁴-(2-o-tolyl-ethyl)-pyrimidine-2,4-diamine;

5-[5-Bromo-4-(2-m-tolyl-ethylamino)-pyrimidin-2-ylamino]-1,3-dihydro-indol-2-one;

5-Bromo-N²-[3-(1,2,3,6-tetrahydro-pyridin-4-yl)-1H-indol-5-yl]-N⁴-(2-m-tolyl-ethyl)-pyrimidine-2,4-diamine;

5-[5-Bromo-4-(2-p-tolyl-ethylamino)-pyrimidin-2-ylamino]-1,3-dihydro-indol-2-one;

5-Bromo-N²-[3-(1,2,3,6-tetrahydro-pyridin-4-yl)-1H-indol-5-yl]-N⁴-(2-p-tolyl-ethyl)-pyrimidine-2,4-diamine;

[5-Bromo-2-(2-oxo-2,3-dihydro-1H-indol-5-ylamino)-pyrimidin-4-ylamino]-acetic acid;

5-{5-Bromo-4-[2-(3-trifluoromethyl-phenyl)-ethylamino]-pyrimidin-2-ylamino}-1,3-dihydro-indol-2-one;

5-[4-(2-Biphenyl-4-yl-ethylamino)-5-bromo-pyrimidin-2-ylamino]-1,3-dihydro-indol-2-one;

5-{5-Bromo-4-[2-(3-fluoro-phenyl)-ethylamino]-pyrimidin-2-ylamino}-1,3-dihydro-indol-2-one;

5-{5-Bromo-4-[2-(2-chloro-phenyl)-ethylamino]-pyrimidin-2-yiamino}-1,3-dihydro-indol-2-one;

5-{5-Bromo-4-[2-(2-methoxy-phenyl)-ethylamino]-pyrimidin-2-yiamino}-1,3-dihydro-indol-2-one;

5-{5-Bromo-4-[2-(4-fluoro-phenyl)-ethylamino]-pyrimidin-2-ylamino}-1,3-dihydro-indol-2-one;

5-{5-Bromo-4-[2-(4-chloro-phenyl)-ethylamino]-pyrimidin-2-ylamino}-1,3-dihydro-indol-2-one;

5-{5-Bromo-4-[2-(2-fluoro-phenyl)-ethylamino]-pyrimidin-2-ylamino}-1,3-dihydro-indol-2-one;

5-[5-Bromo-4-(3-phenyl-allylamino)-pyrimidin-2-ylamino]-1,3-dihydro-indol-2-one;

5-{5-Bromo-4-[(thiophen-2-ylmethyl)-amino]-pyrimidin-2-ylamino}-1,3-dihydro-indol-2-one;

6-{5-Bromo-4-[(thiophen-2-ylmethyl)-amino]-pyrimidin-2-ylamino}-1,3-dihydro-indol-2-one;

5-[5-Bromo-4-(2,3-dimethyl-benzylamino)-pyrimidin-2-ylamino]-1,3-dihydro-indol-2-one;

6-[5-Bromo-4-(2,3-dimethyl-benzylamino)-pyrimidin-2-ylamino]-1,3-dihydro-indol-2-one;

5-[5-Bromo-4-(2,5-dimethyl-benzylamino)-pyrimidin-2-ylamino]-1,3-dihydro-indol-2-one;

6-[5-Bromo-4-(2,5-dimethyl-benzylamino)-pyrimidin-2-ylamino]-1,3-dihydro-indol-2-one;

6-[5-Bromo-4-(2-fluoro-benzylamino)-pyrimidin-2-ylamino]-1,3-dihydro-indol-2-one;

6-[5-Bromo-4-(2-trifluoromethoxy-benzylamino)-pyrimidin-2-ylamino]-1,3-dihydro-indol-2-one;

5-[5-Bromo-4-(3-trifluoromethoxy-benzylamino)-pyrimidin-2-ylamino]-1,3-dihydro-indol-2-one;

6-[5-Bromo-4-(3-trifluoromethoxy-benzylamino)-pyrimidin-2-ylamino]-1,3-dihydro-indol-2-one;

5-[5-Bromo-4-(4-trifluoromethoxy-benzylamino)-pyrimidin-2-ylamino]-1,3-dihydro-indol-2-one;

6-[5-Bromo-4-(4-trifluoromethoxy-benzylamino)-pyrimidin-2-ylamino]-1,3-dihydro-indol-2-one;

6-[5-Bromo-4-(2-methoxy-benzylamino)-pyrimidin-2-ylamino]-1,3-dihydro-indol-2-one;

6-[5-Bromo-4-(3-methoxy-benzylamino)-pyrimidin-2-ylamino]-1,3-dihydro-indol-2-one;

6-[5-Bromo-4-(3-trifluoromethyl-benzylamino)-pyrimidin-2-ylamino]-1,3-dihydro-indol-2-one;

5-{5-Bromo-4-[(thiazol-2-ylimethyl)-amino]-pyrimidin-2-ylamino}-1,3-dihydro-indol-2-one;

5-{5-Bromo-4-[(5-methanesulfonyl-thiophen-2-ylmethyl)-amino]-pyrimidin-2-ylamino}-1,3-dihydro-indol-2-one;

5-[5-Bromo-4-(2,3-difluoro-benzylamino)-pyrimidin-2-ylamino]-1,3-dihydro-indol-2-one;

6-[5-Bromo-4-(2,3-difluoro-benzylamino)-pyrimidin-2-ylamino]-1,3-dihydro-indol-2-one;

5-[5-Bromo-4-(2,4-difluoro-benzylamino)-pyrimidin-2-ylamino]-1,3-dihydro-indol-2-one;

6-[5-Bromo-4-(2,4-difluoro-benzylamino)-pyrimidin-2-ylamino]-1,3-dihydro-indol-2-one;

6-[5-Chloro-4-(2-trifluoromethyl-benzylamino)-pyrimidin-2-yiamino]-1,3-dihydro-indol-2-one;

5-Chloro-N2-(1-methyl-1H-indol-5-yl)-N4-(2-trifluoromethyl-benzyl)-pyrimidine-2,4-diamine;

5-Chloro-N2-(1H-indazol-5-yl)-N4-(2-trifluoromethyl-benzyl)-pyrimidine-2,4-diamine;

5-Chloro-N2-(1-methyl-1H-indol-5-yl)-N4-pyridin-2-ylmethyl-pyrimidine-2,4-diamine;

6-{5-Chloro-4-[(pyridin-2-ylmethyl)-amino]-pyrimidin-2-ylamino}-1,3-dihydro-indol-2-one;

5-Chloro-N2-(1H-indazol-6-yl)-N4-(2-trifluoromethyl-benzyl)-pyrimidine-2,4-diamine;

5-Chloro-N2-(1H-indazol-6-yl)-N4-pyridin-2-ylmethyl-pyrimidine-2,4-diamine;

(5-{5-Bromo-4-[(pyridin-2-ylmethyl)-amino]-pyrimidin-2-ylamino}-indazol-1-yl)-acetic acid; tert-butyl ester;

(6{5-Bromo-4-[(pyridin-2-ylmethyl)-amino]-pyrimidin-2-ylamino}-indazol-2-yl)-acetic acid; tert-butyl ester;

6-{4-[(Pyridin-2-ylmethyl)-amino]-5-trifluoromethyl-pyrimidin-2-ylamino}-1,3-dihydro-indol-2-one;

N2-(1-Methyl-1H-indol-5-yl)-N4-pyridin-2-ylmethyl-5-trifluoromethyl-pyrimidine-2,4-diamine;

(6-{5-Bromo-4-[(pyridin-2-ylmethyl)-amino]-pyrimidin-2-ylamino}-indol-1-yl)-acetic acid; tert-butyl ester;

N4-Pyridin-2-ylmethyl-N2-quinolin-5-yl-5-trifluoromethyl-pyrimidine-2,4-diamine;

2-(6-{5-Bromo-4-[(pyridin-2-ylmethyl)-amino]-pyrimidin-2-ylamino}-indol-1-yl)-N-(2-methoxy-ethyl)-acetamide;

6-{5-Chloro-4-[(3-methyl-pyridin-2-ylmethyl)-amino]-pyrimidin-2-ylamino}-1,3-dihydro-indol-2-one;

(6-{5-Bromo-4-[(pyridin-2-ylmethyl)-amino]-pyrimidin-2-ylamino}-indol-1-yl)-acetic acid;

(6-{5-Bromo-4-[(pyridin-2-ylmethyl)-amino]-pyrimidin-2-ylamino}-indazol-1-yl)-acetic acid; tert-butyl ester;

N2-(1H-indazol-6-yl)-N4-pyridin-2-ylmethyl-5-trifluoromethyl-pyrimidine-2,4-diamine;

(5-{5-Bromo-4-[(pyridin-2-ylmethyl)-amino]-pyrimidin-2-ylamino}-indol-1-yl)-acetic acid; tert-butyl ester;

(6-{5-Bromo-4-[(pyridin-2-ylmethyl)-amino]-pyrimidin-2-ylamino}-indazol-1-yl)-acetic acid;

(5-{5-Bromo-4-[(pyridin-2-ylmethyl)-amino]-pyrimidin-2-ylamino}-indol-1-yl)-acetic acid;

(5-{5-Bromo-4-[(pyridin-2-ylmethyl)-amino]-pyrimidin-2-ylamino}-indazol-1-yl)-acetic acid;

5-{5-Chloro-4-[(3-methyl-pyridin-2-ylmethyl)-amino]-pyrimidin-2-ylamino}-1,3-dihydro-indol-2-one;

5-[5-Chloro-4-(3-methanesulfonyl-benzylamino)-pyrimidin-2-ylamino]-1,3-dihydro-indol-2-one;

6-[5-Chloro-4-(3-methyl-benzylamino)-pyrimidin-2-ylamino]-1,3-dihydro-indol-2-one;

5-[5-Chloro-4-(2-fluoro-benzylamino)-pyrimidin-2-ylamino]-1,3-dihydro-indol-2-one;

6-[5-Chloro-4-(2-fluoro-benzylamino)-pyrimidin-2-ylamino]-1,3-dihydro-indol-2-one;

5-[5-Bromo-4-(2-methoxy-ethylamino)-pyrimidin-2-ylamino]-1,3-dihydro-indol-2-one;

5-[5-Chloro-4-(3-methyl-benzylamino)-pyrimidin-2-ylamino]-1,3-dihydro-indol-2-one;

6-{5-Chloro-4-[(4-methyl-pyridin-2-ylmethyl)-amino]-pyrimidin-2-ylamino}-1,3-dihydro-indol-2-one;

5-(4-Benzylamino-5-chloro-pyrimidin-2-ylamino)-1,3-dihydro-indol-2-one;

5-Bromo-N2-(1H-indol-5-yl)-N4-pyridin-2-ylmethyl-pyrimidine-2,4-diamine;

5-Bromo-N2-(1H-indol-5-yl)-N4-(2-pyridin-2-yl-ethyl)-pyrimidine-2,4-diamine;

5-Bromo-N2-(1H-indol-4-yl)-N4-(2-pyridin-2-yl-ethyl)-pyrimidine-2,4-diamine;

5-Bromo-N2-(1H-indazol-5-yl)-N4-(2-pyridin-2-yl-ethyl)-pyrimidine-2,4-diamine;

5-Bromo-N2-(1H-indazol-6-yl)-N4-(2-pyridin-2-yl-ethyl)-pyrimidine-2,4-diamine;

5-Bromo-N2-(1H-indol-4-yl)-N4-pyridin-2-ylmethyl-pyrimidine-2,4-diamine;

5-Bromo-N2-(1H-indazol-5-yl)-N4-pyridin-2-ylmethyl-pyrimidine-2,4-diamine;

N2-(1H-Indol-5-yl)-N4-pyridin-2-ylmethyl-pyrimidine-2,4-diamine;

N2-(1H-Indazol-6-yl)-N4-pyridin-2-ylmethyl-pyrimidine-2,4-diamine;

N2-(1H-Indol-5-yl)-N4-(2-pyridin-2-yl-ethyl)-pyrimidine-2,4-diamine;

N2-(1H-Indazol-5-yl)-N4-pyridin-2-ylmethyl-pyrimidine-2,4-diamine;

N2-(1H-Indazol-5-yl)-N4-(2-pyridin-2-yl-ethyl)-pyrimidine-2,4-diamine;

N2-(1H-Indazol-6-yl)-N4-(2-pyridin-2-yl-ethyl)-pyrimidine-2,4-diamine;

5-{5-Bromo-4-[(pyridin-2-ylmethyl)-amino]-pyrimidin-2-ylamino}-1,3-dihydro-benzoimidazol-2-one;

5-[5-Bromo-4-(2-pyridin-2-yl-ethylamino)-pyrimidin-2-ylamino]-1,3-dihydro-benzoimidazol-2-one;

5-{4-[(Pyridin-2-ylmethyl)-amino]-pyrimidin-2-ylamino}-1,3-dihydro-benzoimidazol-2-one;

5-[4-(2-Pyridin-2-yl-ethylamino)-pyrimidin-2-ylamino]-1,3-dihydro-benzoimidazol-2-one;

5-Bromo-N2-(1H-indazol-6-yl)-N4-pyridin-2-ylmethyl-pyrimidine-2,4-diamine;

5-{5-Bromo-4-[(pyridin-2-ylmethyl)-amino]-pyrimidin-2-ylamino}-1,3-dihydro-indol-2-one;

5-[5-Bromo-4-(2-pyridin-2-yl-ethylamino)-pyrimidin-2-ylamino]-1,3-dihydro-indol-2-one;

5-[4-(2-Pyridin-2-yl-ethylamino)-pyrimidin-2-ylamino]-1,3-dihydro-indol-2-one;

5-Bromo-N2-(2-methyl-1H-indol-5-yl)-N4-(2-pyridin-2-yl-ethyl)-pyrimidine-2,4-diamine;

N2-(2-Methyl-1H-indol-5-yl)-N4-pyridin-2-ylmethyl-pyrimidine-2,4-diamine;

N2-(1H-Indol-6-yl)-N4-pyridin-72-ylmethyl-pyrimidine-2,4-diamine;

5-Bromo-N2-(2-methyl-1H-indol-5-yl)-N4-pyridin-2-ylmethyl-pyrimidine-2,4-diamine;

5-Bromo-N2-(1H-indol-6-yl)-N4-pyridin-2-ylmethyl-pyrimidine-2,4-diamine;

5-Bromo-N2-(1H-indol-6-yl)-N4-(2-pyridin-2-yl-ethyl)-pyrimidine-2,4-diamine;

N2-(1H-Benzoimidazol-5-yl)-5-bromo-N4-pyridin-2-ylmethyl-pyrimidine-2,4-diamine;

N2-(1H-Benzoimidazol-5-yl)-5-bromo-N4-(2-pyridin-2-yl-ethyl)-pyrimidine-2,4-diamine;

3-[5-Bromo-4-(2-pyridin-2-yl-ethylamino)-pyrimidin-2-yl]-3H-benzoimidazol 5-ylamine

N2-(1H-Benzoimidazol-5-yl)-N4-pyridin-2-ylmethyl-pyrimidine-2,4-diamine;

5-Bromo-N2-(2-methyl-1H-benzoimidazol-5-yl)-N4-pyridin-2-ylmethyl-pyrimidine-2,4-diamine;

N2-(2-Methyl-1H-benzoimidazol-5-yl)-N4-pyridin-2-ylmethyl-pyrimidine-2,4-diamine;

5-Bromo-N2-(2-methyl-1H-benzoimidazol-5-yl)-N4-(2-pyridin-2-yl-ethyl)-pyrimidine-2,4-diamine;

5-Bromo-N2-(2,3-dihydro-1H-indol-5-yl)-N4-(2-pyridin-2-yl-ethyl)-pyrimidine-2,4-diamine;

N2-(2,3-Dihydro-1H-indol-5-yl)-N4-pyridin-2-ylmethyl-pyrimidine-2,4-diamine;

5-Bromo-N2-(1-methyl-1H-indol-5-yl)-N4-(2-pyridin-2-yl-ethyl)-pyrimidine-2,4-diamine;

N2-(1-Methyl-1H-indol-5-yl)-N4-pyridin-2-ylmethyl-pyrimidine-2,4-diamine;

5-Bromo-N2-(2,3-dihydro-1H-indol-5-yl)-N4-pyridin-2-ylmethyl-pyrimidine-2,4-diamine;

5-Bromo-N2-(1-methyl-1H-indol-5-yl)-N4-pyridin-2-ylmethyl-pyrimidine-2,4-diamine;

5-Fluoro-N4-pyridin-2-ylmethyl-N2-quinolin-6-yl-pyrimidine-2,4-diamine;

5-Bromo-N4-pyridin-2-ylmethyl-N2-quinolin-6-yl-pyrimidine-2,4-diamine;

5-Bromo-N2-(1H-indol-7-yl)-N4-(2-pyridin-2-yl-ethyl)-pyrimidine-2,4-diamine;

5-Bromo-N2-(1H-indol-7-yl)-N4-pyridin-2-ylmethyl-pyrimidine-2,4-diamine;

5-Bromo-N2-(1H-indazol-4-yl)-N4-pyridin-2-ylmethyl-pyrimidine-2,4-diamine;

6-{5-Bromo-4-[(pyridin-2-ylmethyl)-amino]-pyrimidin-2-ylamino}-1,3-dihydro-indol-2-one;

5-Bromo-N2-(1H-indazol-4-yl)-N4-(2-pyridin-2-yl-ethyl)-pyrimidine-2,4-diamine;

5-Bromo-N4-(2-pyridin-2-yl-ethyl)-N2-quinolin-6-yl-pyrimidine-2,4-diamine;

5-Bromo-N4-pyridin-2-ylmethyl-N2-quinolin-5-yl-pyrimidine-2,4-diamine;

5-Bromo-N4-(2-pyridin-2-yl-ethyl)-N2-quinolin-5-yl-pyrimidine-2,4-diamine;

6-[5-Bromo-4-(2-pyridin-2-yl-ethylamino)-pyrimidin-2-ylamino]-1,3-dihydro-indol-2-one;

5-Bromo-N4-pyridin-2-ylmethyl-N2-quinolin-8-yl-pyrimidine-2,4-diamine;

5-Bromo-N4-(2-pyridin-2-yl-ethyl)-N2-quinolin-8-yl-pyrimidine-2,4-diamine;

5-{5-Bromo-4-[(pyridin-2-ylmethyl)-amino]-pyrimidin-2-ylamino}-1H-indole-2-carboxylic acid; ethyl ester;

6-[5-Bromo-4-(2-trifluoromethyl-benzylamino)-pyrimidin-2-ylamino]-1,3-dihydro-indol-2-one;

5-Bromo-N2-(1H-indazol-5-yl)-N4-(2-trifluoromethyl-benzyl)-pyrimidine-2,4-diamine;

5-Bromo-N2-(1H-indazol-6-yl)-N4-(2-trifluoromethyl-benzyl)-pyrimidine-2,4-diamine;

5-Bromo-N2-(1-methyl-1H-indol-5-yl)-N4-(2-trifluoromethyl-benzyl)-pyrimidine-2,4-diamine;

5-Bromo-N2-(1H-indazol-7-yl)-N4-pyridin-2-ylmethyl-pyrimidine-2,4-diamine;

5-Bromo-N2-(1H-indazol-4-yl)-N4-(2-trifluoromethyl-benzyl)-pyrimidine-2,4-diamine;

6-{5-Bromo-4-[(pyridin-2-ylmethyl)-amino]-pyrimidin-2-ylamino}-3H-isobenzofuran-1-one;

N2-Benzothiazol-6-yl-5-bromo-N4-pyridin-2-ylmethyl-pyrimidine-2,4-diamine;

5-{5-Bromo-4-[(pyridin-2-ylmethyl)-amino]-pyrimidin-2-ylamino}-2-methyl-1H-indole-3-carbonitrile

5-Bromo-N4-pyridin-2-ylmethyl-N2-(1-pyridin-2-ylmethyl-1H-indazol-5-yl)-pyrimidine-2,4-diamine;

N2-(1-Benzyl-1H-indol-5-yl)-5-bromo-N4-pyridin-2-ylmethyl-pyrimidine-2,4-diamine;

5-Bromo-N4-pyridin-2-ylmethyl-N2-(1-pyridin-2-ylmethyl-1H-indol-5-yl)-pyrimidine-2,4-diamine;

N2-(1-Benzyl-1H-indazol-5-yl)-5-bromo-N4-pyridin-2-ylmethyl-pyrimidine-2,4-diamine;

5-Bromo-N2-(1-methyl-1H-indazol-5-yl)-N4-pyridin-2-ylmethyl-pyrimidine-2,4-diamine;

5-Bromo-N4-(4-methyl-cyclohexyl)-N2-[3-(1,2,3,6-tetrahydro-pyridin-4-yl)-1H-indol-5-yl]-pyrimidine-2,4-diamine;

5-Bromo-N4-(4-methyl-cyclohexyl)-N2-[3-(1,2,3,6-tetrahydro-pyridin-4-yl)-1H-indol-5-yl]-pyrimidine-2,4-diamine;

5-Bromo-N4-cyclohexylmethyl-N2-[3-(1,2,3,6-tetrahydro-pyridin-4-yl)-1H-indol-5-yl]-pyrimidine-2,4-diamine;

1-{5-Fluoro-4-[(pyridin-2-ylmethyl)-amino]-pyrimidin-2-yl}-3-(1,2,3,6-tetrahydro-pyridin-4-yl)-1H-indol-5-ylamine

1-{5-Chloro-4-[(pyridin-2-ylmethyl)-amino]-pyrimidin-2-yl}-3-(1,2,3,6-tetrahydro-pyridin-4-yl)-1H-indol-5-ylamine

5-Fluoro-N2-(1H-indazol-5-yl)-N4-pyridin-2-ylmethyl-pyrimidine-2,4-diamine;

5-{5-Fluoro-4-[(pyridin-2-ylmethyl)-amino]-pyrimidin-2-ylamino}-1,3-dihydro-indol-2-one;

5-Chloro-N2-(1H-indazol-5-yl)-N4-pyridin-2-ylmethyl-pyrimidine-2,4-diamine;

5-{5-Chloro-4-[(pyridin-2-ylmethyl)-amino]-pyrimidin-2-ylamrino}-1,3-dihydro-indol-2-one;

5-Fluoro-N4-(2-pyridin-2-yl-ethyl)-N2-[3-(1,2,3,6-tetrahydro-pyridin-4-yl)-1H-indol-5-yl]-pyrimidine-2,4-diamine;

5-Chloro-N4-(2-pyridin-2-yl-ethyl)-N2-[3-(1,2,3,6-tetrahydro-pyridin-4-yl)-1H-indol-5-yl]-pyrimidine-2,4-diamine;

5-Fluoro-N2-(1H-indazol-5-yl)-N4-(2-pyridin-2-yl-ethyl)-pyrimidine-2,4-diamine;

5-[5-Fluoro-4-(2-pyridin-2-yl-ethylamino)-pyrimidin-2-ylamino]-1,3-dihydro-indol-2-one;

5-Chloro-N2-(1H-indazol-5-yl)-N4-(2-pyridin-2-yl-ethyl)-pyrimidine-2,4-diamine;

5-[5-Chloro-4-(2-pyridin-2-yl-ethylamino)-pyrimidin-2-yiamino]-1,3-dihydro-indol-2-one;

5-{4-[(Pyridin-2-ylmethyl)-amino]-5-trifluoromethyl-pyrimidin-2-ylamino}-1,3-dihydro-indol-2-one;

5-{5-Methoxy-4-[(pyridin-2-ylmethyl)-amino]-pyrimidin-2-ylamino}-1,3-dihydro-indol-2-one;

5-[5-Methoxy-4-(2-pyridin-2-yl-ethylamino)-pyrimidin-2-ylamino]-1,3-dihydro-indol-2-one;

5-[5-Methoxy-4-(2-trifluoromethyl-benzylamino)-pyrimidin-2-ylamino]-1,3-dihydro-indol-2-one;

5-{5-Bromo-4-[(cyclohex-1-enylmethyl)-amino]-pyrimidin-2-yiamino}-1,3-dihydro-indol-2-one;

5-[5-Bromo-4-(methyl-pyridin-2-ylmethyl-amino)-pyrimidin-2-ylamino]-1,3-dihydro-indol-2-one;

5-[5-Bromo-4-(4-methyl-cyclohexylamino)-pyrimidin-2-ylamino]-1,3-dihydro-indol-2-one;

5-[5-Bromo-4-(4-methyl-cyclohexylamino)-pyrimidin-2-ylamino]-1,3-dihydro-indol-2-one;

5-[5-Bromo-4-(cyclohexylmethyl-amino)-pyrimidin-2-ylamino]-1,3-dihydro-indol-2-one;

5-[5-Chloro-4-(2-trifluoromethyl-benzylamino)-pyrimidin-2-ylamino]-1,3-dihydro-indol-2-one;

2-(2-Oxo-2,3-dihydro-1H-indol-5-ylamino)-4-[(pyridin-2-ylmethyl)-amino]-pyrimidine-5-carbonitrile

5-{5-Methyl-4-[(pyridin-2-ylmethyl)-amino]-pyrimidin-2-ylamino}-1,3-dihydro-indol-2-one;

N2-(1H-Indazol-5-yl)-5-methyl-N4-pyridin-2-ylmethyl-pyrimidine-2,4-diamine;

5-Fluoro-N4-pyridin-2-ylmethyl-N2-[3-(1,2,3,6-tetrahydro-pyridin-4-yl)-1H-indol-5-yl]-pyrimidine-2,4-diamine;

5-Chloro-N4-pyridin-2-ylmethyl-N2-[3-(1,2,3,6-tetrahydro-pyridin-4-yl)-1H-indol-5-yl]-pyrimidine-2,4-diamine;

2-(2-Oxo-2,3-dihydro-1H-indol-5-ylamino)-4-(2-trifluoromethyl-benzylamino)-pyrimidine-5-carbonitrile

5-{4-[Methyl-(2-pyridin-2-yl-ethyl)-amino]-pyrimidin-2-ylamino}-1,3-dihydro-indol-2-one;

5-Bromo-N4-cyclohex-1-enylmethyl-N2-[3-(1,2,3,6-tetrahydro-pyridin-4-yl)-1H-indol-5-yl]-pyrimidine-2,4-diamine;

N2-(1H-Indazol-5-yl)-N4-pyridin-2-ylmethyl-5-trifluoromethyl-pyrimidine-2,4-diamine;

5-[5-Trifluoromethyl-4-(2-trifluoromethyl-benzylamino)-pyrimidin-2-ylamino]-1,3-dihydro-indol-2-one;

6-{2-[(Pyridin-2-ylmethyl)-amino]-5-trifluoromethyl-pyrimidin-4-ylamino}-1,3-dihydro-indol-2-one;

5-[5-Bromo-4-(piperidin-4-ylamino)-pyrimidin-2-ylamino]-1,3-dihydro-indol-2-one;

5-[4-(1-Acetyl-piperidin-4-ylamino)-5-bromo-pyrimidin-2-ylamino]-1,3-dihydro-indol-2-one;

2-(2-Oxo-2,3-dihydro-1H-indol-6-ylamino)-4-[(pyridin-2-ylmethyl)-amino]-pyrimidine-5-carbonitrile

5-{4-[(3-Methyl-pyridin-2-ylmethyl)-amino]-5-trifluoromethyl-pyrimidin-2-ylamino}-1,3-dihydro-indol-2-one;

6-{4-[(3-Methyl-pyridin-2-ylmethyl)-amino]-5-trifluoromethyl-pyrimidin-2-ylamino}-1,3-dihydro-indol-2-one;

4-[5-Bromo-2-(2-oxo-2,3-dihydro-1H-indol-5-ylamino)-pyrimidin-4-ylamino]-piperidine-1-carboxylic acid; tert-butyl ester;

5-[5-Bromo-4-(1-methanesulfonyl-piperidin-4-ylamino)-pyrimidin-2-ylamino]-1,3-dihydro-indol-2-one;

5-[5-Bromo-4-(piperidin-3-ylamino)-pyrimidin-2-ylamino]-1,3-dihydro-indol-2-one;

4-[5-Bromo-2-(2-oxo-2,3-dihydro-1H-indol-5-ylamino)-pyrimidin-4-ylamino]-piperidine-1-carboxylic acid; ethylamide

3-[5-Bromo-2-(2-oxo-2,3-dihydro-1H-indol-5-ylamino)-pyrimidin-4-ylamino]-piperidine-1-carboxylic acid; ethylamide

5-[4-(1-Benzoyl-piperidin-4-ylamino)-5-bromo-pyrimidin-2-ylamino]-1,3-dihydro-indol-2-one;

6-[4-(3-Methanesulfonyl-benzylamino)-5-methoxy-pyrimidin-2-ylamino]-1,3-dihydro-indol-2-one;

6-[4-(3-Methanesulfonyl-benzylamino)-5-trifluoromethyl-pyrimidin-2-ylamino]-1,3-dihydro-indol-2-one;

6-[4-(3-Methanesulfonyl-benzylamino)-pyrimidin-2-ylamino]-1,3-dihydro-indol-2-one;

5-[4-(1-Benzenesulfonyl-piperidin-4-ylamino)-5-bromo-pyrimidin-2-ylamino]-1,3-dihydro-indol-2-one;

5-[4-(3-Methanesulfonyl-benzylamino)-5-trifluoromethyl-pyrimidin-2-ylamino]-1,3-dihydro-indol-2-one;

6-{5-Chloro-4-[(piperidin-3-ylmethyl)-amino]-pyrimidin-2-ylamino}-1,3-dihydro-indol-2-one;

6-{5-Chloro-4-[(1-methanesulfonyl-piperidin-3-ylmethyl)-amino]-pyrimidin-2-ylamino}-1,3-dihydro-indol-2-one;

6-{5-Bromo-4-[(piperidin-3-ylmethyl)-amino]-pyrimidin-2-ylamino}-1,3-dihydro-indol-2-one;

6-{5-Bromo-4-[(1-methanesulfonyl-piperidin-3-ylmethyl)-amino]-pyrimidin-2-ylamino}-1,3-dihydro-indol-2-one;

5-[5-Fluoro-4-(3-methanesulfonyl-benzylamino)-pyrimidin-2-ylamino]-1,3-dihydro-indol-2-one;

5-{5-Bromo-4-[(1-hydroxy-cyclohexylmethyl)-amino]-pyrimidin-2-yiamino}-1,3-dihydro-indol-2-one; and pharmaceutically acceptable salt, prodrug, hydrate or solvate of the aforementioned compounds

This invention also relates to a method for the treatment of abnormal cell growth in a mammal, including a human, comprising administering to said mammal an amount of a compound of the formula 1, as defined above, or a pharmaceutically acceptable salt, solvate or prodrug thereof, that is effective in treating abnormal cell growth. In one embodiment of this method, the abnormal cell growth is cancer, including, but not limited to, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. In one embodiment the method comprises comprising administering to a mammal an amount of a compound of formula 1 that is effective in treating said cancer solid tumor. In one preferred embodiment the solid tumor is breast, lung, colon, brain, prostate, stomach, pancreatic, ovarian, skin (melanoma), endocrine, uterine, testicular, and bladder cancer.

In another embodiment of said method, said abnormal cell growth is a benign proliferative disease, including, but not limited to, psoriasis, benign prostatic hypertrophy or restinosis.

This invention also relates to a method for the treatment of abnormal cell growth in a mammal which comprises administering to said mammal an amount of a compound of formula 1, or a pharmaceutically acceptable salt, solvate or prodrug thereof, that is effective in treating abnormal cell growth in combination with an anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, antibodies, cytotoxics, anti-hormones, and anti-androgens.

This invention also relates to a pharmaceutical composition for the treatment of abnormal cell growth in a mammal, including a human, comprising an amount of a compound of the formula 1, as defined above, or a pharmaceutically acceptable salt, solvate or prodrug thereof, that is effective in treating abnormal cell growth, and a pharmaceutically acceptable carrier. In one embodiment of said composition, said abnormal cell growth is cancer, including, but not limited to, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. In another embodiment of said pharmaceutical composition, said abnormal cell growth is a benign proliferative disease, including, but not limited to, psoriasis, benign prostatic hypertrophy or restinosis.

The invention also relates to a pharmaceutical composition for the treatment of abnormal cell growth in a mammal, including a human, which comprises an amount of a compound of formula 1, as defined above, or a pharmaceutically acceptable salt, solvate or prodrug thereof, that is effective in treating abnormal cell growth in combination with a pharmaceutically acceptable carrier and an anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, and anti-androgens.

This invention also relates to a method for the treatment of a disorder associated with angiogenesis in a mammal, including a human, comprising administering to said mammal an amount of a compound of the formula 1, as defined above, or a pharmaceutically acceptable salt, solvate or prodrug thereof, that is effective in treating said disorder. Such disorders include cancerous tumors such as melanoma; ocular disorders such as age-related macular degeneration, presumed ocular histoplasmosis syndrome, and retinal neovascularization from proliferative diabetic retinopathy; rheumatoid arthritis; bone loss disorders such as osteoporosis, Paget's disease, humoral hypercalcemia of malignancy, hypercalcemia from tumors metastatic to bone, and osteoporosis induced by glucocorticoid treatment; coronary restenosis; and certain microbial infections including those associated with microbial pathogens selected from adenovirus, hantaviruses, Borrelia burgdorferi, Yersinia spp., Bordetella pertussis, and group A Streptococcus.

This invention also relates to a method of (and to a pharmaceutical composition for) treating abnormal cell growth in a mammal which comprise an amount of a compound of formula 1, or a pharmaceutically acceptable salt, solvate or prodrug thereof, and an amount of one or more substances selected from anti-angiogenesis agents, signal transduction inhibitors, and antiproliferative agents, which amounts are together effective in treating said abnormal cell growth.

Anti-angiogenesis agents, such as MMP-2 (matrix-metalloprotienase 2) inhibitors, MMP-9 (matrix-metalloprotienase 9) inhibitors, and COX-II (cyclooxygenase II) inhibitors, can be used in conjunction with a compound of formula 1 in the methods and pharmaceutical compositions described herein. Examples of useful COX-II inhibitors include CELEBREX™ (alecoxib), valdecoxib, and rofecoxib. Examples of useful matrix metalloproteinase inhibitors are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931,788 (published Jul. 28, 1999), WO 90/05719 (published May 331, 1990), WO 99/52910 (published Oct. 21, 1999), WO 99/52889 (published Oct. 21, 1999), WO 99/29667 (published Jun. 17, 1999), PCT International Application No. PCT/IB98/01113 (filed Jul. 21, 1998), European Patent Application No. 99302232.1 (filed Mar. 25, 1999), Great Britain patent application number 9912961.1 (filed Jun. 3, 1999), U.S. Provisional Application No. 60/148,464 (filed Aug. 12, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997), all of which are herein incorporated by reference in their entirety. Preferred MMP-2 and MMP-9 inhibitors are those that have little or no activity inhibiting MMP-1. More preferred, are those that selectively inhibit MMP-2 and/or MMP-9 relative to the other matrix-metalloproteinases (i.e. MMP-1, MMP-3, MMP-4, MMP-5, MMP-6, MMP-7, MMP-8, MMP-10, MMP-11, MMP-12, and MMP-13).

Some specific examples of MMP inhibitors useful in combination with the compounds of the present invention are AG-3340, RO 32-3555, RS 13-0830, and the compounds recited in the following list:

3-[[4-(4-fluoro-phenoxy)-benzenesulfonyl]-(1-hydroxycarbamoyl-cyclopentyl)-amino]-propionic acid;

3-exo-3-[4-(4-fluoro-phenoxy)-benzenesulfonylamino]-8-oxa-bicyclo[3.2.1]octane-3-carboxylic acid hydroxyamide;

(2R,3R) 1-[4-(2-chloro-4-fluoro-benzyloxy)-benzenesulfonyl]-3-hydroxy-3-methyl-piperidine-2-carboxylic acid hydroxyamide;

4-[4-(4-fluoro-phenoxy)-benzenesulfonylamino]-tetrahydro-pyran-4-carboxylic acid hydroxyamide;

3-[[4-(4-fluoro-phenoxy)-benzenesulfonyl]-(1-hydroxycarbamoyl-cyclobutyl)-amino]-propionic acid;

4-[4-(4-chloro-phenoxy)-benzenesulfonylamino]-tetrahydro-pyran-4-carboxylic acid hydroxyamide;

3-[4-(4-chloro-phenoxy)-benzenesulfonylamino]-tetrahydro-pyran-3-carboxylic acid hydroxyamide;

(2R,3R) 1-[4-(4-fluoro-2-methyl-benzyloxy)-benzenesulfonyl]-3-hydroxy-3-methyl-piperidine-2-carboxylic acid hydroxyamide;

3-[[4-(4-fluoro-phenoxy)-benzenesulfonyl]-(1-hydroxycarbamoyl-1-methyl-ethyl)-amino]-propionic acid;

3-[[4-(4-fluoro-phenoxy)-benzenesulfonyl]-(4-hydroxycarbamoyl-tetrahydro-pyran-4-yl)-amino]-propionic acid;

3-exo-3-[4-(4-chloro-phenoxy)-benzenesulfonylamino]-8-oxa-bicyclo[3.2.1]octane-3-carboxylic acid hydroxyamide;

3-endo-3-[4-(4-fluoro-phenoxy)-benzenesulfonylamino]-8-oxa-bicyclo[3.2.1]octane-3-carboxylic acid hydroxyamide; and

3-[4-(4-fluoro-phenoxy)-benzenesulfonylamino]-tetrahydro-furan-3-carboxylic acid hydroxyamide;

and pharmaceutically acceptable salts, solvates and prodrugs of said compounds.

The compounds of formula 1, and the pharmaceutically acceptable salts, solvates and prodrugs thereof, can also be used in combination with signal transduction inhibitors, such as agents that can inhibit EGFR (epidermal growth factor receptor) responses, such as EGFR antibodies, EGF antibodies, and molecules that are EGFR inhibitors; VEGF (vascular endothelial growth factor) inhibitors; and erbB2 receptor inhibitors, such as organic molecules or antibodies that bind to the erbB2 receptor, for example, HERCEPTIN™ (Genentech, Inc. of South San Francisco, Calif., USA).

EGFR inhibitors are described in, for example in WO 95/19970 (published Jul. 27, 1995), WO 98/14451 (published Apr. 9, 1998), WO 98/02434 (published Jan. 22, 1998), and U.S. Pat. No. 5,747,498 (issued May 5, 1998). EGFR-inhibiting agents include, but are not limited to, CI-1033 (Pfizer Inc.), the monoclonal antibodies C225 and anti-EGFR 22Mab (ImClone Systems Incorporated of New York, N.Y., USA), the compounds ZD-1839 (AstraZeneca), BIBX-1382 (Boehringer Ingelheim), MDX-447 (Medarex Inc. of Annandale, N.J., USA), and OLX-103 (Merck & Co. of Whitehouse Station, N.J., USA), VRCTC-310 (Ventech Research) and EGF fusion toxin (Seragen Inc. of Hopkinton, Massachusettes).

VEGF inhibitors, for example CP-547,632 and AG-13736 (Pfizer, Inc.), SU-5416 and SU-6668 (Sugen Inc. of South San Francisco, Calif., USA), can also be combined with a compound of formula 1. VEGF inhibitors are described in, for example in WO 99/24440 (published May 20, 1999), PCT International Application PCT/IB99/00797 (filed May 3, 1999), in WO 95/21613 (published Aug. 17, 1995), WO 99/61422 (published Dec. 2, 1999), U.S. Pat. No. 5,834,504 (issued Nov. 10, 1998), WO 98/50356 (published Nov. 12, 1998), U.S. Pat. No. 5,883,113 (issued Mar. 16, 1999), U.S. Pat. No. 5,886,020 (issued Mar. 23, 1999), U.S. Pat. No. 5,792,783 (issued Aug. 11, 1998), WO 99/10349 (published Mar. 4, 1999), WO 97/32856 (published Sep. 12, 1997), WO 97/22596 (published Jun. 26, 1997), WO 98/54093 (published Dec. 3, 1998), WO 98/02438 (published Jan. 22, 1998), WO 99/16755 (published Apr. 8, 1999), and WO 98/02437 (published Jan. 22, 1998), all of which are herein incorporated by reference in their entirety. Other examples of some specific VEGF inhibitors are IM862 (Cytran Inc. of Kirkland, Wash., USA); anti-VEGF monoclonal antibody of Genentech, Inc. of South San Francisco, Calif.; and angiozyme, a synthetic ribozyme from Ribozyme (Boulder, Colo.) and Chiron (Emeryville, Calif.).

ErbB2 receptor inhibitors, such as CP-724,714 (Pfizer, Inc.), GW-282974 (Glaxo Wellcome plc), and the monoclonal antibodies AR-209 (Aronex Pharmaceuticals Inc. of The Woodlands, Tex., USA) and 2B-1 (Chiron), may be administered in combination with a compound of formula 1. Such erbB2 inhibitors include those described in WO 98/02434 (published Jan. 22, 1998), WO 99/35146 (published Jul. 15, 1999), WO 99/35132 (published Jul. 15, 1999), WO 98/02437 (published Jan. 22, 1998), WO 97/13760 (published Apr. 17, 1997), WO 95/19970 (published Jul. 27, 1995), U.S. Pat. No. 5,587,458 (issued Dec. 24, 1996), and U.S. Pat. No. 5,877,305 (issued Mar. 2, 1999), each of which is herein incorporated by reference in its entirety. ErbB2 receptor inhibitors useful in the present invention are also described in U.S. Provisional Application No. 60/117,341, filed Jan. 27, 1999, and in U.S. Provisional Application No. 60/117,346, filed Jan. 27, 1999, both of which are herein incorporated by reference in their entirety.

Other antiproliferative agents that may be used with the compounds of the present invention include inhibitors of HDI (CI-994, Pfizer Inc.), MEK (CI-1040, Pfizer Inc.), the enzyme farnesyl protein transferase and the receptor tyrosine kinase PDGFr, including the compounds disclosed and claimed in the following United States patent applications: Ser. No. 09/221,946 (filed Dec. 28, 1998); Ser. No. 09/454,058 (filed Dec. 2, 1999); Ser. No. 09/501,163 (filed Feb. 9, 2000); Ser. No. 09/539,930 (filed Mar. 31, 2000); Ser. No. 09/202,796 (filed May 22, 1997); Ser. No. 09/384,339 (filed Aug. 26, 1999); and Ser. No. 09/383,755 (filed Aug. 26, 1999); and the compounds disclosed and claimed in the following United States provisional patent applications: 60/168,207 (filed Nov. 30, 1999); 60/170,119 (filed Dec. 10, 1999); 60/177,718 (filed Jan. 21, 2000); 60/168,217 (filed Nov. 30, 1999), and 60/200,834 (filed May 1, 2000). Each of the foregoing patent applications and provisional patent applications is herein incorporated by reference in'their entirety.

A compound of formula 1 may also be used with other agents useful in treating abnormal cell growth or cancer, including, but not limited to, agents capable of enhancing antitumor immune responses, such as CTLA4 (cytotoxic lymphocite antigen 4) antibodies, and other agents capable of blocking CTLA4; and anti-proliferative agents such as other farnesyl protein transferase inhibitors, for example the farnesyl protein transferase inhibitors described in the references cited in the “Background” section, supra. Specific CTLA4 antibodies that can be used in the present invention include those described in United States Provisional Application 60/113,647 (filed Dec. 23, 1998) which is herein incorporated by reference in its entirety.

“Abnormal cell growth”, as used herein, unless otherwise indicated, refers to cell growth that is independent of normal regulatory mechanisms (e.g., loss of contact inhibition). This includes the abnormal growth of: (1) tumor cells (tumors) that proliferate by expressing a mutated tyrosine kinase or overexpression of a receptor tyrosine kinase; (2) benign and malignant cells of other proliferative diseases in which aberrant tyrosine kinase activation occurs; (4) any tumors that proliferate by receptor tyrosine kinases; (5) any tumors that proliferate by aberrant serine/threonine kinase activation; and (6) benign and malignant cells of other proliferative diseases in which aberrant serine/threonine kinase activation occurs.

The term “treating”, as used herein, unless otherwise indicated, means reversing, alleviating, inhibiting the progress of, or preventing the disorder or condition to which such term applies, or one or more symptoms of such disorder or condition. The term ‘treatment’, as used herein, unless otherwise indicated, refers to the act of treating as “treating” is defined immediately above.

The term “halo”, as used herein, unless otherwise indicated, includes fluoro, chloro, bromo or iodo. Preferred halo groups are fluoro and chloro.

The term “alkyl”, as used herein, unless otherwise indicated, includes saturated monovalent hydrocarbon radicals having straight, cyclic (including mono- or multi-cyclic moieties) or branched moieties. It is understood that for said alkyl group to include cyclic moieties it must contain at least three carbon atoms.

The term “cycloalkyl”, as used herein, unless otherwise indicated, includes saturated monovalent hydrocarbon radicals having cyclic (including mono- or multi-cyclic) moieties.

The term “alkenyl”, as used herein, unless otherwise indicated, includes alkyl groups, as defined above, having at least one carbon-carbon double bond.

The term “alkynyl”, as used herein, unless otherwise indicated, includes alkyl groups, as defined above, having at least one carbon-carbon triple bond.

The term “aryl”, as used herein, unless otherwise indicated, includes an organic radical derived from an aromatic hydrocarbon by removal of one hydrogen, such as phenyl or naphthyl.

The term “alkoxy”, as used herein, unless otherwise indicated, includes —O-alkyl groups wherein alkyl is as defined above.

The term “4 to 10 membered heterocyclic”, as used herein, unless otherwise indicated, includes aromatic and non-aromatic heterocyclic groups containing one or more heteroatoms each selected from O, S and N, wherein each heterocyclic group has from 4 to 10 atoms in its ring system. Non-aromatic heterocyclic groups include groups having only 4 atoms in their ring system, but aromatic heterocyclic groups must have at least 5 atoms in their ring system. The heterocyclic groups include benzo-fused ring systems and ring systems substituted with one or more oxo moieties. An example of a 4 membered heterocyclic group is azetidinyl (derived from azetidine). An example of a 5 membered heterocyclic group is thiazolyl and an example of a 10 membered heterocyclic group is quinolinyl. Examples of non-aromatic heterocyclic groups are pyrrolidinyl, tetrahydrofuranyl, tetrahydrothienyl, tetrahydropyranyt, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2 H-pyranyl, 4 H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3 H-indolyl and quinolizinyl. Examples of aromatic heterocyclic groups are pyridinyl, imidazolyl, pyrimidinyl, pyrazolyi, triazolyl, pyrazinyl, tetrazolyl, furyl, thienyl, isoxazolyi, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the compounds listed above, may be C-attached or N-attached where such is possible. For instance, a group derived from pyrrole may be pyrrol-1-yl (N-attached) or pyrrol-3-yl (C-attached).

The term “Me” means methyl, “Et” means ethyl, and “Ac” means acetyl.

In the definition of X¹ above, the —(CR¹R²)ₘ— and (CR¹⁶R¹⁷)ₖ moieties, and other similar moieties, as indicated above, may vary in their definition of R¹, R², R¹⁶ and R¹⁷ for each iteration of the subscript (ie, m, k, etc) above 1. Thus, —(CR¹R²)ₘ— may include —CH₂C(Me)(Et)- where m is 2.

The phrase “pharmaceutically acceptable salt(s)”, as used herein, unless otherwise indicated, includes salts of acidic or basic groups which may be present in the compounds of the present invention. The compounds of the present invention that are basic in nature are capable of forming a wide variety of salts with various inorganic and organic acids. The acids that may be used to prepare pharmaceutically acceptable acid addition salts of such basic compounds of are those that form non-toxic acid addition salts, i.e., salts containing pharmacologically acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and pamoate [i.e., 1,1′-methylene-bis-(2-hydroxy-3-naphthoate)] salts. The compounds of the present invention that include a basic moiety, such as an amino group, may form pharmaceutically acceptable salts with various amino acids, in addition to the acids mentioned above.

Those compounds of the present invention that are acidic in nature are capable of forming base salts with various pharmacologically acceptable cations. Examples of such salts include the alkali metal or alkaline earth metal salts and, particularly, the calcium, magnesium, sodium and potassium salts of the compounds of the present invention.

Certain functional groups contained within the compounds of the present invention can be substituted for bioisosteric groups, that is, groups which have similar spatial or electronic requirements to the parent group, but exhibit differing or improved physicochemical or other properties. Suitable examples are well known to those of skill in the art, and include, but are not limited to moieties described in Patini et al., Chem. Rev, 1996, 96, 3147-3176 and references cited therein.

The compounds of the present invention have asymmetric centers and therefore exist in different enantiomeric and diastereomeric forms. This invention relates to the use of all optical isomers and stereoisomers of the compounds of the present invention, and mixtures thereof, and to all pharmaceutical compositions and methods of treatment that may employ or contain them. The compounds of formula 1 may also exist as tautomers. This invention relates to the use of all such tautomers and mixtures thereof.

The subject invention also includes isotopically-labelled compounds, and the pharmaceutically acceptable salts, solvates and prodrugs thereof, which are identical to those recited in formula 1, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, ³H, ³C, ¹⁴C, ¹⁵N, ¹⁸O, ¹⁷O, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of formula 1 of this invention and prodrugs thereof can generally be prepared by carrying out the procedures disclosed in the Schemes and/or in the Examples and Preparations below, by substituting a readily available isotopically labelled reagent for a non-isotopically labelled reagent.

This invention also encompasses pharmaceutical compositions containing and methods of treating bacterial infections through administering prodrugs of compounds of the formula 1. Compounds of formula 1 having free amino, amido, hydroxy or carboxylic groups can be converted into prodrugs. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of formula 1. The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline homocysteine, homoserine, omithine and methionine sulfone. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10. Free amines can also be derivatized as amides, sulfonamides or phosphonamides. All of these prodrug moieties may incorporate groups including but not limited to ether, amine and carboxylic acid functionalities.

### DETAILED DESCRIPTION OF THE INVENTION

The compounds of formula 1 can be prepared using the following synthetic scheme 1. The substituents in scheme 1 have the same meaning as the substituents defined for formula 1. The substituent Lg in the compounds of formulas 2 and 4 is a leaving group. Leaving groups are well-known to those of ordinary skill in the art. Applicants also direct the reader's attention to the Experimental section for particular examples of leaving group employed in the preparation of the compounds of the present invention.

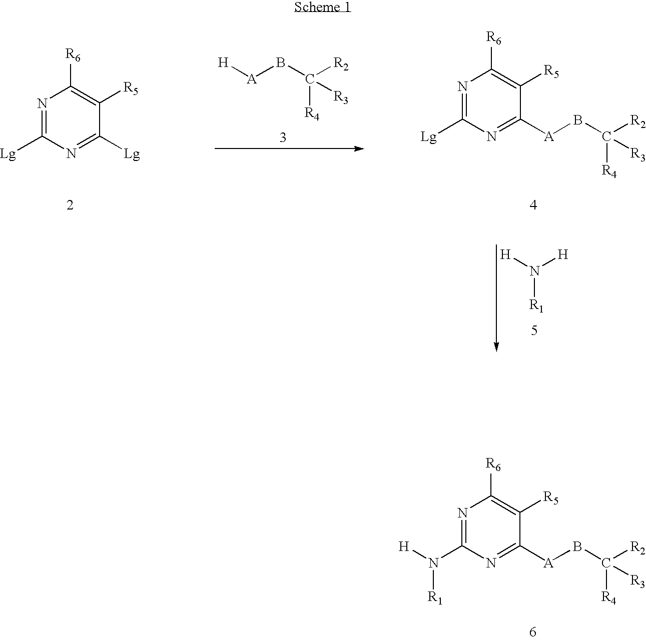

Necessary starting materials may be purchased and used directly or alternatively, starting materials can be prepared by one skilled in the art utilizing known procedures obtained from standard chemistry references (such as, Organic Synthesis (McGraw Hill) Michael Smith). It is understood that starting materials may be optionally protected as to not interfere with a desired chemical reaction (see Protecting Groups in Organic Synthesis (Wiley-Interscience), Green and Wutts). Subsequent de-protection of potentially interfering functional group may be effected at a later appropriate time to obtain the necessary desired material. A pyrimidine of the general formula I may be purchased or prepared from known materials by one skilled in the art. Lg is defined as a displaceable leaving group that includes halogens and sulfonyl groups.

Using methods known in the literature by one skilled in the art, a pyrimidine of formula 2 may be reacted together with a compound of formula 3, optionally in the presence of a suitable base and optionally in the presence of a suitable inert solvent and at a temperature range of 0° C. to 150° C. Suitable bases employed may be the following but not limited to (i) organic bases, for example triethyamine, or diisopropylethylamine and (ii) inorganic bases such as potassium carbonate or cesium carbonate. The reaction may be performed neat or carried out in a suitable inert solvent. Examples of suitable inert solvents are but not limited to tetrahydrofuran, 1,4-dioxane, dimethylformamide, n-methylpyrrolidin-2-one, ethanol, butanol, dichloromethane, or acetonitrile. Followed by the next reaction in which pyrimidine of formula 4 may be reacted together with amine compounds of formula IV optionally in the presence of a suitable base and optionally in the presence of a suitable inert solvent and at a temperature range of 0° C. to 150° C. conveniently at or near reflux to obtain compounds of formula 6. The reaction may be performed neat or optionally carried out in a suitable inert solvent. Examples of suitable inert solvents are but not limited to tetrahydrofuran, 1,4-dioxane, dimethylformamide, n-methylpyrrolidin-2-one, ethanol, butanol, dichloromethane, dimethyl sulfoxide or acetonitrile.

Compounds of formula 6, if optional protecting groups are present would be removed using standard techniques well-known to those of ordinary skill in the art, see for example, Protecting Groups in Organic Synthesis (Wiley-Interscience), Green and Wutts. These methods are known to those skilled in the art and include a) removal of a protecting group by methods outlined in T. W. Greene and P. G. M. Wuts, “Protective Groups in Organic Synthesis”, Second Edition, John Wiley and Sons, New York, 1991; b) displacement of a leaving group (halide, mesylate, tosylate, etc) with a primary or secondary amine, thiol or alcohol to form a secondary or tertiary amine, thioether or ether, respectively, c) treatment of phenyl (or substituted phenyl) carbamates with primary of secondary amines to form the corresponding ureas as in Thavonekham, B et. al. Synthesis (1997), 10, p1189; d) reduction of propargyl or homopropargyl alcohols or N-BOC protected primary amines to the corresponding E-allylic or E-homoallylic derivatives by treatment with sodium bis(2-methoxyethoxy)aluminum hydride (Red-Al) as in Denmark, S. E.; Jones, T. K. J. Org. Chem. (1982) 47, 4595-4597 or van Benthem, R. A. T. M.; Michels, J. J.; Speckamp, W. N. Synlett (1994), 368-370; e) reduction of alkynes to the corresponding Z-alkene derivatives by treatment hydrogen gas and a Pd catalyst as in Tomassy, B. et. al. Synth. Commun. (1998), 28, p1201 f) treatment of primary and secondary amines with an isocyanate, acid chloride (or other activated carboxylic acid derivative), alkyl/aryl chloroformate or sulfonyl chloride to provide the corresponding urea, amide, carbamate or sulfonamide; g) reductive amination of a primary or secondary amine using R¹CH(O); and h) treatment of alcohols with an isocyanate, acid chloride (or other activated carboxylic acid derivative), alkyl/aryl chloroformate or sulfonyl chloride to provide the corresponding carbamate, ester, carbonate or sulfonic acid ester.

The compounds of the present invention may have asymmetric carbon atoms. Diasteromeric mixtures can be separated into their individual diastereomers on the basis of their physical chemical differences by methods known to those skilled in the art, for example, by chromatography or fractional crystallization. Enantiomers can be separated by converting the enantiomeric mixtures into a diastereomric mixture by reaction with an appropriate optically active compound (e.g., alcohol), separating the diastereomers and converting (e.g., hydrolyzing) the individual diastereomers to the corresponding pure enantiomers. All such isomers, including diastereomeric mixtures and pure enantiomers are considered as part of the invention.

The compounds of formulas 1 that are basic in nature are capable of forming a wide variety of different salts with various inorganic and organic acids. Although such salts must be pharmaceutically acceptable for administration to animals, it is often desirable in practice to initially isolate the compound of formula 1 from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent, such as methanol or ethanol. Upon careful evaporation of the solvent, the desired solid salt is readily obtained. The desired acid salt can also be precipitated from a solution of the free base in an organic solvent by adding to the solution an appropriate mineral or organic acid.

Those compounds of formula 1 that are acidic in nature are capable of forming base salts with various pharmacologically acceptable cations. Examples of such salts include the alkali metal or alkaline-earth metal salts and particularly, the sodium and potassium salts. These salts are all prepared by conventional techniques. The chemical bases which are used as reagents to prepare the pharmaceutically acceptable base salts of this invention are those which form non-toxic base salts with the acidic compounds of formula 1. Such non-toxic base salts include those derived from such pharmacologically acceptable cations as sodium, potassium calcium and magnesium, etc. These salts can easily be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum yields of the desired final product. Since a single compound of the present invention may include more than one acidic or basic moieties, the compounds of the present invention may include mono, di or tri-salts in a single compound.

The compounds of the present invention are potent inhibitors of the FAK protein tyrosine kinases, and thus are all adapted to therapeutic use as antiproliferative agents (e., anticancer), antitumor (e.g., effective against solid tumors), antiangiogenesis (e.g., stop or prevent proliferationation of blood vessels) in mammals, particularly in humans. In particular, the compounds of the present invention are useful in the prevention and treatment of a variety of human hyperproliferative disorders such as malignant and benign tumors of the liver, kidney, bladder, breast, gastric, ovarian, colorectal, prostate, pancreatic, lung, vulval, thyroid, hepatic carcinomas, sarcomas, glioblastomas, head and neck, and other hyperplastic conditions such as benign hyperplasia of the skin (e, psoriasis) and benign hyperplasia of the prostate (e.g., BPH). It is, in addition, expected that a compound of the present invention may possess activity against a range of leukemias and lymphoid malignancies.

In one preferred embodiment of the present invention cancer is selected from lung cancer, bone cancer, pancreatic cancer, gastric, skin cancer, cancer of the head or neck, cutaneous or intraocular melanoma, uterine cancer, ovarian cancer, gynecological, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, squamous cell, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain, pituitary adenoma, or a combination of one or more of the foregoing cancers.

In a more preferred embodiment cancer is selected a solid tumor, such as, but not limited to, breast, lung, colon, brain, prostate, stomach, pancreatic, ovarian, skin (melanoma), endocrine, uterine, testicular, and bladder.

The compounds of the present invention may also be useful in the treatment of additional disorders in which aberrant expression ligand/receptor interactions or activation or signalling events related to various protein tyrosine kinases, are involved. Such disorders may include those of neuronal, glial, astrocytal, hypothalamic, and other glandular, macrophagal, epithelial, stromal, and blastocoelic nature in which aberrant function, expression, activation or signalling of the erbB tyrosine kinases are involved. In addition, the compounds of the present invention may have therapeutic utility in inflammatory, angiogenic and immunologic disorders involving both identified and as yet unidentified tyrosine kinases that are inhibited by the compounds of the present invention.

The in vitro activity of the compounds of formula 1 may be determined by the following procedure. More particularly, the following assay provides a method to determine whether compounds of the formula 1 inhibit the tyrosine kinase activity of the catalytic construct FAK(410-689). The assay is an ELISA-based format, measuring the inhibition of poly-glu-tyr phosphorylation by FAK(410-689).

The assay protocol has three parts:

I. Purification and cleavage of His-FAK(410-689)

II. FAK410-689 (a.k.a. FAKcd) Activation

III. FAKcd Kinase ELISA

Materials:

Ni-NTA agarose (Qiagen)

XK-16 column (Amersham-Pharmacia)

300 mM Imidizole

Superdex 200 HiLoad 16/60 prep grade column (Amersham Biotech.)

Antibody: Anti-Phosphotyrosine HRP-Conjugated Py20 (Transduction labs).

FAKcd: Purified and activated in house

TMB Microwell Peroxidase Substrate (Oncogene Research Products #CL07)

BSA: Sigma #A3294

Tween-20: Sigma #P1379

DMSO: Sigma #D-5879

D-PBS: Gibco #14190-037.

Reagents for Purification:

Buffer A: 50 mM HEPES pH 7.0,

500 mM NaCl,

0.1 mM TCEP

Complete™ protease inhibitor cocktail tablets (Roche)

Buffer B: 25 mM HEPES pH 7.0,

400 mM NaCl

0.1 mM TCEP.

Buffer C: 10 mM HEPES pH 7.5,

200 mM Ammonium Sulfate

0.1 mM TCEP.

Reagents for Activation

FAK(410-689): 3 tubes of frozen aliquots at 150 ul/tube for a total of 450 ul at 1.48 mg/ml (660 ug)

His-Src(249-524): -0.74 mg/ml stock in 10 mM HEPES, 200 mM (NH4)2SO4

Src reaction buffer (Upstate Biotech):

100 mM Tris-HCl pH7.2,

125 mM MgCl2,

25 mM MnCl2,

2 mM EDTA,

250 uM Na3VO4,

2 mM DTT

Mn2+/ATP cocktail (Upstate Biotech)

75 mM MnCl2

500 uM ATP

20 mM MOPS pH 7.2

1 mM Na3VO4

25 mM □-glycerol phosphate

5 mM EGTA

1 mM DTT

ATP: 150 mM stock

MgCl₂: 1 M Stock

DTT: 1 M stock

Reagents for FAKcd Kinase ELISA

Phosphorylation Buffer:

50 mM HEPES, pH 7.5,

125 mM NaCl,

48 mM MgCl2

Wash Buffer: TBS+0.1% Tween-20.

Blocking Buffer:

Tris Buffer Saline,

3% BSA,

0.05% Tween-20, filtered.

Plate Coating Buffer:

50 mg/ml Poly-Glu-Tyr (Sigma #P0275) in Phosphate buffer Saline (DPBS).

ATP: 0.1M ATP in H2O or HEPES, pH7.

Note: ATP Assay Buffer:

Make up as 75 uM ATP in PBS, so that 80 ul·in 120 ul reaction volume=50 uM final ATP concentration.

I. Purification of His-FAKcd(410-689)

1. Resuspend 130 g baculovirus cell paste containing the over expressed His-FAKcd410-689 recombinant protein in 3 volumes (400 ml) of Buffer A,

2. Lyse cells with one pass on a microfluidizer

3. Remove cell debris by centrifugation at 40C for 35 minutes at 14,000 rpm in a Sorval SLA-1500 rotor.

4. Transfer the supernatant to a clean tube and add 6.0 ml of Ni-NTA agarose (Qiagen)

5. Incubate the suspension with gentle rocking at 40C for 1 hour

6. Centrifuge suspension at 700×g in a swinging bucket rotor.

7. Discard the supernatant and resuspend the agarose beads in 20.0 ml of Buffer A

8. Transfer the beads to an XK-16 column (Amersham-Pharmacia) connected to a FPLC™.

9. Wash the agarose-beads with 5 column volumes of Buffer A and elute off the column with a step gradient of Buffer A containing 300 mM Imidizole.

10. Perform a buffer exchange of the eluted fractions into Buffer B

11. Following buffer exchange, pool the fractions and add thrombin at a 1:300 (w/w) ratio and incubated overnight at 13° C. to remove the N-terminal His-tag (His-FAK410-698→FAK410-689 (a.k.a. FAKcd)).

12. Add the reaction mixture back onto the Ni-NTA column equilibrated with Buffer A and collect the flow-through.

13. Concentrate the flow-through down to 1.7 ml and load directly onto a Superdex 200 HiLoad 16/60 prep grade column equilibrated with Buffer C. The desired protein elutes between 85-95 ml.

14. Aliquot the FAKcd protein and store frozen at −80° C.

II. FAK Activation

1. To 450 ul of FAK(410-689) at 1.48 mg/ml (660 ug) add the following:

30 ul of 0.037 mg/ml (1 uM) His-Src(249-524)

30 ul of 7.5 mM ATP

12 ul of 20 mM MgCl2

10 ul Mn2+/ATP cocktail (UpState Biotech.)

4 ul of 6.7 mM DTT

60 ul Src Reaction Buffer (UpState Biotech.)

2. Incubate Reaction for at least 3 hours at room temperature

At time t₀, almost all of the FAK(410-689) is singly phosphorylated. The second phosphorylation is slow. At t₁₂₀ (t=120 minutes), add 10 ul of 150 mM ATP.

T₀=(Start) 90% singly phosphorylated FAK(410-689) (1 PO4)

T₄₃=(43 min) 65% singly phosphorylated (1 PO4), 35% doubly phosphorylated (2 PO4)

T₉₀=(90 min) 45% 1 PO4, 55% 2 PO4

T₁₅₀=15% 1 PO4, 85% 2 PO4

T₂₁₀=<10% 1 PO4, >90% 2 PO4 desalted sample

3. Add 180 ul aliquots of the desalted material to NiNTA spin column and incubate on spin column

4. Spin at 10 k rpm (microfuge), for 5 min to isolate and collect flow through (Activated FAK(410-689)) and remove His-Src (captured on column)

III. FAKcd Kinase ELISA

1. Coat 96-well Nunc MaxiSorp plates with poly-glu-tyr (pGT) at 10 ug/well: Prepare 10 ug/ml of pGT in PBS and aliquot 100 ul/well. Incubate the plates at 37° C. overnight, aspirate the supernatant, wash the plates 3 times with Wash Buffer, and flick to dry before storing at 4° C.

2. Prepare compound stock solutions of 2.5 mM in 100% DMSO. The stocks are subsequently diluted to 60× of the final concentration in 100% DMSO, and diluted 1:5 in Kinase Phosphorylation Buffer.

3. Prepare a 75 uM working ATP solution in Kinase phosphorylation buffer. Add 80 ul to each well for a final ATP concentration of 50 uM.

4. Transfer 10 ul of the diluted compounds (0.5 log serial dilutions) to each well of the pGT assay plate, running each compound in triplicates on the same plate.

5. Dilute on ice, FAKcd protein to 1:1000 in Kinase Phosphorylation Buffer. Dispense 30 ul per well.

6. Note: Linearity and the appropriate dilution must be pre-determined for each batch of protein. The enzyme concentration selected should be such that quantitation of the assay signal will be approximately 0.8-1.0 at OD450, and in the linear range of the reaction rate.

7. Prepare both a No ATP control (noise) and a No Compound Control (Signal):

8. (Noise) One blank row of wells receives 10 ul of 1:5 diluted compounds in DMSO, 80 ul of Phosphorylation buffer (minus ATP), and 30 ul FAKcd solution.

9. (Siganl) Control wells receive 10 ul of 1:5 diluted DMSO (minus Compound) in Kinase phosphorylation buffer, 80 ul of 75 uM ATP, and 30 ul of 1:1000 FAKcd enzyme.

10. Incubate reaction at room temperature for 15 minutes with gentle shaking on a plate shaker.

11. Terminate the reaction by aspirating off the reaction mixture and washing 3 times with wash buffer.

12. Dilute phospho-tyrosine HRP-conjugated (pY20HRP) antibody to 0.250 ug/ml (1:1000 of Stock) in blocking buffer. Dispense 100 ul per well, and incubate with shaking for 30 min. at R.T.

13. Aspirate the supernatant and wash the plate 3 times with wash buffer.

14. Add 100 ul per well of room temperature TMB solution to initiate color development. Color development is terminated after approximately 15-30 sec. by the addition of 100 ul of 0.09M H2SO4 per well.

15. The signal is quantitated by measurement of absorbance at 450 nm on the BioRad microplate reader or a microplate reader capable of reading at OD450.

16. Inhibition of tyrosine kinase activity would result in a reduced absorbance signal. The signal is typically 0.8-1.0 OD units. The values are reported as IC₅₀ₛ, uM concentration.

FAK Inducible Cell-Based ELISA: Final Protocol

Materials:

Reacti-Bind Goat Anti-Rabbit Plates 96-well (Pierce Product#15135ZZ @115.00 USD)

FAKpY397 rabbit polyclonal antibody (Biosource #44624 @315.00 USD)

ChromePure Rabbit IgG, whole molecule (Jackson Laboratories #001-000-003 @60/25 mg USD)

UBI αFAK clone 2A7 mouse monoclonal antibody (Upstate#05-182 @289.00 USD)

Peroxidase-conjugated AffiniPure Goat Anti-Mouse IgG (Jackson Labs #115-035-146 @95/1.5 ml USD)

SuperBlock TBS (Pierce Product#37535ZZ @99 USD)

Bovine Serum Albumin (Sigma #A-9647 @117.95/100 g USD)

TMB Peroxidase substrate (Oncogene Research Products #CL07-100 ml @40.00 USD)

Na3VO4 Sodium Orthovanadate (Sigma #S6508 @43.95/50 g USD)

MTT substrate (Sigma # M-2128 @25.95/500 mg USD)

Growth Media: DMEM+10% FBS, P/S, Glu, 750 ug/ml Zeocin and 50 ug/ml Hygromycin (Zeocin InVitrogen #R²⁵⁰-05 @ 725 USD and Hygromycon InVitrogen #R²²⁰-05 @ 150 USD)

Mifepristone InVitrogen # Hi 10-01 @ 125 USD

Complete™ EDTA-free Protease Inhibitor pellet Boehringer Mannheim #1873580 FAK cell-based Protocol for selectivity of kinase-dependent phosphoFAKY397

Procedure

An inducible FAK cell-based assay in ELISA format for the screening of chemical matter to identify tyrosine kinase specific inhibitors was developed. The cell-based assay exploits the mechanism of the GeneSwitch™ system (InVitrogen) to exogenously control the expression and phosphorylation of FAK and the kinase-dependent autophosphorylation site at residue Y397.

Inhibition of the kinase-dependent autophosphorylation at Y397 results in a reduced absorbance signal at OD450. The signal is typically 0.9 to 1.5 OD450 units with the noise falling in the range of 0.08 to 0.1 OD450 units. The values are reported as IC₅₀ₛ, uM concentration.

On day 1, grow A431·FAKwt in T175 flasks. On the day prior to running the FAK cell-assay, seed A431·FAKwt cells in growth media on 96-well U-bottom plates. Allow cells to sit at 37° C., 5% CO₂ for 6 to 8 hours prior to FAK induction. Prepare Mifepristone stock solution of 10 uM in 100% Ethanol. The stock solution is subsequently diluted to 10× of the final concentration in Growth Media. Transfer 10 ul of this dilution (final concentration of 0.1 nM Mifepristone) into each well. Allow cells to sit at 37° C., 5% CO₂ overnight (12 to 16 hours). Also, prepare control wells without Mifepristone induction of FAK expression and phosphorylation.

On day 2, coat Goat Anti-Rabbit plate(s) with 3.5 ug/ml of phosphospecific FAKpY397 polyclonal antibody prepared in SuperBlock TBS buffer, and allow plate(s) to shake on a plate shaker at room temperature for 2 hours. Optionally, control wells may be coated with 3.5 ug/ml of control Capture antibody (Whole Rabbit IgG molecules) prepared in SuperBlock TBS. Wash off excess FAKpY397 antibody 3 times using buffer. Block Anti-FAKpY397 coated plate(s) with 200 ul per well of 3% BSA/0.5% Tween Blocking buffer for 1 hour at room temperature on the plate shaker. While the plate(s) are blocking, prepare compound stock solutions of 5 mM in 100% DMSO. The stock solutions are subsequently serially diluted to 100× of the final concentration in 100% DMSO. Make a 1:10 dilution using the 100× solution into growth media and transfer 10 ul of the appropriate compound dilutions to each well containing either the FAK induced or uninduced control A431 cells for 30 minutes at 37° C., 5% CO₂. Prepare RIPA lysis buffer (50 mM Tris-HCl, pH7.4, 1% NP-40, 0.25% Na-deoxycholate, 150 mM NaCl, 1 mM EDTA, 1 mM Na3VO4, 1 mM NaF, and one Complete™ EDTA-free protease inhibitor pellet per 50 ml solution). At the end of 30 minutes compound treatment, wash off compound 3 times using TBS-T wash buffer. Lyse cells with 100 ul/well of RIPA buffer.

To the coated plate, remove blocking buffer and wash 3 times using TBS-T wash buffer. Using a 96-well automated microdispenser, transfer 100 ul of whole cell-lysate (from step 6) to the Goat Anti-Rabbit FAKpY397 coated plate(s) to capture phosphoFAKY397 proteins. Shake at room temperature for 2 hours. Wash off unbound proteins 3 times using TBS-T wash buffer. Prepare 0.5 ug/ml (1:2000 dilution) of UBI αFAK detection antibody in 3% BSA/0.5% Tween blocking buffer. Dispense 100 ul of UBI αFAK solution per well and shake for 30 minutes at room temperature. Wash off excess UBI αFAK antibody 3 times using TBS-T wash buffer. Prepare 0.08 ug/ml (1:5000 dilution) of secondary Anti-Mouse Peroxidase (Anti-2 MHRP) conjugated antibody. Dispense 100 ul per well of the Anti-2 MHRP solution and shake for 30 minutes at room temperature. Wash off excess Anti-2 MHRP antibody 3 times using TBS-T wash buffer. Add 100 ul per well of room temperature TMB substrate solution to allow for color development. Terminate the TMB reaction with 100 ul per well of TMB stop solution (0.09M H2SO4) and quantitate the signal by measurement of absorbance at 450 nm on the BioRad microplate reader.

Additional FAK cell assays are hereby incorporated by reference from Pfizer Attorney Docket No. PC11699 entitled “INDUCIBLE FOCAL ADHESION KINASE CELL ASSAY”.

Administration of the compounds of the present invention (hereinafter the “active compound(s)”) can be effected by any method that enables delivery of the compounds to the site of action. These methods include oral routes, intraduodenal routes, parenteral injection (including intravenous, subcutaneous, intramuscular, intravascular or infusion), topical, and rectal administration.

The amount of the active compound administered will be dependent on the subject being treated, the severity of the disorder or condition, the rate of administration, the disposition of the compound and the discretion of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to about 7 g/day, preferably about 0.2 to about 2.5 g/day. In some instances, dosage levels below the lower limit of the aforesaid range may be more than adequate, while in other cases still larger doses may be employed without causing any harmful side effect, provided that such larger doses are first divided into several small doses for administration throughout the day.

The active compound may be applied as a sole therapy or may involve one or more other anti-tumour substances, for example those selected from, for example, mitotic inhibitors, for example vinblastine; alkylating agents, for example cis-platin, carboplatin and cyclophosphamide; anti-metabolites, for example 5-fluorouracil, cytosine arabinoside and hydroxyurea, or, for example, one of the preferred anti-metabolites disclosed in European Patent Application No. 239362 such as N-(5-[N-(3,4-dihydro-2-methyl-4-oxoquinazolin-6-ylmethyl)-N-methylamino]-2-thenoyl)-L-glutamic acid; growth factor inhibitors; cell cycle inhibitors; intercalating antibiotics, for example adriamycin and bleomycin; enzymes, for example interferon; and anti-hormones, for example anti-estrogens such as Nolvadex™ (tamoxifen) or, for example anti-androgens such as Casodex™ (4′-cyano-3-(4-fluorophenylsulphonyl)-2-hydroxy-2-methyl-3′-(trifluoromethyl)propionanilide). Such conjoint treatment may be achieved by way of the simultaneous, sequential or separate dosing of the individual components of the treatment.

The pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Exemplary parenteral administration forms include solutions or suspensions of active compounds in sterile aqueous solutions, for example, aqueous propylene glycol or dextrose solutions. Such dosage forms can be suitably buffered, if desired.

Suitable pharmaceutical carriers include inert diluents or fillers, water and various organic solvents. The pharmaceutical compositions may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Preferred materials, therefor, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

Methods of preparing various pharmaceutical compositions with a specific amount of active compound are known, or will be apparent, to those skilled in this art. For examples, see Remington's Pharmaceutical Sciences, Mack Publishing Company, Easter, Pa., 15th Edition (1975).

The examples and preparations provided below further illustrate and exemplify the compounds of the present invention and methods of preparing such compounds. It is to be understood that the scope of the present invention is not limited in any way by the scope of the following examples and preparations. In the following examples molecules with a single chiral center, unless otherwise noted, exist as a racemic mixture. Those molecules with two or more chiral centers, unless otherwise noted, exist as a racemic mixture of diastereomers. Single enantiomers/diastereomers may be obtained by methods known to those skilled in the art.

### General Methods

### Method A

### General Method for Introduction of a Group at C-4

(5-Bromo-2-chloro-pyrimidin-4-yl)-p-tolyl-amine

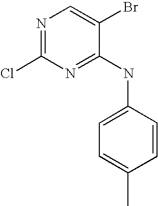

A mixture of 5-Bromo-2,4-dichloropyrimidine (5.00 g, 22.0 mmol), di-isopropyl ethylamine (3.91 mL, 22.4 mmol) and p-toluidine (2.40 g, 22.4 mmol) in n-butanol (50.0 mL) was heated to 105° C. under nitrogen for three hours. The reaction was allowed to cool to room temperature. The resulting mixture was poured into ethyl acetate and extracted with 1 N NaOH. The aqueous layer was removed and the organic layer was washed with water, dried over magnesium sulfate, filtered and evaporated under reduced pressured. To the resulting oily residue, diethyl ether was added and the mixture was then cooled to 0° C. HCl (4.0 M in dioxane) was added dropwise. The resulting white solid was filtered and dried. The salt was suspended in a mixture of water and ethyl acetate. The pH of the aqueous layer was then adjusted to 9 with 1 N NaOH and extracted. The aqueous layer was further extracted with ethyl acetate. The organic layers were combined, dried over magnesium sulfate, filtered and evaporated under reduced pressure to afford 5-Bromo-2-chloro-pyrimidin-4-yl)-p-tolyl-amine (3.62 g, 55%) as a white solid: C₁₁H₉BrClN₃. GC/MS: ret. Time=4.65 min, m/z 296/298/300; g.l.c. purity: 100%; TLC R_{f:}0.58 (20% Ethyl acetate/hexanes); ¹H NMR (d₆-DMSO) δ 9.21 (s, 1 H), 8.39 (s, 1 H), 7.35 (d, J=8.4 Hz, 2 H), 7.16 (d, J=8.4 Hz, 2 H), 2.27 (s, 3 H) ppm.

### Method B

### General Method for Introduction of a Group at C-4

(2-Chloro-5-fluoro-pyrimidin-4-yl)-pyridin-2-ylmethyl-amine

To a solution of 5-fluoro-2,4-dichloropyrimidine (1.5 g; 9 mmol) in THF (25 mL) was added triethylamine (1.1 eq), followed by dropwise addition of 2-(aminomethyl)pyridine (0.973 g; 1 eq). After stirring for one hour the reaction was concentrated and taken up in ethyl acetate, washed with saturated NaHCO₃, dried over Na₂SO₄, and the solvent removed. The resulting solid was re-crystallized from ethyl acetate and hexanes as a white solid (1.74 g; 81%): ¹H NMR (CDCl₃, 400 MHz) δ 4.84 (d, J=4.7 Hz, 2 H), 7.07 (bs, 1 H), 7.35 (t, J=5.1 Hz, 1 H), 7.44 (d, J=7.8, 1H), 7.82 (t, J=7.6, 1H), 7.95 (d, J=2.5 Hz, 1 H), 8.63 (d, J=5.0 Hz, 1 H); HPLC ret. Time: 4.228 min. LRMS (M+): 239.0, 241.0.

### Method C

### General Method for Introduction of a Group at C-4

Using method B, replace the THF solvent with 1,4-dioxane as solvent.

### Method D

### General Method for Introduction of a Group at C-4

5-Fluoro-N²-(1H-indazol-5-yl)-N⁴-pyridin-2-ylmethyl-pyrimidine-2,4-diamine

(2-Chloro-5-fluoro-pyrimidin-4-yl)-pyridin-2-ylmethyl-amine (100 mg; 0.4 mmol) and 5-aminoindazole (56 mg; 1 eq) were combined and heated at 160° C. for 30 minutes. After cooling to room temperature, methanol (1 mL) was added and stirred for 15 minutes, followed by filtration gave the product as a brown solid (29 mg; 21%): ¹H NMR (CD₃OD, 400 MHz) δ 4.80 (s, 2 H), 7.34 (m, 3 H), 7.43 (d, J=7.8 Hz, 1 H), 7.8 (m, 2 H), 7.87 (s, 1 H), 7.90 (s, 1 H), 8.54 (d, J=5 Hz, 1 H); HPLC ret. time: 3.916 min. LRMS (M+): 336.1.

### Method E

### General Method for Introduction of C-2 Group

5-(5-Bromo-4-phenethylamino-pyrimidin-2-ylamino)-1,3-dihydro-indol-2-one

153 mg (0.490 mmol) (5-Bromo-2-chloro-pyrimidin-4-yl)-phenethyl-amine was taken into 0.5 mL 1,4 dioxane with 0.14 mL (1.00 mmol) diisopropylethylamine and 80 mg (0.539 mmol) 5-amino-1,3-dihydro-indol-2-one. The reaction was allowed to heat to 110° C. for sixteen hours. The resulting brown glass was taken into 92.3:7:0.7 CHCl₃:CH₃OH:NH₄OH and washed with 1 N sodium hydroxide. The organic layer was dried over magnesium sulfate and evaporated directly onto silica gel. This adsorbed compound was purified via column chromatography (97.8:2:0.2 CHCl₃:CH₃OH:NH₄OH) over silica to isolate the major product.

The title compound was isolated as a white solid. C₂₀H₁₈BrN₅O: MS: 424.2/426.2 (MH+); ¹H NMR (D₆-DMSO) 10.20 (s, 1 H), 9.01 (s, 1 H), 7.93 (s, 1 H), 7.52 (s, 1 H), 7.44 (d, J=8.4 Hz, 1 H), 7.28-7.16 (m, 5 H), 6.97 (m, 1 H), 6.65 (d, J=8.3 Hz, 1 H), 3.56 (m, 2 H), 3.31 (s, 2 H), 2.82 (t, J=7.9 Hz, 2 H) ppm.

### Method F

### General Method for Introducing Both C-2 and C-4 Amines (“One Pot Method”)

4-{5-[5-Bromo-4-(4-trifluoromethyl-benzylamino)-Pyrimidin-2-ylamino]-1H-indol-3-yl}-3,6-dihydro-2H-pyridine-1-carboxylic acid tert-butyl ester

To a stirred solution of 5-bromo-2,4-dichloropyrimidine (0.222 g, 0.98 mmol) in THF (3 mL) under nitrogen was added triethylamine (0.42 mL, 3 mmol) followed by dropwise addition of p-trifluoromethylbenzyl amine (0.175 g, 1 mmol). After three hours the THF was removed under reduced pressure. To the resulting residue was added dioxane (1 mL) followed by 4-(5-Amino-1H-indol-3-yl)-3,6-dihydro-2H-pyridine-1-carboxylic acid tert-butyl ester (0.345 g 1.1 mmol). The mixture was stirred under nitrogen and then heated to 110° C. for sixteen hours. The reaction was cooled and was then dissolved in a solution of 5% methanol-dichloromethane and extracted with 1 N NaOH. The organic and aqueous layers were separated and the aqueous layer was further extracted with additional 5% methanol-dichloromethane. The organic layers were combined, washed with brine, dried over magnesium sulfate, filtered and evaporated under reduced pressure. The resulting residue was purified by silica gel chromotography (30% ethyl acetate in hexanes) to give 4-{5-[5-Bromo-4-(4-trifluoromethyl-benzylamino)-pyrimidin-2-ylamino]-1H-indol-3-yl}-3,6-dihydro-2H-pyridine-1-carboxylic acid tert-butyl ester (150 mg, 23%):

### Method G

### TFA General De-Protection Method

5-Bromo-N2-[3-(1,2,3,6-tetrahydro-pyridin-4-yl)-1H-indol-5-yl]-N⁴-(4-trifluoromethyl-benzyl)-pyrimidine-2,4-diamine trifluoro acetate salt

To a stirred solution of 4-{5-[5-Bromo-4-(4-trifluoromethyl-benzylamino)-pyrimidin-2-ylamino]-1H-indol-3-yl}-3,6-dihydro-2H-pyridine-1-carboxylic acid tert-butyl ester (0.15 g) in dichloromethane (2 mL) at 0° C. under nitrogen was added trifluoroacetic acid (4 mL). The cooling bath was removed and the reaction mixture was stirred for four hours. The reaction was concentrated under reduced pressure. To the resulting residue was added ethyl acetate (2 mL) followed by concentrating to an oily residue. The ethyl acetate concentration sequence was repeated three times. The resulting residue was suspended in ethyl acetate follow by addition of diethyl ether to precipitate 5-Bromo-N2-[3-(1,2,3,6-tetrahydro-pyridin-4-yl)-1H-indol-5-yl]-N⁴-(4-trifluoromethyl-benzyl)-pyrimidine-2,4-diamine trfluoroacetate salt (0.129 g, 86%) as a white solid: C₂₅H₂₂BrF₃N₆. MS: 542.9/544.7 (MH+). ¹H NMR (D₆-DMSO) δ 11.31 (s, 1 H), 8.82 (s, 2 H), 8.08 (s, 1 H), 7.88 (s, 1 H), 7.53 (s, 3 H), 7.36 (s, 2 H), 7.28 (d, J=8.3 Hz, 1 H), 7.16 (d, J=8.3 Hz, 1 H), 6.05 (bs, 1 H), 4.58 (s, 2 H), 3.75-3.65 (bs, 2 H), 3.35-3.25 (bs, 2H), 2.70-2.60 (bs, 2 H) ppm

### Method H

### HCl General De-Protection Method

5-Bromo-N2-[3-(1,2,3,6-tetrahydro-pyridin-4-yl)-1H-indol-5-yl]-N⁴-p-tolyl-pyrimidine-2,4-diamine hydrochloride salt

To a stirred solution of 4-[5-(5-Bromo-4-p-tolylamino-pyrimidin-2-ylamino)-1H-indol-3-yl]-3,6-dihydro-2H-pyridine-1-carboxylic acid tert-butyl ester (0.1 g, 0.174 mmol) and methanol (3 mL) cooled to 0° C. under nitrogen was added HCl in dioxane (0.2 mL of a 4 M solution). The cooling bath was removed and the reaction was allowed to stir for 6 hours. The mixture was concentrated under reduced pressure and the resultant residue was triturated with dichloromethane. The solid was filtered, washed with dichloromethane and dried to give 5-Bromo-N2-[3-(1,2,3,6-tetrahydro-pyridin-4-yl)-1H-indol-5-yl]-N⁴-p-tolyl-pyrimidine-2,4-diamine hydrochloride salt (0.076 g, 85%) as a white solid: C₂₄H₂₃BrN₆. MS: 475.0/477.0 (MH+); ¹H NMR (D₆-DMSO) δ 10.98 (s, 1 H), 9.01 (s, 1 H), 8.28 (s, 1 H), 8.12 (s, 1 H), 7.89 (s, 1 H), 7.50-7.58 (m, 3 H), 7.41 (d, J=8.7 Hz, 1 H), 7.29 (s, 1 H), 7.18 (d, J=8.7 Hz, 1 H), 7.03 (d, J=8.3 Hz, 2 H), 6.02 (s, 1 H), 4.03 (m, 2 H), 2.47 (m, 2 H), 2.35 (m, 2 H), 2.23 (s, 3 H) ppm.

### EXAMPLE 1

5-Bromo-N²-[3-(1,2,3,6-tetrahydro-pyridin-4-yl)-1H-indol-5-yl]-N⁴-p-tolyl-pyrimidine-2,4-diamine

A. 5-Bromo-2-chloro-pyrimidin-4-yl)-p-tolyl-amine

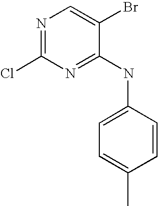

A mixture of 5-Bromo-2,4-dichloropyrimidine (5.00 g, 22.0 mmol), di-isopropyl ethylamine (3.91 mL, 22.4 mmol) and p-toluidine (2.40 g, 22.4 mmol) in n-butanol (50.0 mL) was heated to 105° C. under nitrogen for three hours. The reaction was allowed to cool to room temperature. The resulting mixture was poured into ethyl acetate and extracted with 1 N NaOH. The aqueous layer was removed and the organic layer was washed with water, dried over magnesium sulfate, filtered and evaporated under reduced pressured. To the resulting oily residue, diethyl ether was added and the mixture was then cooled to 0° C. HCl (4.0 M in dioxane) was added dropwise. The resulting white solid was filtered and dried. The salt was suspended in a mixture of water and ethyl acetate. The pH of the aqueous layer was then adjusted to 9 with 1N NaOH and extracted. The aqueous layer was further extracted with ethyl acetate. The organic layers were combined, dried over magnesium sulfate, filtered and evaporated under reduced pressure to afford 5-Bromo-2-chloro-pyrimidin-4-yl)-p-tolyl-amine (3.62 g, 55%) as a white solid: C₁₁H₉BrClN₃. GC/MS: ret. Time=4.65 min, m/z 296/298/300; g.l.c. purity: 100%; TLC R_{f} 0.58 (20% Ethyl acetate/hexanes); ¹H NMR (d₆-DMSO) δ 9.21 (s, 1 H), 8.39 (s, 1 H), 7.35 (d, J=8.4 Hz, 2 H), 7.16 (d, J=8.4 Hz, 2 H), 2.27 (s, 3 H) ppm.

B. 4-(5-Nitro-1H-indol-3-yl)-3,6-dihydro-2H-pyridine-1-carboxylic acid tert-butyl ester

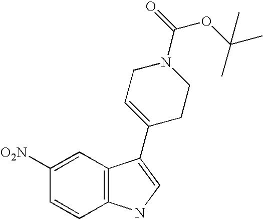

To 600 mL of HPLC-grade methanol was added 60.0 g (1.11 mol) sodium methoxide portion-wise. The resulting white slurry was allowed to stir for ten minutes before adding 30.0 g (185 mmol) 5-nitroindole. This allowed to stir for an additional ten minutes before adding 92.2 g (463 mmol) 4-Oxo-piperidine-1-carboxylic acid tert-butyl ester. After waiting ten minutes, the reaction temperature was ramped to 85° C. which was maintained for thirty-two hours. The black reaction solution was cooled to 0° C. and 250 mL distilled water was added drop-wise under nitrogen via an equalizing pressure addition funnel. The methanol was removed under reduced pressure. To the aqueous residue was added 1.50 L dichloromethane. The organic layer was separated. The pH of the aqueous was adjusted to 9.00 using sodium hydroxide. Dichloromethane was added and the two layers were filtered through diatomaceous earth to alleviate emulsion. The organic layer was separated and combined with the original organic. The combined organic layers were dried over magnesium sulfate. Partial evaporation of the dried organics resulted in a yellow-orange slurry. Filtration of this solid followed by washing with 5:1 diethyl ether:dichloromethane afforded 49.98 g (146 mmol, 79%) of the title compound as a yellow solid. MS: 244.1 (M−BocH+); TLC R_{f}: 0.31 (40% ethyl acetate/hexanes); ¹H NMR (D₆-DMSO) δ 11.90 (s, 1 H), 8.68 (s, 1 H), 7.99 (d, J=8.8 Hz, 1 H), 7.68 (s, 1 H), 7.53 (d, J=8.8 Hz, 1 H), 6.17 (s, 1 H), 4.04 (m, 2 H), 3.54 (m, 1 H), 2.47 (m, 2 H), 1.40 (s, 9 H) ppm.

C. 4-(5-Amino-1H-indol-3-yl)-3,6-dihydro-2H-pyridine-1-carboxylic acid tert-butyl ester

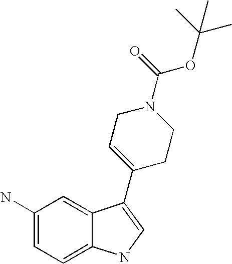

To a solution of 400 mL dioxane, 300 mL ethanol, and 200 mL distilled water was added ten grams of 4-(5-Nitro-1H-indol-3-yl)-3,6-dihydro-2H-pyridine-1-carboxylic acid tert-butyl ester. To this was added 8.13 g (146 mmol) powdered iron (0) and 6.23 g (116 mmol) ammonium chloride. The reaction was heated to 70° C. under nitrogen with the iron eventually becoming a conglomerate around the magnetic stir bar. After three hours, the reaction was removed from the heating source allowed to cool to room temperature and filtered. The filtrate was evaporated under reduced pressure. The aqueous residue was partitioned with ethyl acetate, dried over magnesium sulfate and filtered. Evaporation of the filtrate afforded the title compound as a tan glassy foam which darkens upon exposure to air. C₁₈H₂₃N₃O₂: 8.57 g (27.3 mmol, 94%): MS 214.1 (M-BoCH+); TLC R_{f}: 0.18 (40% Ethyl acetate:hexanes); ¹³C NMR (D₆-DMSO) δ 154.6, 142.5, 131.3, 126.1, 123.4, 115.4, 114.9, 112.6, 112.5, 104.2, 79.3, 44.0, 43.8, 41.5, 28.8, 28.3 ppm; ¹H NMR (D₆-DMSO) δ 10.71(s, 1 H), 7.24 (s, 1 H), 7.09 (d, J=8.4 Hz, 1 H), 7.04 (s, 1 H), 6.53 (d, J=8.4 Hz, 1 H), 6.00 (s, 1 H), 4.54 (s, 2 H), 4.54 (m, 2 H), 4.05 (m, 2 H), 3.56 (m, 2 H), 2.51 (m, 2 H), 1.45 (s, 9 H) ppm.

D. 4-[5-(5-Bromo-4-p-tolylamino-pyrimidin-2-ylamino)-1H-indol-3-yl]-3,6-dihydro-2H-pyridine-1-carboxylic acid tert-butyl ester

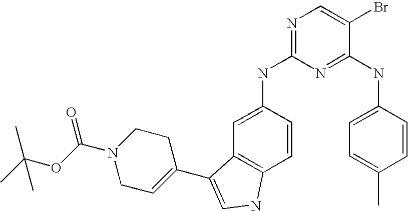

2.32 g (7.77 mmol) (5-Bromo-2-chloro-pyrimidin-4-yl)-p-tolyl-amine was taken into 21.0 mL dioxane with 2.92 g (2.92 mmol) 4-(5-Amino-1H-indol-3-yl)-3,6-dihydro-2H-pyridine-1-carboxylic acid tert-butyl ester and 1.30 mL (9.32 mmol) triethyl amine. The reaction was heated to 100° C. for sixteen hours. The reaction was allowed to cool to room temperature, and the dioxane was removed under reduced pressure. The brown residue was taken into ethyl acetate and 1 N sodium hydroxide mixture. Aqueous work-up gave approximately 3 g brown tar. This brown tar was purified to give 2.43 g (4.21 mmol, 54%) white solid. C₂₉H₃₁BrN₆O₂: MS: 575.0/577.0 (MH+); ¹H NMR (D₆-DMSO) δ 11.00 (s, 1 H), 9.01 (s, 1 H), 8.28 (s, 1 H), 8.13 (s, 1 H), 7.93 (s, 1 H), 7.53 (d, J=8.3 Hz, 2 H), 7.35 (s, 1 H), 7.34 (d, J=8.8 Hz, 1 H), 7.19 (d, J=8.8 Hz, 1 H), 7.02 (d, J=8.3 Hz, 2 H), 5.93 (s, 1 H), 3.89 (m, 2 H), 3.50 (m, 2 H), 3.14 (m, 2 H), 2.21 (s, 3 H), 1.39 (s, 9 H) ppm; TLC R_{f} 0.32 (40% ethyl acetate in hexanes).

E. 5-Bromo-N²-[3-(1,2,3,6-tetrahydro-pyridin-4-yl)-1H-indol-5-yl]-N⁴-D-tolyl-pyrimidine-2,4-diamine

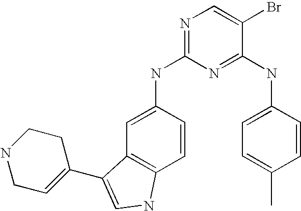

To a stirred solution of 4-[5-(5-Bromo-4-p-tolylamino-pyrimidin-2-ylamino)-1H-indol-3-yl]-3,6-dihydro-2H-pyridine-1-carboxylic acid tert-butyl ester (0.1 g, 0.174 mmol) and methanol (3 mL) cooled to 0° C. under nitrogen was added HCl in dioxane (0.2 mL of a 4 M solution). The cooling bath was removed and the reaction was allowed to stir for 6 hours. The mixture was concentrated under reduced pressure and the resultant residue was triturated with dichloromethane. The solid was filtered, washed with dichloromethane and dried to give 5-Bromo-N2-[3-(1,2,3,6-tetrahydro-pyridin-4-yl)-1H-indol-5-yl]-N⁴-p-tolyl-pyrimidine-2,4-diamine hydrochloride salt (0.076 g, 85%) as a white solid: C₂₄H₂₃BrN₆. MS: 475.0/477.0 (MH+); ¹H NMR (D₆-DMSO) δ 10.98 (s, 1 H), 9.01 (s, 1 H), 8.28 (s, 1 H), 8.12 (s, 1 H), 7.89 (s, 1 H), 7.50-7.58 (m, 3 H), 7.41 (d, J=8.7 Hz, 1 H), 7.29 (s, 1 H), 7.18 (d, J=8.7 Hz, 1 H), 7.03 (d, J=8.3 Hz, 2 H), 6.02 (s, 1 H), 4.03 (m, 2 H), 2.47 (m, 2 H), 2.35 (m, 2 H), 2.23 (s, 3 H) ppm.

### EXAMPLE 2

5-Bromo-N⁴-pyridin-2-yl-N2-[3-(1,2,3,6-tetrahydro-pyridin-4-yl)-1H-indol-5-yl]-pyrimidine-2,4-diamine

A. (5-Bromo-2-chloro-Pyrimidin-4-yl)-pyridin-2-yl-amine

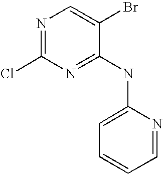

The title compound was prepared from 2-aminopyridine in a 10% yield as a yellow solid in a manner similar to Example 1A. C₉H₆BrClN₄. GC/MS: ret. time=4.19 min. m/z 284/286/288, 205/207, 169, 78; ¹H NMR (D₆-DMSO) δ 9.06 (bs, 1 H), 8.57 (s, 1 H), 8.38 (d, J=4.6 Hz, 1 H), 7.93-7.86 (m, 2 H), 7.20 (dd, J=4.6, 6.2 Hz, 1 H) ppm.

B. 5-Bromo-N⁴-pyridin-2-yl-N²-[3-(1,2,3,6-tetrahydro-pyridin-4-yl)-1H-indol-5-yl]-Pyrimidine-2,4-diamine

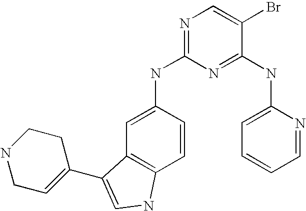

The title compound was made in a manner similar to Examples 1D and 1E. The compound was isolated as its HCl salt in a 29% yield as a yellow solid. C₂₂H₂₀BrN₇. MS: 462.1/464.1 (MH+). ¹H NMR (CD₃OD) δ 8.37 (s, 1 H), 8.2-7.8 (m, 4 H), 7.53 (m, 2 H), 7.29 (m, 2 H), 6.18 (bs, 1 H), 4.93-4.80 (m, 2 H), 3.87-3.48 (m, 2 H), 3.00-2.80 (m, 2 H) ppm.

### EXAMPLE 3

5-Bromo-N⁴-pyridin-2-ylmethyl-N²-[3-(1,2,3,6-tetrahydro-pyridin-4-yl)-1H-indol-5-yl]-pyrimidine-2,4-diamine

A. (5-Bromo-2-chloro-pyrimidin-4-yl)-pyridin-2-ylmethyl-amine

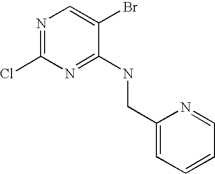

The title compound was made in 82% yield as a yellow oil that solidifies on standing. C₁₀H₈BrClN₄. GC/MS ret. time=4.67 min. m/z 298/300/302, 219/221, 107. ¹H NMR (CDCl₃) δ8.64 (d, J=4.7 Hz, 1 H), 8.19 (s, 1 H), 7.78 (t, J=7.8 Hz, 1 H), 7.41-7.29 (m, 3 H), 4.82 (d, J=4.7 Hz, 2 H) ppm.

B. 5-Bromo-N⁴-pyridin-2-ylmethyl-N²-[3-(1,2,3,6-tetrahydro-pyridin-4-yl)-1H-indol-5-yl]-pyrimidine-2,4-diamine

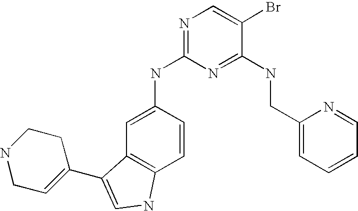

The title compound was made in a manner similar to Example 1D and 1E in 14% yield isolated as a free based white solid. C₂₃H₂₂BrN₇. MS: 447.0/449.0 (MH+), ¹H NMR (D₆-DMSO) δ 10.85 (s, 1 H), 8.91 (s, 1 H), 8.50 (s, 1 H), 8.01-8.00 (m, 2 H), 7.68 (t, J=6.4 Hz, 1 H), 7.42 (t, J=5.7 Hz, 1 H), 7.28-7.20 (m, 4 H), 7.09 (d, J=8.3 Hz, 1 H), 6.07 (s, 1 H), 4.70 (d, J=5.7 Hz, 2 H), 3.40-3.30 (m, 2 H), 2.90-2.87 (m, 2 H), 2.50-2.40 (m, 2 H) ppm.

### EXAMPLE 4

N⁴-Benzyl-5-bromo-N²-[3-(1,2,3,6-tetrahydro-pyridin-4-yl)-1H-indol-5-yl]-pyrimidine-2,4-diamine

A. Benzyl-(5-bromo-2-chloro-pyrimidin-4-yl)-amine

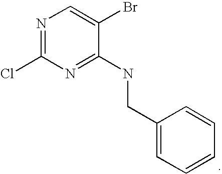

The title compound was synthesized in a manner similar to Example 1A. It was isolated in an 85% yield as a yellow solid. C₁₁H₉BrClN_{3.} MS 296.1/298.0 (MH+). ¹H NMR (CDCl₃) δ 8.19 (s, 1 H), 7.45-7.30 (m, 5 H), 5.85 (bs, 1 H), 4.74 (d, J=5.6 Hz, 2 H) ppm.

B. 4-[5-(4-Benzylamino-5-bromo-pyrimidin-2-ylamino)-1H-indol-3-yl]-3,6-dihydro-2H-pyridine-1-carboxylic acid tert-butyl ester

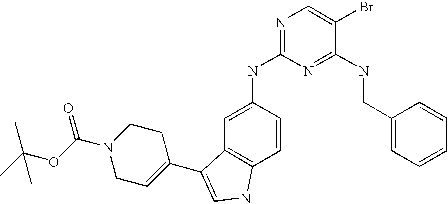

The title compound was made in a manner similar to Example 1D. It was isolated in a 65% yield after chromatography (30% EtOAc in hexanes) as a white solid. C₂₉H₃₁BrN₆O₂. MS: 575.0/576.8 (MH+). ¹H NMR (D₆-DMSO) δ 10.95 (s, 1 H), 8.92 (s, 1 H), 8.14 (s, 1 H), 7.96 (s, 1 H), 7.48-7.14 (m, 9 H), 6.02 (s, 1 H), 4.61 (d, J=6.2 Hz, 2 H), 4.01-3.98 (m, 2 H), 3.51-3.48 (m, 2 H), 2.47-2.45 (m, 2 H), 1.38 (s, 9 H) ppm.

C. N⁴-Benzyl-5-bromo-N2-[3-(1,2,3,6-tetrahydro-pyridin-4-yl)-1H-indol-5-yl]-pyrimidine-2,4-diamine

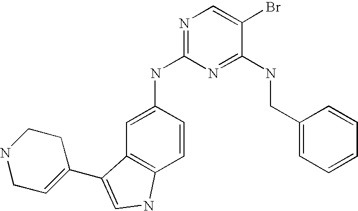

The title compound was synthesized by dissolving 4-[5-(4-Benzylamino-5-bromo-pyrimidin-2-ylamino)-1H-indol-3-yl]-3,6-dihydro-2H-pyridine-1-carboxylic acid tert-butyl ester into 5.00 mL dichloromethane and cooling to 0° C. To this was added 10.0 mL Trifluoroacetic acid. The red solution was allowed to slowly warm to room temperature and stir under N2 for two hours. 5.00 mL ethyl acetate was added. Filtration of the resulting precipitate gave the title compound as a white solid. C₂₄H₂₃BrN₆. MS: 475.0/476.8 (MH+). ¹H NMR (CD₃OD) δ 11.05 (s, 1 H), 7.88 (s, 1 H), 7.81 (s, 1 H), 7.49 (s, 1 H), 7.45 (d, J=8.7 Hz, 1 H), 7.36-7.13 (m, 8H), 6.15 (bs, 1 H), 4.64 (bs, 2 H), 3.90-3.80 (bs, 2 H), 3.49-3.43 (bs, 2 H), 2.85-2.83 (bs, 2 H) ppm.

### EXAMPLE 5

5-Bromo-N4-(1R-Phenyl-ethyl)-N2-[3-(1,2,3,6-tetrahydro-pyridin-4-yl)-1H-indol-5-yl]-pyrimidine-2,4-diamine

A. (5-Bromo-2-chloro-Pyrimidin-4-yl)-(1R-phenyl-ethyl)-amine

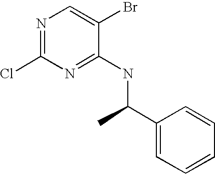

The title compound was made in a manner similar to Example 1A. It was isolated as an orange solid in a nearly quantitative yield. C₁₂H₁₁BrClN₃. MS: 312.1/314.1 (MH+). ¹H NMR (CDCl₃) δ 8.11 (s, 1 H), 7.37-7.14 (m, 5 H), 5.71 (d, J=7.4 Hz, 1 H), 5.35 (dt, J=7.4, 6.7 Hz, 1 H), 1.60 (d, J=6.7 Hz, 3 H) ppm.

B. 5-Bromo-N4-(1R-Phenyl-ethyl)-N2-[3-(1,2,3,6-tetrahydro-pyridin-4-yl)-1H-indol-5-yl]-pyrimidine-2,4-diamine

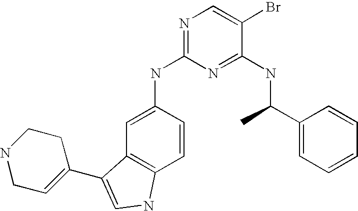

The title compound was made in a manner similar to Example 1D and deprotected similarly to Example 4C to give the desired material as its TFA salt in a 18% yield (tan solid). C₂₅H₂₅BrN₆. MS 489.0/491.1 (MH+); ¹H NMR (D₆-DMSO) δ 11.37 (s, 1 H), 8.91 (s, 1 H), 8.11 (s, 1 H), 7.94 (s, 1 H), 7.57 (s, 1 H), 7.40 (d, J=8.8 Hz, 1 H), 7.30-7.22 (m, 7H), 6.12 (s, 1 H), 4.06 (bs, 1 H), 3.77-3.75 (bs, 2 H), 3.38-3.36 (bs, 2 H), 2.76-2.75 (bs, 2 H), 1.57 (d, J=6.8 Hz, 3 H) ppm.

### EXAMPLE 6

5-Bromo-N⁴-(1rac-phenyl-ethyl)-N²-[3-(1,2,3,6-tetrahydro-pyridin-4-yl)-1H-indol-5-yl]-pyrimidine-2,4-diamine

A. (5-Bromo-2-chloro-pyrimidin-4-yl)-(1 rac-phenyl-ethyl)-amine

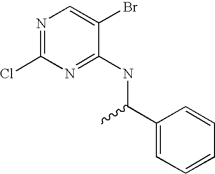

The title compound was made in a manner similar to Example 1A. It was isolated as an orange solid in nearly quantitative yield. C₁₂H₁₁BrClN₃. MS: 312.1/314.1 (MH+). ¹H NMR (CDCl₃) δ 8.11 (s, 1 H), 7.37-7.14 (m, 5 H), 5.71 (d, J=7.4 Hz, 1 H), 5.35 (dt, J=7.4, 6.7 Hz, 1 H), 1.60 (d, J=6.7 Hz, 3 H) ppm

B. 5-Bromo-N4-(1 rac-phenyl-ethyl)-N2-[3-(1,2,3,6-tetrahydro-pyridin-4-yl)-1H-indol-5-yl]-pyrimidine-2,4-diamine

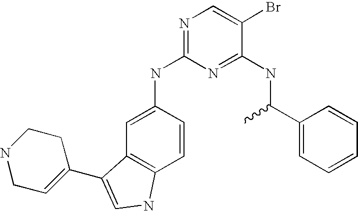

The title compound was made in a manner similar to Example 1D and deprotected similarly to Example 4C to give the desired material as its TFA salt in a 27% yield (tan solid). C₂₅H₂₅BrN₆. MS 489.0/491.1 (MH+); ¹H NMR (d₆-DMSO) δ□11.37 (s, 1 H), 8.91 (s, 1 H), 8.11 (s, 1 H), 7.94 (s, 1 H), 7.57 (s, 1 H), 7.40 (d, J=8.8 Hz, 1 H), 7.30-7.22 (m, 7H), 6.12 (s, 1 H), 4.06 (bs, 1 H), 3.77-3.75 (bs, 2 H), 3.38-3.36 (bs, 2 H), 2.76-2.75 (bs, 2 H), 1.57 (d, J=6.8 Hz, 3 H) ppm.

### EXAMPLE 7

5-Bromo-N4-(1S-phenyl-ethyl)-N2-[3-(1,2,3,6-tetrahydro-pyridin-4-yl)-1H-indol-5-yl]-pyrimidine-2,4-diamine

A. (5-Bromo-2-chloro-pyrimidin-4-yl)-(1S-phenyl-ethyl)-amine

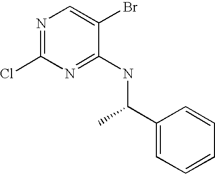

The title compound was made in a manner similar to Example 1A. It was isolated as an yellow solid in a 84% yield. C₁₂H₁₁BrClN₃. MS: 312.1/314.1 (MH+). ¹H NMR (CDCl₃) d 8.11 (s, 1 H), 7.37-7.14 (m, 5 H), 5.71 (d, J=7.4 Hz, 1 H), 5.35 (dt, J=7.4, 6.7 Hz, 1 H), 1.60 (d, J=6.7 Hz, 3 H) ppm

B. 5-Bromo-N4-(1S-phenyl-ethyl)-N2-[3-(1,2,3,6-tetrahydro-pyridin-4-yl)-1H-indol-5-yl]-pyrimidine-2,4-diamine

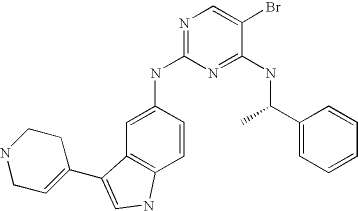

The title compound was made in a manner similar to Example 1D and deprotected similarly to Example 4C to give the desired material as its TFA salt in a 15% yield (tan solid). C₂₅H₂₅BrN₆. MS 489.0/491.1 (MH+); ¹H NMR (d₆-DMSO) 6[111.37 (s, 1 H), 8.91 (s, 1 H), 8.11 (s, 1 H), 7.94 (s, 1 H), 7.57 (s, 1 H), 7.40 (d, J=8.8 Hz, 1 H), 7.30-7.22 (m, 7H), 6.12 (s, 1 H), 4.06 (bs, 1 H), 3.77-3.75 (bs, 2 H), 3.38-3.36 (bs, 2 H), 2.76-2.75 (bs, 2 H), 1.57 (d, J=6.8 Hz, 3 H) ppm.

### EXAMPLE 8

4-({5-Bromo-2-[3-(1,2,3,6-tetrahydro-pyridin-4-yl)-1H-indol-5-ylamino]-pyrimidin-4-ylamino}-methyl)-benzenesulfonamide

A. 4-[(5-Bromo-2-chloro-pyrimidin-4-ylamino)-methyl]-benzenesulfonamide

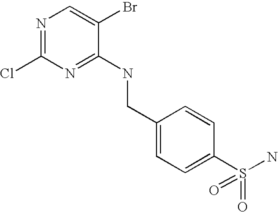

The title compound was made in a manner similar to Example 1A. It was isolated in a 30% yield as a white solid which fell out of solution upon work-up. C₁₁H₁₀BrClN₄O₂S. MS 375/3771378 (MH+). ¹H NMR (d₆-DMSO) δ 8.26 (s, 1 H), 7.74 (d, J=8.6 Hz, 2 H), 7.42 (d, J=8.6 Hz, 2 H), 4.59 (s, 2 H) ppm.

B. 4-({5-Bromo-2-[3-(1,2,3,6-tetrahydro-pyridin-4-yl)-1H-indol-5-ylamino]-pyrimidin-4-ylamino}-methyl)-benzenesulfonamide

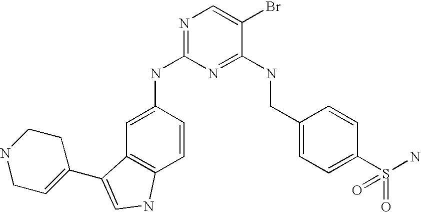

The title compound was made in a manner similar to Example 1D and deprotected similarly to Example 4C. It was isolated as its free base after column chromatography (93:7:0.7 CHCl₃:CH₃OH:NH₄OH) as a brown solid in a 2% yield. C₂₄H₂₄BrN₇O₂S. MS: 554.1/556.0 (MH+). ¹H NMR (CD₃OD) δ□(CD₃OD) δ 7.89 (s, 1 H), 7.68 (d, J=8.3 Hz, 2 H), 7.31 (d, J=8.3 Hz, 2 H), 7.26-7.22 (m, 2 H), 7.16-7.10 (m, 2 H), 6.69 (d, J=8.7 Hz, 1 H), 6.16 (bs, 1 H), 4.61 (bs, 2 H), 3.59-3.57 (bs, 2 H), 3.30-3.21 (bs, 2 H), 2.55-2.53 (bs, 2 H) ppm.

### EXAMPLE 9

5-Bromo-N²-[3-(1,2,3,6-tetrahydro-pyridin-4-yl)-1H-indol-5-yl]-N⁴-(4-trifluoromethyl-benzyl)-pyrimidine-2,4-diamine

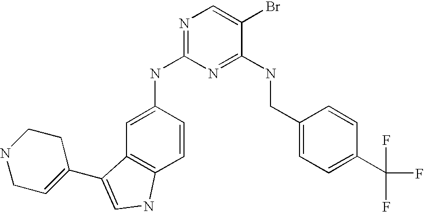

To a stirred solution of 5-bromo-2,4-dichloropyrimidine (0.222 g, 0.98 mmol) in THF (3 mL) under nitrogen was added triethylamine (0.42 mL, 3 mmol) followed by dropwise addition of p-trifluoromethylbenzyl amine (0.175 g, 1 mmol). After three hours the THF was removed under reduced pressure. To the resulting residue was added dioxane (1 mL) followed by 4-(5-Amino-1H-indol-3-yl)-3,6-dihydro-2H-pyridine-1-carboxylic acid tert-butyl ester (0.345 g 1.1 mmol). The mixture was stirred under nitrogen and then heated to 110° C. for sixteen hours. The reaction was cooled and was then dissolved in a solution of 5% methanol-dichloromethane and extracted with 1 N NaOH. The organic and aqueous layers were separated and the aqueous layer was further extracted with additional 5% methanol-dichloromethane. The organic layers were combined, washed with brine, dried over magnesium sulfate, filtered and evaporated under reduced pressure. The resulting residue was purified by silica gel chromotography (30% ethyl acetate in hexanes) to give 4-{5-[5-Bromo-4-(4-trifluoromethyl-benzylamino)-pyrimidin-2-ylamino]-1H-indol-3-yl}-3,6-dihydro-2H-pyridine-1-carboxylic acid tert-butyl ester (150 mg, 23%): (MS: 642.9/644.73 MH+). This material was then taken directly to the next reaction. To a stirred solution of 4-{5-[5-Bromo-4-(4-trifluoromethyl-benzylamino)-pyrimidin-2-ylamino]-1H-indol-3-yl}-3,6-dihydro-2H-pyridine-1-carboxylic acid tert-butyl ester (0.15 g) in dichloromethane (2 mL) at 0° C. under nitrogen was added trifluoroacetic acid (4 mL). The cooling bath was removed and the reaction mixture was stirred for four hours. The reaction was concentrated under reduced pressure. To the resulting residue was added ethyl acetate (2 mL) followed by concentrating to an oily residue. The ethyl acetate concentration sequence was repeated three times. The resulting residue was suspended in ethyl acetate follow by addition of diethyl ether to precipitate 5-Bromo-N-2-[3-(1,2,3,6-tetrahydro-pyridin-4-yl)-1H-indol-5-yl]-N⁴-(4-trifluoromethyl-benzyl)-pyrimidine-2,4-diamine trfluoroacetate salt (0.129 g, 86%) as a white solid: C₂₅H₂₂BrF₃N₆. MS: 542.9/544.7 (MH+). ¹H NMR (D₆-DMSO) δ 11.31 (s, 1 H), 8.82 (s, 2 H), 8.08 (s, 1 H), 7.88 (s, 1 H), 7.53 (s, 3 H), 7.36 (s, 2 H), 7.28-(d, J=8.3 Hz, 1 H), 7.16 (d, J=8.3 Hz, 1 H), 6.05 (bs, 1 H), 4.58 (s, 2 H), 3.75-3.65 (bs, 2 H), 3.35-3.25 (bs, 2 H), 2.70-2.60 (bs, 2 H) ppm

### EXAMPLE 10

5-Bromo-N⁴-(4-methoxy-benzyl)-N²-[3-(1,2,3,6-tetrahydro-pyridin-4-yl)-1H-indol-5-yl]-pyrimidine-2,4-diamine

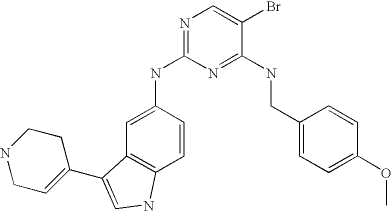

The title compound was synthesized according to the procedure of Example 9. It was isolated in a 21% yield as a white solid TFA salt. C₂₅H₂₅BrN₆O. MS: 505.0/506.8 (MH+); ¹H NMR (D₆-DMSO) δ 11.33 (s, 1 H), 8.84 (s, 2 H), 8.06 (s, 1 H), 7.95 (s, 1 H), 7.53 (s, 1 H), 7.35 (d, J=7.9 Hz, 1 H), 7.23 (d, J=7.9 Hz, 1 H), 7.10 (s, 2 H), 6.74 (s, 1 H), 6.73 (s, 1 H), 6.06 (s, 1 H), 4.26 (s, 2 H), 3.69 (s, 2 H), 3.66 (s, 3 H), 3.30 (s, 2 H), 2.68 (s, 2 H) ppm.

### EXAMPLE 11

5-Bromo-N⁴-(4-fluoro-benzyl)-N²-[3-(1,2,3,6-tetrahydro-Pyridin-4-yl)-1H-indol-5-yl]-pyrimidine-2,4-diamine

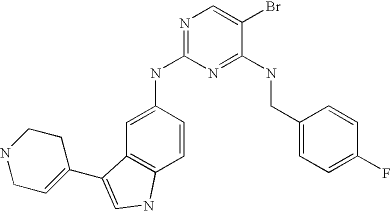

The title compound was synthesized according to the procedure of Example 9. It was isolated in a 12% overall yield as an off-white TFA salt. C₂₄H₂₂BrFN₆. MS: 492.9/494.9 (MH+); ¹H NMR (D₆-DMSO) δ 11.26 (s, 1 H), 8.78 (s, 2 H), 8.03 (s, 1 H), 7.95 (s, 1 H), 7.51 (s, 1 H), 7.31-7.23 (m, 3 H), 7.02 (s, 2 H), 6.05 (s, 1 H), 4.50 (s, 2 H), 3.70 (s, 2 H), 3.29 (s, 2H), 2.68 (s, 2 H) ppm.

### EXAMPLE 12

5-Bromo-N⁴-(3-fluoro-benzyl)-N²-[3-(1,2,3,6-tetrahydro-pyridin-4-yl)-1H-indol-5-yl]-Pyrimidine-2,4-diamine

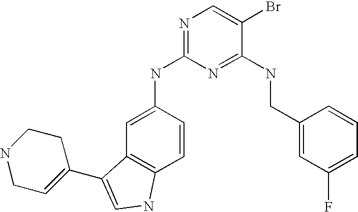

The title compound was synthesized in a manner similar to Example 9 in a 20% yield. It was isolated as an off-white solid TFA salt. C₂₄H₂₂BrFN₆. MS: 492.9/494.9 (MH+); ¹H NMR (D₆-DMSO) δ 11.33 (s, 1 H), 8.66 (s, 2 h), 8.40-8.20 (bs, 1 H), 8.11 (s, 1 H), 7.98 (s, 1 H), 7.57 (s, 1 H), 7.33-7.30 (m, 3 H), 7.10-7.07 (m, 3 H), 6.11 (s, 1 H), 4.60 (d, J=5.6 Hz, 2H), 3.77 (s, 2 H), 3.37 (s, 2 H), 2.73 (s, 2 H) ppm.

### EXAMPLE 13

5-Bromo-N⁴-naphthalen-1-ylmethyl-N²-[3-(1,2,3,6-tetrahydro-pyridin-4-yl)-1H-indol-5-yl]-pyrimidine-2,4-diamine

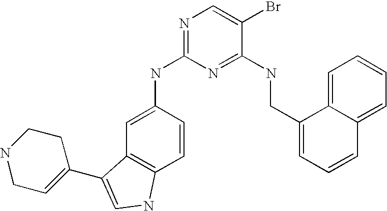

The title compound was made in a manner described in Example 9 in a 16% yield. The isolated TFA salt was characterized as an off-white solid. C₂₈H₂₅BrN₆. MS: 525.1/527.1 (MH+); ¹H NMR (D₆-DMSO) δ 11.21 (s, 1 H), 8.76 (s, 2 H), 8.15 (d, J=9.2 Hz, 1 H), 8.06 (s, 1 H), 7.93 (d, J=8.0 Hz, 1 H), 7.89 (s, 1 H), 7.79 (d, J=7.8 Hz, 1 H), 7.54-7.46 (m, 3 H), 7.34 (s, 1 H), 7.28 (s, 1 H), 7.14 (d, J=8.4 Hz, 1 H), 6.98 (bs, 1 H), 6.02 (s, 1 H), 5.04 (s, 2 H), 3.67 (s, 2 H), 3.28 (s, 2 H), 2.65 (s, 2 H) ppm.

### EXAMPLE 14

5-Bromo-N⁴-(4-fluoro-3-trifluoromethyl-benzyl)-N²-[3-(1,2,3,6-tetrahydro-pyridin-4-yl)-1H-indol-5-yl]-pyrimidine-2,4-diamine

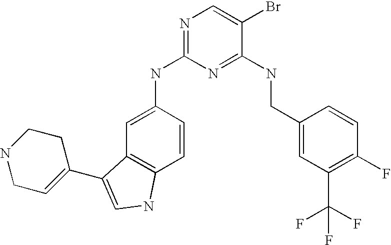

The title compound was made in a manner described in Example 9 in a 12% overall yield. The isolated TFA salt was characterized as an off-white solid. C₂₅H₂₁BrF₄N₆. MS: 560.8/562.4 (MH+); ¹H NMR (D₆-DMSO) 6011.31 (s, 1 H), 8.87 (s, 2 H), 8.24 (bs. 1 H), 8.11 (s, 1 H), 8.01 (s, 1 H), 7.72 (s, 1 H), 7.56 (s, 2 H), 7.36-7.29 (m, 3 H), 6.18 (s, 1 H), 4.62 (d, J=5.6 Hz, 2 H), 3.79 (s, 2 H), 3.39 (s, 2 H), 2.74 (s, 2 H) ppm.

### EXAMPLE 15

5-Bromo-N⁴-(3-fluoro-5-trifluoromethyl-benzyl)-N²-[3-(1,2,3,6-tetrahydro-Pyridin-4-yl)-1H-indol-5-yl]-pyrimidine-2,4-diamine

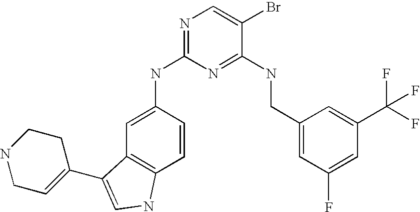

The title compound was synthesized in a manner described in Example 9 in a 16% overall yield. It was characterized as an off-white solid as its TFA salt. C₂₅H₂₁BrF₄N₆. MS: 561.4/563.2 (MH+), ¹H NMR (D₆-DMSO) δ 11.26 (s, 1 H), 8.82 (s, 2 H), 8.21 (bs, 1 H), 8.07 (s, 1 H), 7.94 (s, 1 H), 7.46-7.35 (m, 3 H), 7.24 (s, 1 H), 7.20 (s, 2 H), 6.06 (s, 1 H), 4.61 (d, J=5.4 Hz, 2 H), 3.74 (s, 2 H), 3.30 (s, 2 H), 2.68 (s, 2 H) ppm.

### EXAMPLE 16

5-Bromo-N⁴-(4-phenoxy-benzyl)-N²-[3-(1,2,3,6-tetrahydro-Pyridin-4-yl)-1H-indol-5-yl]-pyrimidine-2,4-diamine

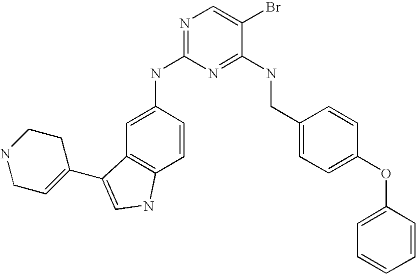

The title compound was synthesized in a 9% overall yield in a manner described in Example 9. It was characterized as an off-white solid isolated as its TFA salt. C₃₀H₂₇BrN₆O. 567.0/568.6 (MH+); ¹H NMR (CD₃OD) δ 7.89 (s, 1 H), 7.84 (s, 1 H), 7.48 (s, 1 H), 7.47 (d, J=7.5 Hz, 1 H), 7.31 (dd, J=7.5, 0.3 Hz, 2 H), 7.17 (d, J=8.7 Hz, 1 H), 7.15 (bs, 2 H), 7.08 (t, J=7.5 Hz, 1 H), 6.90 (d, J=8.3 Hz, 2 H), 6.79 (s, 2 H), 6.15 (s, 1 H), 4.57 (s, 2 H), 3.80 (s, 2 H), 3.42 (s, 2 H), 2.82 (s, 2 H) ppm.

### EXAMPLE 17

5-Bromo-N⁴-(3,4-difluoro-benzyl)-N²-[3-(1,2,3,6-tetrahydro-pyridin-4-yl)-1H-indol-5-yl]-pyrimidine-2,4-diamine

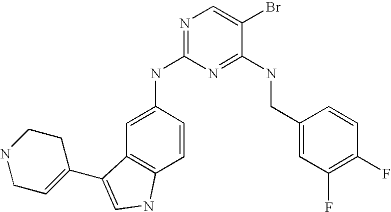

The title compound was synthesized in a 19% overall yield in a manner described in Example 9. It was characterized as an off-white solid isolated as its TFA salt. C₂₄H₂₁BrF₂N₆: 510.9/513.0 (MH+); ¹H NMR (D₆-DMSO) δ 11.26 (s, 1 H), 8.87 (bs, 2 H), 8.09 (s, 2 H), 8.00 (s, 1 H), 7.56 (s, 1 H), 7.33 (m, 3 H), 7.10 (s, 1 H), 6.11 (s, 1 H), 4.54 (s, 2 H), 3.78 (s, 2 H), 3.35 (s, 2 H), 2.74 (s, 2 H) ppm.

### EXAMPLE 18

5-Bromo-N²-[3-(1,2,3,6-tetrahydro-pyridin-4-yl)-1H-indol-5-yl]-N⁴-(3-trifluoromethoxy-benzyl)-pyrimidine-2.4-diamine

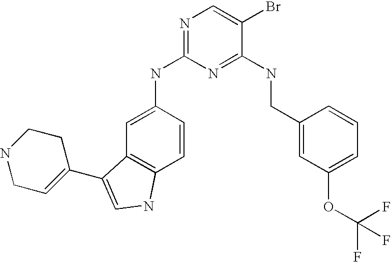

The title compound was synthesized in a 8% ovrall yield in a manner described in Example 9. It was characterized as an off-white solid isolated as its TFA salt. C₂₅H₂BrF₃N₆O. 559.0/561.0 (MH+); ¹H NMR (D₆-DMSO) δ 11.28 (s, 1 H), 8.81 (bs, 2 H), 8.08 (s, 1 H), 8.01 (s, 1 H), 7.55 (s, 1 H), 7.50 (bs, 1 H), 7.40-7.21 (m, 6 H), 6.10 (s, 1 H), 4.63 (s, 2 H), 3.77 (s, 2 H), 3.37 (s, 2 H), 2.73 (s, 2 H) ppm.

### EXAMPLE 19

5-Bromo-N⁴-(4-chloro-benzyl)-N²-[3-(1,2,3,6-tetrahydro-pyridin-4-yl)-1H-indol-5-yl]-Pyrimidine-2,4-diamine

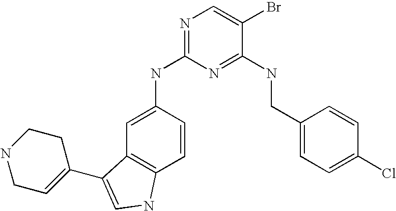

The title compound was synthesized in a 20% overall yield in a manner described in Example 9 from 4-chlorobenzyl amine. It was characterized as an off-white solid isolated as its TFA salt. C₂₄H₂₂BrClN₆. MS: 508.9/510.9/513.0 (MH+); ¹H NMR (D₆-DMSO) δ 11.27 (s, 1 H), 8.85 (bs, 2 H), 8.09 (s, 1 H), 7.98 (s, 1 H), 7.56 (s, 1 H), 7.32-7.29 (m, 6 H), 6.10 (s, 1 H), 4.55 (s, 2 H), 3.77 (s, 2 H), 3.36 (s, 2 H), 2.74 (s, 2 H) ppm.

### EXAMPLE 20

5-Bromo-N²-[3-(1,2,3,6-tetrahydro-pyridin-4-yl)-1H-indol-5-yl]-N⁴-thiophen-2-ylmethyl-pyrimidine-2,4-diamine

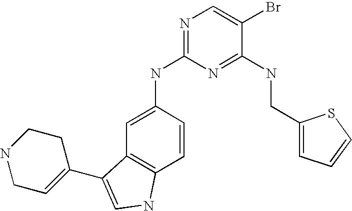

The title compound was synthesized in a 12% overall yield in a manner described in Example 9 from 2-methylaminothiophene. It was characterized as an off-white solid isolated as its TFA salt. C₂₂H₂₁BrN₆S. MS: 481.0/483.0 (MH+); ¹H NMR (D₆-DMSO) δ 11.24 (s, 1 H), 8.77 (s, 2 H), 8.04 (s, 2 H), 7.49 (s, 1 H), 7.32 (s, 3 H), 6.87 (m, 2 H), 6.05 (s, 1 H), 4.71 (s, 2 H), 3.69 (s, 2 H), 3.29 (s, 2 H), 2.67 (s, 2 H) ppm.

### EXAMPLE 21

5-Bromo-N4-furan-2-ylmethyl-N2-[3-(1,2,3,6-tetrahydro-pyridin-4-yl)-1H-indol-5-yl]-pyrimidine-2,4-diamine

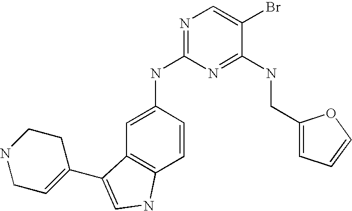

The title compound was made in a manner similar to Example 9. It was isolated in a 1% yield as an off-white solid characterized as its free base. C₂₂H₂₁BrN₆O. MS: 465.1/467.1 (MH+)

### EXAMPLE 22

5-Bromo-N⁴-(2-methyl-benzyl)-N²-[3-(1,2,3,6-tetrahydro-pyridin-4-yl)-1H-indol-5-yl]-pyrimidine-2,4-diamine

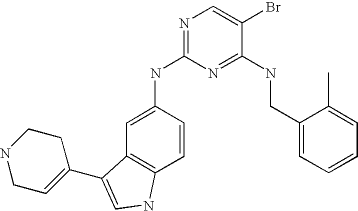

C₂₅H₂₅BrN₆.

### EXAMPLE 23

5-Bromo-N⁴-(3-methyl-benzyl)-N²-[3-(1,2,3,6-tetrahydro-pyridin-4-yl)-1H-indol-5-yl]-Pyrimidine-2,4-diamine

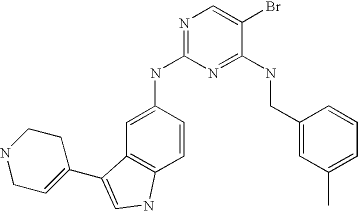

C₂₅H₂₅BrN₆.

### EXAMPLE 24

5-Bromo-N⁴-(4-methyl-benzyl)-N²-[3-(1,2,3,6-tetrahydro-pyridin-4-yl)-1H-indol-5-yl]-Pyrimidine-2,4-diamine

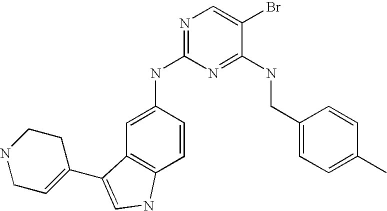

C₂₅H₂₅BrN₆.

### EXAMPLE 25

5-Bromo-N⁴-(2-fluoro-benzyl)-N²-[3-(1,2,3,6-tetrahydro-pyridin-4-yl)-1H-indol-5-yl]-pyrimidine-2,4-diamine

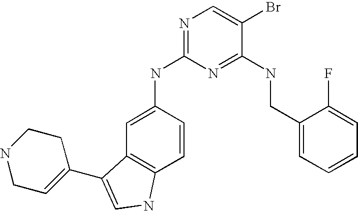

C₂₄H₂₂BrFN₆.

### EXAMPLE 26

N⁴-Biphenyl-2-ylmethyl-5-bromo-N²-[3-(1,2,3,6-tetrahydro-pyridin-4-yl)-1H-indol-5-yl]-pyrimidine-2,4-diamine

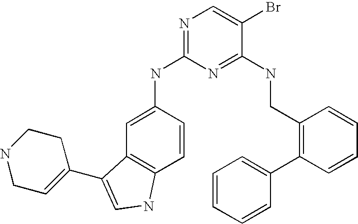

C₃₀H₂₇BrN₆.

### EXAMPLE 27

N⁴-Biphenyl-3-ylmethyl-5-bromo-N²-[3-(1,2,3,6-tetrahydro-pyridin-4-yl)-1H-indol-5-yl]-pyrimidine-2,4-diamine

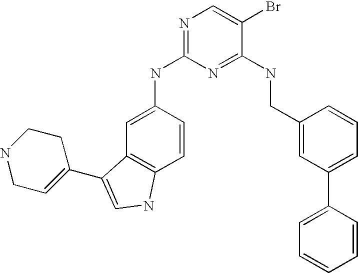

C₃₀H₂₇BrN₆.

### EXAMPLE 28

5-Bromo-N⁴-(2-methoxy-benzyl)-N²-[3-(1,2,3,6-tetrahydro-pyridin-4-yl)-1H-indol-5-yl]-pyrimidine-2,4-diamine

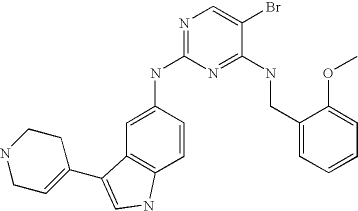

C₂₅H₂₅BrN₆O.

### EXAMPLE 29

5-Bromo-N⁴-(3-methoxy-benzyl)-N²-[3-(1,2,3,6-tetrahydro-pyridin-4-yl)-1H-indol-5-yl]-pyrimidine-2,4-diamine

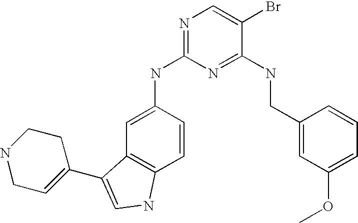

C₂₅H₂₅BrN₆O.

### EXAMPLE 30

3-({5-Bromo-2-[3-(1,2,3,6-tetrahydro-pyridin-4-yl)-1H-indol-5-ylamino]-pyrimidin-4-ylamino]-methyl)-N-methyl-benzamide

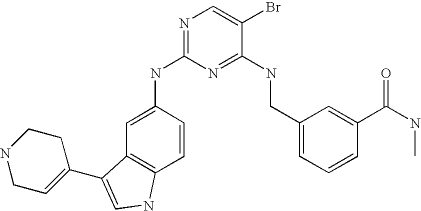

C₂₆H₂₆BrN₇O.

### EXAMPLE 31

5-Bromo-N⁴-(2-chloro-benzyl)-N²-[3-(1,2,3,6-tetrahydro-pyridin-4-yl)-1H-indol-5-yl]-pyrimidine-2,4-diamine

C₂₄H₂₂BrClN₆.

### EXAMPLE 32

5-Bromo-N⁴-Phenethyl-N²-[3-(1,2,3,6-tetrahydro-pyridin-4-yl)-1H-indol-5-yl]-pyrimidine-2,4-diamine

A. (5-Bromo-2-chloro-pyrimidin-4-yl)-phenethyl-amine

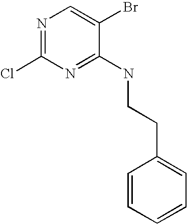

A 5.00 g (22.0 mmol) sample of 5-bromo-2,4-dichloropyrimidine was taken into 40.0 mL tetrahydrofuran with 7.80 mL (44.8 mmol) diisopropylethylamine. 3.53 g (22.4 mmol) phenethyl amine was added drop-wise with a white precipitate noted upon addition. After addition mLetion, the reaction mixture was allowed to stir at ambient temperature under nitrogen for three hours. The volatiles were removed under reduced pressure, and the resulting residue was partitioned between 1 N sodium hydroxide and ethyl acetate. Aqueous work-up afforded the title compound as 5.93 g (19.0 mmol, 95%) of a pale yellow, oily solid. C₁₂H₁₁BrClN₃: GC/MS: ret. Time: 4.77 min.: m/z 311/313/315, 220/222/224, 104; ¹H NMR (CDCl₃) δ 8.09 (s, 1 H), 7.34-7.30 (m, 2 H), 7.28-7.18 (m, 3 H), 5.53 (bs, 1 H), 3.75 (t, J=6.4 Hz, 2 H), 2.92 (t, J=6.4 Hz, 2 H) ppm.

B. 4-[5-(5-Bromo-4-phenethylamino-pyrimidin-2-ylamino)-1H-indol-3-yl]-3,6-dihydro-2H-pyridine-1-carboxylic acid tert-butyl ester

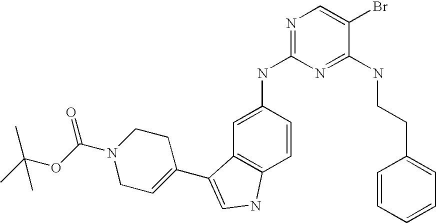

The title compound was made in a 35% yield in a manner similar to Example 1D using (L-bromo-2-chloro-pyrimidin-4-yl)-phenethyl-amine. C₃₀H₃₃BrN₆O₂: MS 589.1/591.1 (MH+);

¹H NMR (D₆-DMSO): δ 11.00 (s, 1 H), 8.92 (s, 1 H), 7.94 (s, 1 H), 7.40 (d, J=8.4 Hz, 1 H), 7.34 (s, 1 H), 7.22 (d, J=8.4 Hz, 1 H), 7.18-7.07 (m, 6 H), 6.90 (m, 1 H), 6.02 (s, 1 H), 3.98 (m, 2 H), 3.56 (m, 2 H), 3.45 (m, 2 H), 2.76 (t, J=7.6 Hz, 2 H), 2.42 (m, 2 H), 1.38 (s, 9 H) ppm.

C. 5-Bromo-N4-phenethyl-N2-[3-(1,2,3,6-tetrahydro-pyridin-4-yl)-1H-indol-5-yl]-pyrimidine-2,4-diamine

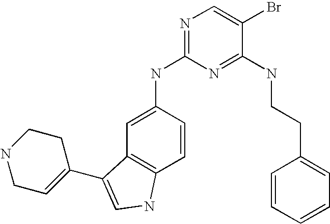

832 mg (1.70 mmol) 4-[5-(5-Bromo-4-phenethylamino-pyrimidin-2-ylamino)-1H-indol-3-yl]-3,6-dihydro-2H-pyridine-1-carboxylic acid tert-butyl ester was taken into 2.00 mL dichloromethane and cooled to 0° C. 4.00 mL trifluoroacetic acid was slowly added. The red reaction mixture was allowed to stir under nitrogen and slowly warm to ambient temperature over three hours. The volatiles were removed under reduced pressure. Ethyl acetate was added and evaporated an additional three times until a nearly clear yellow oil remained. Ethyl acetate was added (app. 1 mL) and stirred. Diethyl ether was added until a white precipitate was noted. Filtration of this precipitate afforded 716 mg of the title compound isolated as its Trifluoroacetate salt. C₂₅H₂₅BrN₆: MS: 489.1/491.1 (MH+); ¹H NMR (D₆-DMSO): δ 11.45 (s, 1 H), 10.32 (s, 1 H), 8.92 (s, 1 H), 8.31 (s, 1 H), 8.16 (s, 1 H), 7.91 (s, 1 H), 7.57 (s, 1 H), 7.40 (d, J=8.3 Hz, 1 H), 7.27 (d, J=8.3 Hz, 1 H), 7.11-6.90 (m, 5 H), 6.19 (bs, 1 H), 3.68 (m, 2 H), 3.46 (m, 2 H), 3.24 (m, 2 H), 2.71-2.66 (m, 4 H) ppm.

### EXAMPLE 33

5-Bromo-N⁴-(2-pyridin-2-yl-ethyl)-N²-[3-(1,2,3,6-tetrahydro-pyridin-4-y)-1H-indol-5-yl]-pyrimidine-2,4-diamine

A. (5-Bromo-2-chloro-pyrimidin-4-yl)-(2-pyridin-2-yl-ethyl)-amine

The title compound was made in a manner similar to Example 32A. It was isolated in an 83% yield as a tan solid. C₁₁H₁₀BrClN₄. MS 313.0/315.0/317.0 (MH+); ¹H NMR (D₆-DMSO) δ 8.53 (d, J=4.9 Hz, 1 H), 8.26 (s, 1 H), 7.92 (t, J=5.5 Hz, 1 H), 7.73 (t, J=7.6 Hz, 1 H), 7.30-7.23 (m, 2 H), 3.78-3.62 (m, 2 H), 3.07-3.02 (m, 2 H) ppm.

B. 5-Bromo-N4-(2-pyridin-2-yl-ethyl)-N2-[3-(1,2,3,6-tetrahydro-pyridin-4-yl)-1H-indol-5-yl]-pyrimidine-2,4-diamine

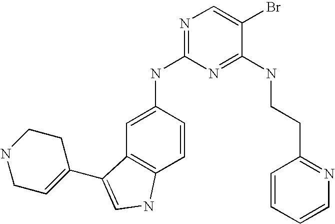

The title compound was synthesized in a manner similar to Example 32B and deprotected similarly to Example 21C. It was made in a 40% yield and isolated as a white solid, TFA salt. C₂₄H₂₄BrN₇. MS: 490.0/491.8 (MH+); ¹H NMR (D₆-DMSO) δ 11.41 (s, 1 H), 8.89 (s, 2 H), 8.59 (s, 1 H), 8.29-8.00 (m, 2 H), 7.91 (s, 2 H), 7.56-7.50 (m, 2 H), 7.38 (d, J=8.3 Hz, 1 H), 7.35-7.20 (m, 2 H), 6.07 (bs, 1 H), 3.98-3.72 (bs, 4 H), 3.37-3.30 (bs, 2 H), 3.10-3.00 (bs, 2 H), 2.67-2.46 (bs, 2 H) ppm.

### EXAMPLE 34

5-Bromo-N⁴-(2-pyridin-4-yl-ethyl)-N²-[3-(1,2,3,6-tetrahydro-pyridin-4-yl)-1H-indol-5-yl]-pyrimidine-2,4-diamine

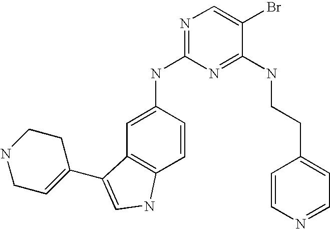

The title compound was made in a 30% yield in the same manner as Example 9 using 4-(2-ethylamino)pyridine. It was noted to be a white solid, isolated as its TFA salt. C₂₄H₂₄BrN₇. MS: 490.0/492.0 (MH+); ¹H NMR (D₆-DMSO) δ□11.37(s, 1 H),8.85 (s, 1 H), 8.50 (s, 2 H), 8.10 (s, 1 H), 7.94 (s, 1 H), 7.54 (s, 1 H), 7.37 (d, J=8.7 Hz, 1 H), 7.35 (bs, 1 H), 7.26 (d, J=9.1 Hz, 1 H), 6.06 (bs, 1 H), 3.75-3.65 (bs, 2 H), 3.60-3.50 (bs, 2 H), 3.35-3.25 (bs, 2 H), 3.00-2.90 (bs, 2 H), 2.70-2.60 (bs, 2 H) ppm.

### EXAMPLE 35

5-Bromo-N⁴-(2-pyridin-3-yl-ethyl)-N²-[3-(1,2,3,6-tetrahydro-pyridin-4-yl)-1H-indol-5-yl]-pyrimidine-2,4-diamine

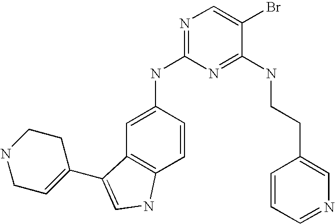

The title compound was made in a 23% overall yield starting from 3(2-ethylamino)pyridine, following the procedure of Example 9. The compound was noted to be an off-white solid isolated as its TFA salt. C₂₄H₂₄BrN₇. MS: 490.2/492.2 (MH+); ¹NMR (D₆-DMSO) δ 11.37 (s, 1 H), 8.82 (s, 2 H), 8.53 (s, 1 H), 8.49 (s, 1 H), 8.09 (s, 1 H), 8.00 (bs, 1 H), 7.97 (s, 1 H), 7.66 (bs, 1 H), 7.54 (s, 1 H), 7.39 (bs, 1 H), 7.37 (d, J=8.8 Hz, 1 H), 7.26 (d, J=8.3 Hz, 1 H), 6.07 (bs, 1 H), 3.70 (s, 2 H), 3.55(s, 2 H), 3.28(s, 2 H), 2.88 (s, 2 H), 2.70-2.60 (bs, 2 H) ppm.

### EXAMPLE 36

5-Bromo-N⁴-[2-(3-fluoro-phenyl)-ethyl]-N²-[3-(1,2,3,6-tetrahydro-pyridin-4-yl)-1H-indol-5-yl]-pyrimidine-2,4-diamine

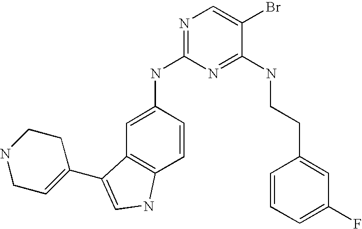

The title compound was isolated in a 4% yield as a white solid according to the procedure of Example 9. It was isolated as its free base after purifying over silica gel (93:7:0.7 CHCl₃:CH₃OH:NH₄OH). C₂₅H₂₄BrFN₆. MS: 507.0/508.8 (MH+); ¹⁹F NMR (D₆-DMSO) δ-114.0 ppm. ¹H NMR (D₆-DMSO) δ 10.90 (s, 1 H), 8.92 (s, 1 H), 8.08 (s, 1 H), 7.93 (s, 1 H), 7.41 (dd, J=1.6, 8.7 Hz, 1 H), 7.32 (s, 1 H), 7.27 (s, 1 H), 7.21-7.19 (m, 2 H), 6.99-6.88 (m, 4H), 6.08 (s, 1 H), 3.59-3.53 (m, 2 H), 3.31 (s, 2 H), 2.85-2.82 (m, 4 H), 2.32 (s, 2 H) ppm.

### EXAMPLE 37

5-Bromo-N⁴-(2-phenyl-cyclopropyl)-N²-[3-(1,2,3,6-tetrahydro-pyridin-4-yl)-1H-indol-5-yl]-pyrimidine-2,4-diamine

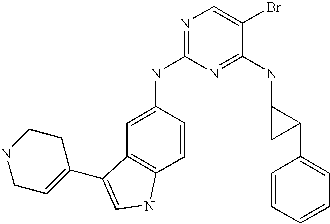

The title compound was synthesized in a 13% overall yield in a manner described in Example 1. C₂₆H₂₅BrN₆. 501.0/503.0 (MH+); ¹H NMR (D₆-DMSO) δ 11.28 (s, 1 H), 8.90 (bs, 2 H), 8.11 (s, 1 H), 7.90 (bs, 1 H), 7.86 (s, 1 H), 7.55 (s, 1 H), 7.43 (d, J=8.1 Hz, 1 H), 7.21-7.09 (m, 6 H), 6.08 (s, 1 H), 3.77 (s, 2 H), 3.34 (m, 3 H), 2.73 (s, 2 H), 2.25 (m, 1 H), 1.58 (m, 1 H), 1.20 (m, 1 H) ppm.

### EXAMPLE 37A

5-Bromo-N⁴-(2-phenyl-cyclopropyl)-N²-[3-(1,2,3,6-tetrahydro-pyridin-4-v)-1H-indol-5-yl]-pyrimidine-2,4-diamine (homo-chiral)

### EXAMPLE 37B

5-Bromo-N⁴-(2-phenyl-cyclopropyl)-N²-[3-(1,2,3,6-tetrahydro-pyridin-4-yl)-1H-indol-5-yl]-Pyrimidine-2,4-diamine (homo-chiral)

### EXAMPLE 38

5-Bromo-N⁴-[2-(4-chloro-phenyl)-ethyl]-N²-[3-(1,2,3,6-tetrahydro-pyridin-4-yl)-1H-indol-5-yl]-pyrimidine-2,4-diamine

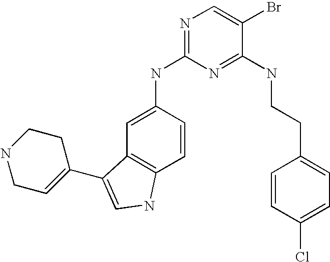

The title compound was isolated in a 10% overall yield in a manner described by Example 9 from 4-chlorophenethyl amine. It was characterized as an off-white solid isolated as its TFA salt. C₂₅H₂₄BrClN₆ MS: 522.9/524.91527.0 (MH+); ¹H NMR (D₆-DMSO) δ 11.37 (s, 1 H), 8.79 (s, 2 H), 8.07 (s, 1 H), 7.93 (s, 1 H), 7.56 (s, 1 H), 7.37 (d, J=8.8 Hz, 1 H), 7.30 (s, 1 H), 7.13 (bs, 2 H), 6.97 (s, 2 H), 6.06 (s, 1 H), 3.69 (s, 2 H), 3.34 (s, 2 H), 3.26 (s, 2 H), 2.67 (m, 4 H) ppm.

### EXAMPLE 39

5-Bromo-N²-[3-(1,2,3,6-tetrahydro-pyridin-4-yl)-1H-indol-5-yl]-N⁴-(2-thiophen-2-yl-ethyl)-pyrimidine-2,4-diamine

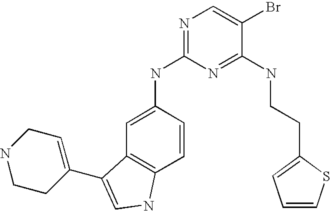

The title compound was isolated in 13% overall yield in a manner described by Example 9 from 2-ethylaminothiophene. It was characterized as an off-white solid isolated as its TFA salt. C₂₃H₂₃BrN₆S. MS: 495.1/497.1 (MH+); ¹H NMR (D₆-DMSO) δ 11.38 (s, 1 H), 8.86 (s, 2 H), 8.11 (s, 1 H), 8.00 (s, 1 H), 7.57 (s, 1 H), 7.39 (s, 2 H), 7.35 (d, J=5.3 Hz, 1 H), 6.94 (m, 1 H), 6.78 (s, 1 H), 6.11 (s, 1 H), 3.75 (s, 2 H), 3.62 (s, 2 H), 3.34 (s, 2 H), 3.09 (s, 2 H), 2.72 (s, 2 H) ppm.

### EXAMPLE 40

5-Bromo-N⁴-[2-(2-fluoro-phenyl)-ethyl]-N²-[3-(1,2,3,6-tetrahydro-pyridin-4-yl)-1H-indol-5-yl]-pyrimidine-2,4-diamine

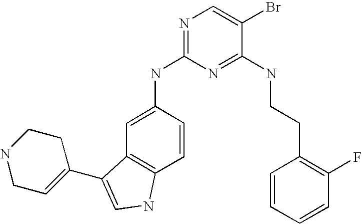

The title compound was made in a 12% yield in a manner described in Example 9. It was characterized as an off-white solid isolated as its HCl salt. C₂₅H₂₄BrFN₆. MS: 507.0/508.9 (MH+); ¹HNMR (D₆-DMSO) δ 11.43 (s, 1 H), 10.37 (s, 1 H), 9.20 (s, 2 H), 8.53 (bs, 1 H), 8.20 (bs, 1 H), 7.90 (s, 1 H), 7.57 (s, 1 H), 7.41 (s, 1 H), 7.18-7.06 (m, 3 H), 6.89 (bs, 1 H), 6.06 (s, 1 H), 3.66 (s, 2 H), 3.46 (s, 2 H), 3.23 (s, 2 H), 2.80 (s, 2 H), 2.67 (s, 2 H) ppm.

### EXAMPLE 41

5-Bromo-N⁴-[2-(2-chloro-phenyl)-ethyl]-N²-[3-(1,2,3,6-tetrahydro-pyridin-4-yl)-1H-indol-5-yl]-pyrimidine-2,4-diamine

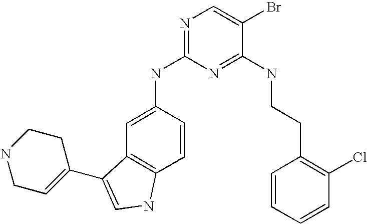

The title compound was made in a 20% yield in a manner described in Example 9. It was characterized as an off-white solid and isolated as its HCl salt. C₂₅H₂₄BrClN₆. MS: 523.1/525.1/527.1 (MH+); ¹HNMR (D₆-DMSO) δ 11.45 (s, 1 H), 10.37 (s, 1 H), 9.17 (bs, 2 H), 8.54 (s, 1 H), 8.28 (s, 1 H), 7.87 (s, 1 H), 7.57 (s, 1 H), 7.42 (d, J=8.7 Hz, 1 H), 7.33 (d, J=7.5 Hz, 1 H), 7.22-7.17 (m, 2 H), 6.98 (bs, 1 H), 6.06 (s, 1 H), 3.66 (s, 2 H), 3.52 (s, 2 H), 3.22 (s, 2 H), 2.90 (s, 2 H), 2.67 (s, 2 H) ppm.

### EXAMPLE 42

5-Bromo-N⁴-[2-(2-methoxy-phenyl)-ethyl]-N²-[3-(1,2,3,6-tetrahydro-pyridin-4-yl)-1H-indol-5-yl]-Pyrimidine-2,4-diamine

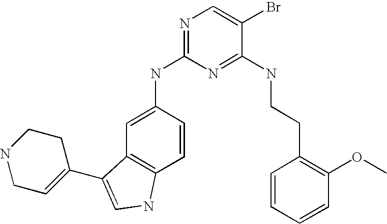

The title compound was made in a 6% yield in a manner described in Example 9. It was characterized as an off-white solid and isolated as its HCl salt. C₂₆H₂₇BrN₆O. MS: 519.0/520.9 (MH+); ¹HNMR (D₆-DMSO) δ 11.47 (s, 1 H), 10.46 (s, 1 H), 9.28 (bs, 2 H), 8.56 (s, 1 H), 7.90 (s, 1 H), 7.57 (s, 1 H), 7.41 (d, J=8.8 Hz, 1 H), 7.20 (s, 1 H), 7.17 (s, 1 H), 6.85 (d, J=7.9 Hz, 1 H), 6.65 (bs, 2 H), 6.05 (s, 1 H), 3.76 (s, 3 H), 3.65 (s, 2 H), 3.53 (s, 2 H), 3.20 (s, 2 H), 2.75 (s, 2 H), 2.67 (s, 2 H) ppm.

### EXAMPLE 43

N⁴-(2-Benzo[1,3]dioxol-5-yl-ethyl)-5-bromo-N²-[3-(1,2,3,6-tetrahydro-pyridin-4-yl)-1H-indol-5-yl]-pyrimidine-2,4-diamine

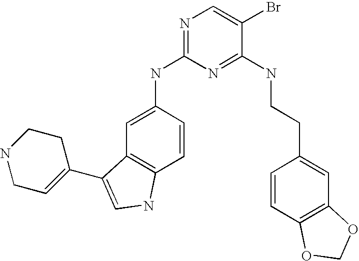

The title compound was made in a 4% yield in a manner described in Example 9. It was characterized as an off-white solid isolated as its HCl salt. C₂₆H₂₅BrN₆O₂. MS: 533.6/535.6 (MH+); ¹HNMR (D₆-DMSO) δ 11.47 (s, 1 H), 10.43 (s, 1 H), 9.29 (bs, 2 H), 8.53 (s, 1 H), 8.34 (s, 1 H), 7.88 (s, 1 H), 7.67 (s, 1 H), 7.42 (s, 1 H), 7.22 (s, 1 H), 6.60 (m, 2 H), 6.05 (s, 1 H), 3.63 (s, 2 H), 3.52 (s, 2 H), 3.45 (s, 2 H), 2.69 (m, 4 H) ppm.

### EXAMPLE 44

5-Bromo-N⁴-(3-phenyl-propyl)-N 2-[3-(1,2,3,6-tetrahydro-pyridin-4-yl)-1H-indol-5-yl]-Pyrimidine-2,4-diamine

A. (5-Bromo-2-chloro-Pyrimidin-4-yl)-(3-phenyl-propyl)-amine

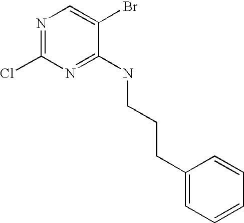

The title compound was made in a manner similar to Example 1A except performing the reaction at ambient temperature. It was isolated as a yellow oil which solidified upon standing in a 84% yield. MS: 324/326/328 (MH+); ¹H NMR (CDCl₃) δ 8.30 (s, 1 H), 7.37-7.23 (m, 5 H), 5.52 (s, 1 H), 3.57 (tt, J=7.5, 7.3 Hz, 2 H), 2.77 (t, J=7.5 Hz, 2 H), 2.04 (t, J=7.3 Hz, 2 H) ppm.

B. 5-Bromo-N⁴-(3-phenyl-propyl)-N²-[3-(1,2,3,6-tetrahydro-pyridin-4-yl)-1H-indol-5-yl-pyrimidine-2,4-diamine

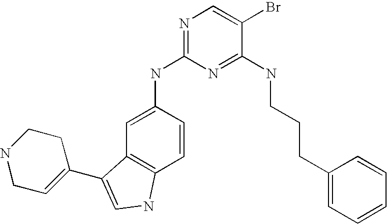

The title compound was isolated as its TFA salt following the procedure of Example 1D and deprotecting according Example 4C in a 34% yield as a white solid. C₂₆H₂₇BrN₆. MS: 503.2/505.1 (MH+); ¹H NMR (D₆-DMSO) δ 11.32 (s, 1 H), 8.90 (s, 1 H), 8.05 (s, 1 H), 7.93 (s, 1 H), 7.53 (s, 1 H), 7.35 (s, 2 H), 7.21-7.05 (m, 7 H), 6.07 (bs, 1 H), 3.80-3.70 (bs, 2 H), 3.37-3.31 (bs, 4 H), 2.70-2.60 (bs, 2 H), 2.47-2.46 (bs, 2 H), 2.00-1.90 (bs, 2 H).

### EXAMPLE 45

5-(5-Bromo-4-phenethylamino-pyrimidin-2-ylamino)-1,3-dihydro-indol-2-one

A. 5-Nitro-1,3-dihydro-indol-2-one

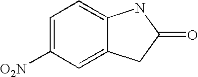

C₈H₆N₂O₃: GC/MS ret. time: 4.12 min., m/z 178, 148, 104; ¹H NMR (D₆-DMSO) d 10.50 (s, 1 H), 8.11 (d, J=8.7 Hz, 1 H), 8.05 (s, 1 H), 6.94 (d, J=8.7 Hz, 1 H), 3.59 (s, 2 H) ppm.

B. 5-Amino-1,3-dihydro-indol-2-one

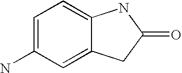

To 250 mL acetic acid was added 7.00 g (39.3 mmol) 5-nitro-1,3-dihydro-indol-2-one and 418 mg (0.393 mmol) palladium on carbon. Exposed the reaction mixture to 40 psi H₂ on parr shaker for 1.5 hours. The reaction was filtered through diatameceous earth, and the acetic acid was removed under reduced pressure. Cooled the reaction mixture to 0° C. and added 10.0 mL of a 94.5:5:0.5 CHCl₃:CH₃OH:NH₄OH solution. The solution was loaded onto a silica gel column and purified via chromatography (97.8:2.0:0.2 CHCl₃:CH₃OH:NH₄OH) to give a white solid which was further crystallized using the eluent as the solvent to give 4.06 g (27.2 mmol, 69%) of the title compound as crystalline white needles. C₈H₉N2O:

C. 5-(5-Bromo-4-phenethylamino-pyrimidin-2-ylamino)-1,3-dihydro-indol-2-one

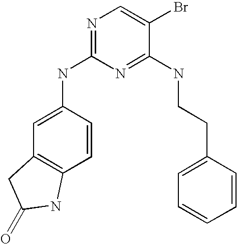

153 mg (0.490 mmol) (5-Bromo-2-chloro-pyrimidin-4-yl)-phenethyl-amine was taken into 500 □L 1,4 dioxane with 140 □L (1.00 mmol) diisopropylethylamine and 80 mg (0.539 mmol) 5-amino-1,3-dihydro-indol-2-one. The reaction was allowed to heat to 110° C. for sixteen hours. The resulting brown glass was taken into 92.3:7:0.7 CHCl₃:CH₃OH:NH₄OH and washed with 1 N sodium hydroxide. The organic layer was dried over magnesium sulfate and evaporated directly onto silica gel. This adsorbed compound was purified via column chromatography (97.8:2:0.2 CHCl₃:CH₃OH:NH₄OH) over silica to isolate the major product. During evaporation of the major fractions, a white precipitate is noted. Filtration of this precipitate prior to mLete evaporation afforded the title compound in 6% yield as a white solid. C₂₀H₁₈BrN₅O: MS: 424.2/426.2 (MH+); ¹H NMR (D₆-DMSO) 10.20 (s, 1 H), 9.01 (s, 1 H), 7.93 (s, 1 H), 7.52 (s, 1 H), 7.44 (d, J=8.4 Hz, 1 H), 7.28-7.16 (m, 5 H), 6.97 (m, 1 H), 6.65 (d, J=8.3 Hz, 1 H), 3.56 (m, 2 H), 3.31 (s, 2 H), 2.82 (t, J=7.9 Hz, 2 H) ppm.

### EXAMPLE 46

5-[5-Bromo-4-(2-chloro-benzylamino)-pyrimidin-2-ylamino]-1,3-dihydro-indol-2-one

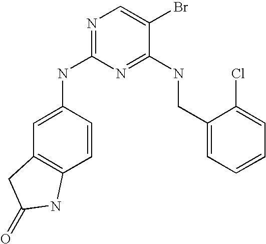

C₁₉H₁₅BrClN₅O.

### EXAMPLE 47

5-(4-Benzylamino-5-bromo-pyrimidin-2-ylamino)-1,3-dihydro-indol-2-one

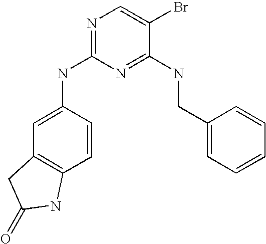

C₁₉H₁₆BrN₅O

### EXAMPLE 48

5-[5-Bromo-4-(1-phenyl-ethylamino)-pyrimidin-2-viamino]-1,3-dihydro-indol-2-one

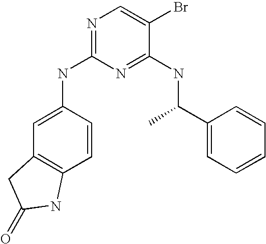

C₂₀H₁₈BrN₅O.

### EXAMPLE 49

5-[5-Bromo-4-(3-Phenyl-propylamino)-pyrimidin-2-ylamino]-1,3-dihydro-indol-2-one

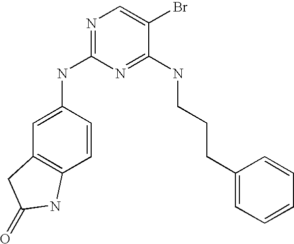

C₂, H₂₀BrN₅O.

### EXAMPLE 50

5-Bromo-N⁴-(2-methanesulfonyl-ethyl)-N²-[3-(1,2,3,6-tetrahydro-pyridin-4-yl)-1H-indol-5-yl]-pyrimidine-2,4-diamine

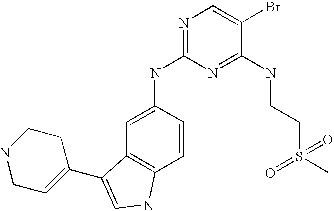

The title compound was made in a 13% yield in a manner described in Example 9. It was characterized as an off-white solid isolated as it TFA salt. C₂₀H₂₃BrN₆O₂S: MS: 491.1/493.1 (MH+); ¹H NMR (D₆-DMSO) δ 11.28 (s, 1 H), 8.84 (s, 2 H), 8.09 (s, 1 H), 7.95 (s, 1 H), 7.83 (s, 1 H), 7.52 (s, 1 H), 7.38 (s, 1 H), 7.36 (s, 1 H), 6.07 (s, 1 H), 3.75 (m, 4 H), 3.34 (m, 4 H), 2.90 (s, 3 H), 2.69 (m, 2 H) ppm.

### EXAMPLE 51

N⁴-Benzyl-N²-[3-(1,2,3,6-tetrahydro-pyridin-4-yl)-1H-indol-5-yl]-pyrimidine-2,4-diamine

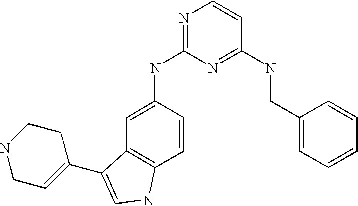

250 mg (0.424 mmol) N⁴-Benzyl-5-bromo-N²-[3-(1,2,3,6-tetrahydro-pyridin-4-yl)-1H-indol-5-yl]-pyrimidine-2,4-diamine trifluoroacetate was suspended in 12.7 mL conc. NH₄OH. To this was added 0.636 g (9.73 mmol) zinc dust. The resulting slurry was heated to reflux for three hours. The gray mixture was filtered through diatomaceous earth. The filtrate was evaporated under reduced pressure to give the title compound in 39% yield isolated as a white solid. C₂₄H₂₄N₆. MS: 397.2 (MH+); ¹H NMR (CD₃OD) δ 8.05 (s, 1 H), 7.66 (d, J=5.8 Hz, 1 H), 7.30-7.17 (m, 7H), 6.15 (s, 1 H), 5.87 (d, J=5.8 Hz, 1 H), 4.55 (s, 2 H), 3.41 (s, 2 H), 3.05 (s, 2 H), 2.53 (s, 2 H) ppm.

### EXAMPLE 52

N⁴-Benzyl-N⁴-methyl-N²-23-(1,2,3,6-tetrahydro-pyridin-4-yl)-1H-indol-5-yl]-pyrimidine-2,4-diamine

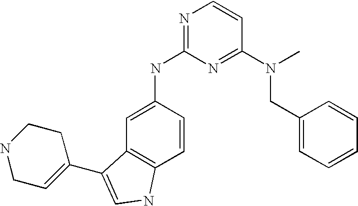

The title compound was synthesized in a 4% overall yield in a manner similar to Example 9 using 2-4-dichloropyrimidine and N-methyl benzyl amine. It was characterized as an off-white solid isolated as its free base. C₂₅H₂₆N₈. MS: 411.2 (MH+); ¹H NMR (D₆-DMSO) δ 10.85 (s, 1 H), 8.23 (s, 1 H), 7.88 (d, J=5.8 Hz, 1 H), 7.35-7.15 (m, 9 H), 6.07 (s, 1 H), 6.04 (d, J=5.8 Hz, 1 H), 4.78 (s, 2 H), 3.32 (s, 2 H), 3.13 (s, 2 H), 2.93 (m, 2 H), 2.47 (s, 3 H) ppm.

### EXAMPLE 53

N⁴-Methyl-N⁴-(2-pyridin-2-yl-ethyl)-N²-[3-(1.,2,3,6-tetrahydro-pyridin-4-yl)-1H-indol-5-yl]-pyrimidine-2,4-diamine

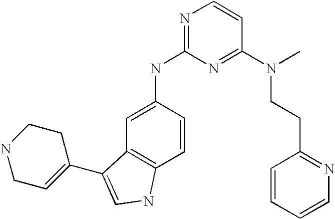

The title compound was made in a 1% yield in a manner described in Example 9. It was characterized as a white solid isolated as its free base after purifying the TFA salt over silica (93:7:0.7 CHCl₃:CH₃OH:NH₄OH). C₂₅H₂₇N₇. MS: 426.1 (MH+); ¹H NMR (CD₃OD) δ 8.37 (s, 1 H), 8.00 (s, 1 H), 7.76 (t, J=7.5 Hz, 1 H), 7.44 (bs, 1 H), 7.33-7.15 (m, 5 H), 6.14 (s, 1 H), 5.97 (d, J=5.8 Hz, 1 H), 5.94 (d, J=7.5 Hz, 1 H), 3.87-3.78 (m, 2 H), 3.52-3.50 (m, 2 H), 3.11-3.06 (m, 2 H), 3.00 (s, 3 H), 2.97 (s, 2 H) ppm.

### EXAMPLE 54

[4-(2-Phenyl-morpholin-4-yl)-pyrimidin-2-yl]-[3-(1,2,3,6-tetrahydro-pyridin-4-yl)-1H-indol-5-yl]-amine

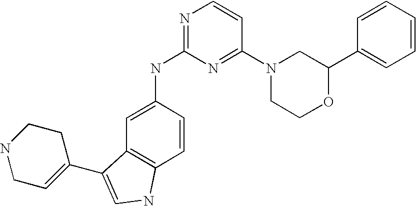

The title compound was synthesized in a 9% overall yield in a manner described by Example 9 using 2-phenylmorpholine and 2,4-dichloropyrimidine. It was characterized as an off-white solid isolated as its TFA salt. C₂₇H₂₈N₆O. MS: 453.3 (MH+); ¹H NMR

### EXAMPLE 55

5-Methyl-N⁴-(2-pyridin-2-yl-ethyl)-N²-[3-(1,2,3,6-tetrahydro-pyridin-4-yl)-1H-indol-5-yl]-Pyrimidine-2,4-diamine

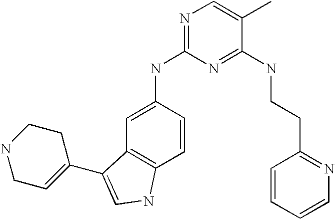

C₂₅H₂₇N₇.

### EXAMPLE 56

5-Bromo-N²-(3-piperidin-4-yl-1H-indol-5-yl)-N⁴-(2-pyridin-2-yl-ethyl)-pyrimidine-2,4-diamine

A. 4-(5-Amino-1H-indol-3-yl)-piperidine-1-carboxylic acid tert-butyl ester

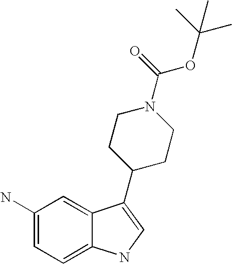

5.00 g 4-(5-Nitro-1H-indol-3-yl)-3,6-dihydro-2H-pyridine-1-carboxylic acid tert-butyl ester (14.6 mmol) was taken into 40.0 mL THF and 160 mL ethyl acetate 2/1.00 mL (5.74 mmol) diisopropylethylamine. 1.56 g (1.46 mmol) Pd/C was added. The reaction was shaken on a parr shaker under 3 atm H₂ for 90 minutes. The reaction vessel was removed from pressure. It was filtered through a bed of diatomaceous earth and was washed thoroughly with ethyl acetate. The clear, colorless filtrate was evaporated under reduced pressure to give an impure white solid. The white solid was taken into a minimum amount of dichloromethane and tritrated with hexanes. Filtration afforded the title copound in 84% yield as a white solid. C₁₈H₂₅N₃O₂. MS: 315.3, 216.1 (MH+); ¹H NMR (D₆-DMSO) δ 10.24 (s, 1 H), 6.99 (d, J=8.3 Hz, 1 H), 6.87 (d, J=2.1 Hz, 1 H), 6.66 (s, 1 H), 6.42 (dd, J=2.1 Hz, 8.3 Hz, 1 H), 4.38 (s, 2 H), 4.00 (m, 2 H), 2.75 (m, 2 H), 2.47 (m, 2 H), 1.86 (m, 2 H), 1.46 (m, 2 H) ppm.

B. 5-Bromo-N²-(3-piperidin-4-yl-1H-indol-5-yl)-N⁴-(2-pyridin-2-yl-ethyl)-pyrimidine-2,4-diamine

The title compound was made in a manner similar to Example 1D and deprotected according to the procedure of Example 1E in a 38% yield. The compound was characterized as an off-white solid and isolated as its HCl salt.

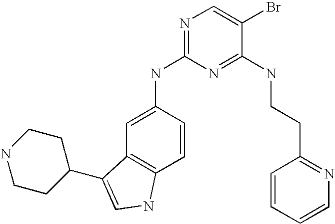

C₂₄H₂₆BrN₇. MS: 492.1/494.0 (MH+); ¹H NMR (D₆-DMSO) δ 11.11 (s, 1 H), 10.57 (s, 1 H), 9.16 (s, 1 H), 9.08 (s, 1 H), 8.69 (s, 1 H), 8.61 (s, 1 H), 8.32 (bs, 1 H), 8.17 (bs, 1 H), 7.74 (s, 2 H), 7.37 (d, J=8.7 Hz, 1 H), 7.15 (s, 1 H), 7.11 (s, 1 H), 3.73 (s, 2 H), 3.26 (s, 4 H), 2.02 (s, 2 H), 1.88 (s, 2 H) ppm.

### EXAMPLE 57

5-Bromo-N²-[1-methanesulfonyl-3-(1,2,3,6-tetrahydro-pyridin-4-yl)-1H-indol-5-yl]-N⁴-(2-pyridin-2-yl-ethyl)-pyrimidine-2,4-diamine

A. 4-(1-Methanesulfonyl-5-nitro-1H-indol-3-yl)-3,6-dihydro-2H-pyridine-1-carboxylic acid tert-butyl ester

2.00 g (5.82 mmol) 4-(5-Nitro-1H-indol-3-yl)-3,6-dihydro-2H-pyridine-1-carboxylic acid tert-butyl ester was suspended in 15.0 mL toluene and 15.0 mL 15% sodium hydroxide solution and cooled to 0° C. To this was added 349 mg (0.874 mmol)ⁿbu₄N(HSO₄) tetra-n-butyl hydrogensulfate. 676 □L (8.74 mmol) methanesulfonyl chloride was slowly dropped in. There was noted an immediate dissolution of the solids and a color change to red. Allowed the reaction to slowly warm to ambient temperature over sixteen hours. Reaction was regularly monitored and aliquots of 6760L (8.74 mmol) methanesulfonyl chloride were added until complete disappearance of starting material by TLC. Ethyl acetate was added and the layers were separated. Aqueous work-up gave a yellow solid which was purified over silica (20%→50% ethyl acetate in hexanes) to give the title compound in a 76% yield as a yellow solid. ¹H NMR (D₆-DMSO) δ 8.69 (d, J=2.3 Hz, 1 H), 8.25 (dd, J=9.1, 2.3 Hz, 1 H), 8.05 (d, J=9.1 Hz, 1 H), 7.84 (s, 1 H), 6.34 (s, 1 H), 4.06 (s, 2 H), 3.56 (s, 3 H), 3.55-3.53 (m, 2 H), 2.51 (s, 2 H), 1.41 (s, 9 H) ppm.

B. 4-(5-Amino-1-methanesulfonyl-1H-indol-3-yl)-3,6-dihydro-2H-pyridine-1-carboxylic acid tert-butyl ester

4-(1-Methanesulfonyl-5-nitro-1H-indol-3-yl)-3,6-dihydro-2H-pyridine-1-carboxylic acid tert-butyl ester was reduced in a manner described in Example 1C in a 89% yield as an orange foam. ¹H NMR (D₆-DMSO) δ 7.48 (d, J=9.0 Hz, 1 H), 7.33 (s, 1 H), 7.05 (s, 1 H), 6.66 (d, J=9.0 Hz, 1 H), 6.16 (s, 1 H), 4.98 (s, 2 H), 4.02-3.96 (m, 2 H), 3.53-3.50 (m, 2 h), 3.23 (s, 3 H), 2.47-2.44 (m, 2 H), 1.40 (s, 9 H) ppm.

C. 5-Bromo-N 2-[1-methanesulfonyl-3-(1,2,3,6-tetrahydro-pyridin-4-yl)-1H-indol-5-yl]-N⁴-(2-pyridin-2-yl-ethyl)-pyrimidine-2,4-diamine

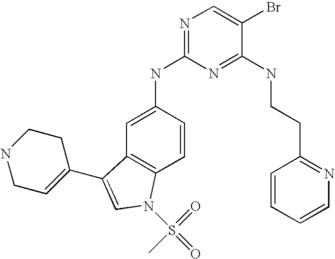

The title compound was made in a manner 30% yield in a manner described in Example 1D and 1E. It was characterized as an off-white solid and isolated as its HCl salt. C₂₅H₂₆BrN₇O₂S. MS: 568.0/569.9 (MH+); ¹H NMR (D₆-DMSO) δ 10.67 (bs, 1 H), 9.52 (s, 2H), 8.64 (d, J=5.4 Hz, 1 H), 8.44 (s, 1 H), 8.27 (s, 1 H), 8.18 (s, 2 H), 7.86 (d, J=9.0 Hz, 1 H), 7.75-7.67 (m, 2 H), 7.52 (d, J=9.0 Hz, 1 H), 6.29 (s, 1 H), 3.77 (s, 2 H), 3.48 (s, 3 H), 3.28 (s, 4 H), 2.73 (s, 2 H) ppm.

### EXAMPLE 58

5-Bromo-N²-[1-methanesulfonyl-3-(1,2,3,6-tetrahydro-pyridin-4-yl)-1H-indol-5-yl]-N⁴-pyridin-2-yl-pyrimidine-2,4-diamine

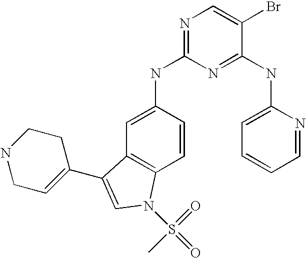

C₂₃H₂BrN₇O₂S.

### EXAMPLE 59

5-Bromo-N²-(2-pyridin-2-yl-ethyl)-N⁴-[3-(1,2,3,6-tetrahydro-pyridin-4-yl)-1H-indol-5-yl]-pyrimidine-2,4-diamine

A. (5-Bromo-2-chloro-pyrimidin-4-yl)-[3-(1,2,3,6-tetrahydro-pyridin-4-yl)-1H-indol-5-yl]-amine

The title compound was made in a quantitative yield following the procedure of Example 1A. It was characterized as an oily, yellow solid without purification. C₁₇H₁₅BrClN₅ MS: 503.1/505.1 (MH+); ¹H NMR (CD₃OD) δ 8.23 (s, 1 H), 8.14 (s, 1 H), 7.35 (d, J=8.5 Hz, 1 H), 7.30 (s, 1 H), 7.22 (d, J=8.5 Hz, 1 H), 6.14 (s, 1 H), 4.10 (s, 2 H), 3.64 (s, 2 H), 2.56 (s, 2H), 1.48 (s, 9 H) ppm.

B. 5-Bromo-N 2-(2-pyridin-2-yl-ethyl)-N⁴-[3-(1,2,3,6-tetrahydro-pyridin-4-yl)-1H-indol-5-yl]-pyrimidine-2,4-diamine

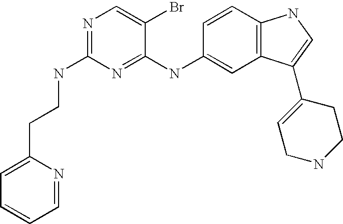

The title compound was made in a 2% yield via the manner described in Example 1D and 1E. It was characterized as an off white solid isolated as its free base after purifying the HCl salt over silica (93:7:0.7 CHCl₃:CH₃OH:NH₄OH). C₂₄H₂₄BrN₇. HPLC ret. time: 3.93 min.; MS: 490.0/492.1 (MH+); ¹H NMR (CD₃OD) δ 8.31 (s, 1 H), 7.94 (bs, 1 H), 7.87 (s, 1 H), 7.37-7.32 (m, 4 H), 7.26 (dt, J=9.0, 2.0 Hz, 1 H), 7.12 (s, 1 H), 6.16 (s, 1 H), 3.67 (s, 2 H), 3.43 (s, 2 H), 3.25-3.24 (m, 2 H), 2.84 (s, 2 H), 2.67 (s, 2 H) ppm.

### EXAMPLE 60

3-{4-(2-Pyridin-2-yl-ethylamino)-2-[3-(1,2,3,6-tetrahydro-pyridin-4-yl)-1H-indol-5-viamino]-pyrimidin-5-yl}-acrylic acid ethyl ester

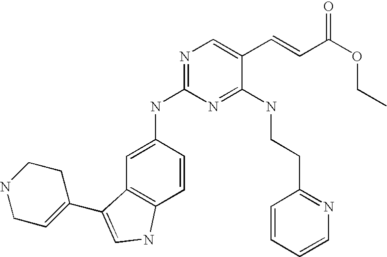

C₂₉H₃₁N₇O₂.

### EXAMPLE 60A

5-[5-Bromo-4-[2-(3-chloro-phenyl)-ethylamino]-pyrimidin-2-ylamino]-1,3-dihydro-indol-2-one

### EXAMPLE 61

5-Bromo-N⁴-[2-(3-chloro-phenyl)-ethyl]-N²-[3-(1,2,3,6-tetrahydro-pyridin-4-yl)-1H-indol-5-yl]-pyrimidine-2,4-diamine

A. (5-Bromo-2-chloro-pyrimidin-4-yl)-[2-(3-chloro-phenyl)-ethyl]-amine

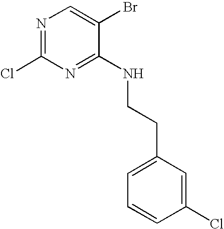

Using method B, the title compound was isolated in a 79% yield (1.37 g, 3.95 mmol) 20 as a white solid. GC/MS: ret. time=5.30, m/z 345/347/349; ¹H NMR (d₆-DMSO) δ 8.20 (s, 1 H), 7.75 (t, 1 H), 7.29-7.12 (m, 4 H), 3.56 (q, 2 H), 2.84 (t, 2 H) ppm.

B. 5-{5-Bromo-4-[2-(3-chloro-phenyl)-ethylamino]-pyrimidin-2-ylamino}-1,3-dihydro-indol-2-one (C₂₀H₁₇BrClN₅O)

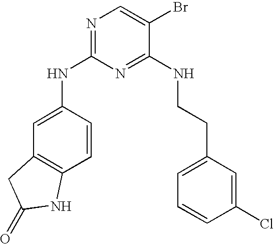

The title compound was isolated as a brown solid in a 14% yield. MS: 459.9/461.2 (MH+). ¹H NMR (d₆-DMSO) δ: 10.19 (s, 1 H), 9.02 (s, 1 H), 8.28 (s, 1 H), 7.93 (s, 1 H), 7.41 (dd, 1 H), 7.30-7.22 (m, 3 H), 7.13-7.11 (m, 1 H), 6.98 (t, 1 H), 6.65 (d, 1 H), 3.56 (q, 2 H), 3.33 (s, 1 H), 2.84 (t, 2 H).

### EXAMPLE 62

5-Bromo-N⁴-[2-(3-chloro-phenyl)-ethyl]-N²-[3-(1,2,3,6-tetrahydro-pyridin-4-yl)-1H-indol-5-yl]-pyrimidine-2,4-diamine

A. (5-Bromo-2-chloro-pyrimidin-4-yl)-[2-(3-chloro-Phenyl)-ethyl]-amine

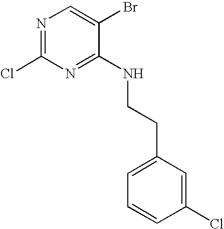

The title compound was prepared according to method b and isolated in a 79% yield (1.37 g, 3.95 mmol) as a white solid (C₁₂H₁₀BrCl₂N₃): GC/MS: ret. time=5.30, m/z 345/347/349; ¹H NMR (d₆-DMSO) δ 8.20 (s, 1 H), 7.75 (t, 1 H), 7.29-7.12 (m, 4 H), 3.56 (q, 2 H), 2.84 (t, 2 H) ppm.

B. 4-(6-[5-Bromo-4-[2-(3-chloro-phenyl)-ethylamino]-pyrimidin-2-ylamino}-1H-indol-3-yl)-3,6-dihydro-2H-pyridine-1-carboxylic acid tert-butyl ester.

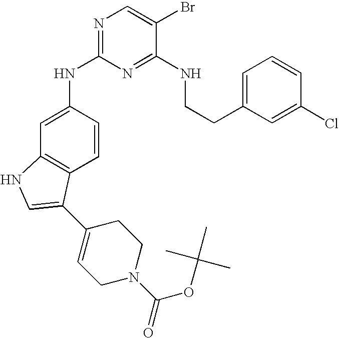

The title compound was prepared according to method E (C₃₀H₃₂BrClN₆O₂): MS: 623.1/625.1 (MH+); ¹H NMR (d₆-DMSO) δ: 10.99 (s, 1 H), 8.92 (s, 1 H), 8.12 (s, 1 H), 7.94 (s, 1 H), 7.40-7.33 (m, 2 H), 7.24-7.16 (m, 4 H), 7.02-7.00 (m, H), 6.92 (t, 1 H), 6.02 (s, 1 H), 3.94 (s, 2 H), 3.56 (q, 2 H), 3.46 (m, 2 H), 3.28 (s, 1 H), 2.81 (t, 2 H), 1.38 (s, 9 H) ppm.

C. 5-Bromo-N⁴-[2-(3-chloro-phenyl)-ethyl]-N²-[3-(1,2,3,6-tetrahydro-pyridin-4-yl)-1H-indol-5-yl]-pyrimidine-2,4-diamine (C₂₅H₂₄BrClN₆).

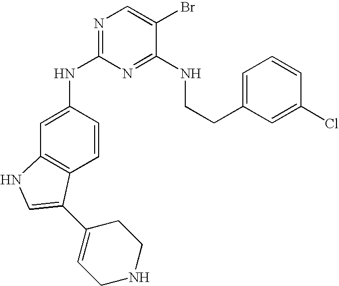

The title compound was prepared according to method G and isolated as the TFA salt in a 15% yield. MS: 522.9/525.1 (MH+). ¹H NMR (CDCl₃) δ: 12.16 (s, 1 H), 9.67 (s, 2 H), 8.90 (s, 1 H), 8.75 (s, 2 H), 8.34 (s, 1 H), 8.19-8.13 (m, 2 H), 8.03-7.94 (m, 3 H), 7.72 (s, 1 H), 6.87 (s, 1 H), 4.51 (s, 2 H), 4.32 (s, 2 H), 4.07 (s, 2 H), 3.59 (s, 2 H), 3.47 (s, 2 H) ppm.

### EXAMPLE 63

5-(5-Bromo-4-[2-(4-methoxy-phenyl)-ethylamino]-pyrimidin-2-ylamino]-1,3-dihydro-indol-2-one

A. (5-Bromo-2-chloro-pyrimidin-4-yl)-[2-(4-methoxy-phenyl)-ethyl]-amine.

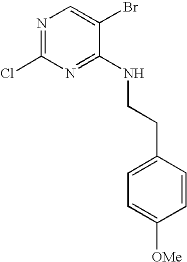

The title compound was prepared according to method b and isolated as a light yellow, viscous oil in an 80% yield (C₁₃H₁₃BrClN₃O). GC/MS: ret. time=5.45. MS: 342.1/344.1/364.1 (MH+). ¹H NMR (d₆-DMSO) δ: 8.18 (s, 1 H), 7.70 (t, 1 H), 7.09 (d, 2 H), 6.81 (d, 2 H), 3.67 (s, 3 H), 3.50 (q, 2 H), 2.75 (t, 2 H) ppm.

B. 5-[5-Bromo-4-[2-(4-methoxy-phenyl)-ethviamino]-pyrimidin-2-ylamino]-1,3-dihydro-indol-2-one (C₂₁H₂₀BrN₅O₂).

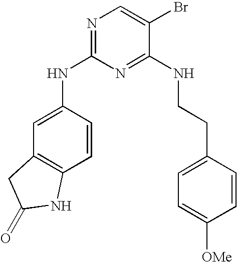

The title compound was prepared according to method E and isolated as a pink solid in a 40% yield. MS: 454.1/456.0 (MH+). ¹H NMR (d₆-DMSO) δ: 10.22 (s, 1 H), 9.01 (s, 1 H), 7.93 (s, 1 H), 7.51 (s, 1 H), 7.44 (d, 1 H), 7.07 (d, 2 H), 6.95 (t, 1 H), 6.81 (d, 2 H), 6.65 (d, 2 H), 3.69 (s, 3 H), 3.52 (q, 2 H), 3.30 (s, 2 H), 2.74 (t, 2 H) ppm.

### EXAMPLE 64

5-Bromo-N⁴-[2-(4-methoxy-phenyl)-ethyl]-N 2-[3-(1,2,3,6-tetrahydro-pyridin-4-yl)-1H-indol-5-yl]-pyrimidine-2,4-diamine

A. (5-Bromo-2-chloro-pyrimidin-4-yl)-[2-(4-methoxy-phenyl)-ethyl]-amine (C₁₃H₁₃BrClN₃O)

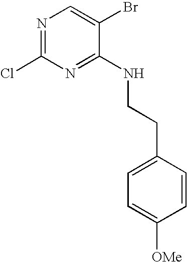

The title compound was isolated as a light yellow, viscous oil in an 80% yield. GC/MS: ret. time=5.45 min. MS: 342.1/344.1/364.1 (MH+). ¹H NMR (d₆-DMSO) δ: 8.18 (s, 1 H), 7.70 (t, 1 H), 7.09 (d, 2 H), 6.81 (d, 2 H), 3.67 (s, 3 H), 3.50 (q, 2 H), 2.75 (t, 2 H) ppm.

B. 5-Bromo-N⁴-[2-(4-methoxy-phenyl)-ethyl]-N²-[3-(1,2,3,6-tetrahydro-pyridin-4-yl)-1H-indol-5-yl]-pyrimidine-2,4-diamine (C₂₆H₂₇BrN₆O).

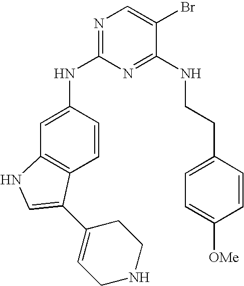

The title compound was isolated as a tan solid in the TFA salt form in a 6.6% yield. MS: 520.4/522.3 (MH+). ¹H NMR (d₆-DMSO) δ: 11.36 (s, 1 H), 8.80, (s, 2 H), 8.07 (s, 1 H), 7.94 (s, 1 H), 7.56 (s, 1 H), 7.38-7.32 (m, 2 H), 6.83 (s, 2 H), 6.65 (s, 2 H), 6.06 (s, 1 H), 3.68-3.25 (m, 10 H), 2.66 (s, 4 H) ppm.

### EXAMPLE 65

5-{5-Bromo-4-[2-(3-methoxy-phenyl)-ethylamino]-pyrimidin-2-ylamino}-1,3-dihydro-indol-2-one

A. (5-Bromo-2-chloro-Pyrimidin-4-yl)-[2-(3-methoxy-phenyl)-ethyl]-amine (C₁₃H₁₃BrclN₃O)

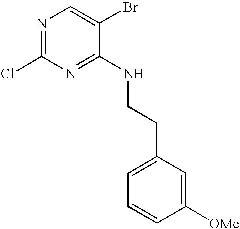

The title intermediate compound was isolated as a colorless oil in an 84% yield. GC/MS: ret. time=5.39 min, m/z=3411343/345. ¹H NMR (d₆-DMSO) δ: 8.19 (s, 1 H), 7.72 (t, 1 H), 7.16 (t, 1 H), 6.76-6.72 (m, 3 H), 3.70 (s, 3 H), 3.55 (q, 2 H), 2.79 (t, 2 H) ppm.

B. 5-{5-Bromo-4-[2-(3-methoxy-phenyl)-ethylamino]-pyrimidin-2-ylamino-1,3-dihydro-indol-2-one (C₂₁H₂₀BrN₅O₂)

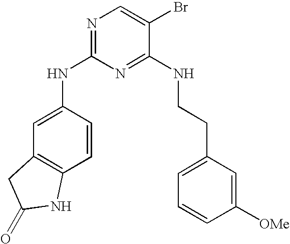

The title compound was isolated as a light pink solid in a 67% yield. MS: 454.1/456.1 (MH+). ¹H NMR (d₆-DMSO) δ: 10.17 (s, 1 H), 9.01 (s, 1 H), 7.93 (s, 1 H), 7.54 (s, 1 H), 7.41 (1 H), 7.17 (t, 1 H), 6.95 (t, 1 H), 6.76-6.72 (m, 3 H), 6.64 (d, 1 H), 3.68 (s, 3 H), 3.56 (q, 2 H), 3.31 (s, 2 H), 2.80 (t, 2 H) ppm.

### EXAMPLE 66

5-Bromo-N⁴-[2-(3-methoxy-phenyl)-ethyl]-N²-[3-(1,2,3,6-tetrahydro-pyridin-4-yl)-1H-indol-5-yl]-pyrimidine-2,4-diamine

A. (5-Bromo-2-chloro-pyrimidin-4-yl)-[2-(3-methoxy-phenyl)-ethyl]-amine (C₁₃H₁₃BrClN₃O)

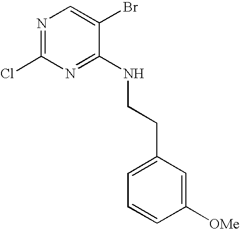

The title intermediate compound was isolated as a colorless oil in an 84% yield. GC/MS: ret. time=5.39 min, m/z=341/3431345. ¹H NMR (d₆-DMSO) δ: 8.19 (s, 1 H), 7.72 (t, 1 H), 7.16 (t, 1 H), 6.76-6.72 (m, 3 H), 3.70 (s, 3 H), 3.55 (q, 2 H), 2.79 (t, 2 H) ppm.

B. 5-Bromo-N⁴-[2-(3-methoxy-phenyl)-ethyl]-N²-[3-(1,2,3,6-tetrahydro-pyridin-4-yl)-1H-indol-5-yl]-pyrimidine-2,4-diamine (C₂₆H₂₇BrN₆O)

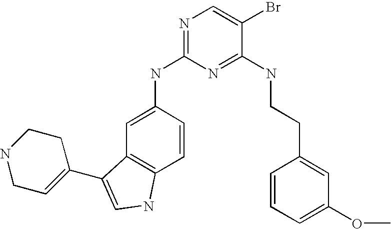

The title compound was isolated as a tan solid in the TFA salt form in a 16% yield. MS: 519.2/521.1 (MH+). ¹H NMR (d₆-DMSO) δ: 11.45 (s, 1 H), 8.82 (s, 2 H), 8.08 (s, 1 H), 8.00 (s, 1 H), 7.56 (s, 1 H), 7.38 (s, 2 H), 7.10 (t, 1 H), 6.77-6.63 (m, 3 H), 6.10 (s, 1 H), 3.72-3.28 (m, 10 H), 2.82-2.80 (m, 2 H), 2.70 (s, 2 H) ppm.

### EXAMPLE 67

5-[5-Bromo-4-(2-o-tolyl-ethylamino)-pyrimidin-2-ylamino]-1,3-dihydro-indol-2-one

A. (5-Bromo-2-chloro-pyrimidin-4-yl)-(2-o-tolyl-ethyl)-amine (C₁₃H₁₃BrClN₃)

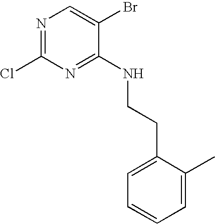

The title intermediate was isolated as a white solid in a 79% yield. MS: 324.2/326.0/328.1 (MH−). ¹H NMR (d₆-DMSO) δ: 8.25 (s, 1 H), 7.91 (t, 1 H), 7.18-7.10 (m, 4 H), 3.56-3.51 (m, 2 H), 2.88-2.82 (m, 2 H), 2.37 (s, 1 H) ppm.

B. 5-[5-Bromo-4-(2-o-tolyl-ethylamino)-pyrimidin-2-ylamino]-1,3-dihydro-indol-2-one (C₂₁H₂₀BrN₅O)

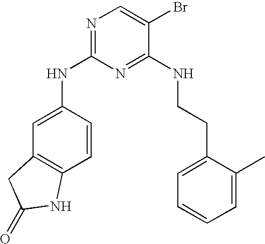

The title compound was isolated as a grey solid in a 28% yield. MS: 438.1/440.0 (MH+). ¹H NMR (d₆-DMSO) δ: 10.20 (s, 1 H), 9.03 (s, 1 H), 7.97 (s, 1 H), 7.56 (s, 1 H), 7.46 (dd, 1 H), 7.13-7.04 (m, 5 H), 6.67 (d, 1 H), 3.59-3.54 (m, 2 H), 3.33 (s, 2 H), 2.84 (t, 2 H), 2.26 (s, 3 H).

### EXAMPLE 68

5-Bromo-N²-[3-(1,2,3,6-tetrahydro-pyridin-4-yl)-1H-indol-5-yl]-N⁴-(2-o-tolyl-ethyl)-pyrimidine-2,4-diamine

A. (5-Bromo-2-chloro-pyrimidin-4-yl)-(2-o-tolyl-ethyl)-amine (C₁₃H₁₃BrClN₃)

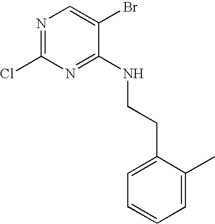

The title intermediate was isolated as a white solid in a 79% yield. MS: 324.2/326.0/328.1 (MH−). ¹H NMR (d₆-DMSO) δ: 8.25 (s, 1 H), 7.91 (t, 1 H), 7.18-7.10 (m, 4 H), 3.56-3.51 (m, 2 H), 2.88-2.82 (m, 2 H), 2.37 (s, 1 H) ppm.

B. 5-Bromo-N²-[3-(1,2,3,6-tetrahydro-pyridin-4-yl)-1H-indol-5-yl]-N⁴-(2-o-tolyl-ethyl)-pyrimidine-2,4-diamine (C₂₆H₂₇BrN₆).

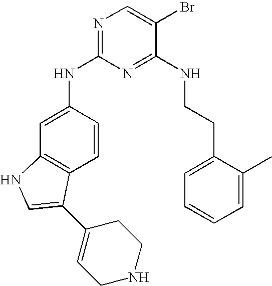

The title compound was isolated in the TFA salt form as a light yellow solid in a 21% yield. HPLC ret. time=5.53 min. MS: 502.9/505.1 (MH+). ¹H NMR (d₆-DMSO)O: 11.36 (S, 1 H), 8.82 (s, 2 H), 8.10 (s, 1 H), 7.98 (s, 1 H), 7.57 (d, 1 H), 7.40-7.32 (m, 2 H), 7.08 (m, 2 H), 6.95 (m, 2 H), 6.09 (s, 1 H), 3.70-3.27 (m, 7H), 2.79-2.70 (m, 4 H), 2.16 (s, 3 H) ppm.

### EXAMPLE 69

5-[5-Bromo-4-(2-m-tolyl-ethylamino)-pyrimidin-2-ylamino]-1,3-dihydro-indol-2-one

A. (5-Bromo-2-chloro-pyrimidin-4-yl)-(2-m-tolyl-ethyl)-amine (C₁₃H₁₃BrClN₃).

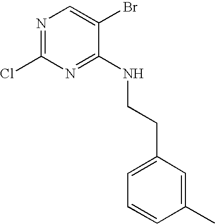

The title intermediate was isolated as a white solid in a 77% yield. MS: 326.1/328.1/330.1 (MH+). ¹H NMR (d₆-DMSO)O: 8.25 (s, 1 H), 7.79 (t, 1 H), 7.19 (t, 1 H), 7.05-7.00 (m, 3 H), 3.61-3.54 (m, 2 H), 2.82 (t, 2 H), 2.29 (s, 3 H) ppm.

B. 5-[5-Bromo-4-(2-m-tolyl-ethylamino)-pyrimidin-2-ylamino]-1,3-dihydro-indol-2-one (C₂₁H₂₀BrN₅O).

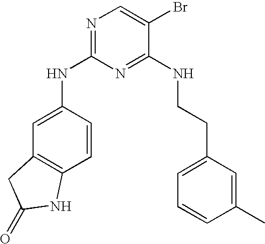

The title compound was isolated as a light pink solid in a 41% yield. MS: 438.1/440.0 (MH+). ¹H NMR (d₆-DMSO) δ: 10.24 (s, 1 H), 9.06 (s, 1 H), 7.97 (s, 1 H), 7.56 (s, 1 H), 7.50 (d, 1 H), 7.20-7.15 (m, 1 H), 7.04-6.98 (m, 3 H), 6.68 (d, 1 H), 3.58 (q, 2 H), 3.33 (s, 2 H), 2.82 (t, 2 H), 2.27 (s, 3 H) ppm.

### EXAMPLE 70

5-Bromo-N²-[3-(1,2,3,6-tetrahydro-pyridin-4-yl)-1H-indol-5-yl]-N⁴-(2-m-tolyl-ethyl)-Pyrimidine-2,4-diamine

A. (5-Bromo-2-chloro-pyrimidin-4-yl)-(2-m-tolyl-ethyl)-amine (C₁₃H₁₃BrClN₃).

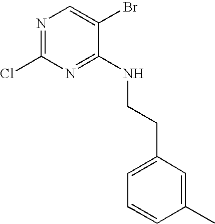

The title intermediate was isolated as a white solid in a 77% yield. MS: 326.1/328.1/330.1 (MH+). ¹H NMR (d₆-DMSO) δ: 8.25 (s, 1 H), 7.79 (t, 1 H), 7.19 (t, 1 H), 7.05-7.00 (m, 3 H), 3.61-3.54 (m, 2 H), 2.82 (t, 2 H), 2.29 (s, 3 H) ppm.

B. 5-Bromo-N²-[3-(1,2,3,6-tetrahydro-pyridin-4-yl)-1H-indol-5-yl]-N⁴-(2-m-tolyl-ethyl)-pyrimidine-2,4-diamine (C₂₆H₂₇BrN₆).

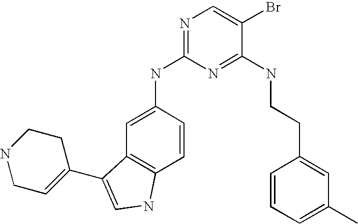

The title compound was isolated as a light yellow solid in a 21% yield. HPLC ret. time=5.61 min. MS: 503.2/505.2 (MH+). ¹H NMR (d₆-DMSO) δ: 11.36 (s, 1 H), 8.83 (s, 2 H), 8.10 (s, 1 H), 7.99 (s, 1 H), 7.56 (s, 1 H), 7.39 (s, 2 H), 7.10-6.83 (m, 4 H), 6.09 (s, 1 H), 3.72 (s, 2 H), 3.53 (s, 2 H), 3.27 (s, 3 H), 2.79-2.69 (m, 4 H), 2.19 (s, 3 H) ppm.

### EXAMPLE 71

5-[5-Bromo-4-(2-p-tolyl-ethylamino)-pyrimidin-2-viamino]-1,3-dihydro-indol-2-one

A. (5-Bromo-2-chloro-pyrimidin-4-yl)-(2-p-tolyl-ethyl)-amine (C₁₃H₁₃BrClN₃).

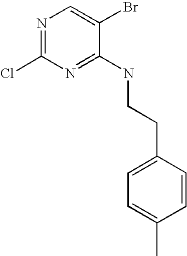

The title intermediate was isolated as a white solid in a 73% yield. MS: 326.1/328.0/330.0 (MH+). ¹H NMR (d₆-DMSO) δ: 8.23 (s, 1 H), 7.76 (t, 1 H), 7.10 (s, 4 H), 3.56 (q, 2 H), 2.82 (t, 2 H), 2.27 (s, 3 H) ppm.

B. 5-[5-Bromo-4-(2-p-tolyl-ethylamino)-pyrimidin-2-ylamino]-1,3-dihydro-indol-2-one (C₂₁H₂₀BrN₅O).

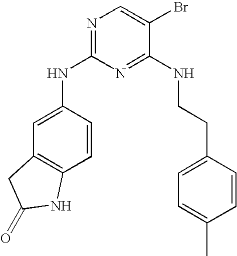

The title compound was isolated as a brown solid in a 14% yield. MS: 438.1/440.0/(MH+). ¹H NMR (d₆-DMSO) δ: 10.22 (s, 1 H), 9.05 (s, 1 H), 7.97 (s, 1 H), 7.55 (s, 1 H), 7.48 (dd, 1 H), 7.09 (s, 1 H), 6.99 (t, 1 H), 6.69 (d, 1 H), 4.03 (q, 2 H), 3.33 (s, 2 H), 2.81 (t, 2 H), 2.23 (s, 3H) ppm.

### EXAMPLE 72

5-Bromo-N²-[3-(1,2,3,6-tetrahydro-pyridin-4-yl)-1H-indol-5-yl]-N⁴-(2-p-tolyl-ethyl)-pyrimidine-2,4-diamine

A. (5-Bromo-2-chloro-Pyrimidin-4-yl)-(2-p-tolyl-ethyl)-amine (C₁₃H₁₃BrClN₃).

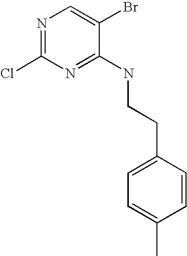

The title intermediate was isolated as a white solid in a 73% yield. MS: 326.1/328.0/330.0 (MH+). ¹H NMR (d₆-DMSO) δ: 8.23 (s, 1 H), 7.76 (t, 1 H), 7.10 (s, 4 H), 3.56 (q, 2 H), 2.82 (t, 2 H), 2.27 (s, 3 H) ppm.

B. 5-Bromo-N²-[3-(1,2,3,6-tetrahydro-pyridin-4-yl)-1H-indol-5-yl]-N⁴-(2-p-tolyl-ethyl)-pyrimidine-2,4-diamine (C₂₆H₂₇BrN₆).

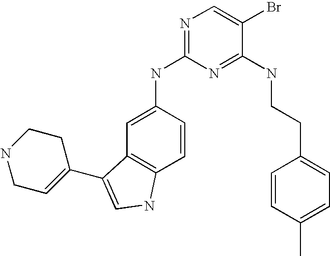

The title compound was isolated as a yellow solid in the TFA salt form in a 13% yield. MS: 503.1/504.1 (MH+). ¹H NMR (d₆-DMSO) δ: 11.34 (s, 1 H), 8.77 (s, 2 H), 8.05 (s, 1 H), 7.95 (s, 1 H), 7.54 (s, 1 H), 7.35 (s, 2 H), 6.94-6.87 (m, 4 H), 6.06 (s, 1 H), 3.68 (s, 4 H), 3.46 (m, 2 H), 3.24 (s, 2 H), 2.66 (s, 3 H), 2.21 (s, 3 H) ppm.

### EXAMPLE 73

[5-Bromo-2-(2-oxo-2,3-dihydro-1H-indol-5-ylamino)-pyrimidin-4-ylamino]-acetic acid

A. [5-Bromo-2-(2-oxo-2,3-dihydro-1H-indol-5-ylamino)-pyrimidin-4-ylamino]-acetic acid tert-butyl ester (C₁₈H₂₀BrN₅O₃).

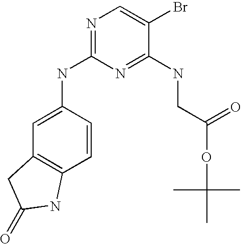

The title intermediate was isolated as a light yellow solid in a 3.5% yield. MS: 434.1/436.1 (MH+). ¹H NMR (d₆-DMSO) δ: 10.18 (s, 1 H), 9.05 (s, 1 H), 7.98 (s, 1 H), 7.43-7.42 (m, 2 H), 7.18 (t, 1 H), 6.65 (d, 1 H), 3.95 (d, 2 H), 3.39 (s, 2 H), 1.29 (s, 9 H) ppm.

B. [5-Bromo-2-(2-oxo-2,3-dihydro-1H-indol-5-ylamino)-pyrimidin-4-ylamino]-acetic acid (C₁₄H₁₂BrN₅O₃).

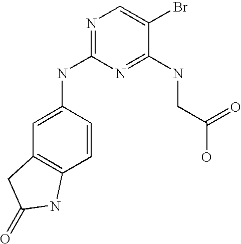

The title compound was isolated as a brown solid. No yield determined. MS: 377.9/380.2 (MH+). ¹H NMR (d₆-DMSO) δ: 10.21 (s, 1 H), 9.10 (s, 1 H), 8.02 (s, 1 H), 7.59 (s, 1 H), 7.38 (dd, 1 H), 7.19 (t, 1 H), 6.69 (d, 1 H), 3.99 (d, 2 H), 3.42 (s, 2 H) ppm.

### EXAMPLE 74

5-(5-Bromo-4-[2-(3-trifluoromethyl-phenyl)-ethylamino]-pyrimidin-2-ylamino}-1,3-dihydro-indol-2-one

A. (5-Bromo-2-chloro-pyrimidin-4-yl)-[2-(3-trifluoromethyl-phenyl)-ethyl]-amine

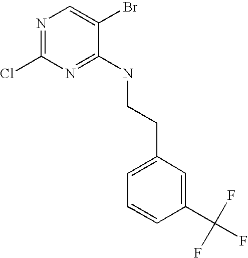

The title intermediate was isolated as a white solid in an 84% yield. GC/MS: ret time=4.65 min, m/z=37913811383. ¹H NMR (d₆-DMSO) δ: 8.25 (s, 1 H), 7.80 (t, 1 H), 7.65-7.52 (m, 4 H), 3.65 (q, 2 H), 2.98 (t, 2 H) ppm.

B. 5-{5-Bromo-4-[2-(3-trifluoromethyl-phenyl)-ethylamino]-pyrimidin-2-ylamino}-1,3-dihydro-indol-2-one (C₂₁H₁₇BrF₃N₅O).

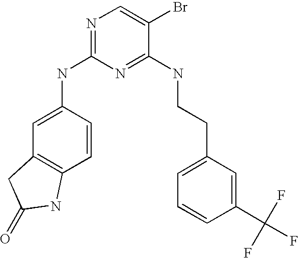

The title compound was isolated as a pink solid in a 37% yield. MS: 492.2/493.5 (MS+). ¹H NMR (d₆-DMSO) δ: 10.18 (s, 1 H), 9.00 (s, 1 H), 7.93 (s, 1 H), 7.54-7.47 (m, 5 H), 7.39 (dd, 1 H), 6.96 (t, 1 H), 6.63 (d, 1 H), 3.60 (q, 2 H), 3.35 (s, 2 H), 2.95 (t, 2 H) ppm.

### EXAMPLE 75

5-[4-(2-Biphenyl-4-yl-ethylamino)-5-bromo-pyrimidin-2-ylamino]-1,3-dihydro-indol-2-one

A. (2-Biphenyl-4-yl-ethyl)-(5-bromo-2-chloro-pyrimidin-4-yl)-amine (C₁₈H₁₅BrClN₃).

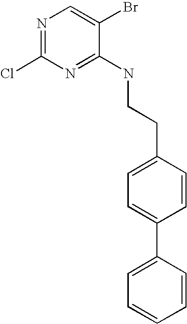

The title intermediate was isolated as a white solid in a 74% yield. GC/MS: ret. time=6.94; m/z=387/389/391. ¹H NMR (d₆-DMSO) δ: 8.24 (s, 1 H), 7.83 (t, 1 H), 7.65-7.58 (m, 4 H), 7.45 (t, 2 H), 7.33 (m, 3 H), 3.62 (q, 2 H), 2.90 (t, 2 H) ppm.

B. 5-[4-(2-Biphenyl-4-yl-ethylamino)-5-bromo-pyrimidin-2-ylamino]-1,3-dihydro-indol-2-one (C₂₆H₂₁BrN₅O)

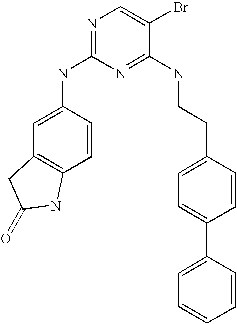

The title compound was isolated as a gray solid in an 11% yield. MS: 500.1/502.3 (MH+). ¹H NMR (d₆-DMSO) δ: 10.25 (s, 1 H), 9.07 (s, 1 H), 7.99 (s, 1 H), 7.68-7.29 (m, 11 H), 7.07 (t, 1 H), 6.72 (d, 1 H), 3.64 (q, 2 H), 3.39 (s, 2 H), 2.91 (t, 2 H) ppm.

### EXAMPLE 76

5-{5-Bromo-4-[2-(3-fluoro-phenyl)-ethylamino]-pyrimidin-2-ylamino}-1,3-dihydro-indol-2-one (C₂₀H₁₇ BrFN₅O).

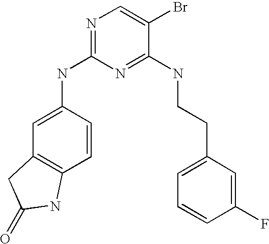

The title compound was isolated as a pink solid in a 52% yield. MS: 442.2/444.1 (MH+). ¹H NMR (d₆-DMSO) δ: 10.22 (s, 1 H), 9.05 (s, 1 H), 7.98 (s, 1 H), 7.57 (s, 1 H), 7.45 (dd, 1 H), 7.38-7.30 (m, 1 H), 7.08-7.00 (m, 4 H), 6.69 (d, 1 H), 3.62 (q, 2 H), 3.37 (s, 2 H), 2.91 (t, 2 H) ppm.

### EXAMPLE 77

5-{5-Bromo-4-[2-(2-chloro-phenyl)-ethyldamino]-pyrimidin-2-ylamino}-1,3-dihydro-indol-2-one

A. (5-Bromo-2-chloro-pyrimidin-4-yl)-[2-(2-chloro-phenyl)-ethyl]-amine

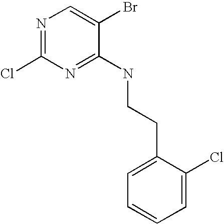

The title intermediate was isolated as a white solid in an 87% yield. GC/MS: ret. Time 5.22 min; m/z: 345/347/349. ¹H NMR (d₆-DMSO) δ: 8.19 (s, 1 H), 7.80 (t, 1 H), 7.39-7.35 (m, 1 H), 7.27-7.18 (m, 3 H), 3.59 (q, 2 H), 2.96 (t, 2 H) ppm.

B. 5-{5-Bromo-4-[2-(2-chloro-Phenyl)-ethylamino]-pyrimidin-2-ylamino}-1,3-dihydro-indol-2-one (C₂₀H₁₇BrClN₅O).

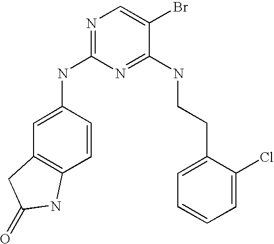

The title compound was isolated as pink solid in a 47% yield. MS: 458.1/460.0/462.1 (MH+). ¹H NMR (d₆-DMSO) δ: 10.17 (s, 1 H), 8.99 (s, 1 H), 7.93 (s, 1 H), 7.51 (s, 1 H), 7.43-7.40 (m, 2 H), 7.40-7.20 (m, 3 H), 7.01 (t, 1 H), 6.63 (d, 1 H), 3.60 (q, 2 H), 3.30 (s, 2 H), 2.97 (t, 2 H) ppm.

### EXAMPLE 78

5-{5-Bromo-4-[2-(2-methoxy-phenyl)-ethylamino]-pyrimidin-2-ylamino}-1,3-dihydro-indol-2-one

A. (5-Bromo-2-chloro-pyrimidin-4-yl)-[2-(2-methoxy-phenyl)-ethyl]-amine (C₁₃H₁₃BrClN₃O.

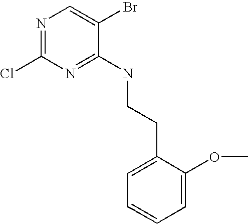

The title intermediate was isolated as a white solid in a 77% yield. GC/MS: ret. Time=5.26 min; m/z: 341/343/345. ¹H NMR (d₆-DMSO) δ: 8.17 (s, 1 H), 7.63 (t, 1 H), 7.17-7.13 (m, 1 H), 7.07 (dd, 1 H), 6.93-6.90 (m, 1 H), 6,83-6.79 (m, 1 H), 3.75 (s, 3 H), 3.53 (q, 2 H), 2.81 (t, 2 H) ppm.

B. 5-{5-Bromo-4-[2-(2-methoxy-phenyl)-ethylamino]-pyrimidin-2-ylamino}-1,3-dihydro-indol-2-one (C₂₁H₂₀BrN₅O₂).

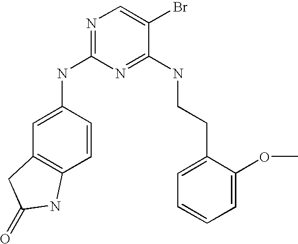

The title compound was isolated as a light pink solid in a 44% yield. MS: 454.1/456.0 (MH+). ¹H NMR (d₆-DMSO) δ: 10.16 (s, 1 H), 8.99 (s, 1 H), 7.92 (s, 1 H), 7.53 (s, 1 H), 7.43 (dd, 1 H), 7.20-7.15 (m, 1 H), 7.09-7.07 (m, 1 H), 6.94-6.92 (m, 1 H), 6.87-6.81 (m, 2 H), 6.62 (d, 1 H), 3.73 (s, 3 H), 3.54 (q, 2 H), 2.83 (t, 2 H) ppm.

### EXAMPLE 79

5-{5-Bromo-4-[2-(4-fluoro-phenyl)-ethylamino]-pyrimidin-2-ylamino}-1,3-dihydro-indol-2-one (C₂₀H₁₇BrFN₅O).

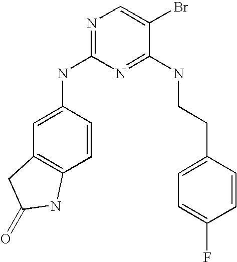

The title compound was isolated as a pink solid in a 44% yield. MS: 442.1/444.0 (MH+). ¹H NMR (d₆-DMSO) δ: 10.22 (s, 1 H), 9.04 (s, 1 H), 7.98 (s, 1 H), 7.55 (s, 1 H), 7.49-7.46 (m, 1 H), 7.26-7.21 (m, 2 H), 7.14-7.08 (m, 2 H), 7.00 (t, 1 H), 6.69 (d, 1 H), 3.59 (q, 2 H), 3.37 (s, 2 H), 2.86 (t, 2 H) ppm.

### EXAMPLE 80

5-[5-Bromo-4-[2-(4-chloro-phenyl)-ethylamino]-pyrimidin-2-ylamino]-1,3-dihydro-indol-2-one

A. (5-Bromo-2-chloro-pyrimidin-4-yl)-[2-(4-chloro-phenyl)-ethyl]-amine (C₁₂H₁₀BrCl₂N₃).

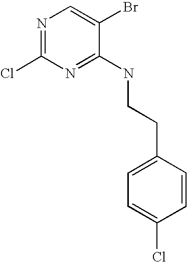

The title intermediate was isolated as a white solid in an 86% yield. GC/MS: ret. time=5.36 min; m/z: 345/347/349. ¹H NMR (d₆-DMSO) δ: 8.25 (s, 1 H), 7.80-7.76 (t, 1 H), 7.38-7.33 (m, 2 H), 7.26-7.23 (m, 2 H), 3.59 (q, 2 H), 2.87 (t, 2 H) ppm.

B. 5-{5-Bromo-4-[2-(4-chloro-phenyl)-ethviamino]-pyrimidin-2-ylamino}-1,3-dihydro-indol-2-one (C₂₀H₁₇BrClN₅O).

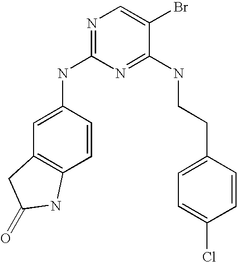

The title compound was isolated as a pink solid in a 39% yield. MS: 458.1/460.0/462.1 (MH+). ¹H NMR (d₆-DMSO) δ: 10.19 (s, 1 H), 9.00 (s, 1 H), 7.93 (s, 1 H), 7.50 (s, 1 H), 7.46 (dd, 1 H), 7.31-7.29 (m, 2 H), 7.19-7.17 (m, 2 H), 6.96 (t, 1 H), 6.65 (d, 1 H), 3.54 (q, 2 H), 3.34 (s, 2 H), 2.82 (t, 2 H) ppm.

### EXAMPLE 81

5-[5-Bromo-4-[2-(2-fluoro-phenyl)-ethylamino]-pyrimidin-2-ylamino]-1,3-dihydro-indol-2-one

A. (5-Bromo-2-chloro-pyrimidin-4-yl)-[2-(2-fluoro-phenyl)-ethyl]-amine (C₁₂H₁₀BrClFN₃).

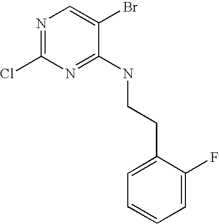

The title intermediate was isolated as a white solid in an 84% yield. GC/MS: ret. time=4.67 min; m/z: 329/331/333. ¹H NMR (d₆-DMSO) δ: 8.23 (s, 1 H), 7.83 (t, 1 H), 7.30-7.23 (m, 2 H), 7.18-7.10 (m, 2 H), 3.62 (q, 2 H), 2.92 (t, 2 H) ppm.

B. 5-{5-Bromo-4-[2-(2-fluoro-phenyl)-ethylamino]-pyrimidin-2-ylamino}-1,3-dihydro-indol-2-one (C₂₀H₁₇BrFN₅O).

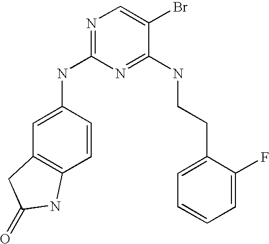

The title compound was isolated as a pink solid in a 19% yield. MS: 442.0/444.0 (MH+). ¹H NMR (d₆-DMSO) 6:10.17 (s, 1 H), 9.00 (s, 1 H), 7.93 (s, 1 H), 7.52 (s, 1 H), 7.42 (dd, 1 H), 7.25-7.20 (m, 2 H), 7.13-7.06 (m, 2 H), 7.01 (t, 1 H), 6.64 (d, 1 H), 3.58 (q, 2 H), 3.32 (s, 2 H), 2.88 (t, 2 H) ppm.

### EXAMPLE 82

5-[5-Bromo-4-(3-phenyl-allylamino)-pyrimidin-2-ylamino]-1,3-dihydro-indol-2-one

A. (3-Phenyl-allyl)-carbamic acid di-tert-but ester (C₁₉H₂₇NO₄).

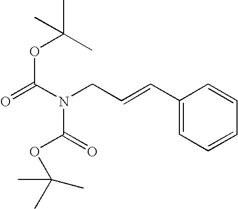

The title intermediate was isolated as a light yellow oil in a 77% yield. GC/MS: ret. time=4.28 min; m/z: 277 (MH-t-Bu), 234 (MH-BOC), 221 (MH-(t-Bu)₂), 177 (MH-BOC-t-Bu), 132 (MH-BOC₂), 116 (bp). ¹H NMR (d₆-DMSO) δ: 7.44-7.41 (m, 2 H), 7.36-7.31 (m, 2 H), 7.28-7.23 (m, 1 H), 6.50 (d, 1 H), 6.25 (dt, 1 H), 4.26 (d, 2 H), 1.46 (s, 18H).

B. 3-Phenyl-allylamine (C₉H₁₁N).

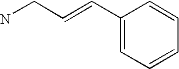

The title intermediate in the crude form as a TFA salt was produced. GC/MS: ret. time=1.54 min; m/z: 133. ¹H NMR (d₆-DMSO) δ: 7.98 (bs, 2 H), 7.42-7.25 (m, 5 H), 6.70 (d, 1 H), 6.23 (dt, 1 H), 3.62-3.57 (m, 2 H) ppm.

C. (5-Bromo-2-chloro-pyrimidin-4-yl)-(3-phenyl-allyl)-amine (C₁₃ H₁₃ BrClN₃).

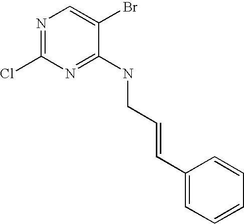

The title intermediate was isolated as a white solid in a 57% yield. GC/MS: ret. time=5.43 min; m/z: 323/325/327. ¹H NMR (d₆-DMSO) δ: 8.29 (s, 1 H), 8.05 (t, 1 H), 7.45-7.22 (m, 5 H), 6.55-6.50 (m, 1 H), 6.34 (dt, 1 H), 4.19-4.15 (m, 2 H) ppm.

D. 5-[5-Bromo-4-(3-phenyl-allylamino)-pyrimidin-2-ylamino]-1,3-dihydro-indol-2-Br

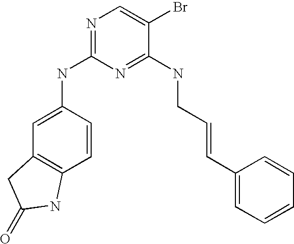

The title compound was isolated as a pink solid in a 42% yield. MS: 436.1/438.0 (MH+). ¹H NMR (d₆-DMSO) δ: 10.20 (s, 1 H), 9.07 (s, 1 H), 8.01 (s, 1 H), 7.61 (s, 1 H), 7.48-7.20 (m, 7H), 6.69 (d, 1 H), 6.54-6.36 (m, 2 H), 4.18 (t, 2 H), 3.39 (s, 2 H) ppm.

### EXAMPLE 83

5-{5-Bromo-4-[(thiophen-2-ylmethyl)-amino]-pyrimidin-2-ylamino}-1,3-dihydro-indol-2-one

A. (5-Bromo-2-chloro-pyrimidin-4-yl)-thiophen-2-ylmethyl-amine (C₉H₇BrClN₃S).

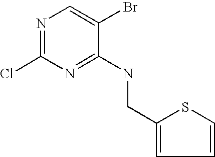

The title intermediate was isolated as a white solid in an 88% yield. GC/MS: ret. time=4.49 min; m/z: 303/305/307. ¹H NMR (d₆-DMSO) δ: 8.36 (t, 1 H), 8.26 (s, 1 H), 7.35 (dd, 1 H), 7.00-6.99 (m, 1 H), 6.93 (dd, 1 H), 4.67 (d, 2 H) ppm.

B. 5-[5-Bromo-4-[(thiophen-2-ylmethyl)-amino]-pyrimidin-2-ylamino]-1,3-dihydro-indol-2-one (C₁₇H₁₄BrN₅OS).

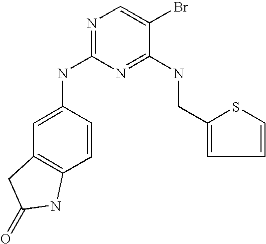

The title compound was isolated as a pink solid in a 29% yield. MS: 416.1/418.1 (MH+). ¹H NMR (d₆-DMSO) δ: 10.15 (s, 1 H), 9.05 (s, 1 H), 7.97 (s, 1 H), 7.57 (t, 1 H), 7.51 (s, 1 H), 7.39-7.30 (m, 2 H), 6.97-6.90 (m, 2 H), 6.62 (d, 1 H), 4.71 (d, 2 H), 3.35 (s, 2 H) ppm.

### EXAMPLE 84

6-[5-Bromo-4-[(thiophen-2-ylmethyl)-amino]-pyrimidin-2-ylamino]-1,3-dihydro-indol-2-one

A. (5-Bromo-2-chloro-pyrimidin-4-yl)-thiophen-2-ylmethyl-amine (C₉H₇BrClN₃S).

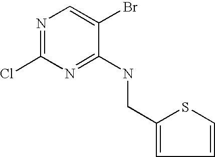

The title intermediate was isolated as a white solid in an 88% yield. GC/MS: ret. time=4.49 min; m/z: 303/305/307. ¹H NMR (d₆-DMSO) δ: 8.36 (t, 1 H), 8.26 (s, 1 H), 7.35 (dd, 1 H), 7.00-6.99 (m, 1 H), 6.93 (dd, 1 H), 4.67 (d, 2 H) ppm.

C. 6-{5-Bromo-4-[(thiophen-2-ylmethyl)-amino]-pyrimidin-2-ylamino}-1,3-dihydro-indol-2-one (C₁₇H₁₄BrN₅OS).

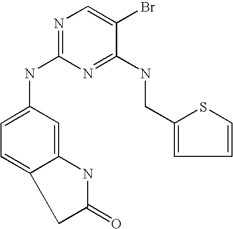

The title compound was isolated as a purple solid in a 27% yield. MS: 416.1/418.1 (MH+). ¹H NMR (d₆-DMSO) δ: 10.32 (s, 1 H), 9.22 (s, 1 H), 8.00 (s, 1 H), 7.59 (t, 1 H), 7.36 (d, 1 H), 7.29 (dd, 1 H), 7.19 (dd, 1 H), 7.00-6.97 (m, 2 H), 6.91-6.88 (m, 1 H), 4.74 (d, 2 H), 3.34 (s, 2 H) ppm.

### EXAMPLE 85

5-[5-Bromo-4-(2.3-dimethyl-benzylamino)-pyrimidin-2-ylamino]-1,3-dihydro-indol-2-one

A. (5-Bromo-2-chloro-pyrimidin-4-yl)-(2,3-dimethyl-benzyl)-amine (C₁₃H₁₃BrClN₃).

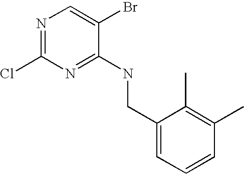

The title intermediate was isolated as a white solid in a 72% yield. GC/MS: ret. time=5.16 min; m/z: 325/327/329. ¹H NMR (d₆-DMSO) δ: 8.25 (s, 1 H), 8.13 (t, 1 H), 7.03-6.95 (m, 3H), 4.52 (d, 2 H), 2.21 (s, 3 H), 2.18 (s, 3 H) ppm.

B. 5-[5-Bromo-4-(2,3-dimethyl-benzylamino)-pyrimidin-2-ylamino]-1,3-dihydro-indol-2-one (C₂₁H₂₀BrN₅O).

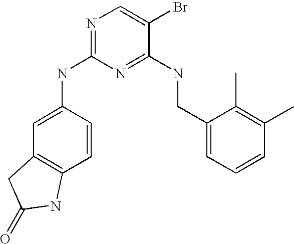

The title compound was isolated as a light pink solid in a 7.7% yield. MS: 438.1/440.1 (MH+). ¹H NMR (d₆-DMSO) δ: 10.12 (s, 1 H), 8.97 (s, 1 H), 7.98 (s, 1 H), 7.32 (s, 2 H), 7.16 (d, 1 H), 7.02-6.91 (m, 3 H), 6.47 (d, 1 H), 4.54 (d, 2 H), 3.21 (s, 2 H), 2.26 (s, 3 H), 2.16 (s, 3 H) ppm.

### EXAMPLE 86

6-[5-Bromo-4-(2,3-dimethyl-benzylamino)-pyrimidin-2-ylamino]-1,3-dihydro-indol-2-one

A. (5-Bromo-2-chloro-pyrimidin-4-yl)-(2,3-dimethyl-benzyl)-amine (C₁₃H₁₃BrClN₃).

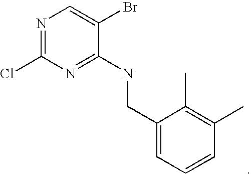

The title intermediate was isolated as a white solid in a 72% yield. GC/MS: ret. time=5.16 min; m/z: 325/327/329. ¹H NMR (d₆-DMSO) δ: 8.25 (s, 1 H), 8.13 (t, 1 H), 7.03-6.95 (m, 3H), 4.52 (d, 2 H), 2.21 (s, 3 H), 2.18 (s, 3 H) ppm.

B. 6-[5-Bromo-4-(2,3-dimethyl-benzylamino)-pyrimidin-2-viamino]-1,3-dihydro-indol-2one (C₂₁H₂₀BrN₅O).

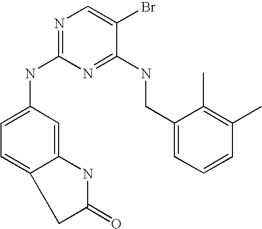

The title compound was isolated as a purple solid in a 21% yield. MS: 438.0/440.2 (MH+). ¹H NMR (d₆-DMSO) δ: 10.24 (s, 1 H), 9.12 (s, 1 H), 8.01 (s, 1 H), 7.26 (t, 1 H), 7.18 (s, 1 H), 7.07 (dd, 1 H), 7.02-6.96 (m, 3 H), 6.84 (d, 1 H), 4.58 (d, 2 H), 3.30 (s, 2 H), 2.23 (s, 3 H), 2.17 (s, 3 H) ppm.

### EXAMPLE 87

5-[5-Bromo-4-(2,5-dimethyl-benzylamino)-pyrimidin-2-ylamino]-1,3-dihydro-indol-2-one

A. (5-Bromo-2-chloro-pyrimidin-4-yl)-(2,5-dimethyl-benzyl)-amine (C₁₃H₁₃BrClN₃).

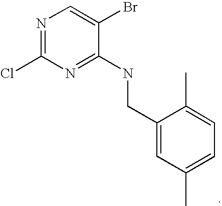

The title intermediate was isolated as a white solid in an 84% yield. GC/MS: ret. time=4.99 min; m/z: 325/327/329. ¹H NMR (d₆-DMSO) δ: 8.24 (s, 1 H), 8.16 (t, 1 H), 7.02-6.91 (m, 3 H), 4.47 (d, 2 H), 2.26 (s, 3 H), 2.18 (s, 3 H) ppm.

B. 5-[5-Bromo-4-(2,5-dimethyl-benzylamino)-pyrimidin-2-ylamino]-1,3-dihydro-indol-2-one (C₂₁H₂₀BrN₅O).

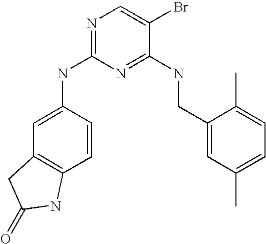

The title compound was isolated as a off white solid in a 9% yield. MS: 438.1/440.1 (MH+). HPLC: ret. time=6.48 min. ¹H NMR (d₆-DMSO) δ: 10.12 (s, 1 H), 8.99 (s, 1 H), 7.98 (s, 1 H), 7.35-7.32 (m, 2 H), 7.23-7.21 (m, 1 H), 7.04-7.02 (m, 1 H), 6.91-6.87 (m, 2 H), 6.52 (d, 1 H), 4.50 (d, 2 H), 3.25 (s, 2 H), 2.22 (s, 3 H), 2.14 (s, 3 H) ppm.

### EXAMPLE 88

6-[5-Bromo-4-(2,5-dimethyl-benzylamino)-pyrimidin-2-ylamino]-1,3-dihydro-indol-2-one

A. (5-Bromo-2-chloro-pyrimidin-4-yl)-(2.5-dimethyl-benzyl)-amine (C₁₃H₁₃BrClN₃).

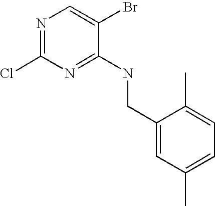

The title intermediate was isolated as a white solid in an 84% yield. GC/MS: ret., time=4.99 min; m/z: 325/327/329. ¹H NMR (d₆-DMSO) δ: 8.24 (s, 1 H), 8.16 (t, 1 H), 7.02-6.91 (m, 3H), 4.47 (d, 2 H), 2.26 (s, 3 H), 2.18 (s, 3 H) ppm.

B. 6-[5-Bromo-4-(2.5-dimethyl-benzylamino)-pyrimidin-2-ylamino]-1,3-dihydro-indol-2-one (C₂₁H₂₀BrN₅O).

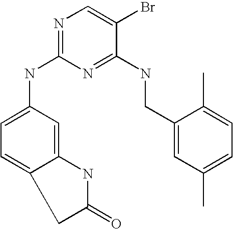

The title compound was isolated as a purple solid in a 4% yield. MS: 438.1/440.1 (MH+). HPLC: ret. time=6.86 min. ¹H NMR (d₆-DMSO) δ: 10.26 (s, 1 H), 9.13 (s, 1 H), 8.01 (s, 1 H), 7.28 (t, 1 H), 7.20-7.18 (m, 1 H), 7.12-7.09 (m, 1 H), 7.03-7.01 (m, 1 H), 6.95-6.86 (m, 3 H), 4.54 (d, 2 H), 3.31 (s, 2 H), 2.23 (s, 3 H), 2.14 (s, 3 H) ppm.

### EXAMPLE 89

6-[5-Bromo-4-(2-fluoro-benzylamino)-pyrimidin-2-ylamino]-1,3-dihydro-indol-2-one

A. (5-Bromo-2-chloro-pyrimidin-4-yl)-(2-fluoro-benzyl)-amine (CH₁₁H₈BrClFN₃).

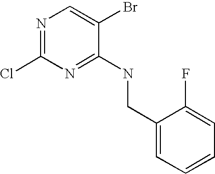

The title intermediate was isolated as a white solid in a 68% yield. GC/MS: ret. time=4.75 min; m/z: 315/317/319. ¹H NMR (d₆-DMSO) δ: 8.28-8.25 (m, 2 H), 7.31-7.22 (m, 2 H), 7.18-7.10 (m, 2 H), 4.58 (d, 2 H) ppm.

B. 6-[5-Bromo-4-(2-fluoro-benzylamino)-pyrimidin-2-ylamino]-1,3-dihydro-indol-2-one (C₁₉H₁₅BrFN₅O).

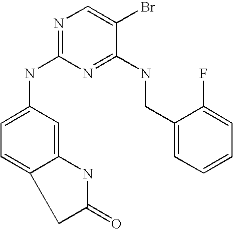

The title compound was isolated as a purple solid in a 6.5% yield. MS: 428.1/430.1 (MH+). ¹H NMR (d₆-DMSO) δ: 10.26 (s, 1 H), 9.15 (s, 1 H), 8.03 (s, 1 H), 7.48 (t, 1 H), 7.26-7.09 (m, 6 H), 6.87 (d, 1 H), 4.65 (d, 2 H), 3.31 (s, 2 H) ppm.

### EXAMPLE 90

6-[5-Bromo-4-(2-trifluoromethoxy-benzylamino)-pyrimidin-2-ylamino]-1,3-dihydro-indol-2-one

A. (5-Bromo-2-chloro-pyrimidin-4-yl)-(2-trifluoromethoxy-benzyl)-amine (C₁₂H₈BrClF₃N₃O).

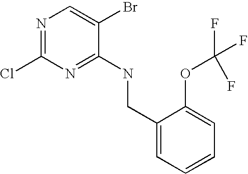

The title intermediate was isolated as a white solid in a 66% yield. GC/MS: ret. time=4.55 min; m/z: 381/383/385. ¹H NMR (d₆-DMSO) δ: 8.28 (m, 2 H), 7.40-7.30 (m, 4 H), 4.63-4.62 (d, 2 H) ppm.

B. 6-[5-Bromo-4-(2-trifluoromethoxy-benzylamino)-pyrimidin-2-ylamino]-1,3-dihydro-indol-2-one (C₂₀H₁₅BrF₃N₅O₂).

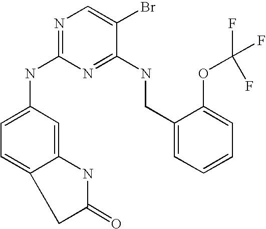

The title compound was isolated as a dark purple solid in a 2% yield. MS: 494.1/496.0 (MH+). ¹H NMR (CD₃OD) δ: 8.00 (s, 1 H), 7.41-7.31 (m, 4 H), 7.15-7.09 (m, 2 H), 7.02-6.99 (m, 1 H), 4.81 (s, 2 H), 3.46 (s, 2 H) ppm.

### EXAMPLE 91

5-[5-Bromo-4-(3-trifluoromethoxy-benzylamino)-pyrimidin-2-ylamino]-1,3-dihydro-indol-2-one

A. (5-Bromo-2-chloro-pyrimidin-4-yl)-(3-trifluoromethoxy-benzyl)-amine (C₁₂H₈BrClF₃N₃O).

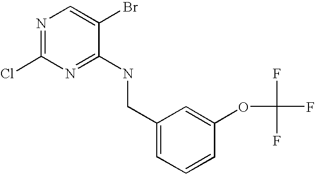

The title intermediate was isolated as a colorless oil in a 68% yield. GC/MS: ret. time 4.75 min; m/z: 381/383/385. ¹H NMR (d₆-DMSO) δ: 8.35 (t, 1 H), 8.26 (s, 1 H), 7.45-7.41 (m, 1 H), 7.31-7.20 (m, 3 H), 4.56 (d, 2 H) ppm.

B. 5-[5-Bromo-4-(3-trifluoromethoxy-benzylamino)-pyrimidin-2-ylamino]-1,3-dihydro-indol-2-one (C₂₀H₁₅BrF₃N₅O₂).

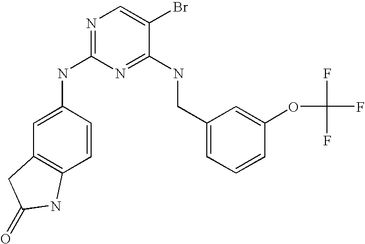

The title compound was isolated as a pink solid in a 38% yield. MS: 494.1/496.1 (MH+). ¹H NMR (d₆-DMSO) δ: 10.16 (s, 1 H), 9.02 (s, 1 H), 7.98 (s, 1 H), 7.61 (t, 1 H), 7.44-7.39 (m, 2 H), 7.32-7.17 (m, 4 H), 6.57 (d, 2 H), 4.59 (d, 2 H), 3.29 (s, 2 H) ppm.

### EXAMPLE 92

6-[5-Bromo-4-(3-trifluoromethoxy-benzylamino)-pyrimidin-2-ylamino]-1,3-dihydro-indol-2-one

A. (5-Bromo-2-chloro-pyrimidin-4-yl)-(3-trifluoromethoxy-benzyl)-amine (C₁₂H₈BrClF₃N₃O).

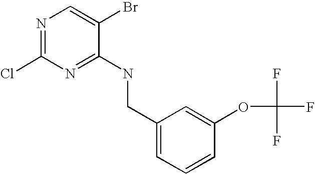

The title intermediate was isolated as a colorless oil in a 68% yield. GC/MS: ret. time=4.75 min; m/z: 381/383/385. ¹H NMR (d₆-DMSO) δ: 8.35 (t, 1 H), 8.26 (s, 1 H), 7.45-7.41 (m, 1 H), 7.31-7.20 (m, 3 H), 4.56 (d, 2 H) ppm.

B. 6-[5-Bromo-4-(3-trifluoromethoxy-benzylamino)-pyrimidin-2-ylamino]-1,3-dihydro-indol-2-one (C₂₀H₁₅BrF₃N₅O₂).

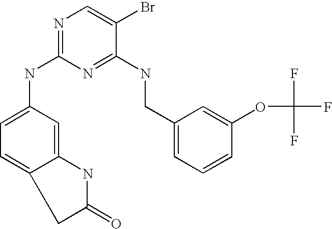

The title compound was isolated as a purple solid in a 4% yield. MS: 494.2/496.2 (MH+). ¹H NMR (d₆-DMSO) δ: 10.32 (s, 1 H), 9.17 (s, 1 H), 8.01 (s, 1 H), 7.65 (t, 1 H), 7.42-7.28 (m, 4 H), 7.17-7.15 (m, 1 H), 7.07 (dd, 1 H), 6.92 (d, 1 H), 4.62 (d, 2 H), 3.31 (s, 2 H) ppm.

### EXAMPLE 93

5-[5-Bromo-4-(4-trifluoromethoxy-benzylamino)-pyrimidin-2-viamino]-1,3-dihydro-indol-2-one

A. (5-Bromo-2-chloro-pyrimidin-4-yl)-(4-trifluoromethoxy-benzyl)-amine (C₁₂H₈BrClF₃N₃O).

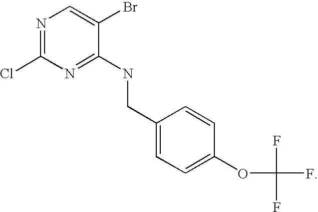

The title intermediate was isolated as a colorless oil in a 76% yield. GC/MS: ret. time=4.88 min; m/z: 381/383/385. ¹H NMR (d₆-DMSO) δ: 8.34 (t, 1 H), 8,25 (s, 1 H), 7.41-7.37 (m, 2 H), 7.30-7.27 (m, 2 H), 4.56 (d, 2 H) ppm.

B. 5-[5-Bromo-4-(4-trifluoromethoxy-benzylamino)-pyrimidin-2-ylamino]-1,3-dihydro-indol-2-one (C₂₀H₁₅BrF₃N₅O₂).

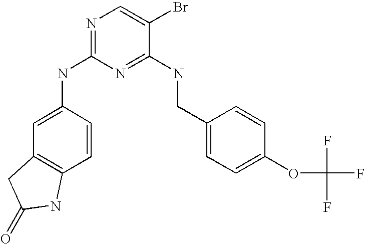

The title compound was isolated as a light gray solid in a 23% yield. MS: 494.0/496.0 (MH+). ¹H NMR (d₆-DMSO) δ: 10.14 (s, 1 H), 9.01 (s, 1 H), 7.98 (s, 1 H), 7.59 (t, 1 H), 7.40-7.21 (m, 6 H), 6.57 (d, 1 H), 4.58 (d, 2 H), 3.29 (s, 2 H) ppm.

### EXAMPLE 94

6-[5-Bromo-4-(4-trifluoromethoxy-benzylamino)-pyrimidin-2-ylamino]-1,3-dihydro-indol-2-one

A. (5-Bromo-2-chloro-pyrimidin-4-yl)-(4-trifluoromethoxy-benzyl)-amine (C₁₂H₈BrClF₃N₃O).

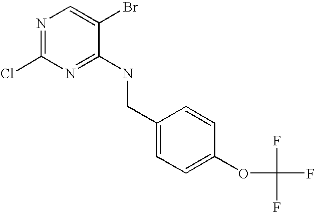

The title intermediate was isolated as a colorless oil in a 76% yield. GC/MS: ret. time=4.88 min; m/z: 381/383/385. ¹H NMR (d₆-DMSO) δ: 8.34 (t, 1 H), 8,25 (s, 1 H), 7.41-7.37 (m, 2 H), 7.30-7.27 (m, 2 H), 4.56 (d, 2 H) ppm.

B. 6-[5-Bromo-4-(4-trifluoromethoxy-benzylamino)-pyrimidin-2-Viamino]-1,3-dihydro-indol-2-one (C₂₀H₁₅BrF₃N₅O₂).

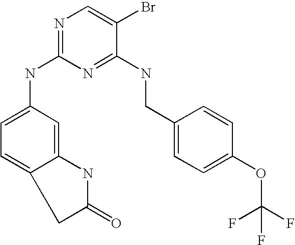

The title compound was isolated as a purple solid in a 25% yield. MS: 494.0/496.0 (MH+). ¹H NMR (d₆-DMSO) δ: 10.31 (s, 1 H), 9.16 (s, 1 H), 8.01 (s, 1 H), 7.61 (t, 1 H), 7.42 (d, 2 H), 7.28-7.25 (m, 3 H), 7.09 (dd, 1 H), 6.92 (d, 1 H), 4.61 (d, 2 H), 3.32 (s, 2 H) ppm.

### EXAMPLE 95

6-[5-Bromo-4-(2-methoxy-benzylamino)-pyrimidin-2-ylamino]-1,3-dihydro-indol-2-one

A. (5-Bromo-2-chloro-pyrimidin-4-yl)-(2-methoxy-benzyl)-amine (C₁₂H₁₁BrClN₃O).

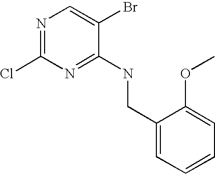

The title intermediate was isolated as a white solid in a 78% yield. GC/MS: ret. time=5.43 min; m/z: 327/329/331. ¹H NMR (d₆-DMSO) δ: 8.26 (s, 1 H), 8.05 (t, 1 H), 7.23-7.19 (m, 1 H), 7.01-6.96 (m, 2 H), 6.88-6.84 (m, 1 H), 4.52 (d, 2 H), 3.80 (s, 3 H) ppm.

B. 6-[5-Bromo-4-(2-methoxy-benzylamino)-pyrimidin-2-ylamino]-1,3-dihydro-indol-2-one (C₂₀H₁₈BrN₅O₂).

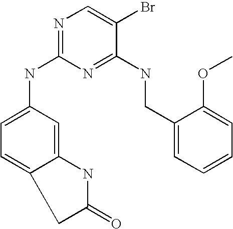

The title compound was isolated as a gray solid in a 12% yield. MS: 440.1/442.1 (MH+). ¹H NMR (d₆-DMSO) δ: 10.31 (s, 1 H), 9.17 (s, 1 H), 8.00 (s, 1 H), 7.54 (t, 1 H), 7.29 (s, 1 H), 7.20-7.11 (m, 2 H), 6.95-6.89 (m, 3 H), 6.75-6.73 (m, 1 H), 4.56 (d, 2 H), 3.63 (s, 3 H), 3.32 (s, 2 H) ppm.

### EXAMPLE 96

6-[5-Bromo-4-(3-methoxy-benzylamino)-pyrimidin-2-ylamino]-1,3-dihydro-indol-2-one

A. (5-Bromo-2-chloro-pyrimidin-4-yl)-(3-methoxy-benzyl)-amine (C₁₂H₁₁BrClN₃O).

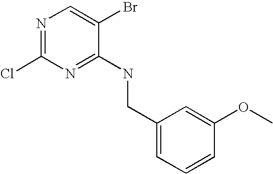

The title intermediate was isolated as a white solid in an 83% yield. GC/MS: ret. time 5.56 min; m/z: 327/329/331. ¹H NMR (d₆-DMSO) δ: 8.28 (t, 1 H), 8.25 (s, 1 H), 7.21 (t, 1 H), 6.86-6.77 (m, 3 H), 4.50 (d, 2 H), 3.70 (s, 3 H) ppm.

B. 6-[5-Bromo-4-(3-methoxy-benzylamino)-pyrimidin-2-ylamino]-1,3-dihydro-indol-2-one (C₂₀H₁₈BrN₅O₂).

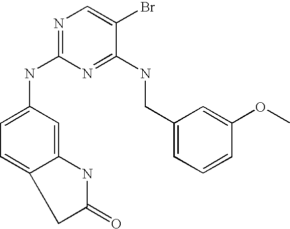

The title compound was isolated as a pink solid in a 5% yield. MS: 440.0/442.0 (MH+). ¹H NMR (d₆-DMSO) δ: 10.23 (s, 1 H), 9.11 (s, 1 H), 8.02 (s, 1 H), 7.21-6.82 (m, 8H), 4.57 (d, 2 H), 3.81 (s, 3 H), 3.30 (s, 2 H) ppm.

### EXAMPLE 97

6-[5-Bromo-4-(3-trifluoromethyl-benzylamino)-pyrimidin-2-ylamino]-1,3-dihydro-indol-2-one

A. (5-Bromo-2-chloro-Pyrimidin-4-yl)-(3-trifluoromethyl-benzyl)-amine (C₁₂H₈BrClF₃N₃).

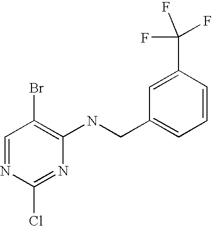

The title intermediate was isolated as a white solid in a 78% yield. GC/MS: ret. time=4.77 min; m/z: 365/367/369. ¹H NMR (d₆-DMSO) δ: 8.38 (t, 1 H), 8.26 (s, 1 H), 7.67 (s, 1 H), 7.59-7.51 (m, 3 H), 4.60 (d, 2 H) ppm.

B. 6-[5-Bromo-4-(3-trifluoromethyl-benzylamino)-pyrimidin-2-ylamino]-1,3-dihydro-indol-2-one (C₂₀H₁₅BrF₃N₅O).

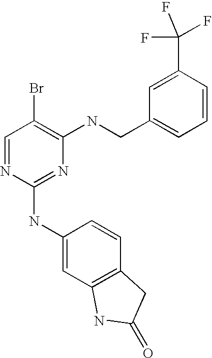

The title compound was isolated as a purple solid in a 10% yield. MS: 478.0/480.0 (MH+). ¹H NMR (d₆-DMSO) δ: 10.33 (s, 1 H), 9.18 (s, 1 H), 8.01 (s, 1 H), 7.71-7.67 (m, 2 H), 7.62-6.60 (m, 1 H), 7.54-7.48 (m, 2 H), 7.29 (d, 1 H), 7.05 (dd, 1 H), 6.91 (d, 1 H), 4.66 (d, 2 H), 3.31 (s, 2 H) ppm.

### EXAMPLE 98

5-{5-Bromo-4-[(thiazol-2-ylmethyl)-amino]-pyrimidin-2-ylamino}-1,3-dihydro-indol-2-one

A. Thiazole-2-carbaldehyde oxime (C₄H₄N₂OS).

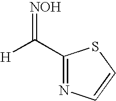

The title intermediate was synthesized following the procedure by Dondoni (Dondoni, A., Fantin, G., Fogagnolo, M., Medici, A., Pedrini, P.; Synthesis 1987, 998-1001) and isolated as a light purple solid in a 50% yield. GC/MS: ret. time=1.55 min and 1.70 min (cis and trans isomers); m/z: 128.

B. C-Thiazol-2-yl-methylamine (C₄H₆N₂S)

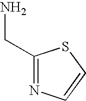

The title intermediate was synthesized following the procedure by Dondoni (Dondoni, A., Merchan, F. L., Merino, P., Rojo, I., Tejero, T.; Synthesis, 1996, 641-646) and isolated as a crude sample in a 21% yield. GC/MS: ret. time=0.99 min; m/z: 114. ¹H NMR (d₆-DMSO) δ: 7.65 (d, 1 H), 7.52 (d, 1 H), 3.95 (s, 2 H), 3.30 (s, 2 H) ppm.

C. (5-Bromo-2-chloro-pyrimidin-4-yl)-thiazol-2-ylmethyl-amine (C₈H₆BrClN₄S).

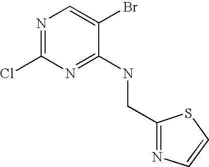

The title intermediate was isolated as a yellow solid in a 45% yield. GC/MS: ret. time=4.91 min; m/z: 304/306/308. ¹H NMR (d₆-DMSO) δ: 8.55 (t, 1 H), 8.33 (s, 1 H), 7.71 (d, 1 H), 7.60 (d, 1 H), 4.81 (d, 2 H) ppm.

D. 5-{5-Bromo-4-[(thiazol-2-ylmethyl)-amino]-pyrimidin-2-ylamino}-1,3-dihydro-indol-2-one (C₁₆H₁₃BrN₆OS)

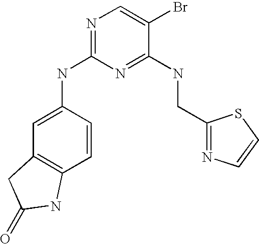

The title compound was isolated as a brown solid in a 43% yield. MS: 417.0/418.9 (MH+). ¹H NMR (d₆-DMSO) δ: 10.15 (s, 1 H), 9.10 (s, 1 H), 8.02 (s, 1 H), 7.82 (sb, 1 H), 7.72 (d, 1 H), 7.54 (d, 1 H), 7.40 (s, 1 H), 7.22 (d, 1 H), 6.56 (d, 1 H), 4.81 (d, 2 H), 3.33 (s, 2 H) ppm.

### EXAMPLE 99

5-[5-Bromo-4-[(5-methanesulfonyl-thiophen-2-ylmethyl)-amino]-pyrimidin-2-ylamino]-1,3-dihydro-indol-2-one

A. 5-Methanesulfonyl-thiophene-2-carbaldehyde (C₆H₆O₃S₂).

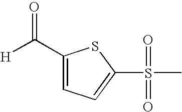

The title intermediate was prepared by adapting the procedure by Archer (Archer, W. J., Cook, R., Taylor, R.; J. Chem. Soc. Perkin Trans. II; 1983, 813-819) and isolated as a light yellow solid in a 26% yield. GC/MS: ret. time=2.96 min; m/z: 190. ¹H NMR (d₆-DMSO) δ: 10.01 (s, 1 H), 8.08 (d, 1 H), 7.94 (d, 1 H), 3.42 (s, 3 H) ppm.

B. (5-Methanesulfonyl-thiophen-2-yl)-methanol (C₆H₈O₃S₂).

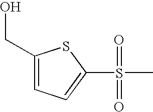

The title intermediate was prepared by adapting the procedure by Lee (Lee, Y. and Silverman, R. B.; Tetrahedron, 2001, 53, 5339-5352) and isolated as a yellow oil in a 74% yield. GC/MS: ret. time=3.55 min; m/z: 192. ¹H NMR (d₆-DMSO) δ: 7.61 (d, 1 H), 7.04 (d, 1 H), 5.83 (t, 1 H), 4.67 (d, 2 H), 3.27 (s, 3 H) ppm.

C. 2-Chloromethyl-5-methanesulfonyl-thiophene (C₁₃H₁₄O₆S₃).

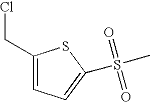

The title intermediate was isolated as a white solid in a 52% yield. GC/MS: ret. time=3.31 min; m/z: 210/212. ¹H NMR (d₆-DMSO) δ: 7.65 (d, 1 H), 7.29 (d, 1 H), 5.08 (s, 2 H), 3.32 (s, 3 H) ppm.

D. C-(5-Methanesulfonyl-thiophen-2-yl)-methylamine (C₆H₉NO₂S₂)

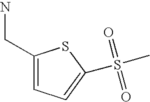

The title intermediate was isolated as a white solid in the TFA salt form in a 75% yield. GC/MS: ret. time=3.53 min; m/z: 191. ¹H NMR (d₆-DMSO) δ: 8.36 (s, 2 H), 7.73 (d, 1 H), 7.32 (d, 1 H), 4.32 (s, 2 H), 3.32 (s, 3 H) ppm.

E. (5-Bromo-2-chloro-pyrimidin-4-yl)-(5-methanesulfonyl-thiophen-2-ylmethyl)-amine (C₁₀H₉BrClN₃O₂S₂).

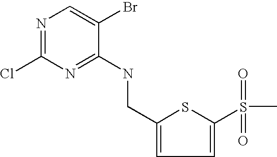

The title intermediate was isolated as a light yellow solid in a 74% yield. GC/MS: ret. time=7.00 min; m/z: 381/383/385. ¹H NMR (d₆-DMSO) δ: 8.49 (t, 1 H), 8.31 (s, 1 H), 7.62 (d, 1 H), 7.14 (d, 1 H), 4.72 (d, 2 H), 3.27 (s, 3 H) ppm.

F. 5-{5-Bromo-4-[(5-methanesulfonyl-thiophen-2-ylmethyl)-amino]-pyrimidin-2-ylamino}-1,3-dihydro-indol-2-one (C₁₈H₁₆BrN₅O₃S₂).

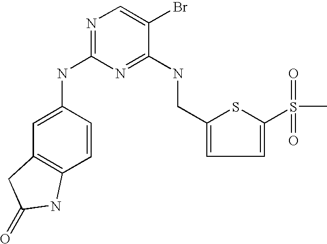

The title compound was isolated as a pink solid in an 18% yield. MS: 494.0/495.9 (MH+). ¹H NMR (d₆-DMSO) δ: 10.18 (s, 1 H), 9.11 (s, 1 H), 8.01 (s, 1 H), 7.74 (t, 1 H), 7.61 (d, 1 H), 7.47 (s, 1 H), 7.34-7.31 (m, 1 H), 7.11 (d, 1 H), 6.63 (d, 1 H), 4.74 (d, 1 H), 3.37 (s, 2 H), 3.30 (s, 3 H) ppm.

### EXAMPLE 100

5-[5-Bromo-4-(2.3-difluoro-benzylamino)-pyrimidin-2-ylamino]-1,3-dihydro-indol-2-one

A. (5-Bromo-2-chloro-pyrimidin-4-yl)-(2,3-difluoro-benzyl)-amine (C₁₁H₇BrClF₂N₃).

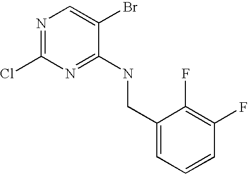

The title intermediate was isolated as a white solid in a 78% yield. GC/MS: ret. time=4.77 min; m/z: 333/335/337. ¹H NMR (d₆-DMSO) δ: 8.33 (t, 1 H), 8.28 (s, 1 H), 7.33-7.26 (m, 1 H), 7.16-7.06 (m, 2 H), 4.61 (d, 2 H) ppm.

B. 5-[5-Bromo-4-(2.3-difluoro-benzylamino)-pyrimidin-2-ylamino]-1,3-dihydro-indol-2-one (C₁₉H₁₄BrF₂N₅O).

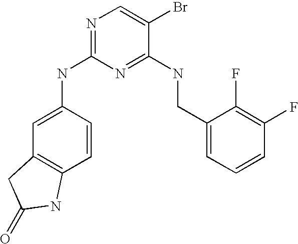

The title compound was isolated as a pink solid in a 33% yield. MS: 446.1/448.0 (MH+). ¹H NMR (d₆-DMSO) δ: 10.16 (s, 1 H), 9.05 (s, 1 H), 8.00 (s, 1 H), 7.58 (t, 1 H), 7.32-7.21 (m, 3 H), 7.13-7.11 (m, 1 H), 7.03-7.01 (m, 1 H), 6.54 (d, 1 H), 4.65 (d, 2 H), 3.28 (s, 2 H) ppm.

### EXAMPLE 101

6-[5-Bromo-4-(2.3-difluoro-benzylamino)-pyrimidin-2-ylamino]-1,3-dihydro-indol-2-one

A. (5-Bromo-2-chloro-pyrimidin-4-yl)-(2,3-difluoro-benzyl)-amine (C₁₃H₇BrClF₃N₃).

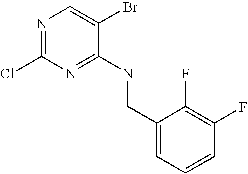

The title intermediate was isolated as a white solid in a 78% yield. GC/MS: ret. time=4.77 min; m/z: 333/335/337. ¹H NMR (d₆-DMSO) δ: 8.33 (t, 1 H), 8.28 (s, 1 H), 7.33-7.26 (m, 1 H), 7.16-7.06 (m, 2 H), 4.61 (d, 2 H) ppm.

B. 6-[5-Bromo-4-(2.3-difluoro-benzylamino)-pyrimidin-2-ylamino]-1,3-dihydro-indol-2-one (C₁₉H₁₄BrF₂N₅O).

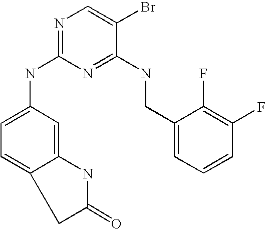

The title compound was isolated as a purple solid in an 8% yield. MS: 446.0/447.9 (MH+). ¹H NMR (d₆-DMSO) δ: 10.28 (s, 1 H), 9.18 (s, 1 H), 8.04 (s, 1 H), 7.57 (t, 1 H), 7.28-7.26 (m, 1 H), 7.13-7.06 (m, 4 H), 6.87 (d, 1 H), 4.68 (d, 1 H), 3.32 (s, 2 H) ppm.

### EXAMPLE 102

5-[5-Bromo-4-(2,4-difluoro-benzylamino)-pyrimidin-2-ylamino]-1,3-dihydro-indol-2-one

A. (5-Bromo-2-chloro-pyrimidin-4-yl)-(2,4-difluoro-benzyl)-amine (C₁₁H₇BrClF₃N₃).

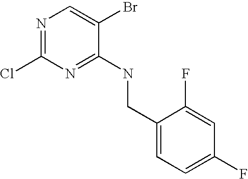

The title intermediate was isolated as a white solid in a 78% yield. GC/MS: ret. time=4.63 min; m/z: 333/335/337. ¹H NMR (d₆-DMSO) δ: 8.28-8.26 (m, 2 H), 7.35-7.29 (m, 1 H), 7.23-7.17 (m, 1 H), 7.04-6.99 (m, 1 H), 4.54 (d, 2 H) ppm.

B. 5-[5-Bromo-4-(2,4-difluoro-benzylamino)-pyrimidin-2-ylamino]-1,3-dihydro-indol-2-one (C₁₉H₁₄BrF₂N₅O).

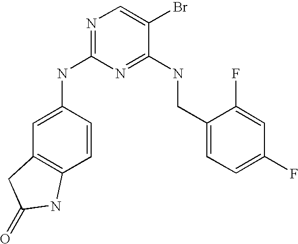

The title compound was isolated as a dark pink solid in a 13% yield. MS: 446.1/448.1 (MH+). ¹H NMR (d₆-DMSO) δ: 10.15 (s, 1 H), 9.04 (s, 1 H), 8.00 (s, 1 H), 7.51 (t, 1 H), 7.39 (s, 1 H), 7.25-7.20 (m, 3 H), 7.03-6.98 (m, 1 H), 6.56 (d, 1 H), 4.58 (d, 2 H), 3.30 (s, 2 H) ppm.

### EXAMPLE 103

6-[5-Bromo-4-(2,4-difluoro-benzylamino)-pyrimidin-2-ylamino]-1,3-dihydro-indol-2-one

A. (5-Bromo-2-chloro-pyrimidin-4-yl)-(2,4-difluoro-benzyl)-amine (C₁₁H₇BrClF₂N₃).

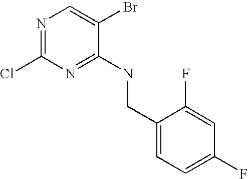

The title intermediate was isolated as a white solid in a 78% yield. GC/MS: ret. time=4.63 min; m/z: 333/335/337. ¹H NMR (d₆-DMSO) δ: 8.28-8.26 (m, 2 H), 7.35-7.29 (m, 1 H), 7.23-7.17 (m, 1 H), 7.04-6.99 (m, 1 H), 4.54 (d, 2 H) ppm.

B. 6-[5-Bromo-4-(2.4-difluoro-benzylamino)-pyrimidin-2-ylamino]-1,3-dihydro-indol-2-one ((C₁₄H₁₄BrF₂N₅O0.

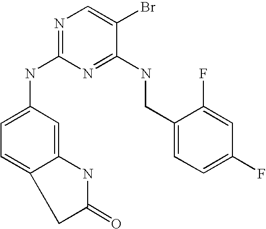

The title compound was isolated as a light purple solid in an 11% yield. MS: 446.2/448.2. ¹H NMR (d₆-DMSO) δ: 10.28 (s, 1 H), 9.18 (s, 1 H), 8.03 (s, 1 H), 7.50 (t, 1 H), 7.32, 7.12 (m, 4 H), 7.02-6.97 (m, 1 H), 6.90 (d, 1 H), 4.61 (d, 2 H), 3.32 (s, 2 H) ppm.

The following compounds were also prepared using the methods described in this application:

6-[5-Chloro-4-(2-trifluoromethyl-benzylamino)-pyrimidin-2-ylamino]-1,3-dihydro-indol-2-one;

5-Chloro-N2-(1-methyl-1H-indol-5-yl)-N4-(2-trifluoromethyl-benzyl)-pyrimidine-2,4-diamine;

5-Chloro-N²-(1H-indazol-5-yl)-N⁴-(2-trifluoromethyl-benzyl)-pyrimidine-2,4-diamine;

5-Chloro-N 2-(1-methyl-1H-indol-5-yl)-N⁴-pyridin-2-ylmethyl-pyrimidine-2,4-diamine;

6-{5-Chloro-4-[(pyridin-2-ylmethyl)-amino]-pyrimidin-2-ylamino}-1,3-dihydro-indol-2-one;

5-Chloro-N2-(1H-indazol-6-yl)-N4-(2-trifluoromethyl-benzyl)-pyrimidine-2,4-diamine;

5-Chloro-N2-(1H-indazol-6-yl)-N4-pyridin-2-ylmethyl-pyrimidine-2,4-diamine;

(5-{5-Bromo-4-[(pyridin-2-ylmethyl)-amino]-pyrimidin-2-ylamino}-indazol-1-yl)-acetic acid tert-butyl ester;

(6-{5-Bromo-4-[(pyridin-2-ylmethyl)-amino]-pyrimidin-2-ylamino}-indazol-2-yl)-acetic acid tert-butyl ester;

6-{4-[(Pyridin-2-ylmethyl)-amino]-5-trifluoromethyl-pyrimidin-2-ylamino}-1,3-dihydro-indol-2-one;

N2-(1-Methyl-1H-indol-5-yl)-N4-pyridin-2-ylmethyl-5-trifluoromethyl-pyrimidine-2,4-diamine;

(6-{5-Bromo-4-[(pyridin-2-ylmethyl)-amino]-pyrimidin-2-ylamino}-indol-1-yl)-acetic acid tert-butyl ester;

N4-Pyridin-2-ylmethyl-N2-quinolin-5-yl-5-trifluoromethyl-pyrimidine-2,4-diamine;

2-(6-{5-Bromo-4-[(pyridin-2-ylmethyl)-amino]-pyrimidin-2-ylamino}-indol-1-yl)-N-(2-methoxy-ethyl)-acetamide;

6-{5-Chloro-4-[(3-methyl-pyridin-2-ylmethyl)-amino]-pyrimidin-2-ylamino}-1,3-dihydro-indol-2-one;

(6-{5-Bromo-4-[(pyridin-2-ylmethyl)-amino]-pyrimidin-2-ylamino}-indol-1-yl)-acetic acid;

(6-{5-Bromo-4-[(pyridin-2-ylmethyl)-amino]-pyrimidin-2-ylamino}-indazol-1-yl)-acetic acid tert-butyl ester;

N2-(1H-Indazol-6-yl)-N4-pyridin-2-ylmethyl-5-trifluoromethyl-pyrimidine-2,4-diamine;

(5-{5-Bromo-4-[(pyridin-2-ylmethyl)-amino]-pyrimidin-2-ylamino}-indol-1-yl)-acetic acid tert-butyl ester;

(6-{5-Bromo-4-[(pyridin-2-ylmethyl)-amino]-pyrimidin-2-ylamino}-indazol-1-yl)-acetic acid;

(5-{5-Bromo-4-[(pyridin-2-ylmethyl)-amino]-pyrimidin-2-ylamino}-indol-1-yl)-acetic acid

(5-{5-Bromo-4-[(pyridin-2-ylmethyl)-amino]-pyrimidin-2-ylamino}-indazol-1-yl)-acetic acid;

5-{5-Chloro-4-[(3-methyl-pyridin-2-ylmethyl)-amino]-pyrimidin-2-ylamino}-1,3-dihydro-indol-2-one;

5-[5-Chloro-4-(3-methanesulfonyl-benzylamino)-pyrimidin-2-ylamino]-1,3-dihydro-indol-2-one;

6-[5-Chloro-4-(3-methyl-benzylamino)-pyrimidin-2-ylamino]-1,3-dihydro-indol-2-one;

5-[5-Chloro-4-(2-fluoro-benzylamino)-pyrimidin-2-yiamino]-1,3-dihydro-indol-2-one;

6-[5-Chloro-4-(2-fluoro-benzylamino)-pyrimidin-2-ylamino]-1,3-dihydro-indol-2-one;

5-[5-Bromo-4-(2-methoxy-ethylamino)-pyrimidin-2-ylamino]-1,3-dihydro-indol-2-one;

5-[5-Chloro-4-(3-methyl-benzylamino)-pyrimidin-2-ylamino]-1,3-dihydro-indol-2-one;

6-{5-Chloro-4-[(4-methyl-pyridin-2-ylmethyl)-amino]-pyrimidin-2-ylamino}-1,3-dihydro-indol-2-one;

5-(4-Benzylamino-5-chloro-pyrimidin-2-ylamino)-1,3-dihydro-indol-2-one;

5-Bromo-N2-(1H-indol-5-yl)-N4-pyridin-2-ylmethyl-pyrimidine-2,4-diamine;

5-Bromo-N2-(1H-indol-5-yl)-N4-(2-pyridin-2-yl-ethyl)-pyrimidine-2,4-diamine;

5-Bromo-N2-(1H-indol-4-yl)-N4-(2-pyridin-2-yl-ethyl)-pyrimidine-2,4-diamine;

5-Bromo-N2-(1H-indazol-5-yl)-N4-(2-pyridin-2-yl-ethyl)-pyrimidine-2,4-diamine;

5-Bromo-N2-(1H-indazol-6-yl)-N4-(2-pyridin-2-yl-ethyl)-pyrimidine-2,4-diamine;

5-Bromo-N2-(1H-indol-4-yl)-N4-pyridin-2-ylmethyl-pyrimidine-2,4-diamine;

5-Bromo-N2-(1H-indazol-5-yl)-N4-pyridin-2-ylmethyl-pyrimidine-2,4-diamine;

N2-(1H-Indol-5-yl)-N4-pyridin-2-ylmethyl-pyrimidine-2,4-diamine;

N2-(1H-Indazol-6-yl)-N4-pyridin-2-ylmethyl-pyrimidine-2,4-diamine;

N2-(1H-Indol-5-yl)-N4-(2-pyridin-2-yl-ethyl)-pyrimidine-2,4-diamine;

N2-(1H-Indazol-5-yl)-N4-pyridin-2-ylmethyl-pyrimidine-2,4-diamine;

N2-(1H-Indazol-5-yl)-N-4-(2-pyridin-2-yl-ethyl)-pyrimidine-2,4-diamine;

N2-(1H-Indazol-6-yl)-N4-(2-pyridin-2-yl-ethyl)-pyrimidine-2,4-diamine;

5-{5-Bromo-4-[(pyridin-2-ylmethyl)-amino]-pyrimidin-2-ylamino}-1,3-dihydro-benzoimidazol-2-one;

5-[5-Bromo-4-(2-pyridin-2-yl-ethylamino)-pyrimidin-2-ylamino]-1,3-dihydro-benzoimidazol-2-one;

5-{4-[(Pyridin-2-ylmethyl)-amino]-pyrimidin-2-ylamino}-1,3-dihydro-benzoimidazol-2-one;

5-[4-(2-Pyridin-2-yl-ethylamino)-pyrimidin-2-ylamino]-1,3-dihydro-benzoimidazol-2-one;

5-Bromo-N2-(1H-indazol-6-yl)-N-4-pyridin-2-ylmethyl-pyrimidine-2,4-diamine;

5-{5-Bromo-4-[(pyridin-2-ylmethyl)-amino]-pyrimidin-2-ylamino}-1,3-dihydro-indol-2-one;

5-[5-Bromo-4-(2-pyridin-2-yl-ethylamino)-pyrimidin-2-ylamino]-1,3-dihydro-indol-2-one;

5-[4-(2-Pyridin-2-yl-ethylamino)-pyrimidin-2-ylamino]-1,3-dihydro-indol-2-one;

5-Bromo-N2-(2-methyl-1H-indol-5-yl)-N4-(2-pyridin-2-yl-ethyl)-pyrimidine-2,4-diamine;

N2-(2-Methyl-1H-indol-5-yl)-N4-pyridin-2-ylmethyl-pyrimidine-2,4-diamine;

N2-(1H-Indol-6-yl)-N4-pyridin-2-ylmethyl-pyrimidine-2,4-diamine;

5-Bromo-N2-(2-methyl-1H-indol-5-yl)-N4-pyridin-2-ylmethyl-pyrimidine-2,4-diamine;

5-Bromo-N2-(1H-indol-6-yl)-N4-pyridin-2-ylmethyl-pyrimidine-2,4-diamine;

5-Bromo-N2-(1H-indol-6-yl)-N4-(2-pyridin-2-yl-ethyl)-pyrimidine-2,4-diamine;

N2-(1H-Benzoimidazol-5-yl)-5-bromo-N4-pyridin-2-ylmethyl-pyrimidine-2,4-diamine;

N2-(1H-Benzoimidazol-5-yl)-5-bromo-N4-(2-pyridin-2-yl-ethyl)-pyrimidine-2,4-diamine;

3-[5-Bromo-4-(2-pyridin-2-yl-ethylamino)-pyrimidin-2-yl]-3H-benzoimidazol-5-ylamine;

N2-(1H-Benzoimidazol-5-yl)-N-4-pyridin-2-ylmethyl-pyrimidine-2,4-diamine;

5-Bromo-N2-(2-methyl-1H-benzoimidazol-5-yl)-N4-pyridin-2-ylmethyl-pyrimidine-2,4-diamine;

N2-(2-Methyl-1H-benzoimidazol-5-yl)-N4-pyridin-2-ylmethyl-pyrimidine-2,4-diamine;

5-Bromo-N2-(2-methyl-1H-benzoimidazol-5-yl)-N4-(2-pyridin-2-yl-ethyl)-pyrimidine-2,4-diamine;

5-Bromo-N2-(2,3-dihydro-1H-indol-5-yl)-N4-(2-pyridin-2-yl-ethyl)-pyrimidine-2,4-diamine;

N2-(2,3-Dihydro-1H-indol-5-yl)-N4-pyridin-2-ylmethyl-pyrimidine-2,4-diamine;

5-Bromo-N2-(1-methyl-1H-indol-5-yl)-N-4-(2-pyridin-2-yl-ethyl)-pyrimidine-2,4-diamine;

N2-(1-Methyl-1H-indol-5-yl)-N4-pyridin-2-ylmethyl-pyrimidine-2,4-diamine;

5-Bromo-N2-(2,3-dihydro-1H-indol-5-yl)-N4-pyridin-2-ylmethyl-pyrimidine-2,4-diamine;

5-Bromo-N2-(1-methyl-1H-indol-5-yl)-N4-pyridin-2-ylmethyl-pyrimidine-2,4-diamine;

5-Fluoro-N4-pyridin-2-ylmethyl-N2-quinolin-6-yl-pyrimidine-2,4-diamine;

5-Bromo-N4-pyridin-2-ylmethyl-N2-quinolin-6-yl-pyrimidine-2,4-diamine;

5-Bromo-N2-(1H-indol-7-yl)-N4-(2-pyridin-2-yl-ethyl)-pyrimidine-2,4-diamine;

5-Bromo-N2-(1H-indol-7-yl)-N4-pyridin-2-ylmethyl-pyrimidine-2,4-diamine;

5-Bromo-N2-(1H-indazol-4-yl)-N4-pyridin-2-ylmethyl-pyrimidine-2,4-diamine;

6-{5-Bromo-4-[(pyridin-2-ylmethyl)-amino]-pyrimidin-2-ylamino}-1,3-dihydro-indol-2-one;

5-Bromo-N2-(1H-indazol-4-yl)-N4-(2-pyridin-2-yl-ethyl)-pyrimidine-2,4-diamine;

5-Bromo-N4-(2-pyridin-2-yl-ethyl)-N-2-quinolin-6-yl-pyrimidine-2,4-diamine;

5-Bromo-N4-pyridin-2-ylmethyl-N2-quinolin-5-yl-pyrimidine-2,4-diamine;

5-Bromo-N4-(2-pyridin-2-yl-ethyl)-N2-quinolin-5-yl-pyrimidine-2,4-diamine;

6-[5-Bromo-4-(2-pyridin-2-yl-ethylamino)-pyrimidin-2-ylamino]-1,3-dihydro-indol-2-one;

5-Bromo-N4-pyridin-2-ylmethyl-N2-quinolin-8-yl-pyrimidine-2,4-diamine;

5-Bromo-N4-(2-pyridin-2-yl-ethyl)-N2-quinolin-8-yl-pyrimidine-2,4-diamine;

5-{5-Bromo-4-[(pyridin-2-ylmethyl)-amino]-pyrimidin-2-ylamino}-1H-indole-2-carboxylic acid ethyl ester;

6-[5-Bromo-4-(2-trifluoromethyl-benzylamino)-pyrimidin-2-ylamino]-1,3-dihydro-indol-2-one;

5-Bromo-N2-(1H-indazol-5-yl)-N4-(2-trifluoromethyl-benzyl)-pyrimidine-2,4-diamine;

5-Bromo-N2-(1H-indazol-6-yl)-N4-(2-trifluoromethyl-benzyl)-pyrimidine-2,4-diamine;

5-Bromo-N2-(1-methyl-1H-indol-5-yl)-N4-(2-trifluoromethyl-benzyl)-pyrimidine-2,4-diamine;

5-Bromo-N2-(1H-indazol-7-yl)-N4-pyridin-2-ylmethyl-pyrimidine-2,4-diamine;

5-Bromo-N2-(1H-indazol-4-yl)-N4-(2-trifluoromethyl-benzyl)-pyrimidine-2,4-diamine;

6-{5-Bromo-4-[(pyridin-2-ylmethyl)-amino]-pyrimidin-2-ylamino}-3H-isobenzofuran-1-one;

N2-Benzothiazol-6-yl-5-bromo-N4-pyridin-2-ylmethyl-pyrimidine-2,4-diamine;

5-{5-Bromo-4-[(pyridin-2-ylmethyl)-amino]-pyrimidin-2-ylamino}-2-methyl-1H-indole-3-carbonitrile;

5-Bromo-N4-pyridin-2-ylmethyl-N2-(1-pyridin-2-ylmethyl-1H-indazol-5-yl)-pyrimidine-2,4-diamine;

N2-(1-Benzyl-1H-indol-5-yl)-5-bromo-N4-pyridin-2-ylmethyl-pyrimidine-2,4-diamine;

5-Bromo-N4-pyridin-2-ylmethyl-N2-(1-pyridin-2-ylmethyl-1H-indol-5-yl)-pyrimidine-2,4-diamine;

N2-(1-Benzyl-1H-indazol-5-yl)-5-bromo-N4-pyridin-2-ylmethyl-pyrimidine-2,4-diamine;

5-Bromo-N2-(1-methyl-1H-indazol-5-yl)-N4-pyridin-2-ylmethyl-pyrimidine-2,4-diamine;

5-Bromo-N4-(4-methyl-cyclohexyl)-N2-[3-(1,2,3,6-tetrahydro-pyridin-4-yl)-1H-indol-5-yl]-pyrimidine-2,4-diamine;

5-Bromo-N4-(4-methyl-cyclohexyl)-N-2-[3-(1,2,3,6-tetrahydro-pyridin-4-yl)-1H-indol-5-yl]-pyrimidine-2,4-diamine;

5-Bromo-N4-cyclohexylmethyl-N2-[3-(1,2,3,6-tetrahydro-pyridin-4-yl)-1H-indol-5-yl]-pyrimidine-2,4-diamine;

1-{5-Fluoro-4-[(pyridin-2-ylmethyl)-amino]-pyrimidin-2-yl}-3-(1,2,3,6-tetrahydro-pyridin-4-yl)-1H-indol-5-ylamine;

1-{5-Chloro-4-[(pyridin-2-ylmethyl)-amino]-pyrimidin-2-yl}-3-(1,2,3,6-tetrahydro-pyridin-4-yl)-1H-indol-5-ylamine;

5-Fluoro-N2-(1H-indazol-5-yl)-N4-pyridin-2-ylmethyl-pyrimidine-2,4-diamine;

5-{5-Fluoro-4-[(pyridin-2-ylmethyl)-amino]-pyrimidin-2-ylamino}-1,3-dihydro-indol-2-one;

5-Chloro-N2-(1H-indazol-5-yl)-N4-pyridin-2-ylmethyl-pyrimidine-2,4-diamine;

5-{5-Chloro-4-[(pyridin-2-ylmethyl)-amino]-pyrimidin-2-ylamino}-1,3-dihydro-indol-2-one;

5-Fluoro-N4-(2-pyridin-2-yl-ethyl)-N2-[3-(1,2,3,6-tetrahydro-pyridin-4-yl)-1H-indol-5-yl]-pyrimidine-2,4-diamine;

5-Chloro-N4-(2-pyridin-2-yl-ethyl)-N-2-[3-(1,2,3,6-tetrahydro-pyridin-4-yl)-1H-indol-5-yl]-pyrimidine-2,4-diamine;

5-Fluoro-N2-(1H-indazol-5-yl)-N4-(2-pyridin-2-yl-ethyl)-pyrimidine-2,4-diamine;

5-[5-Fluoro-4-(2-pyridin-2-yl-ethylamino)-pyrimidin-2-ylamino]-1,3-dihydro-indol-2-one;

5-Chloro-N2-(1H-indazol-5-yl)-N4-(2-pyridin-2-yl-ethyl)-pyrimidine-2,4-diamine;

5-[5-Chloro-4-(2-pyridin-2-yl-ethylamino)-pyrimidin-2-ylamino]-1,3-dihydro-indol-2-one;

5-{4-[(Pyridin-2-ylmethyl)-amino]-5-trifluoromethyl-pyrimidin-2-ylamino}-1,3-dihydro-indol-2-one;

5-{5-Methoxy-4-[(pyridin-2-ylmethyl)-amino]-pyrimidin-2-ylamino}-1,3-dihydro-indol-2-one;

5-[5-Methoxy-4-(2-pyridin-2-yl-ethylamino)-pyrimidin-2-ylamino]-1,3-dihydro-indol-2-one;

5-[5-Methoxy-4-(2-trifluoromethyl-benzylamino)-pyrimidin-2-ylamino]-1,3-dihydro-indol-2-one;

5-{5-Bromo-4-[(cyclohex-1-enylmethyl)-amino]-pyrimidin-2-ylamino}-1,3-dihydro-indol-2-one;

5-[5-Bromo-4-(methyl-pyridin-2-ylmethyl-amino)-pyrimidin-2-ylamino]-1,3-dihydro-indol-2-one;

5-[5-Bromo-4-(4-methyl-cyclohexylamino)-pyrimidin-2-ylamino]-1,3-dihydro-indol-2-one;

5-[5-Bromo-4-(4-methyl-cyclohexylamino)-pyrimidin-2-ylamino]-1,3-dihydro-indol-2-one;

5-[5-Bromo-4-(cyclohexylmethyl-amino)-pyrimidin-2-ylamino]-1,3-dihydro-indol-2-one;

5-[5-Chloro-4-(2-trifluoromethyl-benzylamino)-pyrimidin-2-ylamino]-1,3-dihydro-indol-2-one;

2-(2-Oxo-2,3-dihydro-1H-indol-5-ylamino)-4-[(pyridin-2-ylmethyl)-amino]-pyrimidine-5-carbonitrile;

5-{5-Methyl-4-[(pyridin-2-ylmethyl)-amino]-pyrimidin-2-ylamino}-1,3-dihydro-indol-2-one;

N2-(1H-Indazol-5-yl)-5-methyl-N4-pyridin-2-ylmethyl-pyrimidine-2,4-diamine;

5-Fluoro-N4-pyridin-2-ylmethyl-N2-[3-(1,2,3,6-tetrahydro-pyridin-4-yl)-1H-indol-5-yl]-pyrimidine-2,4-diamine;

5-Chloro-N4-pyridin-2-ylmethyl-N2-[3-(1,2,3,6-tetrahydro-pyridin-4-yl)-1H-indol-5-yl]-pyrimidine-2,4-diamine;

2-(2-Oxo-2,3-dihydro-1H-indol-5-ylamino)₄-(2-trifluoromethyl-benzylamino)-pyrimidine-5-carbonitrile;

5-{4-[Methyl-(2-pyridin-2-yl-ethyl)-amino]-pyrimidin-2-ylamino}-1,3-dihydro-indol-2-one;

5-Bromo-N4-cyclohex-1-enylmethyl-N2-[3-(1,2,3,6-tetrahydro-pyridin-4-yl)-1H-indol-5-yl]-pyrimidine-2,4-diamine;

N2-(1H-Indazol-5-yl)-N4-pyridin-2-ylmethyl-5-trifluoromethyl-pyrimidine-2,4-diamine;

5-[5-Trifluoromethyl-4-(2-trifluoromethyl-benzylamino)-pyrimidin-2-ylamino]-1,3-dihydro-indol-2-one;

6-{2-[(Pyridin-2-ylmethyl)-amino]-5-trifluoromethyl-pyrimidin-4-ylamino}-1,3-dihydro-indol-2-one;

5-[5-Bromo-4-(piperidin-4-ylamino)-pyrimidin-2-ylamino]-1,3-dihydro-indol-2-one;

5-[4-(1-Acetyl-piperidin-4-ylamino)-5-bromo-pyrimidin-2-ylamino]-1,3-dihydro-indol-2-one;

2-(2-Oxo-2,3-dihydro-1H-indol-6-ylamino)-4-[(pyridin-2-ylmethyl)-amino]-pyrimidine-5-carbonitrile;

5-{4-[(3-Methyl-pyridin-2-ylmethyl)-amino]-5-trifluoromethyl-pyrimidin-2-ylamino}-1,3-dihydro-indol-2-one;

6-{4-[(3-Methyl-pyridin-2-ylmethyl)-amino]-5-trifluoromethyl-pyrimidin-2-ylamino}-1,3-dihydro-indol-2-one;

4-[5-Bromo-2-(2-oxo-2,3-dihydro-1H-indol-5-ylamino)-pyrimidin-4-ylamino]-piperidine-1-carboxylic acid tert-butyl ester;

5-[5-Bromo-4-(1-methanesulfonyl-piperidin-4-ylamino)-pyrimidin-2-ylamino]-1,3-dihydro-indol-2-one;

5-[5-Bromo-4-(piperidin-3-ylamino)-pyrimidin-2-ylamino]-1,3-dihydro-indol-2-one;

4-[5-Bromo-2-(2-oxo-2,3-dihydro-1H-indol-5-ylamino)-pyrimidin-4-ylamino]-piperidine-1-carboxylic acid ethylamide;

3-[5-Bromo-2-(2-oxo-2,3-dihydro-1H-indol-5-ylamino)-pyrimidin-4-ylamino]-piperidine-1-carboxylic acid ethylamide;

5-[4-(1-Benzoyl-piperidin-4-ylamino)-5-bromo-pyrimidin-2-ylamino]-1,3-dihydro-indol-2-one;

6-[4-(3-Methanesulfonyl-benzylamino)-5-methoxy-pyrimidin-2-ylamino]-1,3-dihydro-indol-2-one;

6-[4-(3-Methanesulfonyl-benzylamino)-5-trifluoromethyl-pyrimidin-2-ylamino]-1,3-dihydro-indol-2-one; 6-[4-(3-Methanesulfonyl-benzylamino)-pyrimidin-2-ylamino]-1,3-dihydro-indol-2-one;

5-[4-(1-Benzenesulfonyl-piperidin-4-ylamino)-5-bromo-pyrimidin-2-ylamino]-1,3-dihydro-indol-2-one;

5-[4-(3-Methanesulfonyl-benzylamino)-5-trifluoromethyl-pyrimidin-2-ylamino]-1,3-dihydro-indol-2-one;

6-{5-Chloro-4-[(piperidin-3-ylmethyl)-amino]-pyrimidin-2-ylamino}-1,3-dihydro-indol-2-one;

6-{5-Chloro-4-[(1-methanesulfonyl-piperidin-3-ylmethyl)-amino]-pyrimidin-2-ylamino}-1,3-dihydro-indol-2-one;

6{5-Bromo-4-[(piperidin-3-ylmethyl)-amino]-pyrimidin-2-ylamino}-1,3-dihydro-indol-2-one;

6-{5-Bromo-4-[(1-methanesulfonyl-piperidin-3-ylmethyl)-amino]-pyrimidin-2-ylamino}-1,3-dihydro-indol-2-one;

5-[5-Fluoro-4-(3-methanesulfonyl-benzylamino)-pyrimidin-2-ylamino]-1,3-dihydro-indol-2-one;

5-{5-Bromo-4-[(1-hydroxy-cyclohexylmethyl)-amino]-pyrimidin-2-ylamino}-1,3-dihydro-indol-2-one;

1. A compound of the formula 1 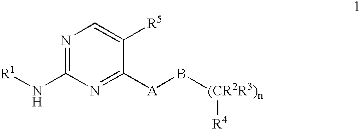
or a pharmaceutically acceptable salt, solvate, hydrate, or prodrug thereof, wherein R¹ has the following formula 2 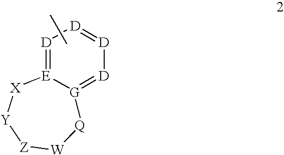
wherein each D is independently selected from the group consisting of CR⁸ and N, with the proviso that R¹ is linked to NH group through a ring carbon atom;
wherein E and G are independently selected from the group consisting of N and C;
wherein X, W and Q are independently selected from the group consisting of N, O, S, SO₂, CO, NR³, CR² and CR²R³;
wherein Y and Z are independently present or absent, if present Y and Z are selected from the group consisting of N, O, S, SO₂, CO, NR³, CR² and CR²R³;
wherein A is present or absent, if present A is selected from the group consisting of O, S and NH and wherein B is present or absent, if present B is selected from the group consisting of CO, SO₂, and NR⁶, with the proviso that when A is O or S that B is absent;
wherein n is an integer from 1 to 3;
wherein each R² is independently selected from the group consisting of H, C₁-C₆ alkyl, C₃-C₇ cycloalkyl, C₄-C₇ heterocycloalkyl, OC₁-C₆ alkyl, OC₃-C₇ cycloalkyl, OC₄-C₇ heterocyloalkyl, NH₂, NHR⁶, NR⁶R⁷, SR⁶, SOR⁶, SO₂R⁶, CO₂R⁶, CONH₂, CONHR⁶, CONR⁶R⁷, SO₂NH₂, SO₂NHR⁶, SO₂NR⁶R⁷, NHCOR⁶, NR⁶CONR⁶, NHCONHR⁶, NR⁶CONHR⁶, NHCONR⁶R⁷, NR⁶CONR⁶R⁷, NHSO₂R⁶, NR⁶SO₂R⁶, with the proviso that O, N or S atom of the foregoing substituents may not be bound to a carbon atom bound to another heteroatom, said alkyl, cycloalkyl, heterocycloalkyl moieties of the foregoing groups are optionally substituted by 1 to 3 substituents independently selected from the group consisting of H, halo, C₁-C₆ alkyl, CN, NH₂, NHR¹⁰, N(R¹⁰)₂, OR¹⁰, C₁-C₆ alkyl, C₃-C₇ cycloalkyl, C₄-C₇ heterocycloalkyl, CO₂R¹¹, CONH₂, CONHR¹¹, and CONR¹¹R¹²;
wherein each R³ is independently selected from the group consisting of H, C₁-C₆ alkyl, C₃-C₇ cycloalkyl, C₄-C₇ heterocycloalkyl, CO₂R⁶, CONH₂, CONHR⁶, CONR⁶R⁷ or R² and R³ taken together with the carbon atom they are linked to can form a 3-7 membered cycloalkyl ring or 4-7 membered heterocycloalkyl ring, wherein each methylene group present in said 3-7 membered cycloalkyl ring and said 4-7 membered heterocycloalkyl ring may be optionally replaced by a C═O group, said alkyl, cycloalkyl, heterocycloalkyl moieties of the foregoing groups are optionally substituted by 1 to 3 substituents independently selected from the group consisting of H, halo, C₁-C₆ alkyl, CN, NH₂, NHR¹⁰, N(R¹⁰)₂, OR¹⁰, C₁-C₆ alkyl, C₃-C₇ cycloalkyl, C₄-C₇ heterocycloalkyl, CO₂R¹¹, CONH₂, CONHR¹¹, and CONR¹¹R¹²;
wherein R⁴ is selected from the group consisting of H, C₁-C₆ alkyl, C₃-C₇ cycloalkyl, C₄-C₇ heterocycloalkyl, C₆-C₁₀ aryl, and 5-10 membered heteroaryl, the alkyl, cycloalkyl, heterocycloalkyl, aryl and heteroaryl moieties of the foregoing groups are optionally substituted by 1 to 3 subsitutents independently selected from the group consisting of H, halo, OH, NO₂, C₁-C₆ alkyl, C(R⁶)═CR⁶R⁷, C═CR⁶, C₃-C₇ cycloalkyl, C₄-C₇ heterocycloalkyl, OC₁-C₆ alkyl, OC₃-C₇ cycloalkyl, OC₄-C₇ heterocyloalkyl, C═N—OH, C═N—O(C₁-C₆ alkyl), NH₂, NHR⁶, NR⁶R⁷, SR⁶, SOR⁶, SO₂R⁶, CO₂R⁶, CONH₂, CONHR⁶, CONR⁶R⁷, SO₂NH₂, SO₂NHR⁶, SO₂NR⁶R⁷, NHCOR⁶, NR⁶CONR⁶, NHCONHR⁶, NR⁶CONHR⁷, NHCONR⁶R⁷, NR⁶CONR⁶R⁷, NHSO₂R⁶, NR⁶ SO₂R⁶, with the proviso that O, N or S atom of the foregoing substituents may not be bound to a carbon atom bound to another heteroatom;
wherein R⁵ is selected from the group consisting of H, Br, Cl, CN, CF₃, CH₂F, CHF₂, SO₂CH₃, CONH₂, cyclopropyl, cyclobutyl, C₆H₅, CONHR⁶, CONR⁶R⁷, CO₂R⁶, C(R⁹)═C(R⁹)₂, and C≡CR⁹;
wherein each R⁵ is independently selected from the group consisting of H, C₁-C₆ alkyl, C₃-C₇ cycloalkyl, C₄-C₇ heterocycloalkyl, C₆-C₁₀ aryl, and 5-10 membered heteroaryl, said alkyl, cycloalkyl, heterocycloalkyl, aryl, and heteroaryl moieties of the foregoing groups are optionally substituted by 1 to 3 substituents independently selected from the group consisting of H, halo, C₁-C₆ alkyl, CN, NH₂, NHR¹⁰, N(R¹⁰)₂, OR¹⁰, C₁-C₆ alkyl, C₃-C₇ cycloalkyl, C₄-C₇ heterocycloalkyl, CO₂R¹¹, CONH₂, CONHR¹¹, and CONR¹¹R¹²;
wherein each R⁷ is independently selected from the group consisting of H, C₁-C₆ alkyl, C₃-C₇ cycloalkyl, C₄-C₇ heterocycloalkyl, C₆-C₁₀ aryl, and 5-10 membered heteroaryl, said alkyl, cycloalkyl, heterocycloalkyl, aryl, and heteroaryl moieties of the foregoing groups are optionally substituted by 1 to 3 substituents independently selected from the group consisting of H, halo, C₁-C₆ alkyl, CN, NH₂, NHR¹⁰, N(R¹⁰)₂, OR¹⁰, C₁-C₆ alkyl, C₃-C₇ cycloalkyl, C₄-C₇ heterocycloalkyl, CO₂R¹¹, CONH₂, CONHR¹¹, and CONR¹¹R¹²;
wherein each R⁸ is independently selected from the group consisting of H, halo, cyano, C₁-C₆ alkyl, C₃-C₇ cycloalkyl, C₄-C₇ heterocycloalkyl, OC₁-C₆ alkyl, OC₃-C₇ cycloalkyl, OC₄-C₇ heterocyloalkyl, NH₂, NHR⁶, NR⁶R⁷, SR⁶, SOR⁶, SO₂R⁶, CO₂R⁶, CONH₂, CONHR⁶, CONR⁶R⁷, SO₂NH₂, SO₂NHR⁶, SO₂NR⁶R⁷, NHCOR⁶, NR⁶CONR⁶, NHCONHR⁶, NR⁶CONHR⁶, NHCONR⁶R⁷, NR⁶CONR⁶R⁷, NHSO₂R⁶, NR⁶SO₂R⁶, said alkyl, cycloalkyl, and heterocycloalkyl moieties of the foregoing groups are optionally substituted by 1 to 3 substituents independently selected from the group consisting of H, halo, C₁-C₆ alkyl, CN, NH₂, NHR³, N(R³)₂, OR³, C₁-C₆ alkyl, C₃-C₇ cycloalkyl, C₄-C₇ heterocycloalkyl, CO₂R⁶, CONH₂, CONHR⁶, and CONR⁶R⁷; and
wherein each R⁹ is independently selected from the group consisting of H, CF₃, and C₁-C₆ alkyl, said C₁-C₆ alkyl is optionally substituted by 1 to 6 halo atoms;
wherein each R¹⁰ is independently selected from the group consisting of H, C₁-C₆ alkyl, C₃-C₇ cycloalkyl, C₄-C₇ heterocycloalkyl, CO₂R¹¹, CONH₂, CONHR¹¹, CONR¹¹R¹², SOR¹¹, SO₂R¹¹, SO₂NH₂, SO₂NHR¹¹, SO₂NR¹¹R¹²; said alkyl, cycloalkyl, heterocycloalkyl moieties of the foregoing groups are optionally substituted by 1 to 3 substituents independently selected from the group consisting of H, halo, C₁-C₆ alkyl, CN, NH₂, NHR¹³, N(R¹³)₂, OR¹³, C₁-C₆ alkyl, C₃-C₇ cycloalkyl, C₄-C₇ heterocycloalkyl, CO₂R¹⁴, CONH₂, CONHR¹⁴, and CONR¹⁴R¹⁵;
wherein each R¹¹ is independently selected from the group consisting of H, C₁-C₆ alkyl, C₃-C₇ cycloalkyl, C₄-C₇ heterocycloalkyl, C₆-C₁₀ aryl, C₅-C₁₀ membered heteroaryl; said alkyl, cycloalkyl, heterocycloalkyl, aryl, and heteroaryl moieties of the foregoing groups are optionally substituted by 1 to 3 substituents independently selected from the group consisting of H, halo, C₁-C₆ alkyl, CN, NH₂, NHR¹³, N(R¹³)₂, OR¹³, C₁-C₆ alkyl, C₃-C₇ cycloalkyl, C₄-C₇ heterocycloalkyl, CO₂R¹⁴, CONH₂, CONHR¹⁴, and CONR¹⁴R¹⁵;
wherein each R¹² is independently selected from the group consisting of H, C₁-C₆ alkyl, C₃-C₇ cycloalkyl, C₄-C₇ heterocycloalkyl, C₆-C₁₀ aryl, C₅-C₁₀ membered heteroaryl; said alkyl, cycloalkyl, heterocycloalkyl, aryl, and heteroaryl moieties of the foregoing groups are optionally substituted by 1 to 3 substituents independently selected from the group consisting of H, halo, C₁-C₆ alkyl, CN, NH₂, NHR¹³, N(R¹³)₂, OR¹³, CJ-C₆ alkyl, C₃-C₇ cycloalkyl, C₄-C₇ heterocycloalkyl, CO₂R¹⁴, CONH₂, CONHR¹⁴, and CONR¹⁴R¹⁵;
wherein each R¹³ is independently selected from the group consisting of H, C₁-C₆ alkyl, C₃-C₇ cycloalkyl, C₄-C₇ heterocycloalkyl, CO₂R¹⁴, CONH₂, CONHR¹⁴, CONR¹⁴R¹⁵, SOR¹⁴, SO₂R¹⁴, SO₂NH₂, SO₂NHR¹⁴, SO₂NR¹⁴R¹⁵;
wherein each R¹⁴ is independently selected from the group consisting of H, C₁-C₆ alkyl, C₃-C₇ cycloalkyl, C₄-C₇ heterocycloalkyl, C₆-C₁₀ aryl, C₅-C₁₀ membered heteroaryl; said alkyl, cycloalkyl, heterocycloalkyl, aryl, and heteroaryl moieties of the foregoing groups are optionally substituted by 1 to 3 substituents independently selected from the group consisting of H, halo, C₁-C₆ alkyl, CN, NH₂, NH C₁-C₆alkyl, N(C₁-C₆alkyl)₂, O—C₁-C₆ alkyl; and
wherein each R¹⁵ is independently selected from the group consisting of H, C₁-C₆ alkyl, C₃-C₇ cycloalkyl, C₄-C₇ heterocycloalkyl, C₆-C₁₀ aryl, C₅-C₁₀ membered heteroaryl; said alkyl, cycloalkyl, heterocycloalkyl, aryl, and heteroaryl moieties of the foregoing groups are optionally substituted by 1 to 3 substituents independently selected from the group consisting of H, halo, C₁-C₆ alkyl, CN, NH₂, NH C₁-C₆alkyl, N(C₁-C₆alkyl)₂, O—C₁-C₆ alkyl.

2. A compound according to claim 1, wherein E and G are independently selected from the group consisting of N and C;
wherein X, W and Q are independently selected from the group consisting of N, O, CO, NR³, CR² and CR²R³; and
wherein Y and Z are independently present or absent, if present Y and Z are selected from the group consisting of N, O, CO, NR³, CR² and CR²R³.

3. A compound according to claim 2, wherein E and G are independently selected from the group consisting of N and C;
wherein X, W and Q are independently selected from the group consisting of N, CO, NR³, CR² and CR²R²R³; and
wherein Y and Z are independently present or absent, if present Y and Z are selected from the group consisting of N, CO, NR³, CR³ and CR²R².

4. A compound according to claim 3, wherein E and G are C;
wherein X, W and Q are independently selected from the group consisting of N, CO, NR³, CR² and CR²R³; and
wherein Y and Z are independently present or absent, if present Y and Z are selected from the group consisting of N, CO, NR³, CR² and CR²R³.

5. A compound according to claim 4, wherein E and G are C;
wherein X, W and Q are independently selected from the group consisting of N, NR³, CR² and CR²R³; and
wherein Y and Z are independently present or absent, if present Y and Z are selected from the group consisting of N, NR³, CR² and CR²R³.

6. A compound according to claim 5, wherein R² is selected from the group consisting of:

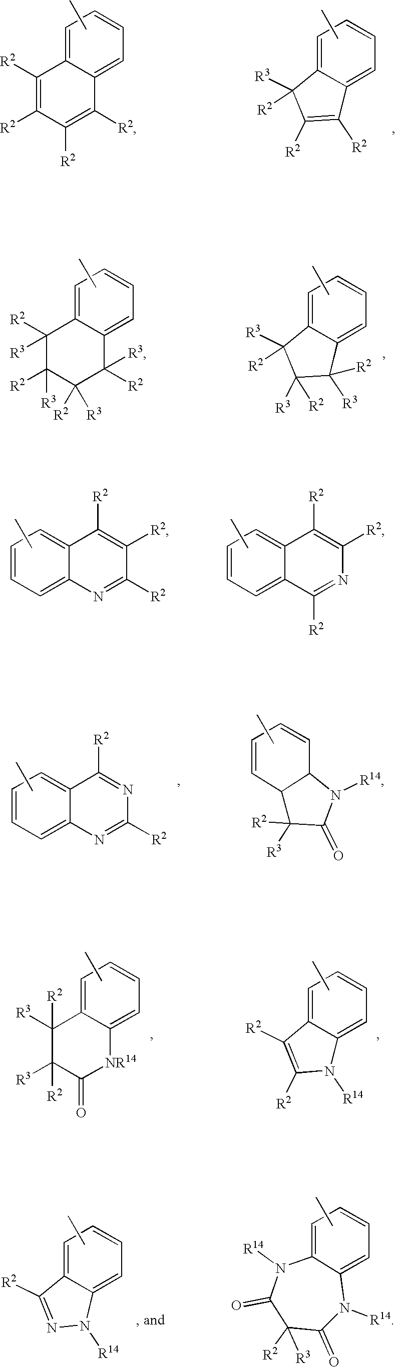

7. A compound according to claim 6, wherein R² is selected from the group consisting of:

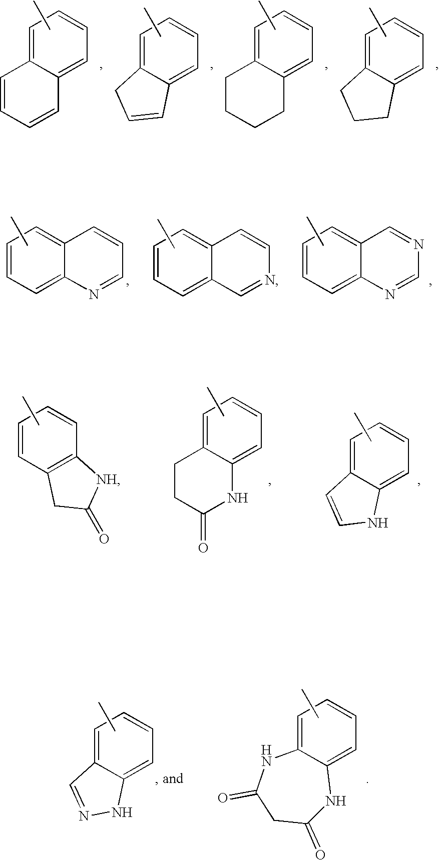

9. A compound according to claim 5, wherein R² is selected from the group consisting of:

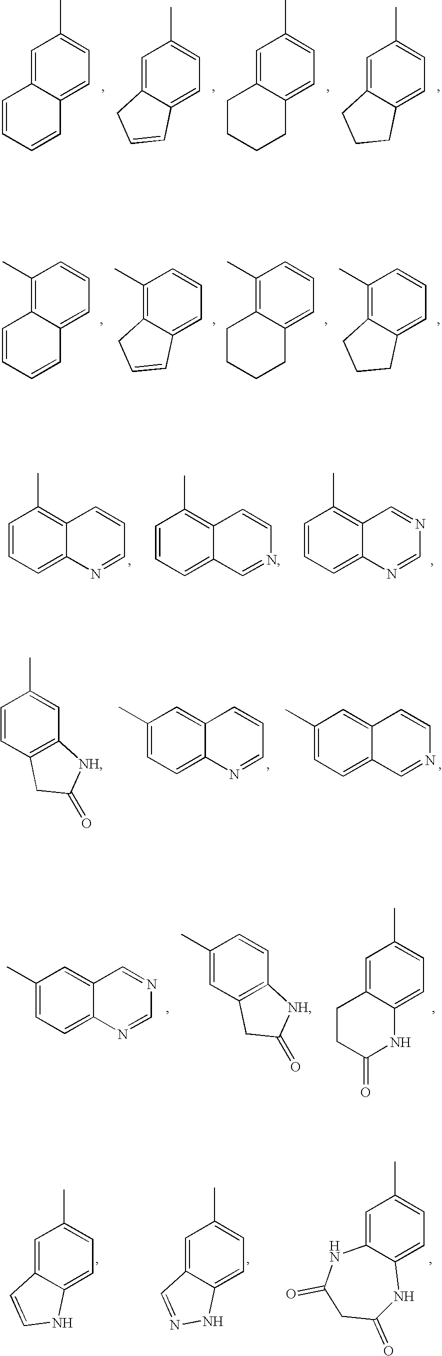

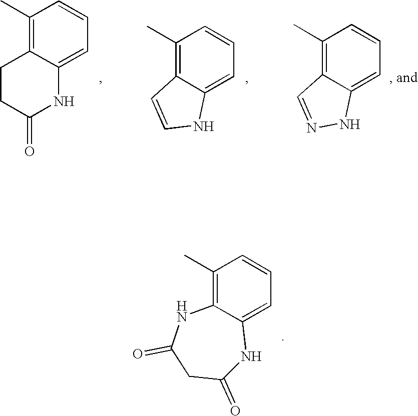

10. A compound according to claim 6, wherein R² is selected from the group consisting of:

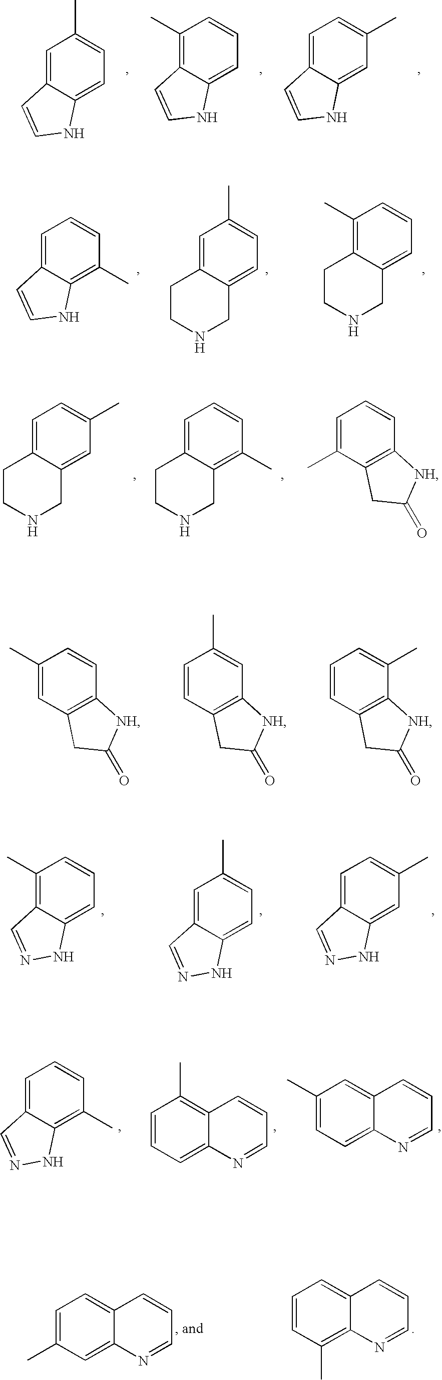

11. The compound according to claim 1, wherein wherein A is present or absent, if present A is selected from the group consisting of O and NH and wherein B is present or absent, if present B is selected from the group consisting of CO, SO₂, and NR⁶, with the proviso that when A is 0 that B is absent.

12. The compound according to claim 11, wherein wherein A is present or absent, if present A is NH and wherein B is present or absent, if present B is selected from the group consisting of CO, SO₂, and NR⁶.

13. The compound according to claim 12, wherein wherein A is present or absent, if present A is NH and wherein B is present or absent, if present B is selected from the group consisting of CO and NR⁶.

14. The compound according to claim 13, wherein wherein A is present or absent, if present A is NH and wherein B is present or absent, if present B is CO.

15. The compound according to claim 14, wherein wherein A is present or absent, if present A is NH and wherein B is absent.

16. The compound according to claim 15, wherein wherein A is NH and wherein B is absent.

17. The compound according to claims 1, and 11-16 wherein each R² is independently selected from the group consisting of H, C₁-C₆ alkyl, C₃-C₇ cycloalkyl, C₄-C₇ heterocycloalkyl, OC₁-C₆ alkyl, OC₃-C₇ cycloalkyl, OC₄-C₇ heterocyloalkyl, NH₂, NHR⁶, NR⁶R⁷, SR⁶, SOR⁶, SO₂R⁶, CO₂R⁶, CONH₂, CONHR⁶, CONR⁶R⁷, NHCOR⁶, NR⁶CONR⁶, NHCONHR⁶, NR⁶CONHR⁶, NHCONR⁶R⁷, NR⁶CONR⁶R⁷, NHSO₂R⁶, NR⁶SO₂R⁶, with the proviso that O, N or S atom of the foregoing substituents may not be bound to a carbon atom bound to another heteroatom, said alkyl, cycloalkyl, heterocycloalkyl moieties of the foregoing groups are optionally substituted by 1 to 3 substituents independently selected from the group consisting of H, halo, C₁-C₆ alkyl, CN, NH₂, NHR¹⁰, N(R¹⁰)₂, OR¹⁰, C₁-C₆ alkyl, C₃-C₇ cycloalkyl, C₄-C₇ heterocycloalkyl, CO₂R″, CONH₂, CONHR″, and CONR¹¹R¹²; and
wherein each R³ is independently selected from the group consisting of H, C₁-C₆ alkyl, C₃-C₇ cycloalkyl, C₄-C₇ heterocycloalkyl, CO₂R⁶, CONH₂, CONHR⁶, CONR⁶R⁷ or R² and R³ taken together with the carbon atom they are linked to can form a 3-7 membered cycloalkyl ring or 4-7 membered heterocycloalkyl ring, wherein each methylene group present in said 3-7 membered cycloalkyl ring and said 4-7 membered heterocycloalkyl ring may be optionally replaced by a C═O group, said alkyl, cycloalkyl, heterocycloalkyl moieties of the foregoing groups are optionally substituted by 1 to 3 substituents independently selected from the group consisting of H, halo, C₁-C₆ alkyl, CN, NH₂, NHR¹⁰, N(R¹⁰)₂, OR¹⁰, C₁-C₆ alkyl, C₃-C₇ cycloalkyl, C₄-C₇ heterocycloalkyl, CO₂R¹¹, CONH₂, CONHR¹¹, and CONR¹¹R¹².

18. The compound according to claim 17 wherein each R² is independently selected from the group consisting of H, C₁-C₆ alkyl, C₃-C₇ cycloalkyl, C₄-C₇ heterocycloalkyl, OC₁-C₆ alkyl, OC₃-C₇ cycloalkyl, OC₄-C₇ heterocyloalkyl, NH₂, NHR⁶, NR⁶R⁷, with the proviso that O, N or S atom of the foregoing substituents may not be bound to a carbon atom bound to another heteroatom, said alkyl, cycloalkyl, heterocycloalkyl moieties of the foregoing groups are optionally substituted by 1 to 3 substituents independently selected from the group consisting of H, halo, C₁-C₆ alkyl, CN, NH₂, NHR¹⁰, N(R¹⁰)₂, OR¹⁰, C₁-C₆ alkyl, C₃-C₇ cycloalkyl, C₄-C₇ heterocycloalkyl, CO₂R¹¹, CONH₂, CONHR¹¹, and CONR¹¹R¹²; and
wherein each R³ is independently selected from the group consisting of H, C₁-C₆ alkyl, C₃-C₇ cycloalkyl, C₄-C₇ heterocycloalkyl, CO₂R⁶, CONH₂, CONHR⁶, CONR⁶R⁷ or R² and R³ taken together with the carbon atom they are linked to can form a 3-7 membered cycloalkyl ring or 4-7 membered heterocycloalkyl ring, wherein each methylene group present in said 3-7 membered cycloalkyl ring and said 4-7 membered heterocycloalkyl ring may be optionally replaced by a C═O group, said alkyl, cycloalkyl, heterocycloalkyl moieties of the foregoing groups are optionally substituted by 1 to 3 substituents independently selected from the group consisting of H, halo, C₁-C₆ alkyl, CN, NH₂, NHR¹⁰, N(R¹⁰)₂, OR¹⁰, C₁-C₆ alkyl, C₃-C₇ cycloalkyl, C₄-C₇ heterocycloalkyl, CO₂R¹¹, CONH₂, CONHR¹¹, and CONR¹¹R¹².

19. The compound according to claim 1. wherein R⁴ is selected from the group consisting of H, C₁-C₆ alkyl, C₆-C₁₀ aryl, and 5-10 membered heteroaryl, the alkyl, aryl and heteroaryl moieties of the foregoing groups are optionally substituted by 1 to 3 subsitutents independently selected from the group consisting of H, halo, OH, NO₂, C₁-C₆ alkyl, C(R⁶)=CR⁶R⁷, C═CR⁶, C₃-C₇ cycloalkyl, C₄-C₇ heterocycloalkyl, OC₁-C₆ alkyl, OC₃-C₇ cycloalkyl, OC₄-C₇ heterocyloalkyl, C═N—OH, C═N—O(C₁-C₆ alkyl), NH₂, NHR⁶, NR⁶R⁷, SR⁶, SOR⁶, SO₂R⁶, CO₂R⁶, CONH₂, CONHR⁶, CONR⁶R⁷, SO₂NH₂, SO₂NHR⁶, SO₂NR⁶R⁷, NHCOR⁶, NR⁶CONR⁶, NHCONHR⁶, NR⁶CONHR⁶, NHCONR⁶R⁷, NR⁶CONR⁶R⁶, NHSO₂R⁶, NR⁶SO₂R⁶, with the proviso that O, N or S atom of the foregoing substituents may not be bound to a carbon atom bound to another heteroatom.

20. The compound according to claim 19, wherein R⁴ is selected from the group consisting of H, C₁-C₆ alkyl, and C₆-C₁₀ aryl, wherein the alkyl, and aryl moieties of the foregoing groups are optionally substituted by 1 to 3 subsitutents independently selected from the group consisting of H, halo, OH, NO₂, C₁-C₆ alkyl, C(R⁶)═CR⁶R⁷, C≡CR⁶, C₃-C₇ cycloalkyl, C₄-C₇ heterocycloalkyl, OC₁-C₆ alkyl, OC₃-C₇ cycloalkyl, OC₄-C₇ heterocyloalkyl, C═N—OH, C═N—O(C₁-C₆ alkyl), NH₂, NHR⁶, NR⁶R⁷, SR⁶, SOR⁶, SO₂R⁶, CO₂R⁶, CONH₂, CONHR⁶, CONR⁶R⁷, SO₂NH₂, SO₂NHR⁶, SO₂NR⁶R⁷, NHCOR⁶, NR⁶CONR⁶, NHCONHR⁶, NR⁶CONHR⁶, NHCONR⁶R⁷, NR⁶CONR⁶R⁷, NHSO₂R⁶, NR⁶SO₂R⁶, with the proviso that O, N or S atom of the foregoing substituents may not be bound to a carbon atom bound to another heteroatom.

21. The compound according to claim 1, wherein R⁵ is selected from the group consisting of H, Br, Cl, CN, CF₃, CH₂F, CHF₂, SO₂CH₃, CONH₂, C₆H₅, CONHR⁶, CONR⁶R⁷, CO₂R⁶, C(R⁹)═C(R⁹)₂, and C═CR⁹.

22. The compound according to claim 21, wherein R⁵ is selected from the group consisting of H, Br, Cl, CN, CF₃, CH₂F, CHF₂, SO₂CH₃, CONH₂, and C₆H₅.

23. The compound according to claim 22, wherein R⁵ is selected from the group consisting of H, Br, Cl, CN, CF₃, CH₂F, CHF₂, SO₂CH₃, and CONH₂.

24. A compound according to claim 1 selected from the group consisting of:
5-Bromo-N²-[3-(1,2,3,6-tetrahydro-pyridin-4-yl)-1H-indol-5-yl]-N⁴-p-tolyl-pyrimidine-2,4-diamine;
5-Bromo-N⁴-pyridin-2-yl-N2-[3-(1,2,3,6-tetrahydro-pyridin-4-yl)-1H-indol-5-yl]-pyrimidine-2,4-diamine;
5-Bromo-N⁴-pyridin-2-ylmethyl-N²-[3-(1,2,3,6-tetrahydro-pyridin-4-yl)-1H-indol-5-yl]-pyrimidine-2,4-diamine;
N⁴-Benzyl-5-bromo-N²-[3-(1,2,3,6-tetrahydro-pyridin-4-yl)-1H-indol-5-yl]-pyrimidine-2,4-diamine;
5-Bromo-N4-(1R-phenyl-ethyl)-N2-[3-(1,2,3,6-tetrahydro-pyridin-4-yl)-1H-indol-5-yl]-pyrimidine-2,4-diamine;
5-Bromo-N⁴-(1rac-phenyl-ethyl)-N²-[3-(1,2,3,6-tetrahydro-pyridin-4-yl)-1H-indol-5-yl]-pyrimidine-2,4-diamine;
5-Bromo-N4-(1S-phenyl-ethyl)-N2-[3-(1,2,3,6-tetrahydro-pyridin-4-yl)-1H-indol-5-yl]-pyrimidine-2,4-diamine;
4-({5-Bromo-2-[3-(1,2,3,6-tetrahydro-pyridin-4-yl)-1H-indol-5-ylamino]-pyrimidin-4-ylamino}-methyl)-benzenesulfonamide
5-Bromo-N²-[3-(1,2,3,6-tetrahydro-pyridin-4-yl)-1H-indol-5-yl]-N⁴-(4-trifluoromethyl-benzyl)-pyrimidine-2,4-diamine;
5-Bromo-N⁴-(4-methoxy-benzyl)-N²-[3-(1,2,3,6-tetrahydro-pyridin-4-yl)-1H-indol-5-yl]-pyrimidine-2,4-diamine;
5-Bromo-N⁴-(4-fluoro-benzyl)-N²-[3-(1,2,3,6-tetrahydro-pyridin-4-yl)-1H-indol-5-yl]-pyrimidine-2,4-diamine;
5-Bromo-N⁴-(3-fluoro-benzyl)-N²-[3-(1,2,3,6-tetrahydro-pyridin-4-yl)-1H-indol-5-yl]-pyrimidine-2,4-diamine;
5-Bromo-N⁴-naphthalen-1-ylmethyl-N²-[3-(1,2,3,6-tetrahydro-pyridin-4-yl)-1H-indol-5-yl]-pyrimidine-2,4-diamine;
5-Bromo-N⁴-(4-fluoro-3-trifluoromethyl-benzyl)-N²-[3-(1,2,3,6-tetrahydro-pyridin-4-yl)-1H-indol-5-yl]-pyrimidine-2,4-diamine;
5-Bromo-N⁴-(3-fluoro-5-trifluoromethyl-benzyl)-N²-[3-(1,2,3,6-tetrahydro-pyridin-4-yl)-1H-indol-5-yl]-pyrimidine-2,4-diamine;
5-Bromo-N⁴-(4-phenoxy-benzyl)-N²-[3-(1,2,3,6-tetrahydro-pyridin-4-yl)-1H-indol-5-yl]-pyrimidine-2,4-diamine;
5-Bromo-N⁴-(3,4-difluoro-benzyl)-N²-[3-(1,2,3,6-tetrahydro-pyridin-4-yl)-1H-indol-5-yl]-pyrimidine-2,4-diamine;
5-Bromo-N²-[3-(1,2,3,6-tetrahydro-pyridin-4-yl)-1H-indol-5-y]-N⁴-(3-trifluoromethoxy-benzyl)-pyrimidine-2,4-diamine;
5-Bromo-N⁴-(4-chloro-benzyl)-N²-[3-(1,2,3,6-tetrahydro-pyridin-4-yl)-1H-indol-5-yl]-pyrimidine-2,4-diamine;
5-Bromo-N²-[3-(1,2,3,6-tetrahydro-pyridin-4-yl)-1H-indol-5-yl]-N⁴-thiophen-2-ylmethyl-pyrimidine-2,4-diamine;
5-Bromo-N4-furan-2-ylmethyl-N2-[3-(1,2,3,6-tetrahydro-pyridin-4-yl)-1H-indol-5-yl]-pyrimidine-2,4-diamine;
5-Bromo-N⁴-(2-methyl-benzyl)-N²-[3-(1,2,3,6-tetrahydro-pyridin-4-yl)-1H-indol-5-yl]-pyrimidine-2,4-diamine;
5-Bromo-N⁴-(3-methyl-benzyl)-N²-[3-(1,2,3,6-tetrahydro-pyridin-4-yl)-1H-indol-5-yl]-pyrimidine-2,4-diamine;
5-Bromo-N⁴-(4-methyl-benzyl)-N²-[3-(1,2,3,6-tetrahydro-pyridin-4-yl)-1H-indol-5-yl]-pyrimidine-2,4-diamine;
5-Bromo-N⁴-(2-fluoro-benzyl)-N²-[3-(1,2,3,6-tetrahydro-pyridin-4-yl)-1H-indol-5-yl]-pyrimidine-2,4-diamine;
N⁴-Biphenyl-2-ylmethyl-5-bromo-N²-[3-(1,2,3,6-tetrahydro-pyridin-4-yl)-1H-indol-5-yl]-pyrimidine-2,4-diamine;
N⁴-Biphenyl-3-ylmethyl-5-bromo-N²-[3-(1,2,3,6-tetrahydro-pyridin-4-yl)-1H-indol-5-yl]-pyrimidine-2,4-diamine;
5-Bromo-N⁴-(2-methoxy-benzyl)-N²-[3-(1,2,3,6-tetrahydro-pyridin-4-yl)-1H-indol-5-yl]-pyrimidine-2,4-diamine;
5-Bromo-N⁴-(3-methoxy-benzyl)-N²-[3-(1,2,3,6-tetrahydro-pyridin-4-yl)-1H-indol-5-yl]-pyrimidine-2,4-diamine;
3-({5-Bromo-2-[3-(1,2,3,6-tetrahydro-pyridin-4-yl)-1H-indol-5-ylamino]-pyrimidin-4-ylamino}-methyl)-N-methyl-benzamide
5-Bromo-N⁴-(2-chloro-benzyl)-N²-[3-(1,2,3,6-tetrahydro-pyridin-4-yl)-1H-indol-5-yl]-pyrimidine-2,4-diamine;
5-Bromo-N⁴-phenethyl-N²-[3-(1,2,3,6-tetrahydro-pyridin-4-yl)-1H-indol-5-yl]-pyrimidine-2,4-diamine;
5-Bromo-N⁴-(2-pyridin-2-yl-ethyl)-N²-[3-(1,2,3,6-tetrahydro-pyridin-4-yl)-1H-indol-5-yl]-pyrimidine-2,4-diamine;
5-Bromo-N⁴-(2-pyridin-4-yl-ethyl)-N²-[3-(1,2,3,6-tetrahydro-pyridin-4-yl)-1H-indol-5-yl]-pyrimidine-2,4-diamine;
5-Bromo-N⁴-(2-pyridin-3-yl-ethyl)-N²-[3-(1,2,3,6-tetrahydro-pyridin-4-yl)-1H-indol-5-yl]-pyrimidine-2,4-diamine;
5-Bromo-N⁴-[2-(3-fluoro-phenyl)-ethyl]-N²-[3-(1,2,3,6-tetrahydro-pyridin-4-yl)-1H-indol-5-yl]-pyrimidine-2,4-diamine;
5-Bromo-N⁴-(2-phenyl-cyclopropyl)-N²-[3-(1,2,3,6-tetrahydro-pyridin-4-yl)-1H-indol-5-yl]-pyrimidine-2,4-diamine;
5-Bromo-N⁴-(2-phenyl-cyclopropyl)-N²-[3-(1,2,3,6-tetrahydro-pyridin-4-yl)-1H-indol-5-yl]-pyrimidine-2,4-diamine; (homo-chiral)
5-Bromo-N⁴-(2-phenyl-cyclopropyl)-N²-[3-(1,2,3,6-tetrahydro-pyridin-4-yl)-1H-indol-5-yl]-pyrimidine-2,4-diamine; (homo-chiral)
5-Bromo-N⁴-[2-(4-chloro-phenyl)-ethyl]-N²-[3-(1,2,3,6-tetrahydro-pyridin-4-yl)-1H-indol-5-yl]-pyrimidine-2,4-diamine;
5-Bromo-N²-[3-(1,2,3,6-tetrahydro-pyridin-4-yl)-1H-indol-5-yl]-N⁴-(2-thiophen-2-yl-ethyl)-pyrimidine-2,4-diamine;
5-Bromo-N⁴-[2-(2-fluoro-phenyl)-ethyl]-N²-[3-(1,2,3,6-tetrahydro-pyridin-4-yl)-1H-indol-5-yl]-pyrimidine-2,4-diamine;
5-Bromo-N⁴-[2-(2-chloro-phenyl)-ethyl]-N²-[3-(1,2,3,6-tetrahydro-pyridin-4-yl)-1H-indol-5-yl]-pyrimidine-2,4-diamine;
5-Bromo-N⁴-[2-(2-methoxy-phenyl)-ethyl]-N²-[3-(1,2,3,6-tetrahydro-pyridin-4-yl)-1H-indol-5-yl]-pyrimidine-2,4-diamine;
N⁴-(2-Benzo[1,3]dioxol-5-yl-ethyl)-5-bromo-N²-[3-(1,2,3,6-tetrahydro-pyridin-4-yl)-1H-indol-5-yl]-pyrimidine-2,4-diamine;
5-Bromo-N⁴-(3-phenyl-propyl)-N²-[3-(1,2,3,6-tetrahydro-pyridin-4-yl)-1H-indol-5-yl]-pyrimidine-2,4-diamine;
5-(5-Bromo-4-phenethylamino-pyrimidin-2-ylamino)-1,3-dihydro-indol-2-one; 5-[5-Bromo-4-(2-chloro-benzylamino)-pyrimidin-2-ylamino]-1,3-dihydro-indol-2-one;
5-(4-Benzylamino-5-bromo-pyrimidin-2-ylamino)-1,3-dihydro-indol-2-one;
5-[5-Bromo-4-(1-phenyl-ethylamino)-pyrimidin-2-ylamino]-1,3-dihydro-indol-2-one;
5-[5-Bromo-4-(3-phenyl-propylamino)-pyrimidin-2-ylamino]-1,3-dihydro-indol-2-one;
5-Bromo-N⁴-(2-methanesulfonyl-ethyl)-N²-[3-(1,2,3,6-tetrahydro-pyridin-4-yl)-1H-indol-5-yl]-pyrimidine-2,4-diamine;
N⁴-Benzyl-N²-[3-(1,2,3,6-tetrahydro-pyridin-4-yl)-1H-indol-5-yl]-pyrimidine-2,4-diamine;
N⁴-Benzyl-N⁴-methyl-N²-[3-(1,2,3,6-tetrahydro-pyridin-4-yl)-1H-indol-5-yl]-pyrimidine-2,4-diamine;
N⁴-Methyl-N⁴-(2-pyridin-2-yl-ethyl)-N²-[3-(1,2,3,6-tetrahydro-pyridin-4-yl)-1H-indol-5-yl]-pyrimidine-2,4-diamine;
[4-(2-Phenyl-morpholin-4-yl)-pyrimidin-2-yl]-[3-(1,2,3,6-tetrahydro-pyridin-4-yl)-1H-indol-5-yl]-amine
5-Methyl-N⁴-(2-pyridin-2-yl-ethyl)-N²-[3-(1,2,3,6-tetrahydro-pyridin-4-yl)-1H-indol-5-yl]-pyrimidine-2,4-diamine;
5-Bromo-N²-(3-piperidin-4-yl-1H-indol-5-yl)-N⁴-(2-pyridin-2-yl-ethyl)-pyrimidine-2,4-diamine;
5-Bromo-N 2-[1-methanesulfonyl-3-(1,2,3,6-tetrahydro-pyridin-4-yl)-1H-indol-5-yl]-N⁴-(2-pyridin-2-yl-ethyl)-pyrimidine-2,4-diamine;
5-Bromo-N 2-[1-methanesulfonyl-3-(1,2,3,6-tetrahydro-pyridin-4-yl)-1H-indol-5-yl]-N⁴-pyridin-2-yl-pyrimidine-2,4-diamine;
5-Bromo-N¹-(2-pyridin-2-yl-ethyl)-N⁴-[3-(1,2,3,6-tetrahydro-pyridin-4-yl)-1H-indol-5-yl]-pyrimidine-2,4-diamine;
3-{4-(2-Pyridin-2-yl-ethylamino)-2-[3-(1,2,3,6-tetrahydro-pyridin-4-yl)-1H-indol-5-ylamino]-pyrimidin-5-yl}-acrylic acid; ethyl ester;
5-{5-Bromo-4-[2-(3-chloro-phenyl)-ethylamino]-pyrimidin-2-ylamino}-1,3-dihydro-indol-2-one;
5-Bromo-N⁴-[2-(3-chloro-phenyl)-ethyl]-N²-[3-(1,2,3,6-tetrahydro-pyridin-4-yl)-1H-indol-5-yl]-pyrimidine-2,4-diamine;
5-Bromo-N⁴-[2-(3-chloro-phenyl)-ethyl]-N²-[3-(1,2,3,6-tetrahydro-pyridin-4-yl)-1H-indol-5-yl]-pyrimidine-2,4-diamine;
5-{5-Bromo-4-[2-(4-methoxy-phenyl)-ethylamino]-pyrimidin-2-ylamino}-1,3-dihydro-indol-2-one;
5-Bromo-N⁴-[2-(4-methoxy-phenyl)-ethyl]-N²-[3-(1,2,3,6-tetrahydro-pyridin-4-yl)-1H-indol-5-yl]-pyrimidine-2,4-diamine;
5-{5-Bromo-4-[2-(3-methoxy-phenyl)-ethylamino]-pyrimidin-2-ylamino}-1,3-dihydro-indol-2-one;
5-Bromo-N⁴-[2-(3-methoxy-phenyl)-ethyl]-N²-[3-(1,2,3,6-tetrahydro-pyridin-4-yl)-1H-indol-5-yl]-pyrimidine-2,4-diamine;
5-[5-Bromo-4-(2-o-tolyl-ethylamino)-pyrimidin-2-ylamino]-1,3-dihydro-indol-2-one;
5-Bromo-N²-[3-(1,2,3,6-tetrahydro-pyridin-4-yl)-1H-indol-5-yl]-N⁴-(2-o-tolyl-ethyl)-pyrimidine-2,4-diamine;
5-[5-Bromo-4-(2-m-tolyl-ethylamino)-pyrimidin-2-ylamino]-1,3-dihydro-indol-2-one;
5-Bromo-N 2-[3-(1,2,3,6-tetrahydro-pyridin-4-yl)-1H-indol-5-yl]-N⁴-(2-m-tolyl-ethyl)-pyrimidine-2,4-diamine;
5-[5-Bromo-4-(2-p-tolyl-ethylamino)-pyrimidin-2-ylamino]-1,3-dihydro-indol-2-one;
5-Bromo-N²-[3-(1,2,3,6-tetrahydro-pyridin-4-yl)-1H-indol-5-yl]-N⁴-(2-p-tolyl-ethyl)-pyrimidine-2,4-diamine;
[5-Bromo-2-(2-oxo-2,3-dihydro-1H-indol-5-ylamino)-pyrimidin-4-ylamino]-acetic acid;
5-{5-Bromo-4-[2-(3-trifluoromethyl-phenyl)-ethylamino]-pyrimidin-2-ylamino}-1,3-dihydro-indol-2-one;
5-[4-(2-Biphenyl-4-yl-ethylamino)-5-bromo-pyrimidin-2-ylamino]-1,3-dihydro-indol-2-one;
5-{5-Bromo-4-[2-(3-fluoro-phenyl)-ethylamino]-pyrimidin-2-ylamino}-1,3-dihydro-indol-2-one;
5-{5-Bromo-4-[2-(2-chloro-phenyl)-ethylamino]-pyrimidin-2-ylamino}-1,3-dihydro-indol-2-one;
5-{5-Bromo-4-[2-(2-methoxy-phenyl)-ethylamino]-pyrimidin-2-ylamino}-1,3-dihydro-indol-2-one;
5-{5-Bromo-4-[2-(4-fluoro-phenyl)-ethylamino]-pyrimidin-2-ylamino}-1,3-dihydro-indol-2-one;
5-{5-Bromo-4-[2-(4-chloro-phenyl)-ethylamino]-pyrimidin-2-ylamino}-1,3-dihydro-indol-2-one;
5-{5-Bromo-4-[2-(2-fluoro-phenyl)-ethylamino]-pyrimidin-2-ylamino}-1,3-dihydro-indol-2-one;
5-[5-Bromo-4-(3-phenyl-allylamino)-pyrimidin-2-ylamino]-1,3-dihydro-indol-2-one;
5-{5-Bromo-4-[(thiophen-2-ylmethyl)-amino]-pyrimidin-2-ylamino}-1,3-dihydro-indol-2-one;
6-{5-Bromo-4-[(thiophen-2-ylmethyl)-amino]-pyrimidin-2-ylamino}-1,3-dihydro-indol-2-one;
5-[5-Bromo-4-(2,3-dimethyl-benzylamino)-pyrimidin-2-ylamino]-1,3-dihydro-indol-2-one;
6-[5-Bromo-4-(2,3-dimethyl-benzylamino)-pyrimidin-2-ylamino]-1,3-dihydro-indol-2-one;
5-[5-Bromo-4-(2,5-dimethyl-benzylamino)-pyrimidin-2-ylamino]-1,3-dihydro-indol-2-one;
6-[5-Bromo-4-(2,5-dimethyl-benzylamino)-pyrimidin-2-ylamino]-1,3-dihydro-indol-2-one;
6-[5-Bromo-4-(2-fluoro-benzylamino)-pyrimidin-2-ylamino]-1,3-dihydro-indol-2-one;
6-[5-Bromo-4-(2-trifluoromethoxy-benzylamino)-pyrimidin-2-ylamino]-1,3-dihydro-indol-2-one;
5-[5-Bromo-4-(3-trifluoromethoxy-benzylamino)-pyrimidin-2-ylamino]-1,3-dihydro-indol-2-one;
6-[5-Bromo-4-(3-trifluoromethoxy-benzylamino)-pyrimidin-2-ylamino]-1,3-dihydro-indol-2-one;
5-[5-Bromo-4-(4-trifluoromethoxy-benzylamino)-pyrimidin-2-ylamino]-1,3-dihydro-indol-2-one;
6-[5-Bromo-4-(4-trifluoromethoxy-benzylamino)-pyrimidin-2-ylamino]-1,3-dihydro-indol-2-one; 6-[5-Bromo-4-(2-methoxy-benzylamino)-pyrimidin-2-ylamino]-1,3-dihydro-indol-2-one; 6-[5-Bromo-4-(3-methoxy-benzylamino)-pyrimidin-2-ylamino]-1,3-dihydro-indol-2-one; 6-[5-Bromo-4-(3-trifluoromethyl-benzylamino)-pyrimidin-2-ylamino]-1,3-dihydro-indol-2-one;
5-{5-Bromo-4-[(thiazol-2-ylmethyl)-amino]-pyrimidin-2-ylamino}-1,3-dihydro-indol-2-one;
5-{5-Bromo-4-[(5-methanesulfonyl-thiophen-2-ylmethyl)-amino]-pyrimidin-2-ylamino}-1,3-dihydro-indol-2-one;
5-[5-Bromo-4-(2,3-difluoro-benzylamino)-pyrimidin-2-ylamino]-1,3-dihydro-indol-2-one;
6-[5-Bromo-4-(2,3-difluoro-benzylamino)-pyrimidin-2-ylamino]-1,3-dihydro-indol-2-one;
5-[5-Bromo-4-(2,4-difluoro-benzylamino)-pyrimidin-2-ylamino]-1,3-dihydro-indol-2-one;
6-[5-Bromo-4-(2,4-difluoro-benzylamino)-pyrimidin-2-ylamino]-1,3-dihydro-indol-2-one;
6-[5-Chloro-4-(2-trifluoromethyl-benzylamino)-pyrimidin-2-ylamino]-1,3-dihydro-indol-2-one;
5-Chloro-N2-(1-methyl-1H-indol-5-yl)-N4-(2-trifluoromethyl-benzyl)-pyrimidine-2,4-diamine;
5-Chloro-N²-(1H-indazol-5-yl)-N⁴-(2-trifluoromethyl-benzyl)-pyrimidine-2,4-diamine;
5-Chloro-N 2-(1-methyl-1H-indol-5-yl)-N⁴-pyridin-2-ylmethyl-pyrimidine-2,4-diamine;
6-{5-Chloro-4-[(pyridin-2-ylmethyl)-amino]-pyrimidin-2-ylamino}-1,3-dihydro-indol-2-one;
5-Chloro-N2-(1H-indazol-6-yl)-N4-(2-trifluoromethyl-benzyl)-pyrimidine-2,4-diamine;
5-Chloro-N2-(1H-indazol-6-yl)-N4-pyridin-2-ylmethyl-pyrimidine-2,4-diamine;
(5-{5-Bromo-4-[(pyridin-2-ylmethyl)-amino]-pyrimidin-2-ylamino}-indazol-1-yl)-acetic acid; tert-butyl ester;
(6-{5-Bromo-4-[(pyridin-2-ylmethyl)-amino]-pyrimidin-2-ylamino}-indazol-2-yl)-acetic acid; tert-butyl ester;
6-{4-[(Pyridin-2-ylmethyl)-amino]-5-trifluoromethyl-pyrimidin-2-ylamino}-1,3-dihydro-indol-2-one;
N2-(1-Methyl-1H-indol-5-yl)-N4-pyridin-2-ylmethyl-5-trifluoromethyl-pyrimidine-2,4-diamine;
(6-{5-Bromo-4-[(pyridin-2-ylmethyl)-amino]-pyrimidin-2-ylamino}-indol-1-yl)-acetic acid; tert-butyl ester;
N4-Pyridin-2-ylmethyl-N2-quinolin-5-yl-5-trifluoromethyl-pyrimidine-2,4-diamine; 2-(6-{5-Bromo-4-[(pyridin-2-ylmethyl)-amino]-pyrimidin-2-ylamino}-indol-1-yl)-N-(2-methoxy-ethyl)-acetamide;
6-{5-Chloro-4-[(3-methyl-pyridin-2-ylmethyl)-amino]-pyrimidin-2-ylamino}-1,3-dihydro-indol-2-one;
(6-{5-Bromo-4-[(pyridin-2-ylmethyl)-amino]-pyrimidin-2-ylamino}-indol-1-yl)-acetic acid;
(6-{5-Bromo-4-[(pyridin-2-ylmethyl)-amino]-pyrimidin-2-ylamino}-indazol-1-yl)-acetic acid; tert-butyl ester;
N2-(1H-Indazol-6-yl)-N-4-pyridin-2-ylmethyl-5-trifluoromethyl-pyrimidine-2,4-diamine;
(5-{5-Bromo-4-[(pyridin-2-ylmethyl)-amino]-pyrimidin-2-ylamino}-indol-1-yl)-acetic acid; tert-butyl ester;
(6-{5-Bromo-4-[(pyridin-2-ylmethyl)-amino]-pyrimidin-2-ylamino}-indazol-1-yl)-acetic acid;
(5-{5-Bromo-4-[(pyridin-2-ylmethyl)-amino]-pyrimidin-2-ylamino}-indol-1-yl)-acetic acid;
(5-{5-Bromo-4-[(pyridin-2-ylmethyl)-amino]-pyrimidin-2-ylamino}-indazol-1-yl)-acetic acid;
5-{5-Chloro-4-(3-methyl-pyridin-2-ylmethyl)-amino-pyrimidin-2-ylamino}-1,3-dihydro-indol-2-one;
5-[5-Chloro-4-(3-methanesulfonyl-benzylamino)-pyrimidin-2-ylamino]-1,3-dihydro-indol-2-one;
6-[5-Chloro-4-(3-methyl-benzylamino)-pyrimidin-2-ylamino]-1,3-dihydro-indol-2-one;
5-[5-Chloro-4-(2-fluoro-benzylamino)-pyrimidin-2-ylamino]-1,3-dihydro-indol-2-one;
6-[5-Chloro-4-(2-fluoro-benzylamino)-pyrimidin-2-ylamino]-1,3-dihydro-indol-2-one;
5-[5-Bromo-4-(2-methoxy-ethylamino)-pyrimidin-2-ylamino]-1,3-dihydro-indol-2-one;
5-[5-Chloro-4-(3-methyl-benzylamino)-pyrimidin-2-ylamino]-1,3-dihydro-indol-2-one;
6-{5-Chloro-4-[(4-methyl-pyridin-2-ylmethyl)-amino]-pyrimidin-2-ylamino}-1,3-dihydro-indol-2-one;
5-(4-Benzylamino-5-chloro-pyrimidin-2-ylamino)-1,3-dihydro-indol-2-one;
5-Bromo-N2-(1H-indol-5-yl)-N4-pyridin-2-ylmethyl-pyrimidine-2,4-diamine;
5-Bromo-N2-(1H-indol-5-yl)-N4-(2-pyridin-2-yl-ethyl)-pyrimidine-2,4-diamine;
5-Bromo-N2-(1H-indol-4-yl)-N4-(2-pyridin-2-yl-ethyl)-pyrimidine-2,4-diamine;
5-Bromo-N2-(1H-indazol-5-yl)-N4-(2-pyridin-2-yl-ethyl)-pyrimidine-2,4-diamine;
5-Bromo-N2-(1H-indazol-6-yl)-N4-(2-pyridin-2-yl-ethyl)-pyrimidine-2,4-diamine;
5-Bromo-N2-(1H-indol-4-yl)-N4-pyridin-2-ylmethyl-pyrimidine-2,4-diamine;
5-Bromo-N2-(1H-indazol-5-yl)-N4-pyridin-2-ylmethyl-pyrimidine-2,4-diamine;
N2-(1H-Indol-5-yl)-N4-pyridin-2-ylmethyl-pyrimidine-2,4-diamine;
N2-(1H-Indazol-6-yl)-N4-pyridin-2-ylmethyl-pyrimidine-2,4-diamine;
N2-(1H-Indol-5-yl)-N4-(2-pyridin-2-yl-ethyl)-pyrimidine-2,4-diamine;
N2-(1H-Indazol-5-yl)-N4-pyridin-2-ylmethyl-pyrimidine-2,4-diamine;
N2-(1H-Indazol-5-yl)-N4-(2-pyridin-2-yl-ethyl)-pyrimidine-2,4-diamine;
N2-(1 H-Indazol-6-yl)-N4-(2-pyridin-2-yl-ethyl)-pyrimidine-2,4-diamine;
5-{5-Bromo-4-[(pyridin-2-ylmethyl)-amino]-pyrimidin-2-ylamino}-1,3-dihydro-benzoimidazol-2-one;
5-[5-Bromo-4-(2-pyridin-2-yl-ethylamino)-pyrimidin-2-ylamino]-1,3-dihydro-benzoimidazol-2-one;
5-{4-[(Pyridin-2-ylmethyl)-amino]-pyrimidin-2-ylamino}-1,3-dihydro-benzoimidazol-2-one;
5-[4-(2-Pyridin-2-yl-ethylamino)-pyrimidin-2-ylamino]-1,3-dihydro-benzoimidazol-2-one;
5-Bromo-N2-(1H-indazol-6-yl)-N4-pyridin-2-ylmethyl-pyrimidine-2,4-diamine;
5-{5-Bromo-4-[(pyridin-2-ylmethyl)-amino]-pyrimidin-2-ylamino}-1,3-dihydro-indol-2-one;
5-[5-Bromo-4-(2-pyridin-2-yl-ethylamino)-pyrimidin-2-ylamino]-1,3-dihydro-indol-2-one;
5-[4-(2-Pyridin-2-yl-ethylamino)-pyrimidin-2-ylamino]-1,3-dihydro-indol-2-one;
5-Bromo-N2-(2-methyl-1H-indol-5-yl)-N4-(2-pyridin-2-yl-ethyl)-pyrimidine-2,4-diamine;
N2-(2-Methyl-1H-indol-5-yl)-N4-pyridin-2-ylmethyl-pyrimidine-2,4-diamine;
N2-(1H-Indol-6-yl)-N4-pyridin-2-ylmethyl-pyrimidine-2,4-diamine;
5-Bromo-N2-(2-methyl-1H-indol-5-yl)-N4-pyridin-2-ylmethyl-pyrimidine-2,4-diamine;
5-Bromo-N2-(1H-indol-6-yl)-N4-pyridin-2-ylmethyl-pyrimidine-2,4-diamine;
5-Bromo-N2-(1H-indol-6-yl)-N4-(2-pyridin-2-yl-ethyl)-pyrimidine-2,4-diamine;
N2-(1H-Benzoimidazol-5-yl)-5-bromo-N4-pyridin-2-ylmethyl-pyrimidine-2,4-diamine;
N2-(1H-Benzoimidazol-5-yl)-5-bromo-N4-(2-pyridin-2-yl-ethyl)-pyrimidine-2,4-diamine;
3-[5-Bromo-4-(2-pyridin-2-yl-ethylamino)-pyrimidin-2-yl]-3H-benzoimidazol-5-ylamine
N2-(1H-Benzoimidazol-5-yl)-N4-pyridin-2-ylmethyl-pyrimidine-2,4-diamine;
5-Bromo-N2-(2-methyl-1H-benzoimidazol-5-yl)-N4-pyridin-2-ylmethyl-pyrimidine-2,4-diamine;
N2-(2-Methyl-1H-benzoimidazol-5-yl)-N4-pyridin-2-ylmethyl-pyrimidine-2,4-diamine;
5-Bromo-N2-(2-methyl-1H-benzoimidazol-5-yl)-N4-(2-pyridin-2-yl-ethyl)-pyrimidine-2,4-diamine;
5-Bromo-N2-(2,3-dihydro-1H-indol-5-yl)-N4-(2-pyridin-2-yl-ethyl)-pyrimidine-2,4-diamine;
N2-(2,3-Dihydro-1H-indol-5-yl)-N4-pyridin-2-ylmethyl-pyrimidine-2,4-diamine;
5-Bromo-N2-(1-methyl-1H-indol-5-yl)-N4-(2-pyridin-2-yl-ethyl)-pyrimidine-2,4-diamine;
N2-(1-Methyl-1H-indol-5-yl)-N4-pyridin-2-ylmethyl-pyrimidine-2,4-diamine;
5-Bromo-N2-(2,3-dihydro-1H-indol-5-yl)-N4-pyridin-2-ylmethyl-pyrimidine-2,4-diamine;
5-Bromo-N2-(1-methyl-1H-indol-5-yl)-N4-pyridin-2-ylmethyl-pyrimidine-2,4-diamine;
5-Fluoro-N4-pyridin-2-ylmethyl-N2-quinolin-6-yl-pyrimidine-2,4-diamine;
5-Bromo-N4-pyridin-2-ylmethyl-N2-quinolin-6-yl-pyrimidine-2,4-diamine;
5-Bromo-N2-(1H-indol-7-yl)-N4-(2-pyridin-2-yl-ethyl)-pyrimidine-2,4-diamine;
5-Bromo-N2-(1H-indol-7-yl)-N4-pyridin-2-ylmethyl-pyrimidine-2,4-diamine;
5-Bromo-N2-(1H-indazol-4-yl)-N4-pyridin-2-ylmethyl-pyrimidine-2,4-diamine;
6-{5-Bromo-4-[(pyridin-2-ylmethyl)-amino]-pyrimidin-2-ylamino}-1,3-dihydro-indol-2-one;
5-Bromo-N2-(1H-indazol-4-yl)-N4-(2-pyridin-2-yl-ethyl)-pyrimidine-2,4-diamine;
5-Bromo-N4-(2-pyridin-2-yl-ethyl)-N2-quinolin-6-yl-pyrimidine-2,4-diamine;
5-Bromo-N4-pyridin-2-ylmethyl-N2-quinolin-5-yl-pyrimidine-2,4-diamine;
5-Bromo-N4-(2-pyridin-2-yl-ethyl)-N2-quinolin-5-yl-pyrimidine-2,4-diamine;
6-[5-Bromo-4-(2-pyridin-2-yl-ethylamino)-pyrimidin-2-ylamino]-1,3-dihydro-indol-2-one;
5-Bromo-N4-pyridin-2-ylmethyl-N2-quinolin-8-yl-pyrimidine-2,4-diamine;
5-Bromo-N4-(2-pyridin-2-yl-ethyl)-N-2-quinolin-8-yl-pyrimidine-2,4-diamine;
5-{5-Bromo-4-[(pyridin-2-ylmethyl)-amino]-pyrimidin-2-ylamino}-1H-indole-2-carboxylic acid; ethyl ester;
6-[5-Bromo-4-(2-trifluoromethyl-benzylamino)-pyrimidin-2-ylamino]-1,3-dihydro-indol-2-one;
5-Bromo-N2-(1H-indazol-5-yl)-N4-(2-trifluoromethyl-benzyl)-pyrimidine-2,4-diamine;
5-Bromo-N2-(1H-indazol-6-yl)-N4-(2-trifluoromethyl-benzyl)-pyrimidine-2,4-diamine;
5-Bromo-N2-(1-methyl-1H-indol-5-yl)-N4-(2-trifluoromethyl-benzyl)-pyrimidine-2,4-diamine;
5-Bromo-N2-(1H-indazol-7-yl)-N4-pyridin-2-ylmethyl-pyrimidine-2,4-diamine;
5-Bromo-N2-(1H-indazol-4-yl)-N4-(2-trifluoromethyl-benzyl)-pyrimidine-2,4-diamine;
6-{5-Bromo-4-[(pyridin-2-ylmethyl)-amino]-pyrimidin-2-ylamino}-3H-isobenzofuran-1-one;
N2-Benzothiazol-6-yl-5-bromo-N4-pyridin-2-ylmethyl-pyrimidine-2,4-diamine;
5-{5-Bromo-4-[(pyridin-2-ylmethyl)-amino]-pyrimidin-2-ylamino}-2-methyl-1H-indole-3-carbonitrile
5-Bromo-N4-pyridin-2-ylmethyl-N2-(1-pyridin-2-ylmethyl-1H-indazol-5-yl)-pyrimidine-2,4-diamine;
N2-(1-Benzyl-1H-indol-5-yl)-5-bromo-N4-pyridin-2-ylmethyl-pyrimidine-2,4-diamine;
5-Bromo-N4-pyridin-2-ylmethyl-N2-(1-pyridin-2-ylmethyl-1H-indol-5-yl)-pyrimidine-2,4-diamine;
N2-(1-Benzyl-1H-indazol-5-yl)-5-bromo-N4-pyridin-2-ylmethyl-pyrimidine-2,4-diamine;
5-Bromo-N2-(1-methyl-1H-indazol-5-yl)-N4-pyridin-2-ylmethyl-pyrimidine-2,4-diamine;
5-Bromo-N4-(4-methyl-cyclohexyl)-N2-[3-(1,2,3,6-tetrahydro-pyridin-4-yl)-1H-indol-5-yl]-pyrimidine-2,4-diamine;
5-Bromo-N4-(4-methyl-cyclohexyl)-N2-[3-(1,2,3,6-tetrahydro-pyridin-4-yl)-1H-indol-5-yl]-pyrimidine-2,4-diamine;
5-Bromo-N4-cyclohexylmethyl-N2-[3-(1,2,3,6-tetrahydro-pyridin-4-yl)-1H-indol-5-yl]-pyrimidine-2,4-diamine;
1-{5-Fluoro-4-[(pyridin-2-ylmethyl)-amino]-pyrimidin-2-yl}-3-(1,2,3,6-tetrahydro-pyridin-4-yl)-1H-indol-5-ylamine
1-{5-Chloro-4-[(pyridin-2-ylmethyl)-amino]-pyrimidin-2-yl}-3-(1,2,3,6-tetrahydro-pyridin-4-yl)-1H-indol-5-ylamine
5-Fluoro-N2-(1H-indazol-5-yl)-N4-pyridin-2-ylmethyl-pyrimidine-2,4-diamine;
5-{5-Fluoro-4-[(pyridin-2-ylmethyl)-amino]-pyrimidin-2-ylamino}-1,3-dihydro-indol-2-one;
5-Chloro-N2-(1H-indazol-5-yl)-N4-pyridin-2-ylmethyl-pyrimidine-2,4-diamine;
5-{5-Chloro-4-[(pyridin-2-ylmethyl)-amino]-pyrimidin-2-ylamino}-1,3-dihydro-indol-2-one;
5-Fluoro-N4-(2-pyridin-2-yl-ethyl)-N2-[3-(1,2,3,6-tetrahydro-pyridin-4-yl)-1H-indol-5-yl]-pyrimidine-2,4-diamine;
5-Chloro-N4-(2-pyridin-2-yl-ethyl)-N2-[3-(1,2,3,6-tetrahydro-pyridin-4-yl)-1H-indol-5-yl]-pyrimidine-2,4-diamine;
5-Fluoro-N2-(1H-indazol-5-yl)-N4-(2-pyridin-2-yl-ethyl)-pyrimidine-2,4-diamine;
5-[5-Fluoro-4-(2-pyridin-2-yl-ethylamino)-pyrimidin-2-ylamino]-1,3-dihydro-indol-2-one;
5-Chloro-N2-(1H-indazol-5-yl)-N4-(2-pyridin-2-yl-ethyl)-pyrimidine-2,4-diamine;
5-[5-Chloro-4-(2-pyridin-2-yl-ethylamino)-pyrimidin-2-ylamino]-1,3-dihydro-indol-2-one;
5-{4-[(Pyridin-2-ylmethyl)-amino]-5-trifluoromethyl-pyrimidin-2-ylamino}-1,3-dihydro-indol-2-one;
5-{5-Methoxy-4-[(pyridin-2-ylmethyl)-amino]-pyrimidin-2-ylamino}-1,3-dihydro-indol-2-one;
5-[5-Methoxy-4-(2-pyridin-2-yl-ethylamino)-pyrimidin-2-ylamino]-1,3-dihydro-indol-2-one;
5-[5-Methoxy-4-(2-trifluoromethyl-benzylamino)-pyrimidin-2-ylamino]-1,3-dihydro-indol-2-one;
5-{5-Bromo-4-[(cyclohex-1-enylmethyl)-amino]-pyrimidin-2-ylamino}-1,3-dihydro-indol-2-one;
5-[5-Bromo-4-(methyl-pyridin-2-ylmethyl-amino)-pyrimidin-2-ylamino]-1,3-dihydro-indol-2-one;
5-[5-Bromo-4-(4-methyl-cyclohexylamino)-pyrimidin-2-ylamino]-1,3-dihydro-indol-2-one;
5-[5-Bromo-4-(4-methyl-cyclohexylamino)-pyrimidin-2-ylamino]-1,3-dihydro-indol-2-one;
5-[5-Bromo-4-(cyclohexylmethyl-amino)-pyrimidin-2-ylamino]-1,3-dihydro-indol-2-one;
5-[5-Chloro-4-(2-trifluoromethyl-benzylamino)-pyrimidin-2-ylamino]-1,3-dihydro-indol-2-one;
2-(2-Oxo-2,3-dihydro-1H-indol-5-ylamino)₄-[(pyridin-2-ylmethyl)-amino]-pyrimidine-5-carbonitrile
5-{5-Methyl-4-[(pyridin-2-ylmethyl)-amino]-pyrimidin-2-ylamino}-1,3-dihydro-indol-2-one;
N2-(1H-Indazol-5-yl)-5-methyl-N4-pyridin-2-ylmethyl-pyrimidine-2,4-diamine;
5-Fluoro-N4-pyridin-2-ylmethyl-N2-[3-(1,2,3,6-tetrahydro-pyridin-4-yl)-1H-indol-5-yl]-pyrimidine-2,4-diamine;
5-Chloro-N4-pyridin-2-ylmethyl-N2-[3-(1,2,3,6-tetrahydro-pyridin-4-yl)-1H-indol-5-yl]-pyrimidine-2,4-diamine;
2-(2-Oxo-2,3-dihydro-1H-indol-5-ylamino)₄-(2-trifluoromethyl-benzylamino)-pyrimidine-5-carbonitrile
5-{4-[Methyl-(2-pyridin-2-yl-ethyl)-amino]-pyrimidin-2-ylamino}-1,3-dihydro-indol-2-one;
5-Bromo-N4-cyclohex-1-enylmethyl-N2-[3-(1,2,3,6-tetrahydro-pyridin-4-yl)-1H-indol-5-yl]-pyrimidine-2,4-diamine;
N2-(1H-Indazol-5-yl)-N4-pyridin-2-ylmethyl-5-trifluoromethyl-pyrimidine-2,4-diamine;
5-[5-Trifluoromethyl-4-(2-trifluoromethyl-benzylamino)-pyrimidin-2-ylamino]-1,3-dihydro-indol-2-one;
6-{2-[(Pyridin-2-ylmethyl)-amino]-5-trifluoromethyl-pyrimidin-4-ylamino}-1,3-dihydro-indol-2-one;
5-[5-Bromo-4-(piperidin-4-ylamino)-pyrimidin-2-ylamino]-1,3-dihydro-indol-2-one;
5-[4-(1-Acetyl-piperidin-4-ylamino)-5-bromo-pyrimidin-2-ylamino]-1,3-dihydro-indol-2-one;
2-(2-Oxo-2,3-dihydro-1H-indol-6-ylamino)₄-[(pyridin-2-ylmethyl)-amino]-pyrimidine-5-carbonitrile
5-{4-[(3-Methyl-pyridin-2-ylmethyl)-amino]-5-trifluoromethyl-pyrimidin-2-ylamino}-1,3-dihydro-indol-2-one;
6-{4-[(3-Methyl-pyridin-2-ylmethyl)-amino]-5-trifluoromethyl-pyrimidin-2-ylamino}-1,3-dihydro-indol-2-one;
4-[5-Bromo-2-(2-oxo-2,3-dihydro-1H-indol-5-ylamino)-pyrimidin-4-ylamino]-piperidine-1-carboxylic acid; tert-butyl ester;
5-[5-Bromo-4-(1-methanesulfonyl-piperidin-4-ylamino)-pyrimidin-2-ylamino]-1,3-dihydro-indol-2-one;
5-[5-Bromo-4-(piperidin-3-ylamino)-pyrimidin-2-ylamino]-1,3-dihydro-indol-2-one;
4-[5-Bromo-2-(2-oxo-2,3-dihydro-1H-indol-5-ylamino)-pyrimidin-4-ylamino]-piperidine-1-carboxylic acid; ethylamide
3-[5-Bromo-2-(2-oxo-2,3-dihydro-1H-indol-5-ylamino)-pyrimidin-4-ylamino]-piperidine-1-carboxylic acid; ethylamide
5-[4-(1-Benzoyl-piperidin-4-ylamino)-5-bromo-pyrimidin-2-ylamino]-1,3-dihydro-indol-2-one;
6-[4-(3-Methanesulfonyl-benzylamino)-5-methoxy-pyrimidin-2-ylamino]-1,3-dihydro-indol-2-one;
6-[4-(3-Methanesulfonyl-benzylamino)-5-trifluoromethyl-pyrimidin-2-ylamino]-1,3-dihydro-indol-2-one;
6-[4-(3-Methanesulfonyl-benzylamino)-pyrimidin-2-ylamino]-1,3-dihydro-indol-2-one;
5-[4-(1-Benzenesulfonyl-piperidin-4-ylamino)-5-bromo-pyrimidin-2-ylamino]-1,3-dihydro-indol-2-one;
5-[4-(3-Methanesulfonyl-benzylamino)-5-trifluoromethyl-3-pyrimidin-2-yiamino]-1,3-dihydro-indol-2-one;
6-{5-Chloro-4-[(piperidin-3-ylmethyl)-amino]-pyrimidin-2-ylamino}-1,3-dihydro-indol-2-one;
6-{5-Chloro-4-[(1-methanesulfonyl-piperidin-3-ylmethyl)-amino]-pyrimidin-2-ylamino}-1,3-dihydro-indol-2-one;
6-{5-Bromo-4-[(piperidin-3-ylmethyl)-amino]-pyrimidin-2-ylamino}-1,3-dihydro-indol-2-one;
6-{5-Bromo-4-[(1-methanesulfonyl-piperidin-3-ylmethyl)-amino]-pyrimidin-2-ylamino}-1,3-dihydro-indol-2-one;
5-[5-Fluoro-4-(3-methanesulfonyl-benzylamino)-pyrimidin-2-ylamino]-1,3-dihydro-indol-2-one;
5-{5-Bromo-4-[(1-hydroxy-cyclohexylmethyl)-amino]-pyrimidin-2-ylamino}-1,3-dihydro-indol-2-one; and pharmaceutically acceptable salt, prodrug, hydrate or solvate of the aforementioned compounds

25. A method for the treatment of abnormal cell growth in a mammal comprising administering to said mammal an amount of a compound of claim 1. that is effective in treating abnormal cell growth.

26. A method according to claim 25 wherein said abnormal cell growth is cancer.

27. A method according to claim 26 wherein said cancer is selected from lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers.

27. A method for the treatment of cancer solid tumor in a mammal comprising administering to said mammal an amount of a compound of claim 1 that is effective in treating said cancer solid tumor.

28. The method according to claim 27, wherein said cancer solid tumor is breast, lung, colon, brain, prostate, stomach, pancreatic, ovarian, skin (melanoma), endocrine, uterine, testicular, and bladder.

29. A method for the treatment of abnormal cell growth in a mammal which comprises administering to said mammal an amount of a compound of claim 1 that is effective in treating abnormal cell growth in combination with an anti-tumor agent selected from the group consisting of mitotic inhibitors, alkylating agents, anti-metabolites, intercalating antibiotics, growth factor inhibitors, radiation, cell cycle inhibitors, enzymes, topoisomerase inhibitors, biological response modifiers, antibodies, cytotoxics, anti-hormones, and anti-androgens.

30. A pharmaceutical composition for the treatment of abnormal cell growth in a mammal comprising an amount of a compound of claim 1 that is effective in treating abnormal cell growth, and a pharmaceutically acceptable carrier.
